(19) United States
(12) Patent Application Publication
(10) Pub. No.: US 2025/0041447 A1
(43) Pub. Date: Feb. 6, 2025

(54) CANCER INTERVENTION BY TARGETING GENOTYPIC DIFFERENCES USING CRISPR-CAS3 MEDIATED DELETION-EDITING

(71) Applicants: Cornell University, Ithaca, NY (US); Health Research, Inc., Buffalo, NY (US)

(72) Inventors: Ailong KE, Ithaca, NY (US); Lei WEI, Williamsville, NY (US)

(21) Appl. No.: 18/719,508

(22) PCT Filed: Dec. 16, 2022
(86) PCT No.: PCT/US2022/081852

§ 371 Date: Jun. 13, 2024

Related U.S. Application Data
(60) Provisional application No. 63/290,489, filed on Dec. 16, 2021.

(51) Int. Cl. A61K 48/00; A61P 35/00; C12N 9/22; C12N 15/11; C12Q 1/6809; C12Q 1/6886; G16B 30/10

(52) U.S. Cl. CPC A61K 48/005; A61P 35/00; C12N 9/22; C12N 15/111; C12Q 1/6809; C12Q 1/6886; G16B 30/10; C12N 2310/20; C12Q 2600/156

(57) ABSTRACT

Provided are compositions and methods for selectively killing cancer cells. The method includes obtaining one or more biological samples from an individual, determining different nucleotide sequences in cancer and non-cancer cells from the biological sample using an algorithm to identify a candidate target sequence that is present in the cancer cells and not present in the non-cancer cells. Based on the different nucleotide sequences in the cancer cells relative to the non-cancer cells a CRISPR Cas3 system that includes a guide RNA targeted to an identified segment of the chromosome that is linked to the target sequence is degraded.

### CROSS REFERENCE TO RELATED APPLICATION

This application claim priority to U.S. provisional application No. 63/290,489, filed Dec. 16, 2021, the entire disclosure of which is incorporated herein by reference.

### SEQUENCE LISTING

The instant application contains a Sequence Listing which has been submitted in.xml format and is hereby incorporated by reference in its entirety. Said.xml file is named “018617_01390_ST26.xml”, was created on Dec. 12, 2022, and is 18,002 bytes in size.

### FIELD OF THE DISCLOSURE

The present disclosure is related to compositions and methods for use in selecting genetic targets for use in specific killing of cancer cells. The selection is based upon genotypic differences between cancer and non-cancer cells.

### BACKGROUND OF THE DISCLOSURE

Cancer remains a leading cause of death among population. Most cancers are difficult to treat because no two cancers are identical. Cancers typically result from the long-term exposure to mutagenic agents, such as tobacco smoking, UV/radiation exposure, repeated tissue damage, chronic viral infection, and the like. Existing cancer therapeutic approaches primarily target the protein product of the mutated genes that either harbor cancer-driver mutations or genes that render conditional lethality in cancer cells when inhibited. Because such protein targets are rare among patients, and because rapidly dividing cancer cells tend to acquire resistant mutations quickly, cancer patients typically succumb to the disease due to the exhaustion of all treatment options.

Cancer cells typically have accumulated thousands of passenger mutations in the non-coding regions of genes before acquiring key driver mutations and undergoing clonal expansion. These truncal mutations are not under selection during tumorigenesis, and they frequently become a bi-allelic from loss-of-heterozygosity (LOH) events. They therefore provide an abundant supply of anti-cancer targets for use with personalized medicine for cancer patients. With the emergence of powerful genome editing tools, targeting genotypic biomarkers in cancer becomes conceptionally feasible. However, the available CRISPR tools such as Cas9 and Cas12 only introduce localized genome damage. The resulting indel is only impactful when the coding sequence or splicing pattern is altered. But a majority of the cancer-specific mutations are noncoding passenger mutations, hence small indels have not been impactful enough to kill a cell. Thus, there is an ongoing and unmet need for improved compositions and methods for targeting cancer cells. The disclosure is related to this need.

### BRIEF SUMMARY

The present disclosure provides methods for treating cancer. In embodiments, a provide method comprises i) obtaining one or more biological samples from an individual; ii) determining different nucleotide sequences in cancer and non-cancer cells from the biological sample to identify a candidate target sequence that is present in the cancer cells and not present in the non-cancer cells, and further characterizing the candidate target sequence to determine it is suitable for use as a target sequence. Characterizing the candidate target sequence comprises determining in the cancer cells but not the non-cancer cells that the candidate target sequence comprises: a) a homozygous mutation in the cancer cells that is a segment of a chromosome that is within 3 Kb-10 kb of an exon of an essential gene, wherein optionally the exon is not an alternatively spliced exon; b) identifying a protospacer adjacent motif (PAM) in the segment of a); and c) identifying a sequence in the chromosome that is preferably separated from the PAM by 13 to 17 nucleotides, and more preferably separated from the PAM by 1 to 11 nucleotides, wherein the identified sequence is approximately 32 nucleotides in length, to thereby identify a suitable target sequence. Based on the different nucleotide sequences in the cancer cells relative to the non-cancer cells, the method includes introducing into cells of the individual a CRISPR Cas3 system comprising a guide RNA targeted to a segment of the chromosome that is linked to the target sequence such that the chromosome comprising the target sequence is degraded, thereby treating the cancer. In embodiments, the characterization of the candidate target sequence also comprises determining that the different nucleotide sequences in the cancer cells comprises a mutation that is at least one of a breakpoint sequence in a chromosomal translocation sequence, a mutation that comprises an insertion or a deletion, a di-nucleotide mutation, or a single nucleotide mutation. The target characterization may include steps carried out by a computer implemented process. The method can include repeated administrations of the CRISPR Cas3 system and the guide RNA to cancer cells to facilitate cancer cell killing.

### BRIEF DESCRIPTION OF THE FIGURES

FIG. 1 provides a representative algorithm for cancer specific target site selection.

FIG. 2 provides additional details of the algorithm and its use to define a CRISPR-Cas3 target.

FIG. 3 depicts organization of the POLR2A gene and locations of guide RNA binding sites.

FIG. 4 provides spacer sequences and guide RNA sequences in DNA form that target the POLR2A. Target selection is as described in FIG. 1 and FIG. 2.

FIG. 5 provides results obtained using guide RNAs and a Cas3 to target the POLR2A gene in haploid eHAP1 cells.

FIG. 6 provides results demonstrating specificity of the Cas3 system such that it can distinguish between a single nucleotide difference between the targeted sequence and the guide RNA.

FIG. 7 provides representative examples of guide RNA and annotations related to targeting risk determined according to an algorithm.

FIG. 8 provides a representation of results obtained from targeting selected sites with selected guide RNAs in haploid eHAP1 and HEK293 cells.

FIG. 9 demonstrates use of repeated targeting of the same site enhances cell death.

FIG. 10 demonstrates analysis of antic-cancer cells in HEK293 cells.

### DETAILED DESCRIPTION OF THE DISCLOSURE

Unless defined otherwise herein, all technical and scientific terms used herein have the same meaning as commonly understood by one of ordinary skill in the art to which this disclosure pertains.

Unless specified to the contrary, it is intended that every maximum numerical limitation given throughout this description includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

All nucleotide sequences described herein include the RNA and DNA equivalents of such sequences, i.e., an RNA sequence includes its cDNA. All nucleotide sequences include their complementary sequences.

All temperatures and ranges of temperatures, all buffers, and other reagents, and all combinations thereof, are included in this disclosure.

All nucleotide and amino acid sequences identified by reference to a database, such as a GenBank database reference number, are incorporated herein by reference as the sequence exists on the filing date of this application or patent.

The disclosure includes all embodiments illustrated in the Figures provided with this

### DISCLOSURE

The disclosure of each cited reference is incorporated herein by reference.

Any component of the editing systems described herein can be provided on the same or different polynucleotides, such as plasmids, or a polynucleotide integrated into a chromosome. In embodiments, at least one component of the system is heterologous to the cells. In eukaryotic cells, all components of the system can be heterologous.

In embodiments, any enzyme or other protein as described herein is introduced into the cell as a recombinant or purified protein, or as an RNA encoding the protein that is expressed once introduced into the cell, or as an expression vector, which is expressed once in the cell. Any suitable expression system can be used and many are commercially available for use with the instant invention, given the benefit of the present description. The disclosure also includes use of ribonucleoproteins (RNPs) to introduce the described systems into cells. Any protein described herein can be provided with a nuclear localization signal.

This disclosure relates to use of CRISPR-Cas3 systems to target 1) cancer-specific passenger mutations near oncogenes and 2) homozygous cancer-specific passenger mutations near essential human genes. Because CRISPR-Cas3 allows repeated targeting and causes kilobases of DNA deletion near the target site, when programmed to target a cancer-specific passenger mutation near an oncogene or essential human gene, long-range damage to the genome is caused, which inactivates the nearby gene and kills the cell. The distinct deletion-editing profile of CRISPR-Cas3 is one aspect of the disclosure that, with the described methods of identifying targets, is believed to render the described anti-cancer method distinct from other genome editing based anti-cancer inventions.

In general, the present disclosure provides compositions, methods, and systems that relate to a process for selecting targets in cancer cells, and killing the cancer cells. The disclosure thus independently and collectively includes methods of selecting the cancer cells, and killing selected cancer cells using selected target sites. The combined process of selecting a target site and killing cancer cells is also encompassed by this disclosure. The selected targets are unique to cancer cells within a particular individual. As such, non-cancer cells are not killed using the described methods. In certain aspects the method is accordingly suitable for personalized medicine approaches.

The method can comprise identifying candidate target sites, and further characterizing target sites to establish they are likely to be sufficient to be used in the described methods of cancer cells. Once a target site is selected as described further herein, a Cas3 and a suitable guide RNA that is designed for specificity for the selected target is introduced into the cancer cells (and may be introduced into non-cancer cells). The operation of at least in part the Cas3 and the guide RNA degrades a segment of a chromosome only in the cancer cells that comprise the selected target site. The process may be used for a single or multiple different selected targets sites in cancer cells. Multiple targets may be targeted concurrently or sequentially. The process may be used a single time to kill cancer cells, or multiple times to increase the efficiency and/or number of targets and thereby increase the efficiency and/or the number of cancer cells that are killed.

In more detail, the described process of selecting a suitable targeting site reveals genetic differences that may be homozygous (such as being due to loss-of-heterozygosity) and near (i.e. <3 kb) essential genetic sequences. This targeting requirement greatly increases targetable sites from possibly none, to hundreds and potentially thousands of sites within a particular type of cancer cells. Further, in an additional embodiments, the selected target can be heterozygous if the product of the targeted genetic sequence is at least in part driving cancer formation, one representative example of which comprises a gene encoding a KRAS mutation which results in a fusion oncogene from chromosomal translocation. In a non-limiting embodiment, the KRAS mutation is any of KRAS G12C, G12D or G12R.

In one embodiment, the disclosure provides a method for killing cancer cells based on genetic information of cancer cells obtained from a biological sample of an individual. Either the whole genome of the cancer cells or genomic regions of the cancer cell genomes are sequenced. From the sequencing results, one or a plurality of targets is determined based on criteria further described herein. In embodiments, a target that is operably linked to an essential gene on the same chromosome is selected. Based on the determination of each target site, a guide RNA is prepared such that it targets a location on the same chromosome that is suitable for Cas3-mediated degradation of the chromosome that comprises the essential gene. Thus, the location of, for example, a mutation unique to the cancer cell that is in complete linkage disequilibrium with an essential gene can be targeted. As such, the disclosure does not require and can exclude directly targeting a site within the essential gene. For example, the target may be located in a non-coding segment of a chromosome, including but not necessarily limited to an intron that is flanked by one or two exons. However, the disclosure does not exclude targeting a coding sequence that is linked to the mutation. In certain embodiments, the identified target site may be considered a “therapeutic sequence.”

Essential human genes are defined as genes indispensable for the survival of any human cells. Inactivation of all alleles of such a gene, whether in wild type cell or cancer cell, is expected to kill the cell. Such genes are typically defined experimentally. A representative and non-limiting list of essential genes that are suitable for use with the described methods identified from HAP1 and KBM7 cells is provided in Table A. In embodiments, certain human genes may be Haplo-insufficient. In such cases, inactivation of only one allele of such genes may cause cell death.

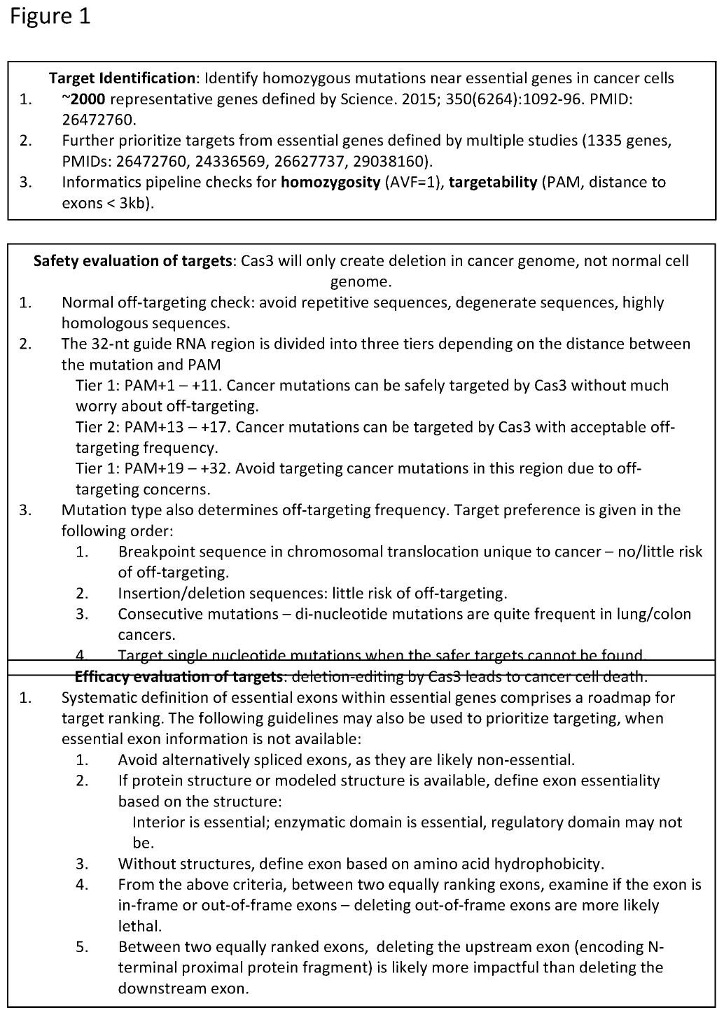

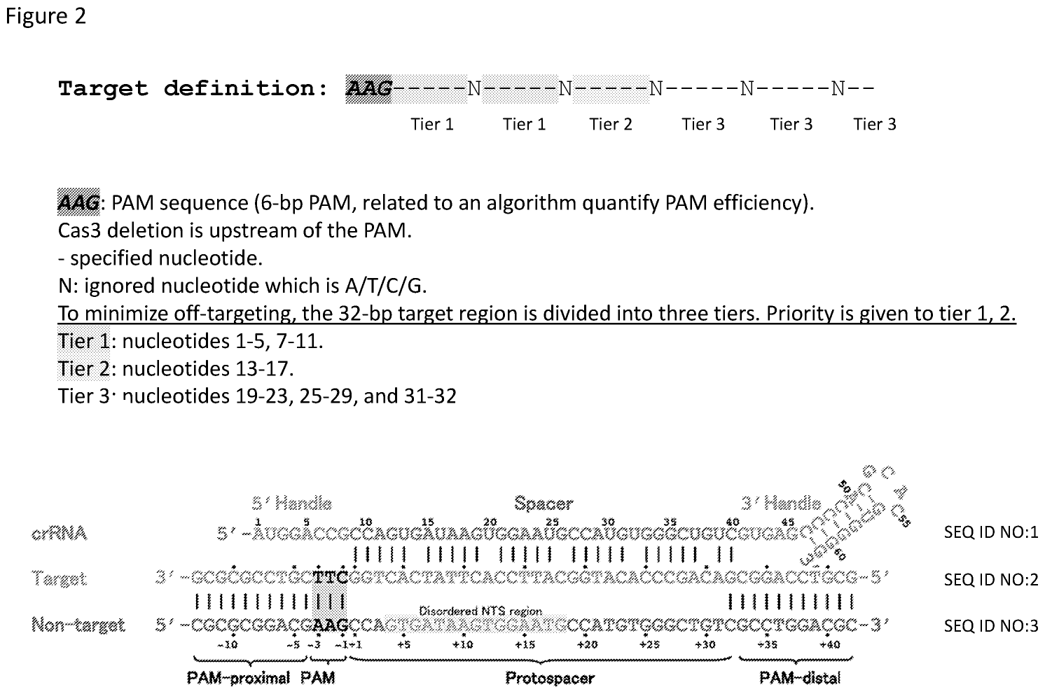

In general, a selected a target sequence may comprise a homozygous cancer-specific mutation. In normal diploid human cells, a spontaneous mutation is typically only present in one allele of the chromosome, the other allele in the homologous chromosome remains wild type. Due to the increased genome instability, cancer cells have high incidence of somatic structural variation (SV) in their genome. Such SV typically render cancer-specific mutations homozygous (loss-of-heterozygosity) through one of the following routes: 1) a gene conversion event that render all alleles of the gene in the cancer genome harboring the same mutation; 2) a deletion event that reduces the copy number of a particular gene to one, which happens to harbor the cancer-specific mutation; and 3) a reciprocal or non-reciprocal chromosomal translocation event that renders the cancer cell to contain only one copy of the functional gene, harboring a hallmark breakpoint sequence unique to cancer cells. In embodiments, the described bioinformatics pipeline identifies such mutations within 3 Kb-10 kb distance to an exon of an essential gene through the comparative analysis of the whole genome sequences of the cancer and normal cells from the same patient. The protospacer adjacent motif (PAM) sequence (an example of which is 5′-AAG) of the target site is oriented towards to the to-be-deleted exon, such that the programmed CRISPR-Cas3 tool will delete towards and into this exon. FIG. 1 provides a description of an algorithm that can be used to select candidate target sequences and target sequences for use with the described Type I CRISPR systems. FIG. 2 provides schematic and text summary of a bioinformatics approach (e.g., a process that can be described by an algorithm) to identify multiple target sequences, e.g., to define a suitable CRISR-Cas3 target site. The target site selected based in part on having a suitable PAM, which may be proximal to the mutation site, e.g., such as within approximately 18 nucleotides of the mutation. In certain embodiments, the target region comprises a 32 bp target region. To classify and minimize potential off-targeting effects, the 32 bp region is divided into three tiers of priority relative to the PAM.: Tier 1: nucleotides 1-5, 7-11 from the PAM Tier 2: nucleotides 13-17 from the PAM; Tier 3, nucleotides 19-23, 25-29, and 31-32 from the PAM. As depicted, the 3-bp PAM is numbered-3, -2 and -1, base pairs are numbered (+)1, 2, - - - 32. If the cancer genome differs from the normal cell genome with a Tier 1 mutation, RNA-guided selective targeting of the cancer genome is not expected to mistarget the normal genome. Targeting a tier 2 mutation may have a small risk of damaging the genome of the normal genome, which can be tested in non-cancer cell lines (i.e. HEK293 cells) by programming CRISPR-Cas3 with normal genome targeting guide and cancer genome targeting guide RNAs. Because CRISRP-based targeting performed using a perfect sequence match but disrupted by sequence mismatches, cell line target experiments can be used to provide an assessment of potential off-targeting risk. It will be recognized in certain embodiments the disclosure illustrates a representative approach by targeting HEK293 cells bearing the normal genome sequence as model of cancer to evaluate the targeting outcome. The selected target site may be characterized as not having one or more repetitive sequences, non-limiting example of which comprises tandem repeat sequences, microsatellite sequences, transposable elements, SINEs and LINEs, and pseudogenes. The selected target site may also be lacking highly homologous sequences elsewhere in the genome, to avoid the risk of off-targeting by CRISPR-Cas3. Highly homologous sequences are defined as a sequence that matches the PAM and Tier 1 and Tier 2 nucleotides combined (nt 1-5, 7-11, and 13-17). The selected target site includes but is not limited to the following, each of which is encompassed by the disclosure: preferably comprises a breakpoint sequence in a translocation unique to the cancer cells tested, less preferably comprising an insertion or a deletion, less preferably comprising a di-nucleotide (consecutive nucleotide) mutation, and less preferably comprising a single nucleotide mutation. In embodiments, a criterion for selecting the target site may exclude or deprioritize alternatively spliced exon sequences where the target site resides, because deletion of such sequences may not inactivate the essential gene. In embodiments, when multiple target sites are available to be selected for CRISPR-Cas3 targeting, the selection algorithm gives a higher ranking to out-of-frame exons (length: 3n+1, 3n+2) over in-frame exons (length: 3n) because deletion of out-of-frame exons has a higher probability to cause loss-of-function mutation in the targeted essential gene. In embodiments, when multiple target sites are available to be selected for CRISPR-Cas3 targeting, the selection algorithm gives a higher ranking to the promoter-proximal region of the essential gene (i.e. less than 1 kb upstream of the promoter, PAM-AAG in the direction of the gene). In embodiments, when multiple target sites are available to be selected for CRISPR-Cas3 targeting, the selection algorithm gives a higher ranking to mutations near the first few out-of-frame exons in the essential gene, the deletion of which will result in frame-shifting in the targeted gene, resulting in the premature termination of the targeted gene. In embodiments, when multiple target sites are available to be selected for CRISPR-Cas3 targeting, the selection algorithm gives a higher ranking to mutations near the exon that encodes indispensable polypeptides as judged by the predicted or experimentally determined structures, or its location in the hydrophobic core of an essential protein.

With respect to identification of target sites, the disclosure provides databases comprising indexed genes and/or mutations that may be found in human cancer cells and targetable using the described approaches. In embodiments, the disclosure provides a system, the system comprising: at least one computer hardware processor; one or more databases that store genomic information, which may include coding and non-coding sequences, entire gene sequences or segments of them, mutations, and may comprise information regarding a cancerous phenotype. A system in communication with the database may include at least one non-transitory computer-readable storage medium storing processor-executable instructions that, when executed by a computer hardware processor, cause the computer hardware processor to perform one or more steps and/or algorithms that are described herein, to generate results that include identification target sites. In embodiments, the disclosure provides a system that includes one or more devices, said devices comprising a DNA sequencing device and/or a computer. In embodiments, one or more components of the device can be connected to or in communication with a described digital processor and/or the computer running software to interpret a DNA sequencing signal. In embodiments, a system described herein may operate in a networked environment using logical connections to one or more remote computers. In embodiments, a result obtained using a device/system/method of this disclosure is fixed in a tangible medium of expression. The result may be communicated to, for example, a health care provider who will perform the chromosome modifying method, and/or an entity which may design, and optionally produce the described guide RNAs based on a determination of target sequences, or an expression vector encoding the guide RNAs for an end user.

In embodiments, the disclosure provides for selecting a target site as described above and in depicted FIGS. 1 and 2, and delivering to cells comprising the selected target site (which may include cells that do not contain the target site) a Cas3 system. The Cas3 system may comprise Cascade. As used herein, the term “Cascade” refers to an RNA-protein complex that is responsible for identifying a DNA target in crRNA-dependent fashion. In this regard, Cascade (CRISPR-Associated Complex for Anti-viral Defense) is a ribonucleoprotein complex comprised of multiple protein subunits and is used naturally in bacteria as a mechanism for nucleic acid-based immune defense. Cascade complexes are characteristic of the Type I CRISPR systems. The Cascade complex recognizes nucleic acid targets via direct base-pairing to an RNA guide contained in the complex. Cas3 may comprise a single protein unit which contains helicase and nuclease domains. After target validation by Cascade, Cas3 nicks the strand of DNA that is looped out by the R-loop formed by Cascade approximately 9-12 nucleotides inward from the PAM site. Cas3 then uses its helicase/nuclease activity to processively degrade substrate nucleic acids, moving in a 3′ to 5′ direction. In embodiments, once a suitable target is selected using the described process, the disclosure comprises selecting and using a crRNA as a guide RNA comprising constant regions at its 5′ and 3′-ends and a variable region in the middle, which comprises a spacer for DNA targeting of a selected position, and participates in R-loop formation. In embodiments, more than one Cascade/Cas3 is provided. In embodiments, more than one crRNA, or guide RNA is provided. In embodiments, 2, 3, 4, 5, or more crRNAs or guide RNAs are provided.

In embodiments, the disclosure provides for increased DNA deletion-editing in cancer cells, relative to a control value. In embodiments, the control value is obtained from using a CRISPR-Cas3 system programmed with the same RNA guide but harboring a sequence mismatch to the same cell line.

In embodiments, the disclosure utilizes a Type I systems protospacer adjacent motifs (PAM) that comprises di- or tri-nucleotide conserved motifs downstream of protospacers opposite of the crRNA 5′-handle. Those skilled in the art will understand that other PAM sequences may be recognized by Cas enzymes from different bacterial types.

In embodiments, Cascade and Cas3 used according to this disclosure generates one or more genome lesions, considered to be long-range deletions, wherein from the lesion(s) are initiated, or are located, from a few nucleotides from a suitable PAM sequence, and to up to 10 kb upstream of the PAM sequence.

In embodiments one or more proteins used in this disclosure has/have between 50-100% identity to a wild type amino acid sequence. In embodiments, any type I CRISPR system proteins are used. In embodiments, the protein comprises a truncation and/or deletion such that only a segment of the protein that is required to achieve a desired effect (i.e., an improvement in DNA editing/deletion relative to a reference) is achieved. In embodiments, a protein used herein comprises an amino acid sequence that includes additional amino acids at the N- or C-terminus, relative to a wild type sequence. Thus, in proteins used herein have an amino acid sequence described herein, and/or are encoded by any of the nucleotide sequences described herein, or any sequence having at least from 50%-100%, inclusive, and including all integers and ranges of integers there between, identity with the foregoing nucleotide and/or amino acid sequences. In embodiments, proteins have 90, 91, 92, 93, 94, 95, 96, 97, 98, or 99% identity across the entire length or a functional segment thereof of the sequences described herein. Thus, variants of the proteins and their nucleotide sequences are included. The term “variant” and its various grammatical forms as used herein refers to a nucleotide sequence or an amino acid sequence with substantial identity to a reference nucleotide sequence or reference amino acid sequence, respectively. The differences in the sequences may be the result of changes, either naturally or by design, in sequence or structure. Designed changes may be specifically designed and introduced into the sequence for specific purposes. Such specific changes may be made in vitro using a variety of mutagenesis techniques. Such sequence variants generated specifically may be referred to as “mutants” or “derivatives” of the original sequence.

In embodiments, the disclosure includes a crRNA, which may be considered a “targeting RNA” that is developed based upon selection of a target sequence as described above. A crRNA, when transcribed from the portion of the CRISPR system encoding it, comprises at least a segment of RNA sequence that is identical to (with the exception of replacing T for U in the case of RNA) or complementary to (and thus “targets”) the selected target sequence in a cell into which the system is introduced.

In embodiments, the disclosure comprises deleting a segment of a chromosome that is in complete linkage disequilibrium with, or contains an essential gene or a segment thereof. The deletion may be single or double stranded. In embodiments, the deletions comprise from 500 nucleotides, to 10K nucleotides, inclusive, and including all ranges of numbers there between, and including base pair deletions.

In embodiments, two or more effective guides can be combined to formulate a cocktail anti-cancer therapeutic. The advantage of this combinatorial strategy is the reduced likelihood of selecting drug-resistant cancer clones. In embodiments, repeated delivery of a described system is used.

In embodiments, kits for making genetic modifications as described herein are provided. A kit comprises one or more suitable vectors that encode Type I Cascade proteins. The kits can also include other components that are suitable for using the expression vectors to edit DNA in any cell type.

The guide RNA-containing Cascade Complexes can be either produced in a cell using DNA or RNA encoding for the protein and/or RNA components or delivered in the form of one or more vectors for expression or delivered in the form of RNA encoding for the proteins and/or RNA components or delivered in the form of fully-formed protein-RNA complexes through mechanisms including but not limited to electroporation, injection, or transfection. The guide RNA-containing Cascade complexes described herein, can be recombinantly expressed and purified through known purification technologies and methods either as whole Cascade complexes or as individual proteins. These proteins can be used in various delivery mechanisms including but not limited to electroporation, injection, or transfection for whole-protein delivery to eukaryotic organisms or can be used for in-vitro applications for sequence targeting of nucleic acid substrates or modification of substrates. Cascade complexes containing guides which target a selected DNA target will hybridize to the target sequence and will, if complementarity is sufficient, open a full R-loop along the length of the target site. This Cascade-marked R-loop region adopts a conformation which allows Cas3 to bind to a site which is PAM-proximal, orienting the nuclease domain to initially attack the non-targeted DNA strand approximately 9-12 nucleotides inside the R-looped region. The helicase domain is loaded with the non-target strand, and the Cas3 then processively unwinds the substrate DNA in an ATP-dependent fashion from 3′ to 5′. In conjunction with this helicase activity, nuclease activity cleaves the non-target strand in a processive fashion.

In embodiments, the disclosure uses wild-type Cas3 proteins, or modifications or derivatives thereof. For example, in a case where either wild-type Cas3 or an otherwise engineered Cas3 is capable of cleaving both strands of DNA during a processive mode, once recruited to a validated target sequence by Cascade, Cas3 inherently produces a 3′ overhang on the target strand. This is because Cascade is protecting the target strand from just after the PAM site to the end of the R-loop. Thus, once Cas3 is loaded on the non-target strand and begins its processive cleavage, the earliest nucleotide on the target strand that is available for cleavage is at the PAM site. In comparison, degradation of the non-target strand occurs 9-12 nucleotides inside the R-loop region.

In a case where either wild-type Cas3 or an otherwise engineered Cas3 is capable of confining its cleavage activity to one or the other strand of substrate DNA, two or more Cascade targeting complexes can be used, such that the PAM sites are facing towards one another, to recruit Cas3 to each target site and degrade the intervening section of DNA on both strands. This will produce 3′ overhangs on both strands of DNA and a degraded segment of DNA between.

As discussed above, variations on Cascade are encompassed in this disclosure. In embodiments, a Cas3/Cascade system is any Cas3 and Cascade described in PCT publication no. WO 2019/246555, from which the entire disclosure is incorporated herein by reference.

As discussed above, the disclosure also includes using Cas3 variants and derivatives. For example, mutations can be made that affect protein stability, R-loop recruitment efficiency, initial nicking efficiency, helicase activity, processive nuclease activity, expression or purification, off-target effects, or other protein functions or properties.

The following examples are presented to illustrate the present disclosure. They are not intended to be limiting in any manner.

### Example 1

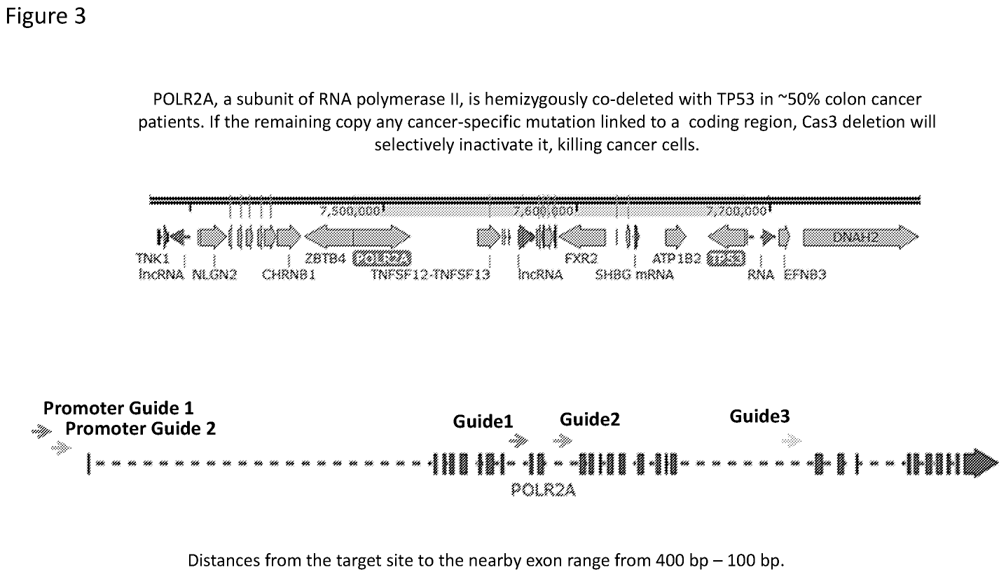

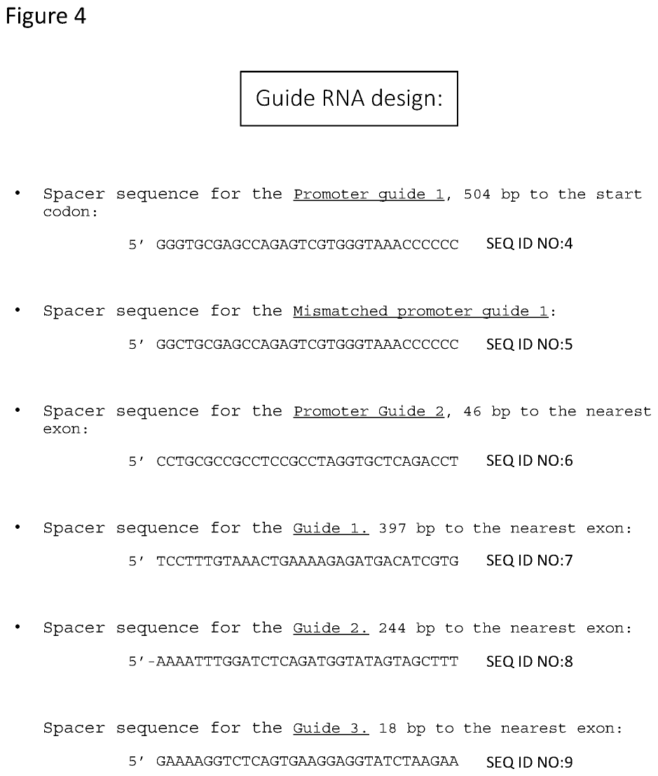

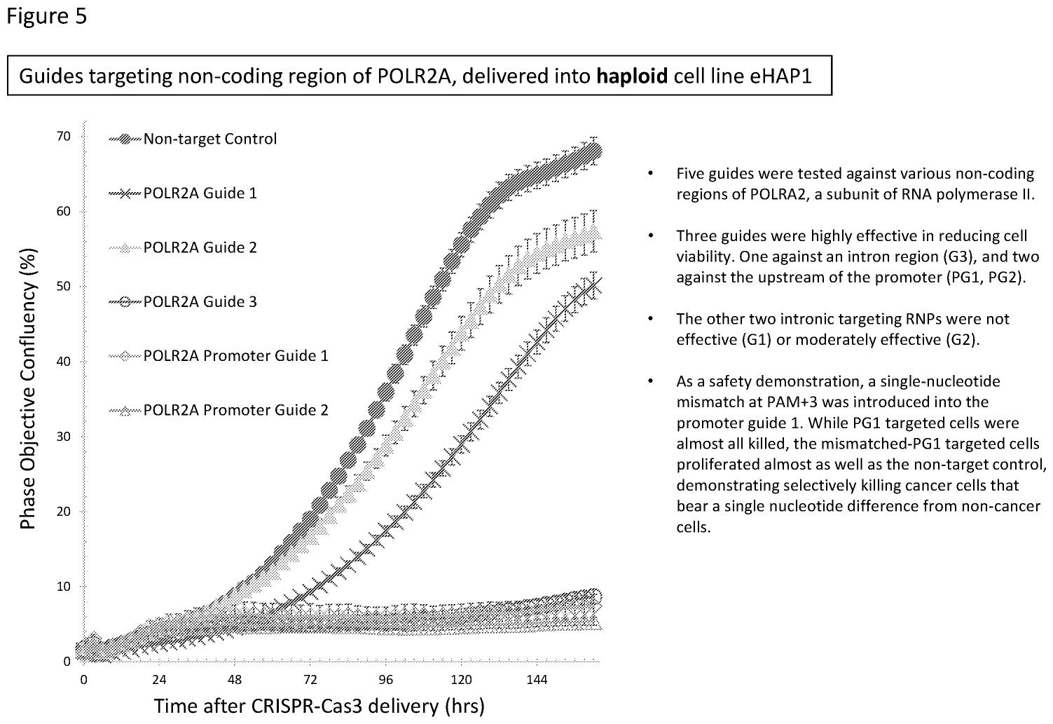

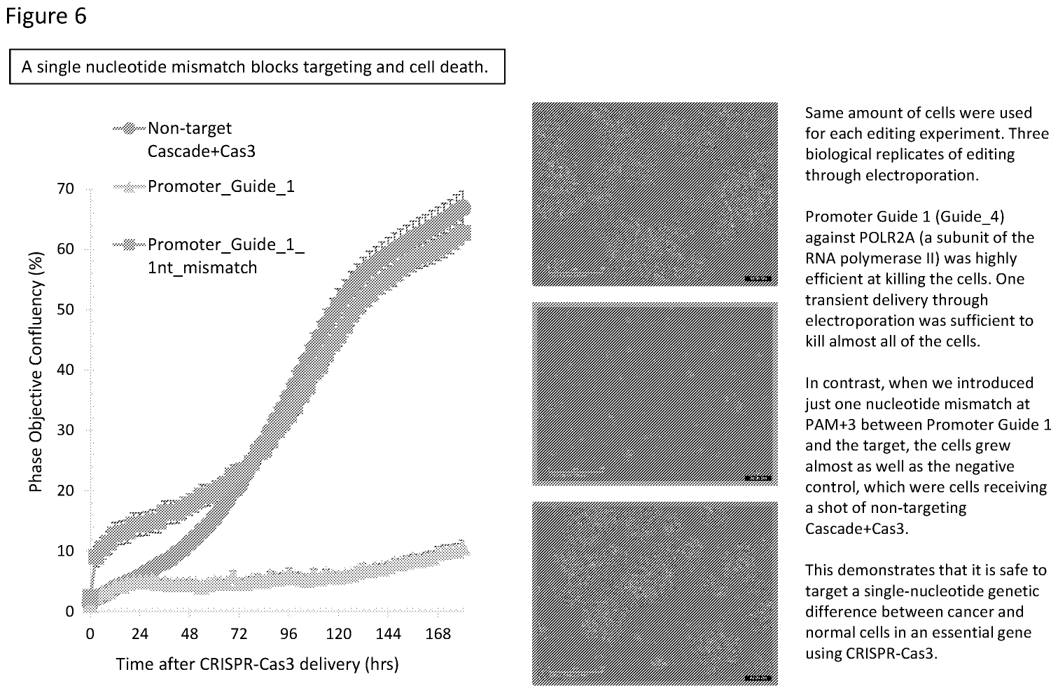

This Example provides a demonstration of using non-coding regions of POLR2A, an essential gene to evaluate 1) whether CRISPR-Cas3 targeting can efficiently affect cell viability, and 2) whether it is safe to rely on a single nucleotide difference to distinguish wild-type cells from diseased cells. The experiments were performed in the haploid cell line eHAP1. The results and related information are depicted in FIG. 3-6. FIG. 3 provides a schematic of the POLR2A gene and locations of promoters and guide RNAs with relative distances from the target site to the nearby exon. FIG. 4 is related to FIG. 3 and provides representative guide RNA sequences (given as DNA sequence) and annotates their relative locations to the nearest POLR2A exon. In embodiments, guide RNAs target the reverse complement strand of the coding sequence, hence the PAMs are found downstream of the target, which may be presented as CTT instead of AAG. The PAM sequence facilitates target-searching but may be excluded from the guide RNA. FIG. 5 provides a graphical summary of data demonstrating use of the guide RNAs from FIG. 4 as tested against non-coding regions of POLR2A. FIG. 6 provides graphical and photographic representations of selective killing and discrimination of target sates based on a single nucleotide mismatch between the selected target site and the guide RNA, demonstrating specificity and safety of the described approach.

### Example 2

This Example provides demonstrates selected target cancer-specific killing of homozygous mutations in noncoding regions of essential genes. The mutations were identified and ranked using the described methods of target selection by comparing the whole genome sequences of COLO829 (skin cancer) and COLO829-BL (normal cell from the same patient). A list of ˜200 targets were identified this way. Five mutations were then selected for experimental testing. Due to technical difficulties in electroporating COLO829 cells, we targeted the same sites (without the mutation) in two different cancer cell lines (haploid eHAP1 cell line described above, and diploid HEK293 cell line used in this Example). The described anti-cancer method is expected to be broadly applicable to all cancer types. The efficacy is expected to only correlate with the targeting efficiency and the functional consequence of the deletion-targeting event. Therefore, validating the targeting efficacy in different cell lines supports a broadly applicable approach. In this regard, the efficacy pattern is consistent in eHAP1 and HEK293 cells. For example, the guide RNA targeted to RPL39 was identified as the most effective guide against both cell lines.

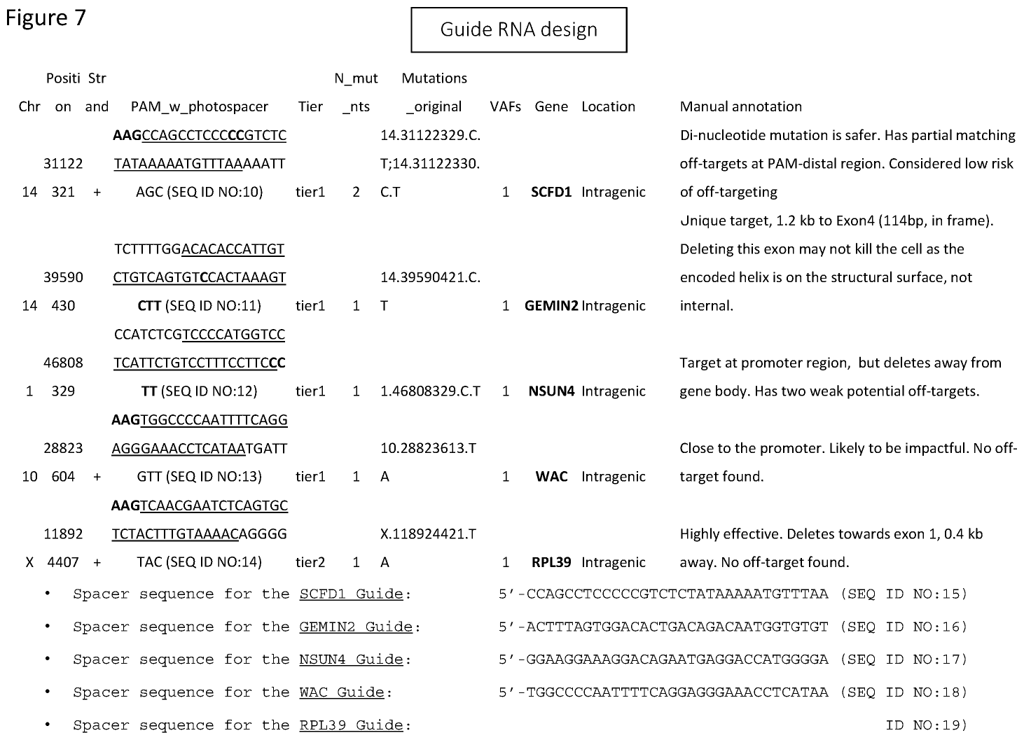

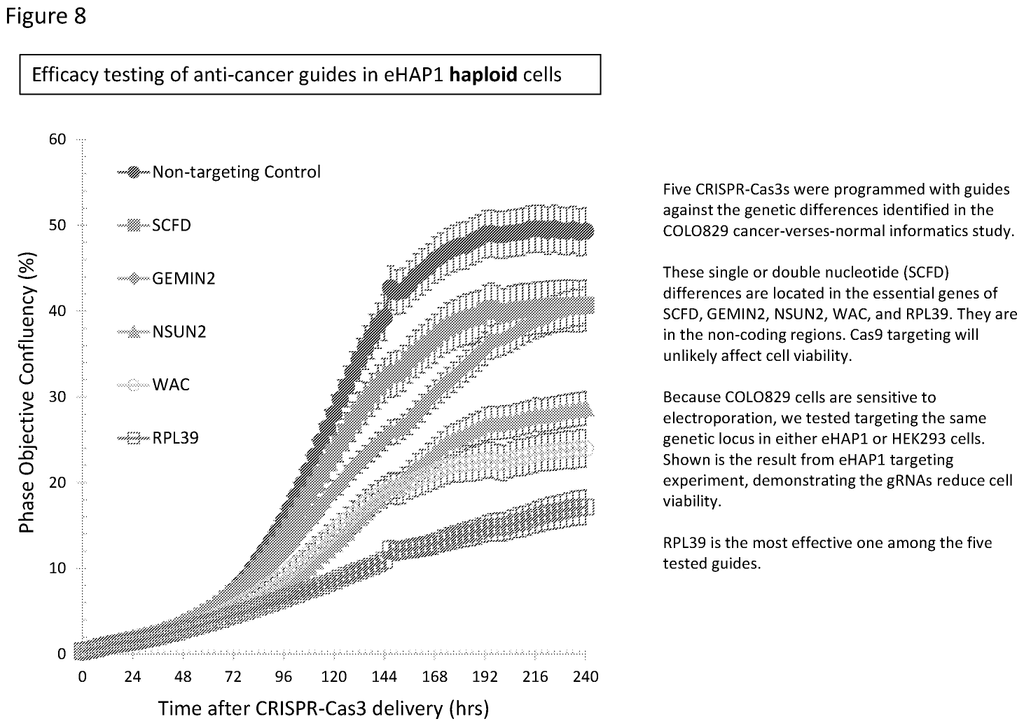

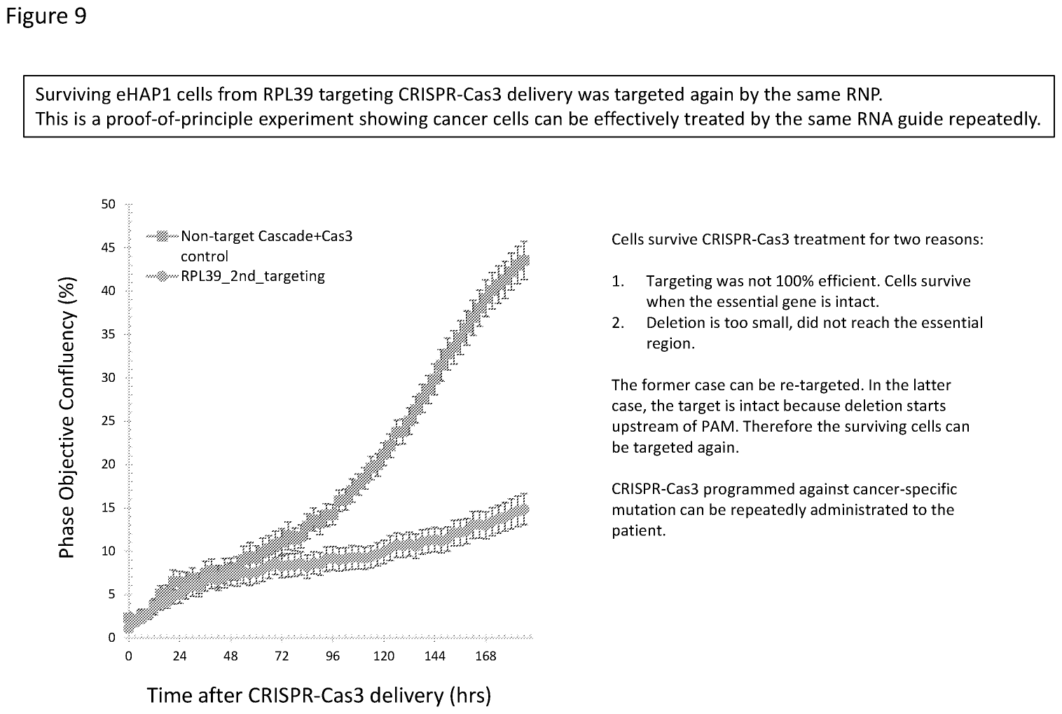

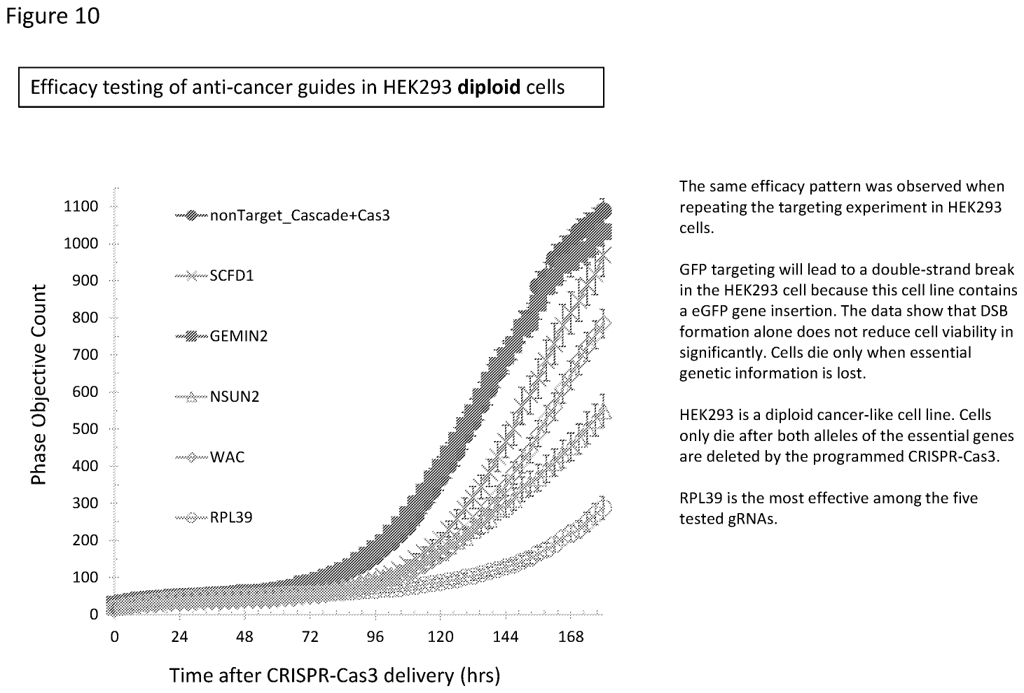

FIG. 7 depicts guide RNA design and different spacers, as indicated. The manual annotations provide information about the mutations. FIG. 8 provides data obtained from the design and administration of CRISPR-Cas3 targeting cancer-specific markers obtained from the well-annotated skin cancer cell line COLO829, namely, targeting cancer mutations with variant allele frequency (VAF) of 1.0-homozygous mutations, but in eHAP1 haploid cells. Of the 32-bp targetable region, targeting mutations between nt 1-18 below PAM avoids significant off-targeting effects, which may kill normal cells. Because COLO829 is sensitive to RNP delivery in general, we chose alternative cell lines (eHAP1 for haploid cells, and HEK293 for diploid cells). We targeted the same chromosomal location in these cell lines, but targeted the wild-type sequence to model the functional consequence of targeting the mutated sequence in COLO829. We observed a very similar surviving profile: guide RNAs that are effective at reducing cell proliferation potential in eHAP1 are similarly effective in HEK239 (compare the proliferation curves in FIGS. 8 and 10, respectively). The described anti-cancer strategy may thus be used in a cell-type independent, sequence-content independent manner and as such relies on proximity relationship to essential genetic regions. FIG. 9 shows Surviving eHAP1 cells from RPL39 targeting CRISPR-Cas3 delivery that were targeted again by the same RNP. This demonstrates cancer cells can be effectively treated by the same guide RNA repeatedly. The T. fusca CRISPR-Cas3 tool has been shown to delete upstream of PAM sequence in the majority of the genome editing events (Dolan et. al, Molecular Cell, 2019). Accordingly, deletion by T. fusca CRISPR-Cas3 does not disrupt the target site in the majority of the cases. Such cells can be targeted again by the same guide RNA. The experimental data in FIG. 9 indicates that the cells survived from the first targeting experiments due to inefficient targeting, e.g., their genomes were either not targeted, or the editing did not delete essential genetic information to cause cell death. Such cells remain sensitive to the CRISPR-Cas3 programmed by the same guide RNA, therefore repeated targeting continues to reduce viability of the cells. FIG. 10 shows efficacy testing of anti-cancer guides in HEK293 diploid cells, showing that targeting of both alleles to achieve killing. Five guides showed similar efficacy in HEK293 cells (FIG. 10) as in the HAP1 cells (FIG. 8).

TABLE A
KBM7

| GENE_SYMBOL | ENSEMBL_ID | tot. sense | tot. anti | p.val | q.val | ratio | selected |
|---|---|---|---|---|---|---|---|
| AAMP | ENSG00000127837 | 2 | 22 | 3.13E−05 | 0.00029615 | 0.08333333 | YES |
| AARS | ENSG00000090861 | 63 | 805 | 1.41E−88 | 2.36E−86 | 0.07258065 | YES |
| AASDHPPT | ENSG00000149313 | 23 | 286 | 1.12E−40 | 3.90E−39 | 0.07443366 | YES |
| AATF | ENSG00000108270 | 31 | 300 | 2.52E−39 | 8.33E−38 | 0.09365559 | YES |
| ABCB7 | ENSG00000131269 | 34 | 545 | 1.72E−70 | 1.76E−68 | 0.05872193 | YES |
| ABCE1 | ENSG00000164163 | 21 | 723 | 2.43E−96 | 5.09E−94 | 0.02822581 | YES |
| ABCF1 | ENSG00000204574 | 3 | 87 | 1.23E−17 | 1.91E−16 | 0.03333333 | YES |
| ABT1 | ENSG00000146109 | 3 | 54 | 2.89E−11 | 3.53E−10 | 0.05263158 | YES |
| ACACA | ENSG00000132142 | 550 | 1399 | 2.05E−40 | 7.11E−39 | 0.282196 | YES |
| ACIN1 | ENSG00000100813 | 106 | 289 | 6.01E−16 | 8.86E−15 | 0.26835443 | YES |
| ACO2 | ENSG00000100412 | 55 | 195 | 5.81E−16 | 8.60E−15 | 0.22 | YES |
| ACTL6A | ENSG00000136518 | 11 | 211 | 8.79E−35 | 2.47E−33 | 0.04954955 | YES |
| ACTR10 | ENSG00000131966 | 9 | 117 | 5.71E−20 | 9.62E−19 | 0.07142857 | YES |
| ACTR2 | ENSG00000138071 | 95 | 304 | 5.11E−20 | 8.65E−19 | 0.23809524 | YES |
| ACTR3 | ENSG00000115091 | 349 | 894 | 3.13E−31 | 7.64E−30 | 0.28077233 | YES |
| ACTR6 | ENSG00000075089 | 49 | 160 | 1.00E−12 | 1.30E−11 | 0.23444976 | YES |
| ACTR8 | ENSG00000113812 | 22 | 192 | 1.09E−26 | 2.28E−25 | 0.10280374 | YES |
| ADAR | ENSG00000160710 | 29 | 212 | 5.61E−27 | 1.18E−25 | 0.12033195 | YES |
| ADAT2 | ENSG00000189007 | 71 | 350 | 3.72E−32 | 9.43E−31 | 0.16864608 | YES |
| ADSL | ENSG00000239900 | 17 | 426 | 6.80E−63 | 5.28E−61 | 0.03837472 | YES |
| ADSS | ENSG00000035687 | 12 | 284 | 1.69E−45 | 7.11E−44 | 0.04054054 | YES |
| AFG3L2 | ENSG00000141385 | 12 | 423 | 7.45E−65 | 6.29E−63 | 0.02758621 | YES |
| AHCTF1 | ENSG00000153207 | 78 | 196 | 7.68E−11 | 9.25E−10 | 0.28467153 | YES |
| AHCY | ENSG00000101444 | 47 | 637 | 3.80E−76 | 4.81E−74 | 0.06871345 | YES |
| AIFM1 | ENSG00000156709 | 231 | 641 | 2.56E−28 | 5.70E−27 | 0.26490826 | YES |
| AK2 | ENSG00000004455 | 14 | 572 | 1.97E−82 | 2.97E−80 | 0.02389078 | YES |
| AKIRIN2 | ENSG00000135334 | 58 | 376 | 9.16E−40 | 3.09E−38 | 0.13364055 | YES |
| ALDOA | ENSG00000149925 | 106 | 265 | 2.61E−13 | 3.46E−12 | 0.28571429 | YES |
| ALG1 | ENSG00000033011 | 23 | 361 | 2.15E−51 | 1.08E−49 | 0.05989583 | YES |
| ALG11 | ENSG00000253710 | 11 | 150 | 9.87E−25 | 1.94E−23 | 0.06832298 | YES |
| ALG13 | ENSG00000101901 | 15 | 205 | 6.83E−32 | 1.71E−30 | 0.06818182 | YES |
| ALG14 | ENSG00000172339 | 9 | 91 | 2.81E−15 | 4.05E−14 | 0.09 | YES |
| ALG2 | ENSG00000119523 | 2 | 35 | 5.77E−08 | 6.22E−07 | 0.05405405 | YES |
| ALG8 | ENSG00000159063 | 292 | 414 | 9.36E−05 | 0.00086009 | 0.41359773 | YES |
| ALYREF | ENSG00000183684 | 2 | 97 | 4.51E−20 | 7.66E−19 | 0.02020202 | YES |
| AMD1 | ENSG00000123505 | 191 | 289 | 6.92E−05 | 0.00064191 | 0.39791667 | YES |
| ANAPC1 | ENSG00000153107 | 191 | 634 | 5.37E−35 | 1.53E−33 | 0.23151515 | YES |
| ANAPC10 | ENSG00000164162 | 13 | 390 | 5.53E−60 | 3.88E−58 | 0.03225806 | YES |
| ANAPC11 | ENSG00000141552 | 8 | 103 | 5.96E−18 | 9.44E−17 | 0.07207207 | YES |
| ANAPC13 | ENSG00000129055 | 227 | 1007 | 2.08E−62 | 1.60E−60 | 0.18395462 | YES |
| ANAPC15 | ENSG00000110200 | 116 | 368 | 9.20E−23 | 1.72E−21 | 0.23966942 | YES |
| ANAPC4 | ENSG00000053900 | 15 | 252 | 3.10E−39 | 1.02E−37 | 0.05617978 | YES |
| ANAPC5 | ENSG00000089053 | 13 | 451 | 5.15E−68 | 4.95E−66 | 0.02801724 | YES |
| ANKLE2 | ENSG00000176915 | 18 | 134 | 7.53E−19 | 1.22E−17 | 0.11842105 | YES |
| ANKRD17 | ENSG00000132466 | 1625 | 2307 | 6.95E−11 | 8.40E−10 | 0.41327569 | YES |
| ANLN | ENSG00000011426 | 13 | 678 | 6.04E−95 | 1.23E−92 | 0.01881331 | YES |
| AP2S1 | ENSG00000042753 | 8 | 175 | 1.91E−30 | 4.56E−29 | 0.04371585 | YES |
| APC | ENSG00000134982 | 154 | 265 | 1.39E−06 | 1.41E−05 | 0.36754177 | YES |
| APEX1 | ENSG00000100823 | 324 | 445 | 0.00022424 | 0.0020125 | 0.4213264 | YES |
| API5 | ENSG00000166181 | 39 | 95 | 2.70E−06 | 2.70E−05 | 0.29104478 | YES |
| AQR | ENSG00000021776 | 22 | 285 | 5.66E−41 | 2.01E−39 | 0.07166124 | YES |
| ARCN1 | ENSG00000095139 | 7 | 242 | 1.12E−41 | 4.08E−40 | 0.02811245 | YES |
| ARGLU1 | ENSG00000134884 | 21 | 167 | 4.48E−23 | 8.43E−22 | 0.11170213 | YES |
| ARL2 | ENSG00000213465 | 74 | 200 | 4.08E−12 | 5.20E−11 | 0.27007299 | YES |
| ARMC7 | ENSG00000125449 | 83 | 465 | 5.24E−43 | 2.01E−41 | 0.15145985 | YES |
| ARMC8 | ENSG00000114098 | 675 | 944 | 9.00E−07 | 9.20E−06 | 0.41692403 | YES |
| ARPC2 | ENSG00000163466 | 541 | 1593 | 6.35E−54 | 3.45E−52 | 0.25351453 | YES |
| ARPC3 | ENSG00000111229 | 113 | 280 | 1.18E−13 | 1.59E−12 | 0.28753181 | YES |
| ARPC4 | ENSG00000241553 | 116 | 313 | 1.20E−16 | 1.82E−15 | 0.27039627 | YES |
| ASCC3 | ENSG00000112249 | 168 | 426 | 6.75E−19 | 1.10E−17 | 0.28282828 | YES |
| ASUN | ENSG00000064102 | 447 | 720 | 7.67E−10 | 8.88E−09 | 0.38303342 | YES |
| ATAD5 | ENSG00000176208 | 117 | 359 | 1.56E−21 | 2.80E−20 | 0.24579832 | YES |
| ATIC | ENSG00000138363 | 40 | 178 | 6.83E−18 | 1.08E−16 | 0.18348624 | YES |
| ATL2 | ENSG00000119787 | 99 | 211 | 7.82E−09 | 8.72E−08 | 0.31935484 | YES |
| ATP13A1 | ENSG00000105726 | 175 | 251 | 0.0007444 | 0.00643927 | 0.41079812 | YES |
| ATP1A1 | ENSG00000163399 | 25 | 170 | 6.08E−22 | 1.11E−20 | 0.12820513 | YES |
| ATP2A2 | ENSG00000174437 | 51 | 886 | 2.71E−101 | 6.97E−99 | 0.05442903 | YES |
| ATP5A1 | ENSG00000152234 | 76 | 423 | 5.84E−40 | 1.99E−38 | 0.15230461 | YES |
| ATP5B | ENSG00000110955 | 39 | 243 | 6.88E−28 | 1.51E−26 | 0.13829787 | YES |
| ATP5C1 | ENSG00000165629 | 77 | 228 | 7.01E−15 | 9.98E−14 | 0.25245902 | YES |
| ATP5E | ENSG00000124172 | 59 | 262 | 5.03E−24 | 9.72E−23 | 0.18380062 | YES |
| ATP5F1 | ENSG00000116459 | 107 | 375 | 1.30E−25 | 2.63E−24 | 0.2219917 | YES |
| ATP5H | ENSG00000167863 | 30 | 117 | 9.39E−12 | 1.17E−10 | 0.20408163 | YES |
| ATP5J2 | ENSG00000241468 | 86 | 393 | 3.11E−33 | 8.16E−32 | 0.17954071 | YES |
| ATP5L | ENSG00000167283 | 93 | 239 | 7.22E−13 | 9.43E−12 | 0.28012048 | YES |
| ATP5O | ENSG00000241837 | 63 | 257 | 3.51E−22 | 6.43E−21 | 0.196875 | YES |
| ATP6AP1 | ENSG00000071553 | 19 | 399 | 2.19E−58 | 1.46E−56 | 0.04545455 | YES |
| ATP6AP2 | ENSG00000182220 | 6 | 80 | 1.04E−14 | 1.48E−13 | 0.06976744 | YES |
| ATP6V0B | ENSG00000117410 | 19 | 147 | 1.30E−20 | 2.25E−19 | 0.11445783 | YES |
| ATP6V0C | ENSG00000185883 | 12 | 273 | 7.35E−44 | 2.95E−42 | 0.04210526 | YES |
| ATP6V0D1 | ENSG00000159720 | 15 | 388 | 9.20E−59 | 6.25E−57 | 0.03722084 | YES |
| ATP6V1A | ENSG00000114573 | 257 | 1412 | 5.72E−90 | 1.02E−87 | 0.15398442 | YES |
| ATP6V1D | ENSG00000100554 | 5 | 82 | 1.33E−15 | 1.94E−14 | 0.05747126 | YES |
| ATP6V1E1 | ENSG00000131100 | 22 | 199 | 8.38E−28 | 1.82E−26 | 0.09954751 | YES |
| ATP6V1F | ENSG00000128524 | 5 | 147 | 2.68E−27 | 5.73E−26 | 0.03289474 | YES |
| ATP6V1G1 | ENSG00000136888 | 4 | 239 | 8.34E−43 | 3.18E−41 | 0.01646091 | YES |
| ATR | ENSG00000175054 | 11 | 385 | 2.83E−60 | 2.00E−58 | 0.02777778 | YES |
| ATRIP | ENSG00000164053 | 6 | 76 | 5.93E−14 | 8.11E−13 | 0.07317073 | YES |
| ATRX | ENSG00000085224 | 319 | 1015 | 2.30E−45 | 9.56E−44 | 0.23913043 | YES |
| ATXN10 | ENSG00000130638 | 104 | 321 | 5.25E−20 | 8.88E−19 | 0.24470588 | YES |
| AURKA | ENSG00000087586 | 13 | 83 | 4.63E−12 | 5.88E−11 | 0.13541667 | YES |
| B4GALT5 | ENSG00000158470 | 694 | 1100 | 9.39E−12 | 1.17E−10 | 0.38684504 | YES |
| BANF1 | ENSG00000175334 | 41 | 139 | 7.05E−12 | 8.90E−11 | 0.22777778 | YES |
| BANP | ENSG00000172530 | 50 | 1267 | 1.50E−136 | 8.47E−134 | 0.03796507 | YES |
| BARD1 | ENSG00000138376 | 26 | 273 | 1.86E−37 | 5.81E−36 | 0.08695652 | YES |
| BCAS2 | ENSG00000116752 | 2 | 80 | 6.91E−17 | 1.05E−15 | 0.02439024 | YES |
| BCCIP | ENSG00000107949 | 5 | 189 | 2.47E−34 | 6.83E−33 | 0.0257732 | YES |
| BCL2 | ENSG00000171791 | 1598 | 7662 | 1.40E−189 | 1.44E−186 | 0.17257019 | YES |
| BCL2L1 | ENSG00000171552 | 606 | 4998 | 3.44E−219 | 6.67E−216 | 0.10813704 | YES |
| BCLAF1 | ENSG00000029363 | 84 | 156 | 1.24E−05 | 0.00012018 | 0.35 | YES |
| BCS1L | ENSG00000074582 | 159 | 279 | 4.05E−07 | 4.19E−06 | 0.3630137 | YES |
| BDP1 | ENSG00000145734 | 123 | 339 | 4.09E−18 | 6.51E−17 | 0.26623377 | YES |
| BECN1 | ENSG00000126581 | 62 | 148 | 2.74E−08 | 3.00E−07 | 0.2952381 | YES |
| BET1 | ENSG00000105829 | 9 | 117 | 5.71E−20 | 9.62E−19 | 0.07142857 | YES |
| BIRC5 | ENSG00000089685 | 5 | 29 | 2.84E−05 | 0.00027015 | 0.14705882 | YES |
| BLM | ENSG00000197299 | 582 | 917 | 1.84E−10 | 2.18E−09 | 0.38825884 | YES |
| BMS1 | ENSG00000165733 | 4 | 110 | 2.56E−21 | 4.55E−20 | 0.03508772 | YES |
| BNIP1 | ENSG00000113734 | 4 | 43 | 1.53E−08 | 1.69E−07 | 0.08510638 | YES |
| BOD1L1 | ENSG00000038219 | 34 | 111 | 1.06E−09 | 1.22E−08 | 0.23448276 | YES |
| BORA | ENSG00000136122 | 51 | 311 | 2.29E−33 | 6.07E−32 | 0.14088398 | YES |
| BPTF | ENSG00000171634 | 952 | 2930 | 4.57E−79 | 6.28E−77 | 0.24523442 | YES |
| BRCA1 | ENSG00000012048 | 249 | 388 | 2.81E−06 | 2.81E−05 | 0.39089482 | YES |
| BRCA2 | ENSG00000139618 | 19 | 584 | 1.37E−81 | 1.98E−79 | 0.03150912 | YES |
| BRD4 | ENSG00000141867 | 101 | 455 | 1.35E−36 | 4.10E−35 | 0.18165468 | YES |
| BRD8 | ENSG00000112983 | 26 | 101 | 1.75E−10 | 2.08E−09 | 0.20472441 | YES |
| BRF1 | ENSG00000185024 | 14 | 249 | 2.97E−39 | 9.78E−38 | 0.05323194 | YES |
| BRIP1 | ENSG00000136492 | 365 | 530 | 7.04E−06 | 6.90E−05 | 0.40782123 | YES |
| BRIX1 | ENSG00000113460 | 3 | 275 | 8.67E−49 | 4.03E−47 | 0.01079137 | YES |
| BTAF1 | ENSG00000095564 | 61 | 253 | 3.27E−22 | 6.01E−21 | 0.19426752 | YES |
| BTF3 | ENSG00000145741 | 84 | 293 | 2.74E−21 | 4.86E−20 | 0.22281167 | YES |
| BUB1 | ENSG00000169679 | 92 | 218 | 1.00E−10 | 1.19E−09 | 0.29677419 | YES |
| BUB1B | ENSG00000156970 | 160 | 463 | 5.36E−24 | 1.03E−22 | 0.25682183 | YES |
| BUB3 | ENSG00000154473 | 11 | 104 | 1.07E−16 | 1.62E−15 | 0.09565217 | YES |
| BUD13 | ENSG00000137656 | 15 | 358 | 1.18E−54 | 6.61E−53 | 0.04021448 | YES |
| BUD31 | ENSG00000106245 | 46 | 157 | 4.30E−13 | 5.67E−12 | 0.22660099 | YES |
| BYSL | ENSG00000112578 | 55 | 465 | 2.29E−52 | 1.18E−50 | 0.10576923 | YES |
| C12orf45 | ENSG00000151131 | 3 | 44 | 2.92E−09 | 3.30E−08 | 0.06382979 | YES |
| C14orf166 | ENSG00000087302 | 20 | 96 | 1.19E−11 | 1.48E−10 | 0.17241379 | YES |
| C15orf41 | ENSG00000186073 | 32 | 121 | 9.15E−12 | 1.15E−10 | 0.20915033 | YES |
| C16orf80 | ENSG00000070761 | 36 | 194 | 1.26E−21 | 2.27E−20 | 0.15652174 | YES |
| C18orf21 | ENSG00000141428 | 54 | 231 | 3.13E−21 | 5.55E−20 | 0.18947368 | YES |
| C19orf43 | ENSG00000123144 | 157 | 255 | 1.46E−05 | 0.00014073 | 0.38106796 | YES |
| C1orf131 | ENSG00000143633 | 13 | 301 | 1.65E−47 | 7.37E−46 | 0.04140127 | YES |
| C21orf59 | ENSG00000159079 | 79 | 270 | 1.20E−19 | 2.01E−18 | 0.22636103 | YES |
| C22orf28 | ENSG00000100220 | 7 | 47 | 6.32E−08 | 6.79E−07 | 0.12962963 | YES |
| C3orf17 | ENSG00000163608 | 13 | 341 | 3.10E−53 | 1.64E−51 | 0.03672316 | YES |
| C6orf48 | ENSG00000204387 | 339 | 456 | 0.00044586 | 0.00391701 | 0.42641509 | YES |
| C6orf57 | ENSG00000154079 | 108 | 225 | 7.42E−09 | 8.30E−08 | 0.32432432 | YES |
| C7orf73 | ENSG00000243317 | 64 | 342 | 2.84E−33 | 7.48E−32 | 0.15763547 | YES |
| C9orf114 | ENSG00000198917 | 3 | 95 | 3.96E−19 | 6.50E−18 | 0.03061224 | YES |
| C9orf78 | ENSG00000136819 | 65 | 187 | 1.87E−12 | 2.41E−11 | 0.25793651 | YES |
| CAB39 | ENSG00000135932 | 389 | 1362 | 1.69E−60 | 1.21E−58 | 0.22215877 | YES |
| CAD | ENSG00000084774 | 20 | 56 | 3.55E−05 | 0.00033566 | 0.26315789 | YES |
| CALM2 | ENSG00000143933 | 103 | 182 | 1.40E−05 | 0.00013526 | 0.36140351 | YES |
| CAMLG | ENSG00000164615 | 16 | 216 | 3.58E−33 | 9.39E−32 | 0.06896552 | YES |
| CAPNS1 | ENSG00000126247 | 248 | 356 | 0.00013298 | 0.00120841 | 0.41059603 | YES |
| CAPZB | ENSG00000077549 | 92 | 2365 | 6.34E−200 | 7.38E−197 | 0.03744404 | YES |
| CARS | ENSG00000110619 | 9 | 341 | 3.36E−55 | 1.93E−53 | 0.02571429 | YES |
| CASC3 | ENSG00000108349 | 345 | 456 | 0.000853 | 0.00733149 | 0.43071161 | YES |
| CASC5 | ENSG00000137812 | 53 | 412 | 2.88E−46 | 1.24E−44 | 0.11397849 | YES |
| CCAR1 | ENSG00000060339 | 325 | 483 | 4.48E−06 | 4.44E−05 | 0.40222772 | YES |
| CCDC115 | ENSG00000136710 | 3 | 25 | 2.32E−05 | 0.00022143 | 0.10714286 | YES |
| CCDC130 | ENSG00000104957 | 36 | 125 | 3.26E−11 | 3.98E−10 | 0.22360248 | YES |
| CCDC174 | ENSG00000154781 | 13 | 211 | 8.39E−34 | 2.28E−32 | 0.05803571 | YES |
| CCDC47 | ENSG00000108588 | 473 | 889 | 5.08E−17 | 7.78E−16 | 0.34728341 | YES |
| CCDC59 | ENSG00000133773 | 14 | 179 | 3.78E−28 | 8.34E−27 | 0.07253886 | YES |
| CCDC84 | ENSG00000186166 | 11 | 82 | 1.00E−12 | 1.30E−11 | 0.11827957 | YES |
| CCDC86 | ENSG00000110104 | 24 | 346 | 7.34E−49 | 3.42E−47 | 0.06486486 | YES |
| CCDC94 | ENSG00000105248 | 7 | 101 | 4.33E−18 | 6.88E−17 | 0.06481481 | YES |
| CCNA2 | ENSG00000145386 | 8 | 78 | 2.38E−13 | 3.16E−12 | 0.09302326 | YES |
| CCNF | ENSG00000162063 | 25 | 57 | 0.00034784 | 0.00309475 | 0.30487805 | YES |
| CCNH | ENSG00000134480 | 41 | 238 | 1.93E−26 | 4.01E−25 | 0.14695341 | YES |
| CCNK | ENSG00000090061 | 17 | 110 | 3.62E−15 | 5.20E−14 | 0.13385827 | YES |
| CCP110 | ENSG00000103540 | 332 | 602 | 2.37E−12 | 3.04E−11 | 0.35546039 | YES |
| CCT2 | ENSG00000166226 | 3 | 74 | 3.60E−15 | 5.19E−14 | 0.03896104 | YES |
| CCT3 | ENSG00000163468 | 6 | 67 | 3.06E−12 | 3.91E−11 | 0.08219178 | YES |
| CCT4 | ENSG00000115484 | 14 | 353 | 1.96E−54 | 1.08E−52 | 0.03814714 | YES |
| CCT5 | ENSG00000150753 | 26 | 654 | 1.34E−86 | 2.14E−84 | 0.03823529 | YES |
| CCT6A | ENSG00000146731 | 3 | 71 | 1.36E−14 | 1.91E−13 | 0.04054054 | YES |
| CCT7 | ENSG00000135624 | 14 | 347 | 1.35E−53 | 7.27E−52 | 0.03878116 | YES |
| CCT8 | ENSG00000156261 | 6 | 333 | 1.36E−55 | 7.97E−54 | 0.01769912 | YES |
| CD3EAP | ENSG00000117877 | 63 | 207 | 1.65E−15 | 2.40E−14 | 0.23333333 | YES |
| CDC123 | ENSG00000151465 | 140 | 1835 | 7.04E−152 | 4.55E−149 | 0.07088608 | YES |
| CDC16 | ENSG00000130177 | 15 | 144 | 7.66E−22 | 1.39E−20 | 0.09433962 | YES |
| CDC20 | ENSG00000117399 | 4 | 20 | 0.00068114 | 0.00590962 | 0.16666667 | YES |
| CDC23 | ENSG00000094880 | 6 | 118 | 1.12E−21 | 2.02E−20 | 0.0483871 | YES |
| CDC26 | ENSG00000176386 | 14 | 118 | 8.10E−18 | 1.27E−16 | 0.10606061 | YES |
| CDC27 | ENSG00000004897 | 8 | 381 | 3.28E−61 | 2.39E−59 | 0.02056555 | YES |
| CDC37 | ENSG00000105401 | 10 | 164 | 1.36E−27 | 2.94E−26 | 0.05747126 | YES |
| CDC40 | ENSG00000168438 | 28 | 371 | 1.37E−50 | 6.74E−49 | 0.07017544 | YES |
| CDC42 | ENSG00000070831 | 916 | 1490 | 5.51E−15 | 7.87E−14 | 0.38071488 | YES |
| CDC45 | ENSG00000093009 | 11 | 400 | 2.73E−62 | 2.08E−60 | 0.02676399 | YES |
| CDC5L | ENSG00000096401 | 23 | 211 | 2.85E−29 | 6.51E−28 | 0.0982906 | YES |
| CDC6 | ENSG00000094804 | 43 | 200 | 3.47E−20 | 5.92E−19 | 0.17695473 | YES |
| CDC7 | ENSG00000097046 | 118 | 472 | 3.79E−34 | 1.04E−32 | 0.2 | YES |
| CDC73 | ENSG00000134371 | 21 | 389 | 3.97E−56 | 2.38E−54 | 0.05121951 | YES |
| CDCA5 | ENSG00000146670 | 6 | 76 | 5.93E−14 | 8.11E−13 | 0.07317073 | YES |
| CDIPT | ENSG00000103502 | 32 | 68 | 0.00030199 | 0.00268957 | 0.32 | YES |
| CDK1 | ENSG00000170312 | 1 | 186 | 4.54E−36 | 1.37E−34 | 0.00534759 | YES |
| CDK12 | ENSG00000167258 | 37 | 185 | 6.39E−20 | 1.07E−18 | 0.16666667 | YES |
| CDK7 | ENSG00000134058 | 20 | 250 | 1.28E−36 | 3.93E−35 | 0.07407407 | YES |
| CDK9 | ENSG00000136807 | 3 | 65 | 1.98E−13 | 2.64E−12 | 0.04411765 | YES |
| CDT1 | ENSG00000167513 | 82 | 131 | 0.00106353 | 0.00908294 | 0.38497653 | YES |
| CEBPD | ENSG00000221869 | 48 | 92 | 0.00024242 | 0.00217008 | 0.34285714 | YES |
| CEBPZ | ENSG00000115816 | 43 | 350 | 1.19E−41 | 4.34E−40 | 0.10941476 | YES |
| CENPA | ENSG00000115163 | 2 | 47 | 2.02E−10 | 2.38E−09 | 0.04081633 | YES |
| CENPC1 | ENSG00000145241 | 44 | 558 | 4.93E−68 | 4.75E−66 | 0.0730897 | YES |
| CENPH | ENSG00000153044 | 30 | 389 | 3.42E−52 | 1.75E−50 | 0.07159905 | YES |
| CENPJ | ENSG00000151849 | 9 | 190 | 2.12E−32 | 5.40E−31 | 0.04522613 | YES |
| CENPL | ENSG00000120334 | 29 | 150 | 2.91E−17 | 4.49E−16 | 0.16201117 | YES |
| CENPM | ENSG00000100162 | 10 | 293 | 8.16E−48 | 3.69E−46 | 0.0330033 | YES |
| CENPN | ENSG00000166451 | 20 | 124 | 2.34E−16 | 3.50E−15 | 0.13888889 | YES |
| CENPW | ENSG00000203760 | 3 | 37 | 7.74E−08 | 8.29E−07 | 0.075 | YES |
| CEP135 | ENSG00000174799 | 110 | 587 | 2.25E−49 | 1.06E−47 | 0.15781923 | YES |
| CEP152 | ENSG00000103995 | 64 | 418 | 3.29E−43 | 1.29E−41 | 0.13278008 | YES |
| CEP192 | ENSG00000101639 | 75 | 1053 | 7.72E−109 | 2.32E−106 | 0.06648936 | YES |
| CEP350 | ENSG00000135837 | 180 | 680 | 7.58E−42 | 2.79E−40 | 0.20930233 | YES |
| CEP44 | ENSG00000164118 | 61 | 133 | 9.72E−07 | 9.93E−06 | 0.31443299 | YES |
| CEP55 | ENSG00000138180 | 23 | 146 | 7.79E−19 | 1.26E−17 | 0.13609467 | YES |
| CEP57 | ENSG00000166037 | 136 | 380 | 5.49E−20 | 9.28E−19 | 0.26356589 | YES |
| CEP63 | ENSG00000182923 | 875 | 1326 | 5.01E−11 | 6.08E−10 | 0.39754657 | YES |
| CEP85 | ENSG00000130695 | 45 | 1130 | 1.55E−126 | 7.72E−124 | 0.03829787 | YES |
| CEP97 | ENSG00000182504 | 163 | 443 | 1.97E−21 | 3.51E−20 | 0.2689769 | YES |
| CFDP1 | ENSG00000153774 | 91 | 449 | 1.12E−38 | 3.61E−37 | 0.16851852 | YES |
| CHAF1A | ENSG00000167670 | 28 | 239 | 1.63E−31 | 4.03E−30 | 0.10486891 | YES |
| CHAF1B | ENSG00000159259 | 26 | 71 | 6.55E−06 | 6.45E−05 | 0.26804124 | YES |
| CHCHD4 | ENSG00000163528 | 55 | 673 | 3.39E−77 | 4.42E−75 | 0.07554945 | YES |
| CHEK1 | ENSG00000149554 | 86 | 304 | 3.69E−22 | 6.76E−21 | 0.22051282 | YES |
| CHERP | ENSG00000085872 | 6 | 92 | 5.99E−17 | 9.15E−16 | 0.06122449 | YES |
| CHMP2A | ENSG00000130724 | 12 | 208 | 8.24E−34 | 2.24E−32 | 0.05454545 | YES |
| CHMP3 | ENSG00000115561 | 9 | 182 | 4.33E−31 | 1.05E−29 | 0.04712042 | YES |
| CHMP4B | ENSG00000101421 | 19 | 404 | 4.68E−59 | 3.21E−57 | 0.04491726 | YES |
| CHMP6 | ENSG00000176108 | 3 | 75 | 2.32E−15 | 3.35E−14 | 0.03846154 | YES |
| CHORDC1 | ENSG00000110172 | 45 | 289 | 2.19E−32 | 5.59E−31 | 0.13473054 | YES |
| CHTF8 | ENSG00000168802 | 165 | 406 | 1.80E−17 | 2.79E−16 | 0.28896673 | YES |
| CHTOP | ENSG00000160679 | 172 | 570 | 1.74E−32 | 4.48E−31 | 0.23180593 | YES |
| CIAO1 | ENSG00000144021 | 9 | 85 | 3.55E−14 | 4.91E−13 | 0.09574468 | YES |
| CIAPIN1 | ENSG00000005194 | 26 | 281 | 1.22E−38 | 3.92E−37 | 0.08469055 | YES |
| CINP | ENSG00000100865 | 4 | 168 | 2.08E−31 | 5.13E−30 | 0.02325581 | YES |
| CIRH1A | ENSG00000141076 | 51 | 313 | 1.21E−33 | 3.24E−32 | 0.14010989 | YES |
| CIT | ENSG00000122966 | 36 | 146 | 2.40E−14 | 3.34E−13 | 0.1978022 | YES |
| CKAP5 | ENSG00000175216 | 16 | 326 | 1.12E−49 | 5.30E−48 | 0.04678363 | YES |
| CLASP2 | ENSG00000163539 | 501 | 666 | 0.00013062 | 0.00118814 | 0.42930591 | YES |
| CLASRP | ENSG00000104859 | 44 | 166 | 6.28E−15 | 8.95E−14 | 0.20952381 | YES |
| CLNS1A | ENSG00000074201 | 53 | 1078 | 6.43E−119 | 2.55E−116 | 0.04686118 | YES |
| CLP1 | ENSG00000172409 | 16 | 64 | 1.27E−07 | 1.35E−06 | 0.2 | YES |
| CLPB | ENSG00000162129 | 1295 | 1838 | 7.59E−10 | 8.80E−09 | 0.41334184 | YES |
| CLSPN | ENSG00000092853 | 10 | 491 | 1.70E−74 | 1.95E−72 | 0.01996008 | YES |
| CLTC | ENSG00000141367 | 51 | 904 | 4.35E−103 | 1.19E−100 | 0.05340314 | YES |
| CMPK1 | ENSG00000162368 | 16 | 203 | 4.17E−31 | 1.01E−29 | 0.07305936 | YES |
| CNOT1 | ENSG00000125107 | 44 | 615 | 8.94E−75 | 1.03E−72 | 0.06676783 | YES |
| CNOT10 | ENSG00000182973 | 211 | 674 | 6.20E−35 | 1.76E−33 | 0.23841808 | YES |
| CNOT11 | ENSG00000158435 | 65 | 125 | 3.00E−05 | 0.00028484 | 0.34210526 | YES |
| CNOT2 | ENSG00000111596 | 90 | 663 | 5.79E−64 | 4.70E−62 | 0.11952191 | YES |
| CNOT3 | ENSG00000088038 | 7 | 58 | 4.89E−10 | 5.69E−09 | 0.10769231 | YES |
| COA5 | ENSG00000183513 | 18 | 186 | 1.85E−27 | 3.97E−26 | 0.08823529 | YES |
| COASY | ENSG00000068120 | 176 | 305 | 2.72E−07 | 2.84E−06 | 0.36590437 | YES |
| COG1 | ENSG00000166685 | 4 | 55 | 6.29E−11 | 7.62E−10 | 0.06779661 | YES |
| COG2 | ENSG00000135775 | 3 | 56 | 1.16E−11 | 1.44E−10 | 0.05084746 | YES |
| COG3 | ENSG00000136152 | 16 | 366 | 2.75E−55 | 1.60E−53 | 0.04188482 | YES |
| COG4 | ENSG00000103051 | 14 | 366 | 3.10E−56 | 1.87E−54 | 0.03684211 | YES |
| COG5 | ENSG00000164597 | 609 | 937 | 9.37E−10 | 1.08E−08 | 0.39391979 | YES |
| COG7 | ENSG00000168434 | 101 | 219 | 2.50E−09 | 2.83E−08 | 0.315625 | YES |
| COG8 | ENSG00000213380 | 130 | 423 | 7.51E−26 | 1.53E−24 | 0.23508137 | YES |
| COPA | ENSG00000122218 | 8 | 242 | 3.68E−41 | 1.31E−39 | 0.032 | YES |
| COPB1 | ENSG00000129083 | 19 | 156 | 4.15E−22 | 7.59E−21 | 0.10857143 | YES |
| COPB2 | ENSG00000184432 | 3 | 104 | 8.73E−21 | 1.52E−19 | 0.02803738 | YES |
| COPE | ENSG00000105669 | 19 | 120 | 4.47E−16 | 6.63E−15 | 0.13669065 | YES |
| COPG1 | ENSG00000181789 | 10 | 345 | 2.91E−55 | 1.68E−53 | 0.02816901 | YES |
| COPS2 | ENSG00000166200 | 4 | 337 | 3.43E−57 | 2.14E−55 | 0.01173021 | YES |
| COPS3 | ENSG00000141030 | 7 | 299 | 3.27E−50 | 1.58E−48 | 0.02287582 | YES |
| COPS4 | ENSG00000138663 | 10 | 335 | 7.39E−54 | 4.01E−52 | 0.02898551 | YES |
| COPS6 | ENSG00000168090 | 18 | 74 | 1.20E−08 | 1.33E−07 | 0.19565217 | YES |
| COPS8 | ENSG00000198612 | 1 | 30 | 1.71E−07 | 1.80E−06 | 0.03225806 | YES |
| COPZ1 | ENSG00000111481 | 4 | 95 | 1.43E−18 | 2.30E−17 | 0.04040404 | YES |
| COQ3 | ENSG00000132423 | 87 | 220 | 7.25E−12 | 9.13E−11 | 0.28338762 | YES |
| COQ4 | ENSG00000167113 | 61 | 146 | 3.05E−08 | 3.33E−07 | 0.29468599 | YES |
| COQ5 | ENSG00000110871 | 120 | 240 | 1.45E−08 | 1.61E−07 | 0.33333333 | YES |
| COX10 | ENSG00000006695 | 76 | 445 | 1.03E−42 | 3.90E−41 | 0.14587332 | YES |
| COX11 | ENSG00000166260 | 9 | 136 | 2.61E−23 | 4.94E−22 | 0.06206897 | YES |
| COX15 | ENSG00000014919 | 55 | 312 | 3.76E−32 | 9.51E−31 | 0.14986376 | YES |
| COX17 | ENSG00000138495 | 65 | 323 | 1.95E−30 | 4.64E−29 | 0.16752577 | YES |
| COX5B | ENSG00000135940 | 6 | 104 | 3.75E−19 | 6.17E−18 | 0.05454545 | YES |
| COX6B1 | ENSG00000126267 | 81 | 189 | 1.72E−09 | 1.96E−08 | 0.3 | YES |
| COX7B | ENSG00000131174 | 63 | 416 | 2.74E−43 | 1.08E−41 | 0.13152401 | YES |
| COX7C | ENSG00000127184 | 13 | 115 | 9.75E−18 | 1.53E−16 | 0.1015625 | YES |
| CPSF2 | ENSG00000165934 | 57 | 481 | 1.17E−53 | 6.31E−52 | 0.10594796 | YES |
| CPSF3 | ENSG00000119203 | 23 | 111 | 4.39E−13 | 5.78E−12 | 0.17164179 | YES |
| CPSF6 | ENSG00000111605 | 11 | 226 | 3.74E−37 | 1.15E−35 | 0.0464135 | YES |
| CRCP | ENSG00000241258 | 56 | 427 | 3.84E−47 | 1.69E−45 | 0.11594203 | YES |
| CREBBP | ENSG00000005339 | 39 | 1067 | 4.10E−123 | 1.79E−120 | 0.03526221 | YES |
| CRLS1 | ENSG00000088766 | 45 | 341 | 1.16E−39 | 3.89E−38 | 0.11658031 | YES |
| CRNKL1 | ENSG00000101343 | 5 | 80 | 3.16E−15 | 4.56E−14 | 0.05882353 | YES |
| CSDE1 | ENSG00000009307 | 59 | 337 | 3.19E−34 | 8.82E−33 | 0.1489899 | YES |
| CSE1L | ENSG00000124207 | 6 | 468 | 1.48E−73 | 1.66E−71 | 0.01265823 | YES |
| CSNK1A1 | ENSG00000113712 | 56 | 520 | 7.37E−59 | 5.03E−57 | 0.09722222 | YES |
| CSNK2B | ENSG00000204435 | 71 | 128 | 0.00010205 | 0.00093362 | 0.35678392 | YES |
| CSTF1 | ENSG00000101138 | 19 | 399 | 2.19E−58 | 1.46E−56 | 0.04545455 | YES |
| CSTF2 | ENSG00000101811 | 38 | 134 | 5.62E−12 | 7.12E−11 | 0.22093023 | YES |
| CSTF3 | ENSG00000176102 | 63 | 835 | 1.07E−91 | 1.99E−89 | 0.0701559 | YES |
| CTC1 | ENSG00000178971 | 7 | 352 | 9.70E−58 | 6.25E−56 | 0.01949861 | YES |
| CTCF | ENSG00000102974 | 109 | 1090 | 3.64E−101 | 9.22E−99 | 0.09090909 | YES |
| CTDP1 | ENSG00000060069 | 49 | 257 | 1.82E−26 | 3.81E−25 | 0.16013072 | YES |
| CTDSPL2 | ENSG00000137770 | 461 | 1162 | 9.96E−36 | 2.95E−34 | 0.2840419 | YES |
| CTNNBL1 | ENSG00000132792 | 237 | 1646 | 9.28E−113 | 3.18E−110 | 0.12586298 | YES |
| CTPS1 | ENSG00000171793 | 86 | 303 | 4.91E−22 | 8.94E−21 | 0.22107969 | YES |
| CTR9 | ENSG00000198730 | 25 | 175 | 9.62E−23 | 1.79E−21 | 0.125 | YES |
| CTU1 | ENSG00000142544 | 33 | 170 | 5.41E−19 | 8.84E−18 | 0.16256158 | YES |
| CTU2 | ENSG00000174177 | 36 | 98 | 2.37E−07 | 2.48E−06 | 0.26865672 | YES |
| CUL1 | ENSG00000055130 | 102 | 550 | 1.91E−47 | 8.42E−46 | 0.15644172 | YES |
| CUL2 | ENSG00000108094 | 40 | 225 | 7.09E−25 | 1.40E−23 | 0.1509434 | YES |
| CWC22 | ENSG00000163510 | 7 | 248 | 1.34E−42 | 5.04E−41 | 0.02745098 | YES |
| CWC25 | ENSG00000108296 | 32 | 87 | 9.30E−07 | 9.51E−06 | 0.26890756 | YES |
| CWF19L2 | ENSG00000152404 | 72 | 186 | 7.58E−11 | 9.14E−10 | 0.27906977 | YES |
| CXorf56 | ENSG00000018610 | 49 | 129 | 1.42E−08 | 1.58E−07 | 0.2752809 | YES |
| CYCS | ENSG00000172115 | 50 | 192 | 6.37E−17 | 9.71E−16 | 0.20661157 | YES |
| DAD1 | ENSG00000129562 | 11 | 248 | 1.45E−40 | 5.05E−39 | 0.04247104 | YES |
| DAP3 | ENSG00000132676 | 164 | 425 | 1.77E−19 | 2.93E−18 | 0.27843803 | YES |
| DARS | ENSG00000115866 | 53 | 1111 | 6.53E−122 | 2.78E−119 | 0.04553265 | YES |
| DARS2 | ENSG00000117593 | 208 | 312 | 5.87E−05 | 0.00054739 | 0.4 | YES |
| DBR1 | ENSG00000138231 | 24 | 220 | 2.97E−30 | 7.01E−29 | 0.09836066 | YES |
| DCAF7 | ENSG00000136485 | 168 | 429 | 3.46E−19 | 5.70E−18 | 0.28140704 | YES |
| DCP2 | ENSG00000172795 | 35 | 198 | 1.38E−22 | 2.57E−21 | 0.15021459 | YES |
| DCPS | ENSG00000110063 | 198 | 1264 | 9.22E−92 | 1.73E−89 | 0.13543092 | YES |
| DCTN1 | ENSG00000204843 | 36 | 105 | 2.52E−08 | 2.77E−07 | 0.25531915 | YES |
| DCTN2 | ENSG00000175203 | 2 | 22 | 3.13E−05 | 0.00029615 | 0.08333333 | YES |
| DCTN3 | ENSG00000137100 | 0 | 32 | 1.55E−08 | 1.71E−07 | 0 | YES |
| DCTN4 | ENSG00000132912 | 31 | 1325 | 3.06E−148 | 1.84E−145 | 0.02286136 | YES |
| DCTN5 | ENSG00000166847 | 27 | 461 | 5.72E−63 | 4.48E−61 | 0.05532787 | YES |
| DDB1 | ENSG00000167986 | 9 | 135 | 3.90E−23 | 7.35E−22 | 0.0625 | YES |
| DDOST | ENSG00000244038 | 6 | 147 | 9.24E−27 | 1.94E−25 | 0.03921569 | YES |
| DDX10 | ENSG00000178105 | 102 | 465 | 1.65E−37 | 5.17E−36 | 0.17989418 | YES |
| DDX18 | ENSG00000088205 | 11 | 172 | 1.96E−28 | 4.38E−27 | 0.06010929 | YES |
| DDX19A | ENSG00000168872 | 8 | 212 | 1.85E−36 | 5.59E−35 | 0.03636364 | YES |
| DDX20 | ENSG00000064703 | 12 | 100 | 1.57E−15 | 2.29E−14 | 0.10714286 | YES |
| DDX21 | ENSG00000165732 | 64 | 424 | 5.59E−44 | 2.25E−42 | 0.13114754 | YES |
| DDX23 | ENSG00000174243 | 12 | 403 | 3.30E−62 | 2.51E−60 | 0.02891566 | YES |
| DDX24 | ENSG00000089737 | 14 | 127 | 2.22E−19 | 3.69E−18 | 0.09929078 | YES |
| DDX27 | ENSG00000124228 | 27 | 299 | 7.52E−41 | 2.65E−39 | 0.08282209 | YES |
| DDX39B | ENSG00000198563 | 206 | 407 | 8.83E−12 | 1.11E−10 | 0.3360522 | YES |
| DDX3X | ENSG00000215301 | 291 | 481 | 1.93E−08 | 2.12E−07 | 0.37694301 | YES |
| DDX46 | ENSG00000145833 | 28 | 303 | 5.25E−41 | 1.87E−39 | 0.08459215 | YES |
| DDX47 | ENSG00000213782 | 2 | 170 | 7.34E−33 | 1.91E−31 | 0.01162791 | YES |
| DDX49 | ENSG00000105671 | 2 | 132 | 2.28E−26 | 4.72E−25 | 0.01492537 | YES |
| DDX51 | ENSG00000185163 | 1 | 20 | 2.38E−05 | 0.00022703 | 0.04761905 | YES |
| DDX52 | ENSG00000141141 | 6 | 193 | 1.89E−34 | 5.24E−33 | 0.03015075 | YES |
| DDX54 | ENSG00000123064 | 5 | 159 | 2.40E−29 | 5.50E−28 | 0.0304878 | YES |
| DDX55 | ENSG00000111364 | 11 | 347 | 4.75E−55 | 2.68E−53 | 0.03072626 | YES |
| DDX56 | ENSG00000136271 | 5 | 187 | 5.24E−34 | 1.43E−32 | 0.02604167 | YES |
| DDX59 | ENSG00000118197 | 240 | 339 | 0.00030033 | 0.00267753 | 0.41450777 | YES |
| DDX6 | ENSG00000110367 | 148 | 551 | 1.05E−35 | 3.10E−34 | 0.21173104 | YES |
| DENR | ENSG00000139726 | 175 | 294 | 1.34E−06 | 1.36E−05 | 0.37313433 | YES |
| DGCR14 | ENSG00000100056 | 1 | 74 | 2.48E−16 | 3.70E−15 | 0.01333333 | YES |
| DGCR8 | ENSG00000128191 | 49 | 318 | 4.93E−35 | 1.41E−33 | 0.13351499 | YES |
| DHDDS | ENSG00000117682 | 92 | 165 | 1.98E−05 | 0.00018965 | 0.35797665 | YES |
| DHFR | ENSG00000228716 | 5 | 115 | 1.13E−21 | 2.04E−20 | 0.04166667 | YES |
| DHX15 | ENSG00000109606 | 74 | 709 | 2.20E−74 | 2.49E−72 | 0.0945083 | YES |
| DHX33 | ENSG00000005100 | 36 | 479 | 1.76E−61 | 1.30E−59 | 0.06990291 | YES |
| DHX35 | ENSG00000101452 | 193 | 356 | 2.64E−09 | 2.99E−08 | 0.35154827 | YES |
| DHX36 | ENSG00000174953 | 72 | 286 | 1.62E−23 | 3.09E−22 | 0.20111732 | YES |
| DHX38 | ENSG00000140829 | 32 | 68 | 0.00030199 | 0.00268957 | 0.32 | YES |
| DHX8 | ENSG00000067596 | 10 | 147 | 1.04E−24 | 2.04E−23 | 0.06369427 | YES |
| DHX9 | ENSG00000135829 | 7 | 189 | 2.86E−33 | 7.52E−32 | 0.03571429 | YES |
| DIDO1 | ENSG00000101191 | 96 | 658 | 1.64E−61 | 1.22E−59 | 0.12732095 | YES |
| DIEXF | ENSG00000117597 | 7 | 98 | 1.53E−17 | 2.38E−16 | 0.06666667 | YES |
| DIMT1 | ENSG00000086189 | 40 | 280 | 6.45E−33 | 1.68E−31 | 0.125 | YES |
| DIS3 | ENSG00000083520 | 49 | 217 | 9.13E−21 | 1.59E−19 | 0.18421053 | YES |
| DKC1 | ENSG00000130826 | 55 | 804 | 2.11E−91 | 3.87E−89 | 0.06402794 | YES |
| DLD | ENSG00000091140 | 92 | 188 | 1.35E−07 | 1.43E−06 | 0.32857143 | YES |
| DLST | ENSG00000119689 | 2 | 90 | 9.02E−19 | 1.46E−17 | 0.02173913 | YES |
| DMAP1 | ENSG00000178028 | 3 | 25 | 2.32E−05 | 0.00022143 | 0.10714286 | YES |
| DNAJA3 | ENSG00000103423 | 26 | 301 | 1.44E−41 | 5.20E−40 | 0.0795107 | YES |
| DNAJC11 | ENSG00000007923 | 29 | 217 | 9.51E−28 | 2.07E−26 | 0.11788618 | YES |
| DNAJC19 | ENSG00000205981 | 0 | 81 | 2.80E−18 | 4.47E−17 | 0 | YES |
| DNAJC2 | ENSG00000105821 | 256 | 506 | 1.51E−13 | 2.02E−12 | 0.33595801 | YES |
| DNAJC8 | ENSG00000126698 | 100 | 274 | 1.88E−15 | 2.72E−14 | 0.26737968 | YES |
| DNAJC9 | ENSG00000213551 | 868 | 1496 | 5.62E−18 | 8.91E−17 | 0.36717428 | YES |
| DNM1L | ENSG00000087470 | 17 | 348 | 2.56E−52 | 1.32E−50 | 0.04657534 | YES |
| DNM2 | ENSG00000079805 | 135 | 2665 | 3.00E−205 | 4.36E−202 | 0.04821429 | YES |
| DNMT1 | ENSG00000130816 | 71 | 639 | 1.39E−67 | 1.31E−65 | 0.1 | YES |
| DNTTIP2 | ENSG00000067334 | 21 | 125 | 3.89E−16 | 5.78E−15 | 0.14383562 | YES |
| DOHH | ENSG00000129932 | 100 | 310 | 1.32E−19 | 2.21E−18 | 0.24390244 | YES |
| DONSON | ENSG00000159147 | 2 | 132 | 2.28E−26 | 4.72E−25 | 0.01492537 | YES |
| DPH5 | ENSG00000117543 | 275 | 420 | 3.66E−06 | 3.64E−05 | 0.39568345 | YES |
| DPY30 | ENSG00000162961 | 41 | 98 | 2.84E−06 | 2.84E−05 | 0.29496403 | YES |
| DR1 | ENSG00000117505 | 130 | 434 | 4.62E−27 | 9.78E−26 | 0.23049645 | YES |
| DSN1 | ENSG00000149636 | 54 | 1234 | 2.49E−132 | 1.36E−129 | 0.04192547 | YES |
| DTL | ENSG00000143476 | 29 | 430 | 4.39E−58 | 2.87E−56 | 0.06318083 | YES |
| DTYMK | ENSG00000168393 | 8 | 142 | 7.44E−25 | 1.47E−23 | 0.05333333 | YES |
| DUT | ENSG00000128951 | 17 | 462 | 1.47E−67 | 1.38E−65 | 0.03549061 | YES |
| DYNC1H1 | ENSG00000197102 | 14 | 332 | 1.75E−51 | 8.84E−50 | 0.04046243 | YES |
| DYNC1I2 | ENSG00000077380 | 92 | 348 | 4.58E−26 | 9.40E−25 | 0.20909091 | YES |
| DYNLRB1 | ENSG00000125971 | 8 | 413 | 1.75E−65 | 1.50E−63 | 0.01900238 | YES |
| DYRK1A | ENSG00000157540 | 158 | 874 | 1.91E−66 | 1.70E−64 | 0.15310078 | YES |
| E4F1 | ENSG00000167967 | 15 | 63 | 8.74E−08 | 9.33E−07 | 0.19230769 | YES |
| EAPP | ENSG00000129518 | 160 | 269 | 2.85E−06 | 2.85E−05 | 0.37296037 | YES |
| EBNA1BP2 | ENSG00000117395 | 7 | 78 | 7.83E−14 | 1.06E−12 | 0.08235294 | YES |
| ECD | ENSG00000122882 | 35 | 220 | 6.43E−26 | 1.31E−24 | 0.1372549 | YES |
| ECT2 | ENSG00000114346 | 20 | 147 | 3.37E−20 | 5.77E−19 | 0.11976048 | YES |
| EDC4 | ENSG00000038358 | 55 | 135 | 4.39E−08 | 4.76E−07 | 0.28947368 | YES |
| EED | ENSG00000074266 | 32 | 313 | 8.76E−41 | 3.08E−39 | 0.09275362 | YES |
| EEF1A1 | ENSG00000156508 | 60 | 314 | 8.51E−31 | 2.05E−29 | 0.16042781 | YES |
| EEF1G | ENSG00000254772 | 22 | 409 | 2.30E−58 | 1.52E−56 | 0.05104408 | YES |
| EEF2 | ENSG00000167658 | 3 | 135 | 2.53E−26 | 5.22E−25 | 0.02173913 | YES |
| EEFSEC | ENSG00000132394 | 169 | 498 | 8.09E−26 | 1.64E−24 | 0.25337331 | YES |
| EFTUD2 | ENSG00000108883 | 46 | 350 | 1.58E−40 | 5.51E−39 | 0.11616162 | YES |
| EHMT1 | ENSG00000181090 | 1974 | 2940 | 7.82E−15 | 1.11E−13 | 0.4017094 | YES |
| EIF1 | ENSG00000173812 | 15 | 103 | 9.02E−15 | 1.28E−13 | 0.12711864 | YES |
| EIF1AD | ENSG00000175376 | 60 | 148 | 1.12E−08 | 1.25E−07 | 0.28846154 | YES |
| EIF1AX | ENSG00000173674 | 10 | 213 | 1.34E−35 | 3.94E−34 | 0.04484305 | YES |
| EIF2B1 | ENSG00000111361 | 15 | 302 | 1.08E−46 | 4.71E−45 | 0.04731861 | YES |
| EIF2B2 | ENSG00000119718 | 7 | 76 | 1.86E−13 | 2.49E−12 | 0.08433735 | YES |
| EIF2B3 | ENSG00000070785 | 27 | 324 | 1.90E−44 | 7.73E−43 | 0.07692308 | YES |
| EIF2B4 | ENSG00000115211 | 19 | 96 | 5.20E−12 | 6.60E−11 | 0.16521739 | YES |
| EIF2B5 | ENSG00000145191 | 19 | 250 | 4.53E−37 | 1.40E−35 | 0.07063197 | YES |
| EIF2S1 | ENSG00000134001 | 40 | 252 | 7.53E−29 | 1.70E−27 | 0.1369863 | YES |
| EIF2S2 | ENSG00000125977 | 10 | 499 | 1.73E−75 | 2.09E−73 | 0.01964637 | YES |
| EIF2S3 | ENSG00000130741 | 60 | 870 | 2.32E−96 | 4.95E−94 | 0.06451613 | YES |
| EIF3A | ENSG00000107581 | 18 | 300 | 5.43E−45 | 2.23E−43 | 0.05660377 | YES |
| EIF3B | ENSG00000106263 | 2 | 162 | 1.61E−31 | 3.97E−30 | 0.01219512 | YES |
| EIF3D | ENSG00000100353 | 95 | 453 | 5.08E−38 | 1.62E−36 | 0.17335766 | YES |
| EIF3I | ENSG00000084623 | 6 | 117 | 1.69E−21 | 3.02E−20 | 0.04878049 | YES |
| EIF3J | ENSG00000104131 | 81 | 375 | 2.08E−32 | 5.32E−31 | 0.17763158 | YES |
| EIF3M | ENSG00000149100 | 9 | 258 | 4.26E−43 | 1.65E−41 | 0.03370787 | YES |
| EIF4A1 | ENSG00000161960 | 19 | 189 | 1.68E−27 | 3.62E−26 | 0.09134615 | YES |
| EIF4A2 | ENSG00000156976 | 117 | 199 | 2.12E−05 | 0.00020319 | 0.37025316 | YES |
| EIF4A3 | ENSG00000141543 | 1 | 35 | 1.53E−08 | 1.69E−07 | 0.02777778 | YES |
| EIF4B | ENSG00000063046 | 841 | 1432 | 5.98E−17 | 9.13E−16 | 0.3699956 | YES |
| EIF4E | ENSG00000151247 | 22 | 544 | 1.70E−75 | 2.07E−73 | 0.03886926 | YES |
| EIF4G1 | ENSG00000114867 | 30 | 108 | 2.27E−10 | 2.67E−09 | 0.2173913 | YES |
| EIF4G2 | ENSG00000110321 | 23 | 282 | 4.34E−40 | 1.49E−38 | 0.07540984 | YES |
| EIF5 | ENSG00000100664 | 4 | 71 | 4.82E−14 | 6.63E−13 | 0.05333333 | YES |
| EIF5A | ENSG00000132507 | 36 | 346 | 8.06E−44 | 3.22E−42 | 0.09424084 | YES |
| EIF5B | ENSG00000158417 | 36 | 393 | 3.00E−50 | 1.46E−48 | 0.08391608 | YES |
| EIF6 | ENSG00000242372 | 29 | 196 | 1.70E−24 | 3.31E−23 | 0.12888889 | YES |
| ELAC2 | ENSG00000006744 | 7 | 101 | 4.33E−18 | 6.88E−17 | 0.06481481 | YES |
| ELL | ENSG00000105656 | 92 | 1018 | 7.44E−100 | 1.86E−97 | 0.08288288 | YES |
| ELP2 | ENSG00000134759 | 60 | 351 | 9.10E−36 | 2.71E−34 | 0.1459854 | YES |
| ELP4 | ENSG00000109911 | 71 | 461 | 2.52E−46 | 1.09E−44 | 0.13345865 | YES |
| ELP5 | ENSG00000170291 | 72 | 212 | 5.08E−14 | 6.98E−13 | 0.25352113 | YES |
| ELP6 | ENSG00000163832 | 195 | 925 | 3.61E−62 | 2.73E−60 | 0.17410714 | YES |
| EMC3 | ENSG00000125037 | 81 | 436 | 4.81E−40 | 1.65E−38 | 0.15667311 | YES |
| EMC4 | ENSG00000128463 | 7 | 106 | 5.34E−19 | 8.75E−18 | 0.0619469 | YES |
| EMC7 | ENSG00000134153 | 24 | 161 | 6.93E−21 | 1.21E−19 | 0.12972973 | YES |
| ENO1 | ENSG00000074800 | 152 | 468 | 4.47E−26 | 9.17E−25 | 0.24516129 | YES |
| EPRS | ENSG00000136628 | 13 | 349 | 2.34E−54 | 1.29E−52 | 0.0359116 | YES |
| ERAL1 | ENSG00000132591 | 78 | 196 | 7.68E−11 | 9.25E−10 | 0.28467153 | YES |
| ERCC2 | ENSG00000104884 | 10 | 107 | 1.06E−17 | 1.66E−16 | 0.08547009 | YES |
| ERCC3 | ENSG00000163161 | 7 | 62 | 8.40E−11 | 1.01E−09 | 0.10144928 | YES |
| ERCC4 | ENSG00000175595 | 26 | 85 | 5.41E−08 | 5.85E−07 | 0.23423423 | YES |
| ERH | ENSG00000100632 | 82 | 231 | 3.72E−14 | 5.14E−13 | 0.26198083 | YES |
| ESF1 | ENSG00000089048 | 48 | 561 | 7.68E−67 | 6.95E−65 | 0.07881773 | YES |
| ETF1 | ENSG00000120705 | 34 | 295 | 2.17E−37 | 6.76E−36 | 0.10334347 | YES |
| EWSR1 | ENSG00000182944 | 149 | 279 | 3.00E−08 | 3.28E−07 | 0.34813084 | YES |
| EXOC1 | ENSG00000090989 | 329 | 620 | 8.28E−14 | 1.12E−12 | 0.34668072 | YES |
| EXOC2 | ENSG00000112685 | 443 | 670 | 1.09E−07 | 1.16E−06 | 0.39802336 | YES |
| EXOC4 | ENSG00000131558 | 252 | 529 | 8.88E−16 | 1.31E−14 | 0.32266325 | YES |
| EXOC5 | ENSG00000070367 | 72 | 415 | 3.30E−40 | 1.14E−38 | 0.14784394 | YES |
| EXOSC1 | ENSG00000171311 | 112 | 169 | 0.00115422 | 0.00982381 | 0.39857651 | YES |
| EXOSC10 | ENSG00000171824 | 87 | 302 | 1.15E−21 | 2.08E−20 | 0.22365039 | YES |
| EXOSC2 | ENSG00000130713 | 8 | 286 | 8.54E−48 | 3.85E−46 | 0.02721088 | YES |
| EXOSC3 | ENSG00000107371 | 3 | 128 | 4.29E−25 | 8.53E−24 | 0.02290076 | YES |
| EXOSC5 | ENSG00000077348 | 20 | 478 | 3.16E−68 | 3.07E−66 | 0.04016064 | YES |
| EXOSC7 | ENSG00000075914 | 7 | 280 | 2.02E−47 | 8.91E−46 | 0.02439024 | YES |
| EXOSC8 | ENSG00000120699 | 86 | 595 | 1.58E−57 | 1.01E−55 | 0.12628488 | YES |
| EXOSC9 | ENSG00000123737 | 16 | 194 | 1.17E−29 | 2.69E−28 | 0.07619048 | YES |
| EZH2 | ENSG00000106462 | 129 | 480 | 2.19E−32 | 5.59E−31 | 0.21182266 | YES |
| FAF2 | ENSG00000113194 | 42 | 243 | 7.99E−27 | 1.69E−25 | 0.14736842 | YES |
| FAM178A | ENSG00000119906 | 50 | 121 | 2.35E−07 | 2.46E−06 | 0.29239766 | YES |
| FAM204A | ENSG00000165669 | 162 | 345 | 2.94E−12 | 3.76E−11 | 0.31952663 | YES |
| FAM210A | ENSG00000177150 | 106 | 441 | 1.30E−33 | 3.47E−32 | 0.19378428 | YES |
| FAM32A | ENSG00000105058 | 6 | 65 | 7.39E−12 | 9.30E−11 | 0.08450704 | YES |
| FAM50A | ENSG00000071859 | 0 | 14 | 0.00012201 | 0.00111159 | 0 | YES |
| FAM60A | ENSG00000139146 | 710 | 890 | 0.00063897 | 0.00555759 | 0.44375 | YES |
| FAM96B | ENSG00000166595 | 8 | 60 | 5.77E−10 | 6.71E−09 | 0.11764706 | YES |
| FAM98B | ENSG00000171262 | 34 | 198 | 6.07E−23 | 1.14E−21 | 0.14655172 | YES |
| FANCB | ENSG00000181544 | 110 | 186 | 4.04E−05 | 0.00038004 | 0.37162162 | YES |
| FANCC | ENSG00000158169 | 471 | 635 | 8.50E−05 | 0.00078213 | 0.42585895 | YES |
| FANCD2 | ENSG00000144554 | 259 | 487 | 7.80E−12 | 9.79E−11 | 0.34718499 | YES |
| FANCL | ENSG00000115392 | 324 | 558 | 2.53E−10 | 2.98E−09 | 0.36734694 | YES |
| FANCM | ENSG00000187790 | 125 | 338 | 1.26E−17 | 1.97E−16 | 0.2699784 | YES |
| FARS2 | ENSG00000145982 | 681 | 1124 | 1.48E−13 | 1.99E−12 | 0.37728532 | YES |
| FARSA | ENSG00000179115 | 21 | 328 | 1.18E−47 | 5.25E−46 | 0.06017192 | YES |
| FARSB | ENSG00000116120 | 19 | 278 | 2.74E−41 | 9.82E−40 | 0.06397306 | YES |
| FAU | ENSG00000149806 | 16 | 139 | 1.50E−20 | 2.59E−19 | 0.10322581 | YES |
| FBL | ENSG00000105202 | 9 | 208 | 2.62E−35 | 7.57E−34 | 0.04147465 | YES |
| FBXO5 | ENSG00000112029 | 16 | 460 | 9.17E−68 | 8.70E−66 | 0.03361345 | YES |
| FCF1 | ENSG00000119616 | 28 | 171 | 5.97E−21 | 1.05E−19 | 0.14070352 | YES |
| FDPS | ENSG00000160752 | 288 | 565 | 2.75E−14 | 3.82E−13 | 0.33763189 | YES |
| FDX1L | ENSG00000267673 | 10 | 77 | 3.00E−12 | 3.84E−11 | 0.11494253 | YES |
| FDXR | ENSG00000161513 | 12 | 40 | 8.09E−05 | 0.0007463 | 0.23076923 | YES |
| FEN1 | ENSG00000168496 | 53 | 367 | 2.65E−40 | 9.18E−39 | 0.12619048 | YES |
| FGFR1OP | ENSG00000213066 | 50 | 218 | 1.33E−20 | 2.30E−19 | 0.18656716 | YES |
| FGFR1OP2 | ENSG00000111790 | 705 | 903 | 0.00020706 | 0.00185931 | 0.43843284 | YES |
| FH | ENSG00000091483 | 14 | 57 | 4.34E−07 | 4.48E−06 | 0.1971831 | YES |
| FIP1L1 | ENSG00000145216 | 17 | 367 | 5.88E−55 | 3.30E−53 | 0.04427083 | YES |
| FIS1 | ENSG00000214253 | 7 | 62 | 8.40E−11 | 1.01E−09 | 0.10144928 | YES |
| FLII | ENSG00000177731 | 40 | 80 | 0.00027842 | 0.00248475 | 0.33333333 | YES |
| FNBP4 | ENSG00000109920 | 250 | 355 | 0.00019897 | 0.00178851 | 0.41322314 | YES |
| FNTB | ENSG00000257365 | 46 | 1130 | 3.62E−126 | 1.75E−123 | 0.03911565 | YES |
| FTSJ1 | ENSG00000068438 | 175 | 255 | 0.00044163 | 0.00388179 | 0.40697674 | YES |
| FTSJ3 | ENSG00000108592 | 24 | 85 | 1.45E−08 | 1.61E−07 | 0.22018349 | YES |
| FUBP1 | ENSG00000162613 | 230 | 599 | 9.69E−25 | 1.91E−23 | 0.2774427 | YES |
| FUS | ENSG00000089280 | 57 | 129 | 5.48E−07 | 5.63E−06 | 0.30645161 | YES |
| FXN | ENSG00000165060 | 118 | 190 | 0.00013177 | 0.00119797 | 0.38311688 | YES |
| G3BP2 | ENSG00000138757 | 307 | 520 | 1.84E−09 | 2.10E−08 | 0.37122128 | YES |
| GAB2 | ENSG00000033327 | 183 | 14895 | 0 | 0 | 0.01213689 | YES |
| GABPA | ENSG00000154727 | 125 | 1249 | 1.79E−110 | 5.68E−108 | 0.09097525 | YES |
| GABPB1 | ENSG00000104064 | 250 | 1088 | 1.44E−64 | 1.21E−62 | 0.18684604 | YES |
| GADD45GIP1 | ENSG00000179271 | 95 | 292 | 1.36E−18 | 2.19E−17 | 0.24547804 | YES |
| GAPDH | ENSG00000111640 | 186 | 300 | 5.42E−06 | 5.36E−05 | 0.38271605 | YES |
| GAR1 | ENSG00000109534 | 134 | 275 | 7.71E−10 | 8.92E−09 | 0.32762836 | YES |
| GARS | ENSG00000106105 | 41 | 614 | 7.62E−76 | 9.51E−74 | 0.06259542 | YES |
| GART | ENSG00000159131 | 101 | 974 | 9.77E−93 | 1.92E−90 | 0.09395349 | YES |
| GATC | ENSG00000257218 | 84 | 223 | 8.48E−13 | 1.10E−11 | 0.27361564 | YES |
| GBF1 | ENSG00000107862 | 42 | 332 | 1.55E−39 | 5.13E−38 | 0.11229947 | YES |
| GEMIN2 | ENSG00000092208 | 17 | 194 | 3.34E−29 | 7.62E−28 | 0.08056872 | YES |
| GEMIN4 | ENSG00000179409 | 78 | 491 | 9.43E−48 | 4.24E−46 | 0.1370826 | YES |
| GEMIN5 | ENSG00000082516 | 20 | 239 | 6.21E−35 | 1.76E−33 | 0.07722008 | YES |
| GEMIN8 | ENSG00000046647 | 217 | 345 | 3.11E−06 | 3.10E−05 | 0.386121 | YES |
| GET4 | ENSG00000239857 | 22 | 111 | 1.94E−13 | 2.60E−12 | 0.16541353 | YES |
| GFER | ENSG00000127554 | 5 | 23 | 0.00043004 | 0.00378752 | 0.17857143 | YES |
| GFM1 | ENSG00000168827 | 38 | 141 | 5.25E−13 | 6.89E−12 | 0.2122905 | YES |
| GGPS1 | ENSG00000152904 | 86 | 328 | 3.86E−25 | 7.70E−24 | 0.20772947 | YES |
| GINS1 | ENSG00000101003 | 16 | 121 | 1.79E−17 | 2.77E−16 | 0.11678832 | YES |
| GINS2 | ENSG00000131153 | 3 | 98 | 1.10E−19 | 1.84E−18 | 0.02970297 | YES |
| GINS3 | ENSG00000181938 | 29 | 248 | 1.81E−32 | 4.65E−31 | 0.10469314 | YES |
| GLE1 | ENSG00000119392 | 10 | 215 | 6.43E−36 | 1.93E−34 | 0.04444444 | YES |
| GLTSCR2 | ENSG00000105373 | 10 | 208 | 8.39E−35 | 2.36E−33 | 0.04587156 | YES |
| GMNN | ENSG00000112312 | 48 | 177 | 2.28E−15 | 3.30E−14 | 0.21333333 | YES |
| GMPS | ENSG00000163655 | 19 | 313 | 2.04E−46 | 8.86E−45 | 0.05722892 | YES |
| GNB1L | ENSG00000185838 | 26 | 76 | 1.22E−06 | 1.24E−05 | 0.25490196 | YES |
| GNB2L1 | ENSG00000204628 | 92 | 410 | 1.19E−33 | 3.20E−32 | 0.18326693 | YES |
| GNL2 | ENSG00000134697 | 10 | 119 | 7.75E−20 | 1.30E−18 | 0.07751938 | YES |
| GNL3 | ENSG00000163938 | 29 | 557 | 4.92E−74 | 5.54E−72 | 0.04948805 | YES |
| GNL3L | ENSG00000130119 | 41 | 524 | 4.40E−65 | 3.73E−63 | 0.07256637 | YES |
| GOLT1B | ENSG00000111711 | 19 | 182 | 2.27E−26 | 4.71E−25 | 0.09452736 | YES |
| GON4L | ENSG00000116580 | 30 | 419 | 3.30E−56 | 1.98E−54 | 0.06681514 | YES |
| GOSR2 | ENSG00000108433 | 4 | 135 | 9.16E−26 | 1.85E−24 | 0.02877698 | YES |
| GPATCH1 | ENSG00000076650 | 110 | 209 | 4.44E−07 | 4.58E−06 | 0.34482759 | YES |
| GPKOW | ENSG00000068394 | 12 | 85 | 7.74E−13 | 1.01E−11 | 0.12371134 | YES |
| GPN1 | ENSG00000198522 | 12 | 258 | 1.34E−41 | 4.87E−40 | 0.04444444 | YES |
| GPN2 | ENSG00000142751 | 18 | 77 | 3.77E−09 | 4.25E−08 | 0.18947368 | YES |
| GPN3 | ENSG00000111231 | 101 | 1962 | 6.16E−172 | 4.89E−169 | 0.04895783 | YES |
| GRB2 | ENSG00000177885 | 509 | 4605 | 2.06E−219 | 4.49E−216 | 0.0995307 | YES |
| GRPEL1 | ENSG00000109519 | 3 | 162 | 5.90E−31 | 1.43E−29 | 0.01818182 | YES |
| GRWD1 | ENSG00000105447 | 23 | 153 | 5.59E−20 | 9.44E−19 | 0.13068182 | YES |
| GSPT1 | ENSG00000103342 | 48 | 389 | 4.65E−45 | 1.92E−43 | 0.10983982 | YES |
| GTF2A1 | ENSG00000165417 | 82 | 594 | 1.05E−58 | 7.13E−57 | 0.12130178 | YES |
| GTF2A2 | ENSG00000140307 | 38 | 158 | 1.56E−15 | 2.27E−14 | 0.19387755 | YES |
| GTF2B | ENSG00000137947 | 15 | 277 | 5.24E−43 | 2.01E−41 | 0.05136986 | YES |
| GTF2E1 | ENSG00000153767 | 130 | 543 | 3.98E−39 | 1.30E−37 | 0.19316493 | YES |
| GTF2F1 | ENSG00000125651 | 39 | 145 | 2.63E−13 | 3.48E−12 | 0.21195652 | YES |
| GTF2F2 | ENSG00000188342 | 49 | 590 | 6.57E−70 | 6.63E−68 | 0.07668232 | YES |
| GTF2H1 | ENSG00000110768 | 301 | 1130 | 6.05E−58 | 3.94E−56 | 0.21034242 | YES |
| GTF2H3 | ENSG00000111358 | 19 | 496 | 6.12E−71 | 6.44E−69 | 0.0368932 | YES |
| GTF3A | ENSG00000122034 | 35 | 719 | 4.49E−90 | 8.08E−88 | 0.0464191 | YES |
| GTF3C1 | ENSG00000077235 | 9 | 82 | 1.27E−13 | 1.71E−12 | 0.0989011 | YES |
| GTF3C3 | ENSG00000119041 | 10 | 157 | 2.06E−26 | 4.29E−25 | 0.05988024 | YES |
| GTF3C4 | ENSG00000125484 | 19 | 227 | 1.59E−33 | 4.24E−32 | 0.07723577 | YES |
| GTF3C5 | ENSG00000148308 | 15 | 195 | 2.76E−30 | 6.53E−29 | 0.07142857 | YES |
| GTF3C6 | ENSG00000155115 | 102 | 597 | 7.93E−53 | 4.16E−51 | 0.14592275 | YES |
| GTPBP10 | ENSG00000105793 | 271 | 448 | 4.22E−08 | 4.58E−07 | 0.37691238 | YES |
| GTPBP4 | ENSG00000107937 | 11 | 382 | 7.22E−60 | 5.04E−58 | 0.02798982 | YES |
| GTPBP5 | ENSG00000101181 | 343 | 563 | 4.60E−09 | 5.18E−08 | 0.3785872 | YES |
| GUK1 | ENSG00000143774 | 72 | 125 | 0.00025729 | 0.00229971 | 0.36548223 | YES |
| HARS | ENSG00000170445 | 3 | 68 | 5.17E−14 | 7.10E−13 | 0.04225352 | YES |
| HARS2 | ENSG00000112855 | 10 | 108 | 7.02E−18 | 1.11E−16 | 0.08474576 | YES |
| HAUS1 | ENSG00000152240 | 14 | 366 | 3.10E−56 | 1.87E−54 | 0.03684211 | YES |
| HAUS2 | ENSG00000137814 | 34 | 614 | 1.12E−78 | 1.53E−76 | 0.05246914 | YES |
| HAUS3 | ENSG00000214367 | 55 | 194 | 7.97E−16 | 1.17E−14 | 0.22088353 | YES |
| HAUS4 | ENSG00000092036 | 173 | 602 | 1.63E−35 | 4.77E−34 | 0.22322581 | YES |
| HAUS5 | ENSG00000249115 | 3 | 21 | 0.00015738 | 0.00142194 | 0.125 | YES |
| HAUS6 | ENSG00000147874 | 55 | 208 | 9.50E−18 | 1.49E−16 | 0.20912548 | YES |
| HAUS7 | ENSG00000213397 | 3 | 88 | 7.99E−18 | 1.26E−16 | 0.03296703 | YES |
| HAUS8 | ENSG00000131351 | 3 | 180 | 5.91E−34 | 1.61E−32 | 0.01639344 | YES |
| HBS1L | ENSG00000112339 | 24 | 184 | 1.39E−24 | 2.72E−23 | 0.11538462 | YES |
| HCFC1 | ENSG00000172534 | 261 | 657 | 5.73E−25 | 1.14E−23 | 0.28431373 | YES |
| HEATR1 | ENSG00000119285 | 34 | 97 | 1.10E−07 | 1.17E−06 | 0.25954198 | YES |
| HEATR3 | ENSG00000155393 | 39 | 111 | 1.95E−08 | 2.14E−07 | 0.26 | YES |
| HELQ | ENSG00000163312 | 135 | 291 | 3.49E−11 | 4.26E−10 | 0.31690141 | YES |
| HINFP | ENSG00000172273 | 20 | 85 | 8.12E−10 | 9.39E−09 | 0.19047619 | YES |
| HJURP | ENSG00000123485 | 3 | 32 | 8.23E−07 | 8.43E−06 | 0.08571429 | YES |
| HMGCR | ENSG00000113161 | 48 | 135 | 1.40E−09 | 1.61E−08 | 0.26229508 | YES |
| HMGCS1 | ENSG00000112972 | 27 | 139 | 3.24E−16 | 4.82E−15 | 0.1626506 | YES |
| HNRNPA1 | ENSG00000135486 | 160 | 368 | 1.17E−14 | 1.65E−13 | 0.3030303 | YES |
| HNRNPA2B1 | ENSG00000122566 | 147 | 317 | 7.47E−12 | 9.39E−11 | 0.31681034 | YES |
| HNRNPC | ENSG00000092199 | 30 | 694 | 2.21E−89 | 3.82E−87 | 0.04143646 | YES |
| HNRNPF | ENSG00000169813 | 1071 | 1445 | 5.40E−07 | 5.55E−06 | 0.42567568 | YES |
| HNRNPH1 | ENSG00000169045 | 19 | 82 | 1.19E−09 | 1.36E−08 | 0.18811881 | YES |
| HNRNPK | ENSG00000165119 | 56 | 245 | 1.40E−22 | 2.60E−21 | 0.18604651 | YES |
| HNRNPL | ENSG00000104824 | 21 | 543 | 8.13E−76 | 1.01E−73 | 0.03723404 | YES |
| HNRNPM | ENSG00000099783 | 120 | 381 | 2.34E−23 | 4.45E−22 | 0.23952096 | YES |
| HNRNPU | ENSG00000153187 | 8 | 87 | 5.07E−15 | 7.25E−14 | 0.08421053 | YES |
| HSCB | ENSG00000100209 | 6 | 28 | 0.00011212 | 0.00102255 | 0.17647059 | YES |
| HSD17B10 | ENSG00000072506 | 35 | 121 | 6.69E−11 | 8.09E−10 | 0.22435897 | YES |
| HSD17B12 | ENSG00000149084 | 126 | 1346 | 2.04E−118 | 7.93E−116 | 0.08559783 | YES |
| HSPA14 | ENSG00000187522 | 154 | 281 | 7.93E−08 | 8.49E−07 | 0.35402299 | YES |
| HSPA5 | ENSG00000044574 | 2 | 66 | 3.42E−14 | 4.74E−13 | 0.02941176 | YES |
| HSPA8 | ENSG00000109971 | 11 | 74 | 2.88E−11 | 3.53E−10 | 0.12941176 | YES |
| HSPA9 | ENSG00000113013 | 21 | 503 | 6.50E−71 | 6.80E−69 | 0.04007634 | YES |
| HSPD1 | ENSG00000144381 | 8 | 204 | 3.51E−35 | 1.01E−33 | 0.03773585 | YES |
| HSPE1 | ENSG00000115541 | 8 | 246 | 8.92E−42 | 3.26E−40 | 0.03149606 | YES |
| HTATSF1 | ENSG00000102241 | 11 | 60 | 1.04E−08 | 1.15E−07 | 0.15492958 | YES |
| HUS1 | ENSG00000136273 | 8 | 186 | 2.93E−32 | 7.45E−31 | 0.04123711 | YES |
| IARS | ENSG00000196305 | 296 | 784 | 3.14E−30 | 7.41E−29 | 0.27407407 | YES |
| IDI1 | ENSG00000067064 | 100 | 951 | 8.07E−91 | 1.47E−88 | 0.09514748 | YES |
| IGBP1 | ENSG00000089289 | 38 | 113 | 5.87E−09 | 6.59E−08 | 0.25165563 | YES |
| IK | ENSG00000113141 | 3 | 77 | 9.61E−16 | 1.41E−14 | 0.0375 | YES |
| IKBKAP | ENSG00000070061 | 45 | 107 | 1.32E−06 | 1.34E−05 | 0.29605263 | YES |
| ILF2 | ENSG00000143621 | 15 | 182 | 3.57E−28 | 7.90E−27 | 0.07614213 | YES |
| ILF3 | ENSG00000129351 | 91 | 466 | 9.82E−41 | 3.44E−39 | 0.16337522 | YES |
| IMMT | ENSG00000132305 | 8 | 724 | 3.61E−102 | 9.42E−100 | 0.01092896 | YES |
| IMP4 | ENSG00000136718 | 63 | 187 | 6.56E−13 | 8.58E−12 | 0.252 | YES |
| IMPDH2 | ENSG00000178035 | 19 | 123 | 1.39E−16 | 2.10E−15 | 0.13380282 | YES |
| INCENP | ENSG00000149503 | 7 | 30 | 0.00011023 | 0.00100585 | 0.18918919 | YES |
| INO80 | ENSG00000128908 | 136 | 1130 | 1.04E−96 | 2.28E−94 | 0.10742496 | YES |
| INO80C | ENSG00000153391 | 273 | 578 | 5.56E−17 | 8.50E−16 | 0.32079906 | YES |
| INSIG1 | ENSG00000186480 | 34 | 70 | 0.0003927 | 0.00347043 | 0.32692308 | YES |
| INTS12 | ENSG00000138785 | 55 | 202 | 6.37E−17 | 9.71E−16 | 0.21400778 | YES |
| INTS2 | ENSG00000108506 | 165 | 367 | 8.13E−14 | 1.10E−12 | 0.31015038 | YES |
| INTS3 | ENSG00000143624 | 187 | 271 | 0.00038836 | 0.00343602 | 0.40829694 | YES |
| INTS4 | ENSG00000149262 | 13 | 244 | 5.72E−39 | 1.86E−37 | 0.05058366 | YES |
| INTS5 | ENSG00000185085 | 24 | 95 | 3.75E−10 | 4.39E−09 | 0.20168067 | YES |
| INTS7 | ENSG00000143493 | 15 | 202 | 2.06E−31 | 5.09E−30 | 0.06912442 | YES |
| IPO11 | ENSG00000086200 | 135 | 1289 | 5.86E−111 | 1.93E−108 | 0.09480337 | YES |
| IPO7 | ENSG00000205339 | 20 | 343 | 3.12E−50 | 1.51E−48 | 0.05509642 | YES |
| IPO9 | ENSG00000198700 | 62 | 189 | 2.11E−13 | 2.82E−12 | 0.24701195 | YES |
| IPPK | ENSG00000127080 | 108 | 194 | 5.20E−06 | 5.15E−05 | 0.35761589 | YES |
| ISCA1 | ENSG00000135070 | 80 | 171 | 1.03E−07 | 1.10E−06 | 0.3187251 | YES |
| ISCU | ENSG00000136003 | 2 | 114 | 3.59E−23 | 6.76E−22 | 0.01724138 | YES |
| ISG20L2 | ENSG00000143319 | 44 | 348 | 5.35E−41 | 1.90E−39 | 0.1122449 | YES |
| ISY1 | ENSG00000240682 | 41 | 325 | 6.14E−39 | 1.99E−37 | 0.11202186 | YES |
| IWS1 | ENSG00000163166 | 293 | 399 | 0.00050639 | 0.00443317 | 0.4234104 | YES |
| KANSL1 | ENSG00000120071 | 370 | 1531 | 2.77E−76 | 3.55E−74 | 0.1946344 | YES |
| KANSL2 | ENSG00000139620 | 76 | 284 | 3.46E−22 | 6.35E−21 | 0.21111111 | YES |
| KANSL3 | ENSG00000114982 | 135 | 295 | 1.46E−11 | 1.81E−10 | 0.31395349 | YES |
| KARS | ENSG00000065427 | 12 | 434 | 2.72E−66 | 2.40E−64 | 0.02690583 | YES |
| KAT7 | ENSG00000136504 | 41 | 133 | 5.07E−11 | 6.15E−10 | 0.23563218 | YES |
| KAT8 | ENSG00000103510 | 5 | 264 | 4.46E−46 | 1.89E−44 | 0.01858736 | YES |
| KCMF1 | ENSG00000176407 | 496 | 805 | 1.01E−10 | 1.20E−09 | 0.3812452 | YES |
| KDM8 | ENSG00000155666 | 204 | 366 | 7.16E−09 | 8.02E−08 | 0.35789474 | YES |
| KIAA0391 | ENSG00000100890 | 469 | 849 | 4.47E−15 | 6.41E−14 | 0.35584219 | YES |
| KIAA0947 | ENSG00000164151 | 6 | 134 | 1.66E−24 | 3.24E−23 | 0.04285714 | YES |
| KIAA1731 | ENSG00000166004 | 3004 | 3599 | 8.28E−05 | 0.00076314 | 0.45494472 | YES |
| KIF11 | ENSG00000138160 | 13 | 551 | 2.12E−80 | 2.97E−78 | 0.02304965 | YES |
| KIF14 | ENSG00000118193 | 22 | 64 | 7.15E−06 | 7.00E−05 | 0.25581395 | YES |
| KIF18A | ENSG00000121621 | 36 | 785 | 9.88E−97 | 2.18E−94 | 0.04384896 | YES |
| KIF4A | ENSG00000090889 | 124 | 348 | 6.82E−19 | 1.11E−17 | 0.26271186 | YES |
| KIN | ENSG00000151657 | 42 | 199 | 2.32E−20 | 3.99E−19 | 0.17427386 | YES |
| KPNA4 | ENSG00000186432 | 137 | 201 | 0.00106716 | 0.00910503 | 0.40532544 | YES |
| KPNB1 | ENSG00000108424 | 56 | 612 | 7.54E−70 | 7.57E−68 | 0.08383234 | YES |
| KRI1 | ENSG00000129347 | 18 | 46 | 0.00035754 | 0.00317787 | 0.28125 | YES |
| KRR1 | ENSG00000111615 | 8 | 172 | 6.04E−30 | 1.41E−28 | 0.04444444 | YES |
| LAGE3 | ENSG00000196976 | 21 | 47 | 0.00117032 | 0.00995117 | 0.30882353 | YES |
| LAMTOR1 | ENSG00000149357 | 935 | 2162 | 1.95E−43 | 7.72E−42 | 0.30190507 | YES |
| LAMTOR2 | ENSG00000116586 | 131 | 305 | 3.69E−13 | 4.88E−12 | 0.30045872 | YES |
| LAMTOR5 | ENSG00000134248 | 83 | 207 | 3.79E−11 | 4.61E−10 | 0.2862069 | YES |
| LARS | ENSG00000133706 | 35 | 506 | 2.76E−65 | 2.37E−63 | 0.06469501 | YES |
| LARS2 | ENSG00000011376 | 215 | 473 | 4.89E−16 | 7.25E−15 | 0.3125 | YES |
| LAS1L | ENSG00000001497 | 8 | 81 | 6.57E−14 | 8.97E−13 | 0.08988764 | YES |
| LDLR | ENSG00000130164 | 650 | 946 | 5.61E−08 | 6.06E−07 | 0.40726817 | YES |
| LENG1 | ENSG00000105617 | 27 | 140 | 2.24E−16 | 3.35E−15 | 0.16167665 | YES |
| LENG8 | ENSG00000167615 | 52 | 107 | 2.41E−05 | 0.00022948 | 0.32704403 | YES |
| LENG9 | ENSG00000182909 | 7 | 33 | 3.01E−05 | 0.0002854 | 0.175 | YES |
| LEO1 | ENSG00000166477 | 10 | 344 | 4.01E−55 | 2.28E−53 | 0.02824859 | YES |
| LETM1 | ENSG00000168924 | 48 | 358 | 6.95E−41 | 2.46E−39 | 0.1182266 | YES |
| LIAS | ENSG00000121897 | 458 | 702 | 2.97E−08 | 3.25E−07 | 0.39482759 | YES |
| LIG1 | ENSG00000105486 | 73 | 238 | 4.61E−17 | 7.09E−16 | 0.23472669 | YES |
| LIN52 | ENSG00000205659 | 10 | 190 | 6.77E−32 | 1.70E−30 | 0.05 | YES |
| LIN54 | ENSG00000189308 | 130 | 885 | 3.30E−75 | 3.87E−73 | 0.12807882 | YES |
| LIN9 | ENSG00000183814 | 73 | 592 | 1.80E−61 | 1.33E−59 | 0.10977444 | YES |
| LONP1 | ENSG00000196365 | 62 | 384 | 1.82E−39 | 6.03E−38 | 0.13901345 | YES |
| LRPPRC | ENSG00000138095 | 153 | 348 | 7.47E−14 | 1.02E−12 | 0.30538922 | YES |
| LRR1 | ENSG00000165501 | 69 | 595 | 3.37E−63 | 2.68E−61 | 0.10391566 | YES |
| LRRFIP1 | ENSG00000124831 | 972 | 1215 | 0.00023261 | 0.00208549 | 0.44444444 | YES |
| LSG1 | ENSG00000041802 | 8 | 115 | 4.13E−20 | 7.02E−19 | 0.06504065 | YES |
| LSM10 | ENSG00000181817 | 113 | 297 | 1.77E−15 | 2.57E−14 | 0.27560976 | YES |
| LSM11 | ENSG00000155858 | 2 | 71 | 3.66E−15 | 5.26E−14 | 0.02739726 | YES |
| LSM2 | ENSG00000204392 | 9 | 36 | 4.41E−05 | 0.00041405 | 0.2 | YES |
| LSM3 | ENSG00000170860 | 6 | 300 | 7.12E−51 | 3.52E−49 | 0.01960784 | YES |
| LSM4 | ENSG00000130520 | 75 | 531 | 1.42E−53 | 7.65E−52 | 0.12376238 | YES |
| LSM5 | ENSG00000106355 | 1 | 183 | 1.40E−35 | 4.13E−34 | 0.00543478 | YES |
| LSM6 | ENSG00000164167 | 5 | 60 | 2.16E−11 | 2.66E−10 | 0.07692308 | YES |
| LTV1 | ENSG00000135521 | 2 | 173 | 2.33E−33 | 6.17E−32 | 0.01142857 | YES |
| LUC7L3 | ENSG00000108848 | 19 | 424 | 1.04E−61 | 7.73E−60 | 0.04288939 | YES |
| LYRM4 | ENSG00000214113 | 30 | 362 | 1.68E−48 | 7.75E−47 | 0.07653061 | YES |
| MAGOH | ENSG00000162385 | 6 | 64 | 1.15E−11 | 1.43E−10 | 0.08571429 | YES |
| MALSU1 | ENSG00000156928 | 60 | 127 | 3.07E−06 | 3.07E−05 | 0.32085561 | YES |
| MAPK14 | ENSG00000112062 | 794 | 1184 | 8.33E−10 | 9.62E−09 | 0.40141557 | YES |
| MARCH5 | ENSG00000198060 | 40 | 217 | 1.08E−23 | 2.07E−22 | 0.15564202 | YES |
| MARS | ENSG00000166986 | 13 | 335 | 2.17E−52 | 1.13E−50 | 0.03735632 | YES |
| MASTL | ENSG00000120539 | 11 | 348 | 3.44E−55 | 1.97E−53 | 0.03064067 | YES |
| MAT2A | ENSG00000168906 | 7 | 172 | 1.82E−30 | 4.36E−29 | 0.03910615 | YES |
| MATR3 | ENSG00000015479 | 1468 | 2121 | 2.42E−11 | 2.97E−10 | 0.40902758 | YES |
| MAU2 | ENSG00000129933 | 21 | 121 | 1.81E−15 | 2.62E−14 | 0.14788732 | YES |
| MAX | ENSG00000125952 | 44 | 702 | 1.25E−84 | 1.92E−82 | 0.05898123 | YES |
| MBNL1 | ENSG00000152601 | 1376 | 2114 | 1.64E−14 | 2.30E−13 | 0.39426934 | YES |
| MCL1 | ENSG00000143384 | 1 | 15 | 0.00029838 | 0.00266151 | 0.0625 | YES |
| MCM10 | ENSG00000065328 | 14 | 166 | 5.34E−26 | 1.09E−24 | 0.07777778 | YES |
| MCM2 | ENSG00000073111 | 277 | 785 | 2.82E−33 | 7.46E−32 | 0.26082863 | YES |
| MCM3 | ENSG00000112118 | 12 | 295 | 4.06E−47 | 1.78E−45 | 0.03908795 | YES |
| MCM5 | ENSG00000100297 | 33 | 310 | 6.00E−40 | 2.04E−38 | 0.09620991 | YES |
| MCM6 | ENSG00000076003 | 16 | 238 | 1.30E−36 | 3.98E−35 | 0.06299213 | YES |
| MCM7 | ENSG00000166508 | 15 | 156 | 7.25E−24 | 1.39E−22 | 0.0877193 | YES |
| MCMBP | ENSG00000197771 | 27 | 169 | 5.20E−21 | 9.13E−20 | 0.1377551 | YES |
| MCRS1 | ENSG00000187778 | 30 | 266 | 9.40E−35 | 2.64E−33 | 0.10135135 | YES |
| MCTS1 | ENSG00000232119 | 165 | 419 | 1.00E−18 | 1.62E−17 | 0.28253425 | YES |
| MDC1 | ENSG00000137337 | 463 | 732 | 2.32E−09 | 2.64E−08 | 0.3874477 | YES |
| MDN1 | ENSG00000112159 | 54 | 658 | 7.07E−76 | 8.89E−74 | 0.0758427 | YES |
| MED1 | ENSG00000125686 | 56 | 239 | 9.48E−22 | 1.72E−20 | 0.18983051 | YES |
| MED10 | ENSG00000133398 | 2 | 84 | 1.21E−17 | 1.88E−16 | 0.02325581 | YES |
| MED12 | ENSG00000184634 | 17 | 232 | 3.24E−35 | 9.35E−34 | 0.06827309 | YES |
| MED13 | ENSG00000108510 | 247 | 336 | 0.0010646 | 0.00908759 | 0.42367067 | YES |
| MED13L | ENSG00000123066 | 1173 | 2899 | 1.12E−56 | 6.80E−55 | 0.28806483 | YES |
| MED14 | ENSG00000180182 | 25 | 705 | 1.10E−92 | 2.12E−90 | 0.03424658 | YES |
| MED15 | ENSG00000099917 | 64 | 1642 | 4.16E−161 | 3.16E−158 | 0.03751465 | YES |
| MED17 | ENSG00000042429 | 29 | 114 | 1.35E−11 | 1.68E−10 | 0.2027972 | YES |
| MED18 | ENSG00000130772 | 122 | 660 | 4.42E−54 | 2.42E−52 | 0.15601023 | YES |
| MED19 | ENSG00000156603 | 3 | 29 | 3.43E−06 | 3.42E−05 | 0.09375 | YES |
| MED20 | ENSG00000124641 | 3 | 188 | 2.88E−35 | 8.33E−34 | 0.01570681 | YES |
| MED21 | ENSG00000152944 | 0 | 79 | 6.70E−18 | 1.06E−16 | 0 | YES |
| MED22 | ENSG00000148297 | 56 | 143 | 7.20E−09 | 8.06E−08 | 0.28140704 | YES |
| MED23 | ENSG00000112282 | 15 | 70 | 5.39E−09 | 6.05E−08 | 0.17647059 | YES |
| MED26 | ENSG00000105085 | 140 | 2612 | 5.96E−201 | 7.44E−198 | 0.05087209 | YES |
| MED27 | ENSG00000160563 | 14 | 207 | 1.10E−32 | 2.84E−31 | 0.06334842 | YES |
| MED28 | ENSG00000118579 | 10 | 305 | 1.45E−49 | 6.85E−48 | 0.03174603 | YES |
| MED29 | ENSG00000063322 | 10 | 63 | 1.15E−09 | 1.32E−08 | 0.1369863 | YES |
| MED31 | ENSG00000108590 | 4 | 132 | 3.07E−25 | 6.13E−24 | 0.02941176 | YES |
| MED4 | ENSG00000136146 | 6 | 120 | 4.92E−22 | 8.94E−21 | 0.04761905 | YES |
| MED6 | ENSG00000133997 | 4 | 81 | 5.96E−16 | 8.81E−15 | 0.04705882 | YES |
| MED7 | ENSG00000155868 | 31 | 84 | 1.43E−06 | 1.45E−05 | 0.26956522 | YES |
| MED8 | ENSG00000159479 | 1 | 65 | 1.37E−14 | 1.93E−13 | 0.01515152 | YES |
| MED9 | ENSG00000141026 | 3 | 134 | 3.78E−26 | 7.79E−25 | 0.02189781 | YES |
| METAP1 | ENSG00000164024 | 214 | 531 | 3.24E−21 | 5.72E−20 | 0.28724832 | YES |
| METAP2 | ENSG00000111142 | 29 | 282 | 1.59E−37 | 4.97E−36 | 0.09324759 | YES |
| METTL1 | ENSG00000037897 | 44 | 168 | 3.23E−15 | 4.66E−14 | 0.20754717 | YES |
| METTL14 | ENSG00000145388 | 7 | 61 | 1.30E−10 | 1.55E−09 | 0.10294118 | YES |
| METTL16 | ENSG00000127804 | 188 | 900 | 1.81E−61 | 1.33E−59 | 0.17279412 | YES |
| METTL3 | ENSG00000165819 | 36 | 320 | 3.58E−40 | 1.24E−38 | 0.1011236 | YES |
| MFAP1 | ENSG00000140259 | 8 | 302 | 3.88E−50 | 1.86E−48 | 0.02580645 | YES |
| MFN2 | ENSG00000116688 | 72 | 150 | 8.94E−07 | 9.15E−06 | 0.32432432 | YES |
| MGEA5 | ENSG00000198408 | 182 | 339 | 3.18E−09 | 3.59E−08 | 0.34932821 | YES |
| MINOS1 | ENSG00000173436 | 10 | 293 | 8.16E−48 | 3.69E−46 | 0.0330033 | YES |
| MIPEP | ENSG00000027001 | 117 | 244 | 2.30E−09 | 2.61E−08 | 0.32409972 | YES |
| MIS12 | ENSG00000167842 | 1 | 103 | 9.17E−22 | 1.66E−20 | 0.00961538 | YES |
| MIS18A | ENSG00000159055 | 5 | 161 | 1.10E−29 | 2.54E−28 | 0.03012048 | YES |
| MIS18BP1 | ENSG00000129534 | 417 | 1424 | 1.84E−60 | 1.31E−58 | 0.22650733 | YES |
| MKI67IP | ENSG00000155438 | 11 | 222 | 1.59E−36 | 4.82E−35 | 0.0472103 | YES |
| MMS19 | ENSG00000155229 | 54 | 254 | 1.92E−24 | 3.75E−23 | 0.17532468 | YES |
| MMS22L | ENSG00000146263 | 114 | 344 | 1.75E−20 | 3.01E−19 | 0.2489083 | YES |
| MNAT1 | ENSG00000020426 | 80 | 332 | 3.04E−27 | 6.49E−26 | 0.19417476 | YES |
| MNF1 | ENSG00000137288 | 234 | 342 | 8.66E−05 | 0.00079613 | 0.40625 | YES |
| MOB4 | ENSG00000115540 | 8 | 171 | 8.87E−30 | 2.05E−28 | 0.04469274 | YES |
| MPDU1 | ENSG00000129255 | 539 | 732 | 2.73E−05 | 0.00025994 | 0.42407553 | YES |
| MPHOSPH10 | ENSG00000124383 | 33 | 250 | 3.54E−31 | 8.62E−30 | 0.11660777 | YES |
| MPHOSPH6 | ENSG00000135698 | 26 | 134 | 9.10E−16 | 1.34E−14 | 0.1625 | YES |
| MRPL10 | ENSG00000159111 | 69 | 297 | 8.46E−26 | 1.72E−24 | 0.18852459 | YES |
| MRPL11 | ENSG00000174547 | 54 | 170 | 7.94E−13 | 1.04E−11 | 0.24107143 | YES |
| MRPL12 | ENSG00000262814 | 111 | 286 | 1.16E−14 | 1.64E−13 | 0.27959698 | YES |
| MRPL16 | ENSG00000166902 | 37 | 101 | 1.57E−07 | 1.65E−06 | 0.26811594 | YES |
| MRPL17 | ENSG00000158042 | 80 | 277 | 2.80E−20 | 4.80E−19 | 0.22408964 | YES |
| MRPL18 | ENSG00000112110 | 47 | 179 | 6.19E−16 | 9.12E−15 | 0.2079646 | YES |
| MRPL19 | ENSG00000115364 | 50 | 178 | 5.85E−15 | 8.36E−14 | 0.21929825 | YES |
| MRPL2 | ENSG00000112651 | 57 | 253 | 2.19E−23 | 4.15E−22 | 0.18387097 | YES |
| MRPL20 | ENSG00000242485 | 86 | 176 | 2.71E−07 | 2.83E−06 | 0.32824427 | YES |
| MRPL21 | ENSG00000197345 | 37 | 156 | 1.53E−15 | 2.23E−14 | 0.19170984 | YES |
| MRPL22 | ENSG00000082515 | 55 | 185 | 1.34E−14 | 1.89E−13 | 0.22916667 | YES |
| MRPL23 | ENSG00000214026 | 324 | 669 | 1.05E−17 | 1.64E−16 | 0.32628399 | YES |
| MRPL24 | ENSG00000143314 | 264 | 465 | 8.14E−10 | 9.40E−09 | 0.36213992 | YES |
| MRPL27 | ENSG00000108826 | 87 | 347 | 3.06E−27 | 6.52E−26 | 0.20046083 | YES |
| MRPL3 | ENSG00000114686 | 126 | 462 | 4.40E−31 | 1.06E−29 | 0.21428571 | YES |
| MRPL32 | ENSG00000106591 | 49 | 177 | 4.31E−15 | 6.18E−14 | 0.21681416 | YES |
| MRPL33 | ENSG00000243147 | 77 | 244 | 7.27E−17 | 1.10E−15 | 0.23987539 | YES |
| MRPL34 | ENSG00000130312 | 31 | 78 | 9.61E−06 | 9.37E−05 | 0.28440367 | YES |
| MRPL35 | ENSG00000132313 | 94 | 427 | 3.66E−35 | 1.05E−33 | 0.18042226 | YES |
| MRPL37 | ENSG00000116221 | 30 | 90 | 1.12E−07 | 1.19E−06 | 0.25 | YES |
| MRPL38 | ENSG00000204316 | 54 | 150 | 3.46E−10 | 4.05E−09 | 0.26470588 | YES |
| MRPL39 | ENSG00000154719 | 41 | 169 | 3.00E−16 | 4.46E−15 | 0.1952381 | YES |
| MRPL4 | ENSG00000105364 | 65 | 211 | 1.54E−15 | 2.25E−14 | 0.23550725 | YES |
| MRPL40 | ENSG00000185608 | 254 | 400 | 1.36E−06 | 1.38E−05 | 0.3883792 | YES |
| MRPL42 | ENSG00000198015 | 218 | 402 | 4.92E−10 | 5.72E−09 | 0.3516129 | YES |
| MRPL44 | ENSG00000135900 | 28 | 107 | 8.23E−11 | 9.89E−10 | 0.20740741 | YES |
| MRPL45 | ENSG00000174100 | 46 | 164 | 4.41E−14 | 6.07E−13 | 0.21904762 | YES |
| MRPL47 | ENSG00000136522 | 81 | 207 | 1.56E−11 | 1.93E−10 | 0.28125 | YES |
| MRPL48 | ENSG00000175581 | 738 | 1546 | 7.66E−30 | 1.78E−28 | 0.32311734 | YES |
| MRPL49 | ENSG00000149792 | 54 | 159 | 2.31E−11 | 2.83E−10 | 0.25352113 | YES |
| MRPL50 | ENSG00000136897 | 163 | 257 | 3.70E−05 | 0.00034887 | 0.38809524 | YES |
| MRPL51 | ENSG00000111639 | 51 | 120 | 4.89E−07 | 5.03E−06 | 0.29824561 | YES |
| MRPL52 | ENSG00000172590 | 63 | 242 | 3.64E−20 | 6.22E−19 | 0.20655738 | YES |
| MRPL54 | ENSG00000183617 | 105 | 274 | 1.77E−14 | 2.48E−13 | 0.27704485 | YES |
| MRPL9 | ENSG00000143436 | 30 | 119 | 4.60E−12 | 5.85E−11 | 0.20134228 | YES |
| MRPS10 | ENSG00000048544 | 72 | 302 | 1.33E−25 | 2.68E−24 | 0.19251337 | YES |
| MRPS12 | ENSG00000128626 | 111 | 182 | 0.00010587 | 0.0009681 | 0.37883959 | YES |
| MRPS14 | ENSG00000120333 | 27 | 137 | 6.76E−16 | 9.96E−15 | 0.16463415 | YES |
| MRPS15 | ENSG00000116898 | 30 | 99 | 5.22E−09 | 5.87E−08 | 0.23255814 | YES |
| MRPS16 | ENSG00000182180 | 136 | 201 | 0.0008911 | 0.0076552 | 0.40356083 | YES |
| MRPS17 | ENSG00000239789 | 41 | 108 | 1.43E−07 | 1.51E−06 | 0.27516779 | YES |
| MRPS18A | ENSG00000096080 | 33 | 120 | 2.55E−11 | 3.12E−10 | 0.21568627 | YES |
| MRPS18B | ENSG00000204568 | 326 | 825 | 3.88E−29 | 8.81E−28 | 0.28323197 | YES |
| MRPS18C | ENSG00000163319 | 58 | 215 | 6.97E−18 | 1.10E−16 | 0.21245421 | YES |
| MRPS23 | ENSG00000181610 | 66 | 204 | 2.16E−14 | 3.02E−13 | 0.24444444 | YES |
| MRPS24 | ENSG00000062582 | 35 | 103 | 2.74E−08 | 3.00E−07 | 0.25362319 | YES |
| MRPS25 | ENSG00000131368 | 68 | 244 | 4.30E−19 | 7.05E−18 | 0.21794872 | YES |
| MRPS27 | ENSG00000113048 | 595 | 1509 | 5.34E−42 | 1.98E−40 | 0.28279468 | YES |
| MRPS30 | ENSG00000112996 | 108 | 331 | 2.74E−20 | 4.70E−19 | 0.24601367 | YES |
| MRPS31 | ENSG00000102738 | 44 | 171 | 1.19E−15 | 1.75E−14 | 0.20465116 | YES |
| MRPS33 | ENSG00000090263 | 70 | 229 | 1.22E−16 | 1.84E−15 | 0.23411371 | YES |
| MRPS35 | ENSG00000061794 | 92 | 246 | 7.38E−14 | 1.00E−12 | 0.27218935 | YES |
| MRPS36 | ENSG00000134056 | 35 | 214 | 5.18E−25 | 1.03E−23 | 0.14056225 | YES |
| MRPS5 | ENSG00000144029 | 70 | 159 | 4.31E−08 | 4.68E−07 | 0.30567686 | YES |
| MRPS6 | ENSG00000243927 | 93 | 350 | 4.69E−26 | 9.60E−25 | 0.20993228 | YES |
| MRPS7 | ENSG00000125445 | 56 | 159 | 6.68E−11 | 8.09E−10 | 0.26046512 | YES |
| MRPS9 | ENSG00000135972 | 43 | 150 | 6.55E−13 | 8.58E−12 | 0.22279793 | YES |
| MRTO4 | ENSG00000053372 | 60 | 320 | 1.32E−31 | 3.27E−30 | 0.15789474 | YES |
| MSL1 | ENSG00000188895 | 192 | 333 | 1.05E−07 | 1.11E−06 | 0.36571429 | YES |
| MSL2 | ENSG00000174579 | 224 | 436 | 6.34E−12 | 8.01E−11 | 0.33939394 | YES |
| MTERFD2 | ENSG00000122085 | 53 | 96 | 0.00049339 | 0.00432582 | 0.3557047 | YES |
| MTG1 | ENSG00000148824 | 219 | 397 | 1.45E−09 | 1.67E−08 | 0.35551948 | YES |
| MTHFD1 | ENSG00000100714 | 43 | 729 | 5.60E−88 | 9.31E−86 | 0.05569948 | YES |
| MTOR | ENSG00000198793 | 13 | 140 | 4.50E−22 | 8.22E−21 | 0.08496732 | YES |
| MTPAP | ENSG00000107951 | 168 | 864 | 6.67E−63 | 5.20E−61 | 0.1627907 | YES |
| MVD | ENSG00000167508 | 8 | 82 | 4.28E−14 | 5.91E−13 | 0.08888889 | YES |
| MYBBPIA | ENSG00000132382 | 44 | 255 | 7.19E−28 | 1.57E−26 | 0.14715719 | YES |
| MYBL2 | ENSG00000101057 | 19 | 74 | 2.53E−08 | 2.78E−07 | 0.20430108 | YES |
| MZT1 | ENSG00000204899 | 25 | 222 | 3.82E−30 | 8.98E−29 | 0.10121457 | YES |
| N6AMT1 | ENSG00000156239 | 103 | 782 | 9.57E−73 | 1.05E−70 | 0.11638418 | YES |
| NAA10 | ENSG00000102030 | 14 | 75 | 3.05E−10 | 3.58E−09 | 0.15730337 | YES |
| NAA15 | ENSG00000164134 | 145 | 806 | 4.16E−63 | 3.29E−61 | 0.15247108 | YES |
| NAA20 | ENSG00000173418 | 29 | 173 | 6.81E−21 | 1.19E−19 | 0.14356436 | YES |
| NAA25 | ENSG00000111300 | 148 | 689 | 2.74E−50 | 1.34E−48 | 0.17682198 | YES |
| NAA30 | ENSG00000139977 | 16 | 131 | 3.45E−19 | 5.69E−18 | 0.10884354 | YES |
| NAA38 | ENSG00000128534 | 1 | 110 | 4.89E−23 | 9.20E−22 | 0.00900901 | YES |
| NAA50 | ENSG00000121579 | 62 | 414 | 2.27E−43 | 8.97E−42 | 0.1302521 | YES |
| NACA | ENSG00000196531 | 81 | 933 | 2.71E−95 | 5.57E−93 | 0.07988166 | YES |
| NAE1 | ENSG00000159593 | 12 | 364 | 6.39E−57 | 3.90E−55 | 0.03191489 | YES |
| NAF1 | ENSG00000145414 | 55 | 298 | 3.15E−30 | 7.42E−29 | 0.15580737 | YES |
| NAPA | ENSG00000105402 | 5 | 89 | 6.47E−17 | 9.85E−16 | 0.05319149 | YES |
| NAPG | ENSG00000134265 | 191 | 270 | 0.00082078 | 0.0070755 | 0.4143167 | YES |
| NARFL | ENSG00000103245 | 3 | 35 | 1.99E−07 | 2.08E−06 | 0.07894737 | YES |
| NARS | ENSG00000134440 | 44 | 478 | 3.69E−58 | 2.43E−56 | 0.08429119 | YES |
| NARS2 | ENSG00000137513 | 453 | 1150 | 6.48E−36 | 1.94E−34 | 0.28259513 | YES |
| NAT10 | ENSG00000135372 | 56 | 490 | 3.79E−55 | 2.17E−53 | 0.1025641 | YES |
| NBAS | ENSG00000151779 | 5048 | 11102 | 5.42E−77 | 7.01E−75 | 0.31256966 | YES |
| NCAPD2 | ENSG00000010292 | 39 | 230 | 5.73E−26 | 1.17E−24 | 0.14498141 | YES |
| NCAPG | ENSG00000109805 | 22 | 380 | 1.88E−54 | 1.04E−52 | 0.05472637 | YES |
| NCAPH | ENSG00000121152 | 33 | 604 | 6.32E−78 | 8.55E−76 | 0.05180534 | YES |
| NCBP1 | ENSG00000136937 | 13 | 314 | 2.16E−49 | 1.02E−47 | 0.03975535 | YES |
| NCBP2 | ENSG00000114503 | 4 | 257 | 1.49E−45 | 6.25E−44 | 0.01532567 | YES |
| NCL | ENSG00000115053 | 5 | 212 | 4.86E−38 | 1.55E−36 | 0.02304147 | YES |
| NCOA1 | ENSG00000084676 | 264 | 384 | 5.27E−05 | 0.00049215 | 0.40740741 | YES |
| NCOA2 | ENSG00000140396 | 2092 | 2489 | 0.00043411 | 0.00381949 | 0.45666885 | YES |
| NDC80 | ENSG00000080986 | 7 | 674 | 3.13E−97 | 7.20E−95 | 0.010279 | YES |
| NDUFA9 | ENSG00000139180 | 108 | 182 | 5.13E−05 | 0.00048019 | 0.37241379 | YES |
| NDUFAB1 | ENSG00000004779 | 10 | 125 | 6.79E−21 | 1.19E−19 | 0.07407407 | YES |
| NDUFB4 | ENSG00000065518 | 197 | 284 | 0.00036384 | 0.0032305 | 0.40956341 | YES |
| NDUFV2 | ENSG00000178127 | 1367 | 1814 | 4.35E−07 | 4.48E−06 | 0.42973908 | YES |
| NEDD1 | ENSG00000139350 | 25 | 124 | 1.67E−14 | 2.34E−13 | 0.16778523 | YES |
| NEDD8 | ENSG00000129559 | 12 | 123 | 1.35E−19 | 2.26E−18 | 0.08888889 | YES |
| NELFA | ENSG00000185049 | 98 | 312 | 2.75E−20 | 4.72E−19 | 0.23902439 | YES |
| NELFB | ENSG00000188986 | 14 | 64 | 2.64E−08 | 2.89E−07 | 0.17948718 | YES |
| NELFE | ENSG00000204356 | 26 | 65 | 4.63E−05 | 0.00043445 | 0.28571429 | YES |
| NFAT5 | ENSG00000102908 | 990 | 1322 | 2.14E−06 | 2.14E−05 | 0.42820069 | YES |
| NFS1 | ENSG00000244005 | 14 | 244 | 1.74E−38 | 5.58E−37 | 0.05426357 | YES |
| NFYA | ENSG00000001167 | 220 | 1222 | 8.05E−83 | 1.22E−80 | 0.15256588 | YES |
| NFYB | ENSG00000120837 | 17 | 225 | 4.00E−34 | 1.10E−32 | 0.07024793 | YES |
| NFYC | ENSG00000066136 | 171 | 3926 | 1.46E−257 | 5.11E−254 | 0.04173786 | YES |
| NGDN | ENSG00000129460 | 1 | 84 | 3.10E−18 | 4.94E−17 | 0.01176471 | YES |
| NGRN | ENSG00000182768 | 108 | 248 | 3.65E−11 | 4.45E−10 | 0.30337079 | YES |
| NHLRC2 | ENSG00000196865 | 157 | 759 | 3.17E−55 | 1.83E−53 | 0.17139738 | YES |
| NHP2 | ENSG00000145912 | 19 | 151 | 2.80E−21 | 4.97E−20 | 0.11176471 | YES |
| NHP2L1 | ENSG00000100138 | 20 | 774 | 3.53E−102 | 9.34E−100 | 0.02518892 | YES |
| NIP7 | ENSG00000132603 | 11 | 277 | 5.96E−45 | 2.44E−43 | 0.03819444 | YES |
| NIPBL | ENSG00000164190 | 144 | 1265 | 2.62E−106 | 7.63E−104 | 0.10220014 | YES |
| NKAP | ENSG00000101882 | 15 | 96 | 1.51E−13 | 2.03E−12 | 0.13513514 | YES |
| NLE1 | ENSG00000073536 | 6 | 170 | 1.17E−30 | 2.80E−29 | 0.03409091 | YES |
| NMD3 | ENSG00000169251 | 89 | 209 | 2.83E−10 | 3.32E−09 | 0.29865772 | YES |
| NMNAT1 | ENSG00000173614 | 209 | 667 | 1.18E−34 | 3.31E−33 | 0.23858447 | YES |
| NOB1 | ENSG00000141101 | 36 | 402 | 1.86E−51 | 9.37E−50 | 0.08219178 | YES |
| NOC3L | ENSG00000173145 | 10 | 169 | 1.98E−28 | 4.42E−27 | 0.05586592 | YES |
| NOC4L | ENSG00000184967 | 0 | 23 | 1.28E−06 | 1.30E−05 | 0 | YES |
| NOL10 | ENSG00000115761 | 37 | 199 | 4.91E−22 | 8.94E−21 | 0.15677966 | YES |
| NOL11 | ENSG00000130935 | 7 | 269 | 8.81E−46 | 3.73E−44 | 0.02536232 | YES |
| NOL12 | ENSG00000256872 | 19 | 442 | 4.58E−64 | 3.78E−62 | 0.04121475 | YES |
| NOL6 | ENSG00000165271 | 16 | 231 | 1.59E−35 | 4.67E−34 | 0.06477733 | YES |
| NOL7 | ENSG00000225921 | 6 | 41 | 3.35E−07 | 3.48E−06 | 0.12765957 | YES |
| NOL8 | ENSG00000198000 | 105 | 707 | 3.97E−64 | 3.29E−62 | 0.12931034 | YES |
| NOL9 | ENSG00000162408 | 12 | 95 | 1.23E−14 | 1.74E−13 | 0.11214953 | YES |
| NOLC1 | ENSG00000166197 | 127 | 598 | 5.91E−46 | 2.50E−44 | 0.17517241 | YES |
| NOM1 | ENSG00000146909 | 0 | 27 | 1.77E−07 | 1.86E−06 | 0 | YES |
| NONO | ENSG00000147140 | 244 | 402 | 1.56E−07 | 1.65E−06 | 0.37770898 | YES |
| NOP10 | ENSG00000182117 | 15 | 237 | 6.29E−37 | 1.93E−35 | 0.05952381 | YES |
| NOP14 | ENSG00000087269 | 23 | 441 | 3.90E−62 | 2.93E−60 | 0.04956897 | YES |
| NOP16 | ENSG00000048162 | 12 | 381 | 3.02E−59 | 2.09E−57 | 0.03053435 | YES |
| NOP2 | ENSG00000111641 | 68 | 224 | 1.73E−16 | 2.60E−15 | 0.23287671 | YES |
| NOP56 | ENSG00000101361 | 55 | 172 | 7.55E−13 | 9.86E−12 | 0.24229075 | YES |
| NOP58 | ENSG00000055044 | 51 | 563 | 6.16E−66 | 5.35E−64 | 0.08306189 | YES |
| NOP9 | ENSG00000196943 | 9 | 48 | 2.74E−07 | 2.86E−06 | 0.15789474 | YES |
| NPAT | ENSG00000149308 | 18 | 630 | 2.38E−87 | 3.89E−85 | 0.02777778 | YES |
| NPLOC4 | ENSG00000182446 | 20 | 151 | 7.33E−21 | 1.28E−19 | 0.11695906 | YES |
| NPM1 | ENSG00000181163 | 21 | 166 | 6.52E−23 | 1.22E−21 | 0.11229947 | YES |
| NRDE2 | ENSG00000119720 | 6 | 343 | 5.37E−57 | 3.30E−55 | 0.01719198 | YES |
| NRF1 | ENSG00000106459 | 24 | 459 | 5.07E−64 | 4.14E−62 | 0.04968944 | YES |
| NSA2 | ENSG00000164346 | 3 | 129 | 2.86E−25 | 5.71E−24 | 0.02272727 | YES |
| NSF | ENSG00000073969 | 5 | 110 | 8.99E−21 | 1.57E−19 | 0.04347826 | YES |
| NSL1 | ENSG00000117697 | 13 | 555 | 7.08E−81 | 1.00E−78 | 0.02288732 | YES |
| NSMCE1 | ENSG00000169189 | 1256 | 6953 | 8.48E−203 | 1.14E−199 | 0.1530028 | YES |
| NSMCE4A | ENSG00000107672 | 0 | 47 | 1.25E−11 | 1.55E−10 | 0 | YES |
| NSRP1 | ENSG00000126653 | 105 | 770 | 7.29E−71 | 7.54E−69 | 0.12 | YES |
| NSUN4 | ENSG00000117481 | 196 | 278 | 0.00065617 | 0.00569864 | 0.41350211 | YES |
| NUBP1 | ENSG00000103274 | 5 | 151 | 5.52E−28 | 1.21E−26 | 0.03205128 | YES |
| NUBP2 | ENSG00000095906 | 3 | 51 | 1.14E−10 | 1.36E−09 | 0.05555556 | YES |
| NUDC | ENSG00000090273 | 21 | 334 | 1.65E−48 | 7.65E−47 | 0.05915493 | YES |
| NUDCD3 | ENSG00000015676 | 40 | 362 | 1.89E−44 | 7.70E−43 | 0.09950249 | YES |
| NUDT21 | ENSG00000167005 | 20 | 218 | 1.14E−31 | 2.83E−30 | 0.08403361 | YES |
| NUF2 | ENSG00000143228 | 8 | 109 | 4.91E−19 | 8.05E−18 | 0.06837607 | YES |
| NUFIP1 | ENSG00000083635 | 59 | 273 | 1.57E−25 | 3.17E−24 | 0.17771084 | YES |
| NUP107 | ENSG00000111581 | 17 | 215 | 1.49E−32 | 3.85E−31 | 0.07327586 | YES |
| NUP133 | ENSG00000069248 | 11 | 131 | 1.84E−21 | 3.29E−20 | 0.07746479 | YES |
| NUP153 | ENSG00000124789 | 48 | 1146 | 7.48E−127 | 3.84E−124 | 0.04020101 | YES |
| NUP155 | ENSG00000113569 | 16 | 379 | 4.54E−57 | 2.80E−55 | 0.04050633 | YES |
| NUP160 | ENSG00000030066 | 17 | 340 | 3.41E−51 | 1.69E−49 | 0.04761905 | YES |
| NUP188 | ENSG00000095319 | 147 | 253 | 2.10E−06 | 2.11E−05 | 0.3675 | YES |
| NUP205 | ENSG00000155561 | 14 | 275 | 3.46E−43 | 1.35E−41 | 0.04844291 | YES |
| NUP214 | ENSG00000126883 | 94 | 493 | 4.29E−43 | 1.65E−41 | 0.16013629 | YES |
| NUP43 | ENSG00000120253 | 338 | 1531 | 2.71E−82 | 4.02E−80 | 0.18084537 | YES |
| NUP50 | ENSG00000093000 | 55 | 191 | 2.05E−15 | 2.96E−14 | 0.22357724 | YES |
| NUP54 | ENSG00000138750 | 6 | 203 | 4.56E−36 | 1.37E−34 | 0.02870813 | YES |
| NUP85 | ENSG00000125450 | 36 | 348 | 4.25E−44 | 1.71E−42 | 0.09375 | YES |
| NUP88 | ENSG00000108559 | 9 | 307 | 2.34E−50 | 1.15E−48 | 0.02848101 | YES |
| NUP93 | ENSG00000102900 | 17 | 297 | 5.11E−45 | 2.10E−43 | 0.05414013 | YES |
| NUP98 | ENSG00000110713 | 26 | 415 | 2.14E−57 | 1.36E−55 | 0.05895692 | YES |
| NUPL1 | ENSG00000139496 | 20 | 295 | 2.42E−43 | 9.53E−42 | 0.06349206 | YES |
| NUPL2 | ENSG00000136243 | 259 | 381 | 3.55E−05 | 0.00033527 | 0.4046875 | YES |
| NUS1 | ENSG00000153989 | 5 | 88 | 9.94E−17 | 1.51E−15 | 0.05376344 | YES |
| NUSAP1 | ENSG00000137804 | 688 | 952 | 1.70E−06 | 1.72E−05 | 0.4195122 | YES |
| NUTF2 | ENSG00000102898 | 9 | 185 | 1.39E−31 | 3.45E−30 | 0.04639175 | YES |
| NVL | ENSG00000143748 | 31 | 466 | 6.67E−62 | 5.00E−60 | 0.06237425 | YES |
| OGDH | ENSG00000105953 | 26 | 934 | 9.74E−116 | 3.62E−113 | 0.02708333 | YES |
| OGT | ENSG00000147162 | 261 | 1146 | 3.28E−67 | 3.03E−65 | 0.18550107 | YES |
| OIP5 | ENSG00000104147 | 24 | 297 | 7.47E−42 | 2.76E−40 | 0.07476636 | YES |
| OPA1 | ENSG00000198836 | 125 | 268 | 1.76E−10 | 2.08E−09 | 0.31806616 | YES |
| ORAOV1 | ENSG00000149716 | 3 | 51 | 1.14E−10 | 1.36E−09 | 0.05555556 | YES |
| ORC1 | ENSG00000085840 | 62 | 387 | 7.37E−40 | 2.49E−38 | 0.13808463 | YES |
| ORC2 | ENSG00000115942 | 48 | 221 | 1.20E−21 | 2.16E−20 | 0.17843866 | YES |
| ORC3 | ENSG00000135336 | 5 | 280 | 1.83E−48 | 8.41E−47 | 0.01754386 | YES |
| ORC4 | ENSG00000115947 | 187 | 296 | 1.21E−05 | 0.00011711 | 0.38716356 | YES |
| ORC5 | ENSG00000164815 | 5 | 230 | 7.13E−41 | 2.52E−39 | 0.0212766 | YES |
| ORC6 | ENSG00000091651 | 5 | 221 | 1.83E−39 | 6.06E−38 | 0.02212389 | YES |
| OSBP | ENSG00000110048 | 10 | 80 | 8.44E−13 | 1.10E−11 | 0.11111111 | YES |
| OSGEP | ENSG00000092094 | 82 | 290 | 2.06E−21 | 3.67E−20 | 0.22043011 | YES |
| OXA1L | ENSG00000155463 | 20 | 152 | 5.01E−21 | 8.81E−20 | 0.11627907 | YES |
| PA2G4 | ENSG00000170515 | 419 | 637 | 1.29E−07 | 1.36E−06 | 0.3967803 | YES |
| PABPC1 | ENSG00000070756 | 184 | 267 | 0.00040105 | 0.00354114 | 0.40798226 | YES |
| PABPN1 | ENSG00000100836 | 4 | 72 | 3.10E−14 | 4.30E−13 | 0.05263158 | YES |
| PAF1 | ENSG00000006712 | 6 | 63 | 1.79E−11 | 2.21E−10 | 0.08695652 | YES |
| PAFAH1B1 | ENSG00000007168 | 114 | 1836 | 1.89E−159 | 1.38E−156 | 0.05846154 | YES |
| PAICS | ENSG00000128050 | 38 | 956 | 4.10E−113 | 1.43E−110 | 0.03822938 | YES |
| PAK1IP1 | ENSG00000111845 | 21 | 290 | 3.71E−42 | 1.38E−40 | 0.06752412 | YES |
| PALB2 | ENSG00000083093 | 49 | 761 | 4.12E−89 | 6.99E−87 | 0.06049383 | YES |
| PAM16 | ENSG00000217930 | 10 | 151 | 2.15E−25 | 4.32E−24 | 0.0621118 | YES |
| PAPOLG | ENSG00000115421 | 102 | 388 | 2.31E−28 | 5.15E−27 | 0.20816327 | YES |
| PARN | ENSG00000140694 | 717 | 895 | 0.00076517 | 0.00661243 | 0.44478908 | YES |
| PARS2 | ENSG00000162396 | 58 | 135 | 1.66E−07 | 1.75E−06 | 0.30051813 | YES |
| PAXBP1 | ENSG00000159086 | 2 | 197 | 2.76E−37 | 8.54E−36 | 0.01005025 | YES |
| PCBP2 | ENSG00000197111 | 69 | 169 | 2.00E−09 | 2.28E−08 | 0.28991597 | YES |
| PCF11 | ENSG00000165494 | 19 | 247 | 1.30E−36 | 3.98E−35 | 0.07142857 | YES |
| PCID2 | ENSG00000126226 | 6 | 238 | 1.39E−41 | 5.03E−40 | 0.02459016 | YES |
| PCNA | ENSG00000132646 | 553 | 826 | 3.27E−08 | 3.57E−07 | 0.40101523 | YES |
| PCNP | ENSG00000081154 | 278 | 416 | 9.71E−06 | 9.46E−05 | 0.40057637 | YES |
| PCNT | ENSG00000160299 | 208 | 653 | 1.61E−33 | 4.28E−32 | 0.24157956 | YES |
| PCYT1A | ENSG00000161217 | 214 | 830 | 5.16E−49 | 2.42E−47 | 0.20498084 | YES |
| PDAP1 | ENSG00000106244 | 226 | 438 | 7.56E−12 | 9.50E−11 | 0.34036145 | YES |
| PDCD11 | ENSG00000148843 | 52 | 353 | 9.05E−39 | 2.93E−37 | 0.12839506 | YES |
| PDCD2 | ENSG00000071994 | 24 | 142 | 8.48E−18 | 1.33E−16 | 0.14457831 | YES |
| PDCL | ENSG00000136940 | 54 | 115 | 6.59E−06 | 6.48E−05 | 0.31952663 | YES |
| PDPK1 | ENSG00000140992 | 155 | 296 | 6.19E−09 | 6.94E−08 | 0.34368071 | YES |
| PDRG1 | ENSG00000088356 | 2 | 69 | 8.93E−15 | 1.27E−13 | 0.02816901 | YES |
| PDS5A | ENSG00000121892 | 272 | 716 | 2.97E−28 | 6.58E−27 | 0.27530364 | YES |
| PDSS1 | ENSG00000148459 | 144 | 584 | 3.63E−40 | 1.25E−38 | 0.1978022 | YES |
| PDSS2 | ENSG00000164494 | 269 | 746 | 2.94E−31 | 7.21E−30 | 0.26502463 | YES |
| PES1 | ENSG00000100029 | 4 | 315 | 4.44E−54 | 2.42E−52 | 0.01253918 | YES |
| PET112 | ENSG00000059691 | 50 | 115 | 1.27E−06 | 1.29E−05 | 0.3030303 | YES |
| PET117 | ENSG00000232838 | 80 | 313 | 7.88E−25 | 1.55E−23 | 0.20356234 | YES |
| PFAS | ENSG00000178921 | 318 | 931 | 9.61E−39 | 3.11E−37 | 0.25460368 | YES |
| PFDN1 | ENSG00000113068 | 407 | 610 | 4.39E−07 | 4.52E−06 | 0.40019666 | YES |
| PFDN2 | ENSG00000143256 | 16 | 539 | 1.40E−77 | 1.85E−75 | 0.02882883 | YES |
| PFDN5 | ENSG00000123349 | 122 | 199 | 6.99E−05 | 0.00064734 | 0.38006231 | YES |
| PFDNG | ENSG00000204220 | 13 | 65 | 7.59E−09 | 8.48E−08 | 0.16666667 | YES |
| PGAM1 | ENSG00000171314 | 177 | 496 | 3.88E−24 | 7.51E−23 | 0.26300149 | YES |
| PGD | ENSG00000142657 | 55 | 1209 | 7.88E−130 | 4.17E−127 | 0.04351266 | YES |
| PGGT1B | ENSG00000164219 | 32 | 352 | 2.79E−46 | 1.20E−44 | 0.08333333 | YES |
| PGK1 | ENSG00000102144 | 287 | 1067 | 2.29E−55 | 1.33E−53 | 0.21196455 | YES |
| PGM3 | ENSG00000013375 | 21 | 63 | 5.56E−06 | 5.49E−05 | 0.25 | YES |
| PGS1 | ENSG00000087157 | 309 | 1554 | 8.11E−90 | 1.43E−87 | 0.16586151 | YES |
| PHB | ENSG00000167085 | 2 | 140 | 9.19E−28 | 2.00E−26 | 0.01408451 | YES |
| PHF23 | ENSG00000040633 | 445 | 604 | 8.02E−05 | 0.00074077 | 0.42421354 | YES |
| PHF5A | ENSG00000100410 | 3 | 68 | 5.17E−14 | 7.10E−13 | 0.04225352 | YES |
| PIK3C3 | ENSG00000078142 | 93 | 527 | 2.04E−47 | 8.96E−46 | 0.15 | YES |
| PIK3R4 | ENSG00000196455 | 100 | 206 | 3.48E−08 | 3.79E−07 | 0.32679739 | YES |
| PLK1 | ENSG00000166851 | 4 | 61 | 4.18E−12 | 5.31E−11 | 0.06153846 | YES |
| PLK4 | ENSG00000142731 | 15 | 80 | 9.68E−11 | 1.15E−09 | 0.15789474 | YES |
| PLRG1 | ENSG00000171566 | 1 | 29 | 2.78E−07 | 2.90E−06 | 0.03333333 | YES |
| PMF1 | ENSG00000160783 | 20 | 230 | 1.53E−33 | 4.07E−32 | 0.08 | YES |
| PMM2 | ENSG00000140650 | 86 | 378 | 2.28E−31 | 5.59E−30 | 0.18534483 | YES |
| PMPCA | ENSG00000165688 | 7 | 133 | 8.33E−24 | 1.59E−22 | 0.05 | YES |
| PMPCB | ENSG00000105819 | 0 | 70 | 3.55E−16 | 5.28E−15 | 0 | YES |
| PMVK | ENSG00000163344 | 133 | 459 | 3.80E−29 | 8.65E−28 | 0.22466216 | YES |
| PNISR | ENSG00000132424 | 27 | 291 | 1.10E−39 | 3.70E−38 | 0.08490566 | YES |
| PNN | ENSG00000100941 | 15 | 323 | 1.01E−49 | 4.80E−48 | 0.0443787 | YES |
| PNO1 | ENSG00000115946 | 4 | 276 | 2.12E−48 | 9.70E−47 | 0.01428571 | YES |
| PNPT1 | ENSG00000138035 | 12 | 165 | 8.83E−27 | 1.86E−25 | 0.06779661 | YES |
| POLA1 | ENSG00000101868 | 24 | 669 | 3.90E−89 | 6.68E−87 | 0.03463203 | YES |
| POLA2 | ENSG00000014138 | 105 | 290 | 2.94E−16 | 4.38E−15 | 0.26582278 | YES |
| POLD1 | ENSG00000062822 | 63 | 231 | 1.08E−18 | 1.75E−17 | 0.21428571 | YES |
| POLD2 | ENSG00000106628 | 96 | 366 | 3.08E−27 | 6.56E−26 | 0.20779221 | YES |
| POLD3 | ENSG00000077514 | 10 | 259 | 9.61E−43 | 3.66E−41 | 0.03717472 | YES |
| POLE | ENSG00000177084 | 13 | 100 | 4.33E−15 | 6.20E−14 | 0.11504425 | YES |
| POLE2 | ENSG00000100479 | 24 | 585 | 1.64E−79 | 2.28E−77 | 0.03940887 | YES |
| POLG2 | ENSG00000256525 | 143 | 261 | 1.77E−07 | 1.86E−06 | 0.3539604 | YES |
| POLR1A | ENSG00000068654 | 50 | 845 | 1.62E−97 | 3.76E−95 | 0.05586592 | YES |
| POLR1B | ENSG00000125630 | 21 | 517 | 1.21E−72 | 1.31E−70 | 0.03903346 | YES |
| POLR1C | ENSG00000171453 | 5 | 360 | 7.30E−60 | 5.08E−58 | 0.01369863 | YES |
| POLR1E | ENSG00000137054 | 41 | 219 | 1.21E−23 | 2.30E−22 | 0.15769231 | YES |
| POLR2A | ENSG00000181222 | 5 | 363 | 2.83E−60 | 2.00E−58 | 0.01358696 | YES |
| POLR2B | ENSG00000047315 | 15 | 358 | 1.18E−54 | 6.61E−53 | 0.04021448 | YES |
| POLR2C | ENSG00000102978 | 17 | 93 | 3.06E−12 | 3.91E−11 | 0.15454545 | YES |
| POLR2D | ENSG00000144231 | 7 | 168 | 8.50E−30 | 1.97E−28 | 0.04 | YES |
| POLR2E | ENSG00000099817 | 2 | 88 | 2.14E−18 | 3.42E−17 | 0.02222222 | YES |
| POLR2F | ENSG00000100142 | 28 | 186 | 2.57E−23 | 4.86E−22 | 0.13084112 | YES |
| POLR2I | ENSG00000105258 | 1 | 13 | 0.00083217 | 0.00716888 | 0.07142857 | YES |
| POLR2L | ENSG00000177700 | 1 | 39 | 2.26E−09 | 2.57E−08 | 0.025 | YES |
| POLR3A | ENSG00000148606 | 17 | 171 | 1.86E−25 | 3.74E−24 | 0.09042553 | YES |
| POLR3B | ENSG00000013503 | 12 | 236 | 3.21E−38 | 1.03E−36 | 0.0483871 | YES |
| POLR3C | ENSG00000186141 | 32 | 214 | 3.97E−26 | 8.17E−25 | 0.1300813 | YES |
| POLR3E | ENSG00000058600 | 16 | 229 | 3.26E−35 | 9.38E−34 | 0.06530612 | YES |
| POLR3F | ENSG00000132664 | 10 | 84 | 1.56E−13 | 2.09E−12 | 0.10638298 | YES |
| POLR3H | ENSG00000100413 | 45 | 100 | 9.45E−06 | 9.22E−05 | 0.31034483 | YES |
| POLR3K | ENSG00000161980 | 48 | 303 | 2.69E−33 | 7.12E−32 | 0.13675214 | YES |
| POMP | ENSG00000132963 | 0 | 153 | 3.67E−31 | 8.93E−30 | 0 | YES |
| POP4 | ENSG00000105171 | 147 | 235 | 4.30E−05 | 0.00040439 | 0.38481675 | YES |
| POP7 | ENSG00000172336 | 75 | 147 | 5.20E−06 | 5.15E−05 | 0.33783784 | YES |
| PPA1 | ENSG00000180817 | 14 | 198 | 3.06E−31 | 7.49E−30 | 0.06603774 | YES |
| PPA2 | ENSG00000138777 | 243 | 454 | 3.68E−11 | 4.48E−10 | 0.34863702 | YES |
| PPAN | ENSG00000130810 | 36 | 612 | 1.26E−77 | 1.68E−75 | 0.05555556 | YES |
| PPAT | ENSG00000128059 | 47 | 856 | 8.92E−100 | 2.19E−97 | 0.05204873 | YES |
| PPIE | ENSG00000084072 | 7 | 159 | 2.79E−28 | 6.20E−27 | 0.04216867 | YES |
| PPIL1 | ENSG00000137168 | 431 | 555 | 0.00108831 | 0.00927642 | 0.43711968 | YES |
| PPIL2 | ENSG00000100023 | 37 | 321 | 6.41E−40 | 2.17E−38 | 0.10335196 | YES |
| PPIL4 | ENSG00000131013 | 34 | 213 | 3.15E−25 | 6.30E−24 | 0.13765182 | YES |
| PPME1 | ENSG00000214517 | 163 | 343 | 6.09E−12 | 7.70E−11 | 0.32213439 | YES |
| PPP1CB | ENSG00000213639 | 93 | 335 | 3.14E−24 | 6.11E−23 | 0.21728972 | YES |
| PPP1R10 | ENSG00000204569 | 54 | 393 | 2.07E−43 | 8.20E−42 | 0.12080537 | YES |
| PPP1R11 | ENSG00000204619 | 2 | 52 | 1.99E−11 | 2.45E−10 | 0.03703704 | YES |
| PPP1R15B | ENSG00000158615 | 25 | 69 | 7.32E−06 | 7.16E−05 | 0.26595745 | YES |
| PPP1R2 | ENSG00000184203 | 58 | 150 | 2.52E−09 | 2.85E−08 | 0.27884615 | YES |
| PPP1R7 | ENSG00000115685 | 44 | 166 | 6.28E−15 | 8.95E−14 | 0.20952381 | YES |
| PPP1R8 | ENSG00000117751 | 13 | 417 | 1.38E−63 | 1.11E−61 | 0.03023256 | YES |
| PPP2CA | ENSG00000113575 | 22 | 315 | 2.40E−45 | 9.96E−44 | 0.0652819 | YES |
| PPP2R1A | ENSG00000105568 | 2 | 209 | 3.31E−39 | 1.08E−37 | 0.00947867 | YES |
| PPP2R2A | ENSG00000221914 | 644 | 857 | 3.43E−05 | 0.00032474 | 0.4290473 | YES |
| PPP2R3C | ENSG00000092020 | 148 | 631 | 3.38E−44 | 1.37E−42 | 0.18998716 | YES |
| PPP2R4 | ENSG00000119383 | 1462 | 2102 | 4.65E−11 | 5.65E−10 | 0.41021324 | YES |
| PPP4C | ENSG00000149923 | 33 | 133 | 2.71E−13 | 3.59E−12 | 0.19879518 | YES |
| PPP6C | ENSG00000119414 | 61 | 196 | 1.48E−14 | 2.07E−13 | 0.23735409 | YES |
| PPRC1 | ENSG00000148840 | 329 | 843 | 4.10E−30 | 9.61E−29 | 0.28071672 | YES |
| PPWD1 | ENSG00000113593 | 6 | 149 | 4.19E−27 | 8.88E−26 | 0.03870968 | YES |
| PRC1 | ENSG00000198901 | 18 | 465 | 1.75E−67 | 1.64E−65 | 0.03726708 | YES |
| PRCC | ENSG00000143294 | 72 | 446 | 4.03E−44 | 1.63E−42 | 0.13899614 | YES |
| PRDM10 | ENSG00000170325 | 393 | 1576 | 1.92E−75 | 2.28E−73 | 0.1995937 | YES |
| PREB | ENSG00000138073 | 11 | 67 | 5.47E−10 | 6.36E−09 | 0.14102564 | YES |
| PRELID1 | ENSG00000169230 | 28 | 168 | 1.78E−20 | 3.07E−19 | 0.14285714 | YES |
| PRIM1 | ENSG00000198056 | 14 | 506 | 1.80E−74 | 2.05E−72 | 0.02692308 | YES |
| PRKRIR | ENSG00000137492 | 148 | 247 | 7.45E−06 | 7.28E−05 | 0.37468354 | YES |
| PRMT1 | ENSG00000126457 | 11 | 190 | 2.13E−31 | 5.24E−30 | 0.05472637 | YES |
| PRMT5 | ENSG00000100462 | 46 | 221 | 2.78E−22 | 5.13E−21 | 0.17228464 | YES |
| PRPF18 | ENSG00000165630 | 4 | 70 | 7.51E−14 | 1.02E−12 | 0.05405405 | YES |
| PRPF19 | ENSG00000110107 | 4 | 177 | 6.61E−33 | 1.72E−31 | 0.02209945 | YES |
| PRPF3 | ENSG00000117360 | 18 | 234 | 4.58E−35 | 1.31E−33 | 0.07142857 | YES |
| PRPF31 | ENSG00000105618 | 2 | 31 | 3.93E−07 | 4.07E−06 | 0.06060606 | YES |
| PRPF38A | ENSG00000134748 | 9 | 188 | 4.49E−32 | 1.13E−30 | 0.04568528 | YES |
| PRPF38B | ENSG00000134186 | 33 | 681 | 1.05E−86 | 1.70E−84 | 0.04621849 | YES |
| PRPF39 | ENSG00000185246 | 241 | 494 | 2.14E−14 | 2.99E−13 | 0.32789116 | YES |
| PRPF4 | ENSG00000136875 | 2 | 115 | 2.37E−23 | 4.49E−22 | 0.01709402 | YES |
| PRPF40A | ENSG00000196504 | 55 | 427 | 1.69E−47 | 7.49E−46 | 0.11410788 | YES |
| PRPF4B | ENSG00000112739 | 38 | 230 | 2.52E−26 | 5.21E−25 | 0.14179104 | YES |
| PRPF6 | ENSG00000101161 | 9 | 50 | 1.16E−07 | 1.24E−06 | 0.15254237 | YES |
| PRPF8 | ENSG00000174231 | 19 | 210 | 7.43E−31 | 1.79E−29 | 0.08296943 | YES |
| PSMA1 | ENSG00000129084 | 29 | 438 | 3.93E−59 | 2.70E−57 | 0.0620985 | YES |
| PSMA2 | ENSG00000106588 | 1 | 222 | 8.09E−42 | 2.97E−40 | 0.0044843 | YES |
| PSMA3 | ENSG00000100567 | 14 | 470 | 5.72E−70 | 5.81E−68 | 0.02892562 | YES |
| PSMA5 | ENSG00000143106 | 4 | 167 | 3.06E−31 | 7.49E−30 | 0.02339181 | YES |
| PSMA7 | ENSG00000101182 | 1 | 98 | 7.59E−21 | 1.32E−19 | 0.01010101 | YES |
| PSMB1 | ENSG00000008018 | 1 | 100 | 3.25E−21 | 5.74E−20 | 0.00990099 | YES |
| PSMB2 | ENSG00000126067 | 98 | 1279 | 2.24E−121 | 9.09E−119 | 0.07116921 | YES |
| PSMB3 | ENSG00000108294 | 7 | 68 | 6.05E−12 | 7.66E−11 | 0.09333333 | YES |
| PSMB4 | ENSG00000159377 | 1 | 46 | 8.32E−11 | 9.98E−10 | 0.0212766 | YES |
| PSMB5 | ENSG00000100804 | 109 | 291 | 1.38E−15 | 2.02E−14 | 0.2725 | YES |
| PSMB6 | ENSG00000142507 | 1 | 100 | 3.25E−21 | 5.74E−20 | 0.00990099 | YES |
| PSMB7 | ENSG00000136930 | 5 | 143 | 1.31E−26 | 2.75E−25 | 0.03378378 | YES |
| PSMC1 | ENSG00000100764 | 5 | 87 | 1.53E−16 | 2.30E−15 | 0.05434783 | YES |
| PSMC3 | ENSG00000165916 | 13 | 177 | 2.71E−28 | 6.02E−27 | 0.06842105 | YES |
| PSMC4 | ENSG00000013275 | 2 | 50 | 5.01E−11 | 6.08E−10 | 0.03846154 | YES |
| PSMC5 | ENSG00000087191 | 2 | 182 | 7.60E−35 | 2.15E−33 | 0.01086957 | YES |
| PSMC6 | ENSG00000100519 | 7 | 236 | 9.46E−41 | 3.33E−39 | 0.02880658 | YES |
| PSMD1 | ENSG00000173692 | 19 | 420 | 3.49E−61 | 2.53E−59 | 0.04328018 | YES |
| PSMD11 | ENSG00000108671 | 5 | 239 | 2.87E−42 | 1.07E−40 | 0.0204918 | YES |
| PSMD12 | ENSG00000197170 | 10 | 360 | 2.39E−57 | 1.51E−55 | 0.02702703 | YES |
| PSMD13 | ENSG00000185627 | 6 | 99 | 3.07E−18 | 4.91E−17 | 0.05714286 | YES |
| PSMD14 | ENSG00000115233 | 55 | 481 | 2.19E−54 | 1.21E−52 | 0.10261194 | YES |
| PSMD2 | ENSG00000175166 | 43 | 96 | 1.23E−05 | 0.00011876 | 0.30935252 | YES |
| PSMD3 | ENSG00000108344 | 1 | 46 | 8.32E−11 | 9.98E−10 | 0.0212766 | YES |
| PSMD4 | ENSG00000159352 | 29 | 150 | 2.91E−17 | 4.49E−16 | 0.16201117 | YES |
| PSMD6 | ENSG00000163636 | 3 | 56 | 1.16E−11 | 1.44E−10 | 0.05084746 | YES |
| PSMD7 | ENSG00000103035 | 5 | 65 | 2.32E−12 | 2.98E−11 | 0.07142857 | YES |
| PSMD8 | ENSG00000099341 | 5 | 137 | 1.45E−25 | 2.91E−24 | 0.03521127 | YES |
| PSMF1 | ENSG00000125818 | 206 | 294 | 0.00042194 | 0.00371806 | 0.412 | YES |
| PSMG1 | ENSG00000183527 | 44 | 257 | 3.70E−28 | 8.16E−27 | 0.1461794 | YES |
| PSMG2 | ENSG00000128789 | 51 | 551 | 1.71E−64 | 1.43E−62 | 0.08471761 | YES |
| PSMG4 | ENSG00000180822 | 2 | 122 | 1.33E−24 | 2.60E−23 | 0.01612903 | YES |
| PSTK | ENSG00000179988 | 48 | 224 | 4.45E−22 | 8.13E−21 | 0.17647059 | YES |
| PTAR1 | ENSG00000188647 | 109 | 285 | 6.30E−15 | 8.97E−14 | 0.27664975 | YES |
| PTCD3 | ENSG00000132300 | 137 | 419 | 6.10E−24 | 1.17E−22 | 0.24640288 | YES |
| PTMA | ENSG00000187514 | 42 | 115 | 2.78E−08 | 3.04E−07 | 0.26751592 | YES |
| PTP4A1 | ENSG00000112245 | 193 | 303 | 1.38E−05 | 0.00013297 | 0.3891129 | YES |
| PTPMT1 | ENSG00000110536 | 88 | 322 | 6.95E−24 | 1.33E−22 | 0.21463415 | YES |
| PTTG1 | ENSG00000164611 | 10 | 99 | 2.91E−16 | 4.35E−15 | 0.09174312 | YES |
| PUM1 | ENSG00000134644 | 728 | 982 | 7.05E−06 | 6.91E−05 | 0.42573099 | YES |
| PWP1 | ENSG00000136045 | 7 | 120 | 1.64E−21 | 2.94E−20 | 0.05511811 | YES |
| PWP2 | ENSG00000241945 | 8 | 97 | 7.37E−17 | 1.12E−15 | 0.07619048 | YES |
| PYROXD1 | ENSG00000121350 | 107 | 593 | 6.54E−51 | 3.24E−49 | 0.15285714 | YES |
| QARS | ENSG00000172053 | 53 | 197 | 8.82E−17 | 1.34E−15 | 0.212 | YES |
| QRSL1 | ENSG00000130348 | 68 | 310 | 8.31E−28 | 1.81E−26 | 0.17989418 | YES |
| RABGGTB | ENSG00000137955 | 9 | 496 | 1.35E−75 | 1.65E−73 | 0.01782178 | YES |
| RAC1 | ENSG00000136238 | 484 | 627 | 0.00054021 | 0.00471976 | 0.43564356 | YES |
| RACGAP1 | ENSG00000161800 | 19 | 207 | 2.22E−30 | 5.27E−29 | 0.0840708 | YES |
| RAD1 | ENSG00000113456 | 72 | 462 | 4.03E−46 | 1.72E−44 | 0.13483146 | YES |
| RAD17 | ENSG00000152942 | 38 | 237 | 2.30E−27 | 4.93E−26 | 0.13818182 | YES |
| RAD23B | ENSG00000119318 | 118 | 197 | 3.89E−05 | 0.00036623 | 0.37460317 | YES |
| RAD50 | ENSG00000113522 | 166 | 860 | 5.13E−63 | 4.04E−61 | 0.16179337 | YES |
| RAD51 | ENSG00000051180 | 56 | 376 | 1.87E−40 | 6.51E−39 | 0.12962963 | YES |
| RAD51AP1 | ENSG00000111247 | 161 | 290 | 9.45E−08 | 1.01E−06 | 0.35698448 | YES |
| RAD51B | ENSG00000182185 | 1189 | 1881 | 2.74E−15 | 3.96E−14 | 0.38729642 | YES |
| RAD51C | ENSG00000108384 | 9 | 235 | 1.45E−39 | 4.82E−38 | 0.03688525 | YES |
| RAE1 | ENSG00000101146 | 5 | 69 | 3.94E−13 | 5.21E−12 | 0.06756757 | YES |
| RAN | ENSG00000132341 | 1 | 18 | 6.51E−05 | 0.00060439 | 0.05263158 | YES |
| RANBP2 | ENSG00000153201 | 38 | 117 | 1.60E−09 | 1.83E−08 | 0.24516129 | YES |
| RANBP3 | ENSG00000031823 | 237 | 641 | 2.51E−27 | 5.37E−26 | 0.26993166 | YES |
| RANGAP1 | ENSG00000100401 | 16 | 146 | 9.80E−22 | 1.77E−20 | 0.09876543 | YES |
| RAPGEF6 | ENSG00000158987 | 1735 | 2302 | 8.43E−08 | 9.02E−07 | 0.42977459 | YES |
| RARS | ENSG00000113643 | 6 | 44 | 8.71E−08 | 9.30E−07 | 0.12 | YES |
| RARS2 | ENSG00000146282 | 30 | 316 | 4.88E−42 | 1.81E−40 | 0.0867052 | YES |
| RBBP4 | ENSG00000162521 | 20 | 466 | 1.06E−66 | 9.52E−65 | 0.04115226 | YES |
| RBBP5 | ENSG00000117222 | 6 | 73 | 2.20E−13 | 2.92E−12 | 0.07594937 | YES |
| RBBP6 | ENSG00000122257 | 8 | 110 | 3.25E−19 | 5.36E−18 | 0.06779661 | YES |
| RBBP8 | ENSG00000101773 | 73 | 586 | 9.04E−61 | 6.53E−59 | 0.1107739 | YES |
| RBM10 | ENSG00000182872 | 201 | 396 | 1.66E−11 | 2.05E−10 | 0.33668342 | YES |
| RBM12 | ENSG00000244462 | 154 | 400 | 9.59E−19 | 1.55E−17 | 0.27797834 | YES |
| RBM14 | ENSG00000239306 | 7 | 59 | 3.14E−10 | 3.69E−09 | 0.10606061 | YES |
| RBM17 | ENSG00000134453 | 51 | 438 | 2.22E−50 | 1.09E−48 | 0.10429448 | YES |
| RBM19 | ENSG00000122965 | 11 | 145 | 7.05E−24 | 1.35E−22 | 0.07051282 | YES |
| RBM22 | ENSG00000086589 | 6 | 90 | 1.41E−16 | 2.12E−15 | 0.0625 | YES |
| RBM25 | ENSG00000119707 | 38 | 283 | 4.22E−34 | 1.16E−32 | 0.11838006 | YES |
| RBM28 | ENSG00000106344 | 462 | 3487 | 2.29E−177 | 2.00E−174 | 0.11699164 | YES |
| RBM33 | ENSG00000184863 | 1039 | 1387 | 1.60E−06 | 1.62E−05 | 0.428277 | YES |
| RBM39 | ENSG00000131051 | 77 | 517 | 3.14E−51 | 1.57E−49 | 0.12962963 | YES |
| RBM42 | ENSG00000126254 | 14 | 42 | 0.00014216 | 0.00128909 | 0.25 | YES |
| RBM48 | ENSG00000127993 | 4 | 109 | 3.89E−21 | 6.85E−20 | 0.03539823 | YES |
| RBM8A | ENSG00000131795 | 1 | 56 | 8.14E−13 | 1.06E−11 | 0.01754386 | YES |
| RBMX | ENSG00000147274 | 61 | 396 | 2.26E−41 | 8.15E−40 | 0.13347921 | YES |
| RBMX2 | ENSG00000134597 | 2 | 59 | 8.07E−13 | 1.05E−11 | 0.03278689 | YES |
| RBPJ | ENSG00000168214 | 490 | 634 | 0.00054462 | 0.00475592 | 0.43594306 | YES |
| RBX1 | ENSG00000100387 | 15 | 659 | 5.99E−92 | 1.14E−89 | 0.02225519 | YES |
| RCC1 | ENSG00000180198 | 1099 | 3126 | 2.61E−73 | 2.91E−71 | 0.26011834 | YES |
| RCL1 | ENSG00000120158 | 85 | 591 | 2.19E−57 | 1.39E−55 | 0.12573964 | YES |
| REV3L | ENSG00000009413 | 437 | 985 | 1.15E−26 | 2.41E−25 | 0.30731364 | YES |
| RFC1 | ENSG00000035928 | 13 | 706 | 5.36E−98 | 1.26E−95 | 0.01808067 | YES |
| RFC2 | ENSG00000049541 | 12 | 364 | 6.39E−57 | 3.90E−55 | 0.03191489 | YES |
| RFC3 | ENSG00000133119 | 8 | 260 | 6.55E−44 | 2.63E−42 | 0.02985075 | YES |
| RFC4 | ENSG00000163918 | 12 | 269 | 2.93E−43 | 1.15E−41 | 0.04270463 | YES |
| RFC5 | ENSG00000111445 | 9 | 65 | 1.83E−10 | 2.16E−09 | 0.12162162 | YES |
| RFT1 | ENSG00000163933 | 120 | 646 | 4.11E−53 | 2.17E−51 | 0.15665796 | YES |
| RFWD3 | ENSG00000168411 | 152 | 343 | 1.56E−13 | 2.08E−12 | 0.30707071 | YES |
| RHOA | ENSG00000067560 | 58 | 689 | 7.12E−78 | 9.56E−76 | 0.07764391 | YES |
| RIF1 | ENSG00000080345 | 96 | 249 | 2.04E−13 | 2.72E−12 | 0.27826087 | YES |
| RINT1 | ENSG00000135249 | 40 | 175 | 1.91E−17 | 2.95E−16 | 0.18604651 | YES |
| RIOK1 | ENSG00000124784 | 16 | 128 | 1.12E−18 | 1.82E−17 | 0.11111111 | YES |
| RIOK2 | ENSG00000058729 | 5 | 157 | 5.24E−29 | 1.19E−27 | 0.0308642 | YES |
| RMI1 | ENSG00000178966 | 555 | 1192 | 3.65E−27 | 7.74E−26 | 0.31768746 | YES |
| RNASEH2A | ENSG00000104889 | 31 | 77 | 1.31E−05 | 0.00012684 | 0.28703704 | YES |
| RNASEH2B | ENSG00000136104 | 612 | 1598 | 1.97E−45 | 8.22E−44 | 0.27692308 | YES |
| RNF168 | ENSG00000163961 | 797 | 1178 | 1.88E−09 | 2.14E−08 | 0.4035443 | YES |
| RNF20 | ENSG00000155827 | 3 | 38 | 4.83E−08 | 5.23E−07 | 0.07317073 | YES |
| RNF4 | ENSG00000063978 | 69 | 522 | 1.67E−54 | 9.27E−53 | 0.11675127 | YES |
| RNF40 | ENSG00000103549 | 22 | 75 | 1.43E−07 | 1.51E−06 | 0.22680412 | YES |
| RNF8 | ENSG00000112130 | 72 | 194 | 8.29E−12 | 1.04E−10 | 0.27067669 | YES |
| RNGTT | ENSG00000111880 | 53 | 488 | 5.34E−56 | 3.17E−54 | 0.09796673 | YES |
| RNMT | ENSG00000101654 | 56 | 311 | 1.10E−31 | 2.75E−30 | 0.15258856 | YES |
| RNPC3 | ENSG00000185946 | 21 | 177 | 1.06E−24 | 2.08E−23 | 0.10606061 | YES |
| RNPS1 | ENSG00000205937 | 12 | 154 | 6.27E−25 | 1.24E−23 | 0.07228916 | YES |
| RP11-93B14.6 | ENSG00000167046 | 40 | 75 | 0.00099662 | 0.00852398 | 0.34782609 | YES |
| RPA1 | ENSG00000132383 | 15 | 194 | 4.00E−30 | 9.39E−29 | 0.07177033 | YES |
| RPA2 | ENSG00000117748 | 15 | 59 | 4.22E−07 | 4.36E−06 | 0.2027027 | YES |
| RPAIN | ENSG00000129197 | 6 | 245 | 1.16E−42 | 4.37E−41 | 0.02390438 | YES |
| RPAP1 | ENSG00000103932 | 24 | 414 | 3.85E−58 | 2.52E−56 | 0.05479452 | YES |
| RPAP3 | ENSG00000005175 | 30 | 623 | 2.23E−81 | 3.19E−79 | 0.04594181 | YES |
| RPE | ENSG00000197713 | 26 | 150 | 2.40E−18 | 3.85E−17 | 0.14772727 | YES |
| RPF1 | ENSG00000117133 | 5 | 82 | 1.33E−15 | 1.94E−14 | 0.05747126 | YES |
| RPF2 | ENSG00000197498 | 22 | 368 | 8.29E−53 | 4.33E−51 | 0.05641026 | YES |
| RPIA | ENSG00000153574 | 99 | 198 | 1.49E−07 | 1.57E−06 | 0.33333333 | YES |
| RPL10A | ENSG00000198755 | 2 | 87 | 3.29E−18 | 5.24E−17 | 0.02247191 | YES |
| RPL11 | ENSG00000142676 | 5 | 400 | 2.90E−65 | 2.47E−63 | 0.01234568 | YES |
| RPL12 | ENSG00000197958 | 4 | 126 | 3.50E−24 | 6.79E−23 | 0.03076923 | YES |
| RPL13 | ENSG00000167526 | 6 | 56 | 4.02E−10 | 4.70E−09 | 0.09677419 | YES |
| RPL13A | ENSG00000142541 | 14 | 840 | 1.70E−111 | 5.73E−109 | 0.01639344 | YES |
| RPL14 | ENSG00000188846 | 5 | 159 | 2.40E−29 | 5.50E−28 | 0.0304878 | YES |
| RPL15 | ENSG00000174748 | 1 | 168 | 4.22E−33 | 1.11E−31 | 0.00591716 | YES |
| RPL17 | ENSG00000265681 | 5 | 371 | 2.29E−61 | 1.67E−59 | 0.01329787 | YES |
| RPL18 | ENSG00000063177 | 4 | 341 | 9.47E−58 | 6.13E−56 | 0.0115942 | YES |
| RPL18A | ENSG00000105640 | 6 | 486 | 8.30E−76 | 1.02E−73 | 0.01219512 | YES |
| RPL19 | ENSG00000108298 | 6 | 420 | 2.14E−67 | 1.99E−65 | 0.01408451 | YES |
| RPL21 | ENSG00000122026 | 20 | 806 | 1.71E−105 | 4.83E−103 | 0.02421308 | YES |
| RPL22 | ENSG00000116251 | 256 | 418 | 1.63E−07 | 1.72E−06 | 0.37982196 | YES |
| RPL22L1 | ENSG00000163584 | 382 | 558 | 3.60E−06 | 3.58E−05 | 0.40638298 | YES |
| RPL23 | ENSG00000125691 | 5 | 277 | 5.09E−48 | 2.32E−46 | 0.0177305 | YES |
| RPL23A | ENSG00000198242 | 107 | 509 | 2.73E−41 | 9.82E−40 | 0.1737013 | YES |
| RPL24 | ENSG00000114391 | 3 | 396 | 9.38E−66 | 8.11E−64 | 0.0075188 | YES |
| RPL26 | ENSG00000161970 | 5 | 414 | 4.11E−67 | 3.76E−65 | 0.01193317 | YES |
| RPL27 | ENSG00000131469 | 6 | 162 | 2.57E−29 | 5.89E−28 | 0.03571429 | YES |
| RPL27A | ENSG00000166441 | 73 | 279 | 2.46E−22 | 4.54E−21 | 0.20738636 | YES |
| RPL28 | ENSG00000108107 | 8 | 41 | 2.27E−06 | 2.27E−05 | 0.16326531 | YES |
| RPL29 | ENSG00000162244 | 216 | 546 | 3.05E−22 | 5.61E−21 | 0.28346457 | YES |
| RPL3 | ENSG00000100316 | 6 | 268 | 3.76E−46 | 1.61E−44 | 0.02189781 | YES |
| RPL31 | ENSG00000071082 | 5 | 275 | 1.01E−47 | 4.53E−46 | 0.01785714 | YES |
| RPL32 | ENSG00000144713 | 1 | 269 | 5.49E−49 | 2.57E−47 | 0.0037037 | YES |
| RPL34 | ENSG00000109475 | 1 | 423 | 8.57E−69 | 8.45E−67 | 0.00936768 | YES |
| RPL35 | ENSG00000136942 | 2 | 200 | 9.08E−38 | 2.87E−36 | 0.00990099 | YES |
| RPL37 | ENSG00000145592 | 6 | 423 | 8.67E−68 | 8.27E−66 | 0.01398601 | YES |
| RPL37A | ENSG00000197756 | 187 | 455 | 1.51E−18 | 2.43E−17 | 0.29127726 | YES |
| RPL38 | ENSG00000172809 | 4 | 139 | 1.84E−26 | 3.84E−25 | 0.02797203 | YES |
| RPL39 | ENSG00000198918 | 12 | 441 | 3.36E−67 | 3.09E−65 | 0.02649007 | YES |
| RPL4 | ENSG00000174444 | 782 | 1156 | 2.27E−09 | 2.58E−08 | 0.40350877 | YES |
| RPL41 | ENSG00000229117 | 232 | 351 | 2.00E−05 | 0.00019183 | 0.39794168 | YES |
| RPL5 | ENSG00000122406 | 6 | 223 | 3.02E−39 | 9.95E−38 | 0.02620087 | YES |
| RPL6 | ENSG00000089009 | 4 | 389 | 2.62E−64 | 2.18E−62 | 0.01017812 | YES |
| RPL7A | ENSG00000148303 | 2 | 169 | 1.08E−32 | 2.79E−31 | 0.01169591 | YES |
| RPL7L1 | ENSG00000146223 | 93 | 466 | 3.74E−40 | 1.28E−38 | 0.16636852 | YES |
| RPL9 | ENSG00000163682 | 9 | 495 | 1.80E−75 | 2.15E−73 | 0.01785714 | YES |
| RPLP0 | ENSG00000089157 | 7 | 205 | 7.33E−36 | 2.19E−34 | 0.03301887 | YES |
| RPLP1 | ENSG00000137818 | 114 | 198 | 1.20E−05 | 0.00011609 | 0.36538462 | YES |
| RPLP2 | ENSG00000177600 | 53 | 121 | 8.84E−07 | 9.05E−06 | 0.3045977 | YES |
| RPN1 | ENSG00000163902 | 23 | 426 | 3.58E−60 | 2.52E−58 | 0.05122494 | YES |
| RPN2 | ENSG00000118705 | 47 | 490 | 1.64E−58 | 1.10E−56 | 0.08752328 | YES |
| RPP14 | ENSG00000163684 | 22 | 138 | 6.58E−18 | 1.04E−16 | 0.1375 | YES |
| RPP30 | ENSG00000148688 | 10 | 88 | 2.90E−14 | 4.02E−13 | 0.10204082 | YES |
| RPP38 | ENSG00000152464 | 55 | 229 | 1.17E−20 | 2.04E−19 | 0.19366197 | YES |
| RPP40 | ENSG00000124787 | 2 | 35 | 5.77E−08 | 6.22E−07 | 0.05405405 | YES |
| RPRD1B | ENSG00000101413 | 157 | 440 | 3.16E−22 | 5.80E−21 | 0.26298157 | YES |
| RPRD2 | ENSG00000163125 | 843 | 1837 | 1.54E−35 | 4.52E−34 | 0.31455224 | YES |
| RPS10 | ENSG00000124614 | 20 | 296 | 1.72E−43 | 6.86E−42 | 0.06329114 | YES |
| RPS11 | ENSG00000142534 | 9 | 343 | 1.76E−55 | 1.03E−53 | 0.02556818 | YES |
| RPS12 | ENSG00000112306 | 2 | 81 | 4.46E−17 | 6.86E−16 | 0.02409639 | YES |
| RPS13 | ENSG00000110700 | 8 | 252 | 1.08E−42 | 4.07E−41 | 0.03076923 | YES |
| RPS14 | ENSG00000164587 | 16 | 670 | 9.99E−93 | 1.94E−90 | 0.02332362 | YES |
| RPS15 | ENSG00000115268 | 4 | 41 | 3.87E−08 | 4.21E−07 | 0.08888889 | YES |
| RPS15A | ENSG00000134419 | 7 | 448 | 1.59E−70 | 1.64E−68 | 0.01538462 | YES |
| RPS16 | ENSG00000105193 | 11 | 131 | 1.84E−21 | 3.29E−20 | 0.07746479 | YES |
| RPS18 | ENSG00000231500 | 8 | 185 | 4.28E−32 | 1.08E−30 | 0.04145078 | YES |
| RPS19 | ENSG00000105372 | 39 | 189 | 7.42E−20 | 1.24E−18 | 0.17105263 | YES |
| RPS19BP1 | ENSG00000187051 | 17 | 246 | 2.24E−37 | 6.96E−36 | 0.06463878 | YES |
| RPS2 | ENSG00000140988 | 1 | 62 | 5.31E−14 | 7.28E−13 | 0.01587302 | YES |
| RPS21 | ENSG00000171858 | 5 | 54 | 3.21E−10 | 3.76E−09 | 0.08474576 | YES |
| RPS23 | ENSG00000186468 | 8 | 548 | 2.20E−82 | 3.29E−80 | 0.01438849 | YES |
| RPS24 | ENSG00000138326 | 10 | 425 | 4.42E−66 | 3.88E−64 | 0.02298851 | YES |
| RPS25 | ENSG00000118181 | 118 | 338 | 5.43E−19 | 8.87E−18 | 0.25877193 | YES |
| RPS26 | ENSG00000197728 | 9 | 187 | 6.54E−32 | 1.65E−30 | 0.04591837 | YES |
| RPS27 | ENSG00000177954 | 56 | 255 | 5.76E−24 | 1.11E−22 | 0.18006431 | YES |
| RPS27A | ENSG00000143947 | 10 | 367 | 2.60E−58 | 1.72E−56 | 0.0265252 | YES |
| RPS29 | ENSG00000213741 | 50 | 272 | 2.95E−28 | 5.55E−27 | 0.1552795 | YES |
| RPS3 | ENSG00000149273 | 7 | 530 | 1.05E−80 | 1.48E−78 | 0.01303538 | YES |
| RPS3A | ENSG00000145425 | 1 | 325 | 4.29E−57 | 2.66E−55 | 0.00306748 | YES |
| RPS4X | ENSG00000198034 | 8 | 490 | 2.48E−75 | 2.93E−73 | 0.01606426 | YES |
| RPS6 | ENSG00000137154 | 3 | 303 | 6.94E−53 | 3.65E−51 | 0.00980392 | YES |
| RPS6KB1 | ENSG00000108443 | 196 | 292 | 0.00010959 | 0.00100053 | 0.40163934 | YES |
| RPS7 | ENSG00000171863 | 2 | 96 | 6.91E−20 | 1.16E−18 | 0.02040816 | YES |
| RPS8 | ENSG00000142937 | 5 | 262 | 8.91E−46 | 3.76E−44 | 0.01872659 | YES |
| RPS9 | ENSG00000170889 | 5 | 77 | 1.17E−14 | 1.65E−13 | 0.06097561 | YES |
| RPSA | ENSG00000168028 | 9 | 271 | 4.67E−45 | 1.92E−43 | 0.03214286 | YES |
| RPTOR | ENSG00000141564 | 1927 | 12949 | 1.91E−286 | 8.32E−283 | 0.12953751 | YES |
| RPUSD4 | ENSG00000165526 | 45 | 112 | 3.08E−07 | 3.20E−06 | 0.2866242 | YES |
| RREB1 | ENSG00000124782 | 2109 | 3873 | 8.66E−34 | 2.34E−32 | 0.35255767 | YES |
| RRM1 | ENSG00000167325 | 23 | 490 | 2.12E−68 | 2.06E−66 | 0.04483431 | YES |
| RRM2 | ENSG00000171848 | 1 | 40 | 1.40E−09 | 1.61E−08 | 0.02439024 | YES |
| RRN3 | ENSG00000085721 | 15 | 141 | 2.48E−21 | 4.40E−20 | 0.09615385 | YES |
| RRP1 | ENSG00000160214 | 14 | 182 | 1.22E−28 | 2.73E−27 | 0.07142857 | YES |
| RRP12 | ENSG00000052749 | 557 | 759 | 1.85E−05 | 0.00017711 | 0.42325228 | YES |
| RRP15 | ENSG00000067533 | 18 | 139 | 1.08E−19 | 1.81E−18 | 0.11464968 | YES |
| RRP36 | ENSG00000124541 | 28 | 103 | 3.44E−10 | 4.03E−09 | 0.21374046 | YES |
| RRP7A | ENSG00000189306 | 16 | 63 | 1.88E−07 | 1.97E−06 | 0.20253165 | YES |
| RRP9 | ENSG00000114767 | 37 | 135 | 2.09E−12 | 2.68E−11 | 0.21511628 | YES |
| RSL1D1 | ENSG00000171490 | 19 | 550 | 1.50E−77 | 1.97E−75 | 0.03339192 | YES |
| RSL24D1 | ENSG00000137876 | 4 | 322 | 4.47E−55 | 2.53E−53 | 0.01226994 | YES |
| RSRC2 | ENSG00000111011 | 28 | 872 | 7.44E−109 | 2.28E−106 | 0.03111111 | YES |
| RTEL1 | ENSG00000258366 | 26 | 95 | 1.54E−09 | 1.76E−08 | 0.21487603 | YES |
| RTF1 | ENSG00000137815 | 13 | 237 | 6.90E−38 | 2.19E−36 | 0.052 | YES |
| RTFDC1 | ENSG00000022277 | 9 | 154 | 2.10E−26 | 4.37E−25 | 0.05521472 | YES |
| RTTN | ENSG00000176225 | 32 | 670 | 6.91E−86 | 1.09E−83 | 0.04558405 | YES |
| RUVBL1 | ENSG00000175792 | 7 | 252 | 3.28E−43 | 1.28E−41 | 0.02702703 | YES |
| RUVBL2 | ENSG00000183207 | 26 | 117 | 4.95E−13 | 6.50E−12 | 0.18181818 | YES |
| SACM1L | ENSG00000211456 | 43 | 1463 | 8.33E−155 | 5.59E−152 | 0.02855246 | YES |
| SAE1 | ENSG00000142230 | 29 | 894 | 1.25E−110 | 4.03E−108 | 0.03141928 | YES |
| SAMD4B | ENSG00000179134 | 12 | 42 | 3.70E−05 | 0.00034887 | 0.22222222 | YES |
| SAMM50 | ENSG00000100347 | 8 | 176 | 1.31E−30 | 3.13E−29 | 0.04347826 | YES |
| SAP130 | ENSG00000136715 | 14 | 73 | 6.89E−10 | 8.00E−09 | 0.16091954 | YES |
| SAP18 | ENSG00000150459 | 3 | 142 | 1.53E−27 | 3.32E−26 | 0.02068966 | YES |
| SAP30BP | ENSG00000161526 | 6 | 317 | 2.53E−53 | 1.35E−51 | 0.01857585 | YES |
| SARNP | ENSG00000205323 | 300 | 541 | 2.03E−11 | 2.49E−10 | 0.35671819 | YES |
| SARS | ENSG00000031698 | 3 | 179 | 8.64E−34 | 2.34E−32 | 0.01648352 | YES |
| SARS2 | ENSG00000104835 | 52 | 280 | 1.02E−28 | 2.30E−27 | 0.15662651 | YES |
| SART1 | ENSG00000175467 | 0 | 77 | 1.61E−17 | 2.50E−16 | 0 | YES |
| SART3 | ENSG00000075856 | 11 | 285 | 3.87E−46 | 1.65E−44 | 0.03716216 | YES |
| SASS6 | ENSG00000156876 | 26 | 502 | 1.37E−68 | 1.35E−66 | 0.04924242 | YES |
| SBDS | ENSG00000126524 | 22 | 95 | 8.45E−11 | 1.01E−09 | 0.18803419 | YES |
| SCAF11 | ENSG00000139218 | 604 | 883 | 7.70E−08 | 8.25E−07 | 0.40618695 | YES |
| SCAMP4 | ENSG00000227500 | 52 | 135 | 1.06E−08 | 1.18E−07 | 0.27807487 | YES |
| SCD | ENSG00000099194 | 55 | 336 | 2.00E−35 | 5.84E−34 | 0.14066496 | YES |
| SCFD1 | ENSG00000092108 | 39 | 1450 | 3.22E−155 | 2.25E−152 | 0.02619208 | YES |
| SCO1 | ENSG00000133028 | 81 | 278 | 3.71E−20 | 6.32E−19 | 0.22562674 | YES |
| SDAD1 | ENSG00000198301 | 16 | 272 | 8.72E−42 | 3.19E−40 | 0.05555556 | YES |
| SDE2 | ENSG00000143751 | 16 | 340 | 1.16E−51 | 5.87E−50 | 0.04494382 | YES |
| SDHA | ENSG00000073578 | 7 | 184 | 1.88E−32 | 4.82E−31 | 0.03664921 | YES |
| SDHB | ENSG00000117118 | 11 | 294 | 1.83E−47 | 8.11E−46 | 0.03606557 | YES |
| SDHC | ENSG00000143252 | 19 | 405 | 3.44E−59 | 2.38E−57 | 0.04481132 | YES |
| SDHD | ENSG00000204370 | 8 | 202 | 7.37E−35 | 2.09E−33 | 0.03809524 | YES |
| SEC13 | ENSG00000157020 | 1 | 140 | 2.41E−28 | 5.39E−27 | 0.0070922 | YES |
| SEC16A | ENSG00000148396 | 8 | 29 | 0.00037165 | 0.00329655 | 0.21621622 | YES |
| SEC61A1 | ENSG00000058262 | 5 | 159 | 2.40E−29 | 5.50E−28 | 0.0304878 | YES |
| SEC61B | ENSG00000106803 | 14 | 42 | 0.00014216 | 0.00128909 | 0.25 | YES |
| SEC62 | ENSG00000008952 | 126 | 189 | 0.00081392 | 0.00701985 | 0.4 | YES |
| SEC63 | ENSG00000025796 | 295 | 573 | 4.24E−14 | 5.86E−13 | 0.33986175 | YES |
| SEH1L | ENSG00000085415 | 81 | 970 | 6.58E−99 | 1.57E−96 | 0.07706946 | YES |
| SEL1L | ENSG00000071537 | 474 | 924 | 5.48E−19 | 8.94E−18 | 0.33905579 | YES |
| SENP6 | ENSG00000112701 | 78 | 1145 | 3.18E−116 | 1.21E−113 | 0.0637776 | YES |
| SEPSECS | ENSG00000109618 | 194 | 584 | 1.47E−29 | 3.38E−28 | 0.24935733 | YES |
| SERBP1 | ENSG00000142864 | 254 | 440 | 4.28E−09 | 4.82E−08 | 0.36599424 | YES |
| SF1 | ENSG00000168066 | 10 | 86 | 6.72E−14 | 9.16E−13 | 0.10416667 | YES |
| SF3A1 | ENSG00000099995 | 7 | 104 | 1.23E−18 | 1.99E−17 | 0.06306306 | YES |
| SF3A2 | ENSG00000104897 | 12 | 215 | 6.33E−35 | 1.79E−33 | 0.05286344 | YES |
| SF3B1 | ENSG00000115524 | 9 | 405 | 6.21E−64 | 5.02E−62 | 0.02173913 | YES |
| SF3B14 | ENSG00000115128 | 6 | 154 | 5.85E−28 | 1.28E−26 | 0.0375 | YES |
| SF3B2 | ENSG00000087365 | 3 | 143 | 1.03E−27 | 2.24E−26 | 0.02054795 | YES |
| SF3B3 | ENSG00000189091 | 71 | 630 | 1.49E−66 | 1.33E−64 | 0.10128388 | YES |
| SFPQ | ENSG00000116560 | 2 | 27 | 2.72E−06 | 2.72E−05 | 0.06896552 | YES |
| SFSWAP | ENSG00000061936 | 81 | 208 | 1.20E−11 | 1.49E−10 | 0.28027682 | YES |
| SGOL1 | ENSG00000129810 | 41 | 156 | 2.43E−14 | 3.38E−13 | 0.20812183 | YES |
| SHFM1 | ENSG00000127922 | 1 | 81 | 1.14E−17 | 1.78E−16 | 0.01219512 | YES |
| SHMT2 | ENSG00000182199 | 137 | 286 | 1.99E−10 | 2.35E−09 | 0.32387707 | YES |
| SHOC2 | ENSG00000108061 | 41 | 296 | 7.71E−35 | 2.18E−33 | 0.12166172 | YES |
| SHQ1 | ENSG00000144736 | 38 | 238 | 1.64E−27 | 3.53E−26 | 0.13768116 | YES |
| SKA1 | ENSG00000154839 | 27 | 81 | 4.01E−07 | 4.15E−06 | 0.25 | YES |
| SKA2 | ENSG00000182628 | 94 | 1298 | 2.88E−124 | 1.32E−121 | 0.06752874 | YES |
| SKA3 | ENSG00000165480 | 17 | 305 | 3.48E−46 | 1.49E−44 | 0.05279503 | YES |
| SKIV2L2 | ENSG00000039123 | 93 | 1101 | 2.89E−107 | 8.54E−105 | 0.07788945 | YES |
| SKP1 | ENSG00000113558 | 38 | 116 | 2.22E−09 | 2.52E−08 | 0.24675325 | YES |
| SLBP | ENSG00000163950 | 46 | 262 | 3.46E−28 | 7.67E−27 | 0.14935065 | YES |
| SLC25A19 | ENSG00000125454 | 257 | 367 | 0.00013667 | 0.00124123 | 0.41185897 | YES |
| SLC25A26 | ENSG00000144741 | 75 | 260 | 2.26E−19 | 3.74E−18 | 0.2238806 | YES |
| SLC25A3 | ENSG00000075415 | 67 | 158 | 1.61E−08 | 1.77E−07 | 0.29777778 | YES |
| SLC35B1 | ENSG00000121073 | 10 | 65 | 4.91E−10 | 5.72E−09 | 0.13333333 | YES |
| SLC39A10 | ENSG00000196950 | 796 | 1090 | 1.32E−06 | 1.34E−05 | 0.42205726 | YES |
| SLC7A6OS | ENSG00000103061 | 6 | 189 | 8.47E−34 | 2.29E−32 | 0.03076923 | YES |
| SLIRP | ENSG00000119705 | 186 | 277 | 0.00014893 | 0.00134842 | 0.40172786 | YES |
| SLMAP | ENSG00000163681 | 444 | 739 | 4.94E−11 | 6.00E−10 | 0.37531699 | YES |
| SLMO2 | ENSG00000101166 | 14 | 218 | 1.97E−34 | 5.46E−33 | 0.06034483 | YES |
| SLU7 | ENSG00000164609 | 48 | 151 | 9.66E−12 | 1.21E−10 | 0.24120603 | YES |
| SLX4 | ENSG00000188827 | 192 | 384 | 1.23E−11 | 1.52E−10 | 0.33333333 | YES |
| SMARCA5 | ENSG00000153147 | 27 | 167 | 1.08E−20 | 1.88E−19 | 0.13917526 | YES |
| SMARCB1 | ENSG00000099956 | 50 | 727 | 4.65E−85 | 7.19E−83 | 0.06435006 | YES |
| SMARCE1 | ENSG00000073584 | 12 | 256 | 2.70E−41 | 9.73E−40 | 0.04477612 | YES |
| SMC1A | ENSG00000072501 | 9 | 327 | 3.18E−53 | 1.68E−51 | 0.02678571 | YES |
| SMC2 | ENSG00000136824 | 30 | 215 | 4.76E−27 | 1.01E−25 | 0.12244898 | YES |
| SMC3 | ENSG00000108055 | 10 | 201 | 1.12E−33 | 3.00E−32 | 0.04739336 | YES |
| SMC4 | ENSG00000113810 | 18 | 374 | 1.87E−55 | 1.09E−53 | 0.04591837 | YES |
| SMC5 | ENSG00000198887 | 5 | 167 | 1.08E−30 | 2.59E−29 | 0.02906977 | YES |
| SMC6 | ENSG00000163029 | 31 | 172 | 5.23E−20 | 8.85E−19 | 0.15270936 | YES |
| SMG1 | ENSG00000157106 | 107 | 404 | 5.20E−29 | 1.18E−27 | 0.20939335 | YES |
| SMG5 | ENSG00000198952 | 429 | 2018 | 1.91E−99 | 4.63E−97 | 0.17531671 | YES |
| SMG6 | ENSG00000070366 | 1171 | 7052 | 2.26E−215 | 3.95E−212 | 0.14240545 | YES |
| SMG7 | ENSG00000116698 | 263 | 471 | 2.58E−10 | 3.03E−09 | 0.35831063 | YES |
| SMNDC1 | ENSG00000119953 | 21 | 83 | 3.67E−09 | 4.14E−08 | 0.20192308 | YES |
| SMU1 | ENSG00000122692 | 3 | 156 | 6.11E−30 | 1.42E−28 | 0.01886792 | YES |
| SNAPC1 | ENSG00000023608 | 30 | 418 | 4.47E−56 | 2.67E−54 | 0.06696429 | YES |
| SNAPC3 | ENSG00000164975 | 8 | 302 | 3.88E−50 | 1.86E−48 | 0.02580645 | YES |
| SNF8 | ENSG00000159210 | 29 | 134 | 9.85E−15 | 1.40E−13 | 0.17791411 | YES |
| SNIP1 | ENSG00000163877 | 3 | 50 | 1.81E−10 | 2.14E−09 | 0.05660377 | YES |
| SNRNP200 | ENSG00000144028 | 3 | 128 | 4.29E−25 | 8.53E−24 | 0.02290076 | YES |
| SNRNP27 | ENSG00000124380 | 185 | 473 | 1.52E−20 | 2.62E−19 | 0.28115502 | YES |
| SNRNP35 | ENSG00000184209 | 325 | 903 | 2.05E−35 | 5.97E−34 | 0.26465798 | YES |
| SNRNP40 | ENSG00000060688 | 122 | 325 | 8.42E−17 | 1.28E−15 | 0.27293065 | YES |
| SNRNP48 | ENSG00000168566 | 1 | 30 | 1.71E−07 | 1.80E−06 | 0.03225806 | YES |
| SNRNP70 | ENSG00000104852 | 23 | 115 | 9.67E−14 | 1.31E−12 | 0.16666667 | YES |
| SNRPA | ENSG00000077312 | 367 | 601 | 2.40E−09 | 2.73E−08 | 0.37913223 | YES |
| SNRPA1 | ENSG00000131876 | 10 | 241 | 5.46E−40 | 1.87E−38 | 0.03984064 | YES |
| SNRPB | ENSG00000125835 | 13 | 443 | 5.54E−67 | 5.04E−65 | 0.02850877 | YES |
| SNRPC | ENSG00000124562 | 4 | 158 | 1.01E−29 | 2.33E−28 | 0.02469136 | YES |
| SNRPD1 | ENSG00000167088 | 12 | 806 | 6.32E−109 | 1.97E−106 | 0.01466993 | YES |
| SNRPD2 | ENSG00000125743 | 4 | 61 | 4.18E−12 | 5.31E−11 | 0.06153846 | YES |
| SNRPD3 | ENSG00000100028 | 58 | 262 | 2.52E−24 | 4.91E−23 | 0.18125 | YES |
| SNRPE | ENSG00000182004 | 2 | 169 | 1.08E−32 | 2.79E−31 | 0.01169591 | YES |
| SNRPF | ENSG00000139343 | 5 | 154 | 1.70E−28 | 3.81E−27 | 0.03144654 | YES |
| SNRPG | ENSG00000143977 | 7 | 351 | 1.33E−57 | 8.57E−56 | 0.01955307 | YES |
| SNW1 | ENSG00000100603 | 4 | 133 | 2.05E−25 | 4.11E−24 | 0.02919708 | YES |
| SOD1 | ENSG00000142168 | 12 | 113 | 7.74E−18 | 1.22E−16 | 0.096 | YES |
| SOD2 | ENSG00000112096 | 66 | 396 | 1.05E−39 | 3.52E−38 | 0.14285714 | YES |
| SON | ENSG00000159140 | 11 | 325 | 6.04E−52 | 3.07E−50 | 0.0327381 | YES |
| SP1 | ENSG00000185591 | 201 | 419 | 2.22E−13 | 2.95E−12 | 0.32419355 | YES |
| SPAG5 | ENSG00000076382 | 39 | 149 | 6.76E−14 | 9.21E−13 | 0.20744681 | YES |
| SPATA5 | ENSG00000145375 | 132 | 866 | 9.68E−73 | 1.06E−70 | 0.13226453 | YES |
| SPATA5L1 | ENSG00000171763 | 6 | 100 | 2.02E−18 | 3.24E−17 | 0.05660377 | YES |
| SPC24 | ENSG00000161888 | 6 | 44 | 8.71E−08 | 9.30E−07 | 0.12 | YES |
| SPC25 | ENSG00000152253 | 1 | 35 | 1.53E−08 | 1.69E−07 | 0.02777778 | YES |
| SPCS2 | ENSG00000118363 | 30 | 626 | 1.00E−81 | 1.46E−79 | 0.04573171 | YES |
| SPCS3 | ENSG00000129128 | 34 | 275 | 1.76E−34 | 4.91E−33 | 0.11003236 | YES |
| SPDL1 | ENSG00000040275 | 1 | 55 | 1.29E−12 | 1.67E−11 | 0.01785714 | YES |
| SPRTN | ENSG00000010072 | 52 | 171 | 1.83E−13 | 2.44E−12 | 0.23318386 | YES |
| SRBD1 | ENSG00000068784 | 135 | 3227 | 1.88E−234 | 4.69E−231 | 0.04015467 | YES |
| SRCAP | ENSG00000080603 | 79 | 276 | 2.11E−20 | 3.63E−19 | 0.22253521 | YES |
| SRD5A3 | ENSG00000128039 | 294 | 557 | 4.33E−13 | 5.70E−12 | 0.34547591 | YES |
| SREK1 | ENSG00000153914 | 371 | 671 | 4.26E−13 | 5.62E−12 | 0.35604607 | YES |
| SRF | ENSG00000112658 | 82 | 145 | 6.98E−05 | 0.00064713 | 0.36123348 | YES |
| SRFBP1 | ENSG00000151304 | 22 | 179 | 1.33E−24 | 2.60E−23 | 0.10945274 | YES |
| SRP14 | ENSG00000140319 | 8 | 82 | 4.28E−14 | 5.91E−13 | 0.08888889 | YES |
| SRP19 | ENSG00000153037 | 6 | 99 | 3.07E−18 | 4.91E−17 | 0.05714286 | YES |
| SRP68 | ENSG00000167881 | 17 | 237 | 5.44E−36 | 1.64E−34 | 0.06692913 | YES |
| SRP72 | ENSG00000174780 | 208 | 888 | 9.84E−56 | 5.81E−54 | 0.18978102 | YES |
| SRP9 | ENSG00000143742 | 19 | 252 | 2.24E−37 | 6.98E−36 | 0.0701107 | YES |
| SRPR | ENSG00000182934 | 10 | 137 | 5.48E−23 | 1.03E−21 | 0.06802721 | YES |
| SRPRB | ENSG00000144867 | 41 | 111 | 5.67E−08 | 6.12E−07 | 0.26973684 | YES |
| SRRD | ENSG00000100104 | 98 | 175 | 1.42E−05 | 0.00013676 | 0.35897436 | YES |
| SRRM1 | ENSG00000133226 | 7 | 192 | 9.25E−34 | 2.49E−32 | 0.03517588 | YES |
| SRRM2 | ENSG00000167978 | 60 | 300 | 6.71E−29 | 1.52E−27 | 0.16666667 | YES |
| SRRT | ENSG00000087087 | 37 | 136 | 1.48E−12 | 1.92E−11 | 0.21387283 | YES |
| SRSF1 | ENSG00000136450 | 11 | 265 | 3.75E−43 | 1.46E−41 | 0.03985507 | YES |
| SRSF10 | ENSG00000188529 | 161 | 464 | 6.57E−24 | 1.26E−22 | 0.2576 | YES |
| SRSF11 | ENSG00000116754 | 825 | 1096 | 9.57E−06 | 9.34E−05 | 0.42946382 | YES |
| SRSF2 | ENSG00000161547 | 2 | 21 | 5.11E−05 | 0.0004783 | 0.08695652 | YES |
| SRSF3 | ENSG00000112081 | 8 | 311 | 1.94E−51 | 9.72E−50 | 0.02507837 | YES |
| SRSF7 | ENSG00000115875 | 5 | 144 | 8.82E−27 | 1.86E−25 | 0.03355705 | YES |
| SSB | ENSG00000138385 | 31 | 193 | 2.84E−23 | 5.36E−22 | 0.13839286 | YES |
| SSBP1 | ENSG00000106028 | 115 | 286 | 6.21E−14 | 8.48E−13 | 0.28678304 | YES |
| SSRP1 | ENSG00000149136 | 65 | 220 | 1.03E−16 | 1.55E−15 | 0.22807018 | YES |
| SSU72 | ENSG00000160075 | 25 | 287 | 5.92E−40 | 2.02E−38 | 0.08012821 | YES |
| STIL | ENSG00000123473 | 66 | 324 | 2.91E−30 | 6.89E−29 | 0.16923077 | YES |
| STRAP | ENSG00000023734 | 50 | 291 | 6.33E−31 | 1.53E−29 | 0.14662757 | YES |
| STRIP1 | ENSG00000143093 | 15 | 96 | 1.51E−13 | 2.03E−12 | 0.13513514 | YES |
| STX18 | ENSG00000168818 | 17 | 321 | 1.71E−48 | 7.87E−47 | 0.05029586 | YES |
| STX5 | ENSG00000162236 | 23 | 97 | 8.52E−11 | 1.02E−09 | 0.19166667 | YES |
| SUDS3 | ENSG00000111707 | 8 | 49 | 7.00E−08 | 7.51E−07 | 0.14035088 | YES |
| SUGP1 | ENSG00000105705 | 23 | 170 | 9.69E−23 | 1.80E−21 | 0.11917098 | YES |
| SUGT1 | ENSG00000165416 | 42 | 290 | 1.30E−33 | 3.47E−32 | 0.12650602 | YES |
| SUMO2 | ENSG00000188612 | 14 | 146 | 1.23E−22 | 2.29E−21 | 0.0875 | YES |
| SUPT16H | ENSG00000092201 | 97 | 761 | 2.08E−72 | 2.24E−70 | 0.11305361 | YES |
| SUPT4H1 | ENSG00000213246 | 16 | 47 | 7.92E−05 | 0.00073172 | 0.25396825 | YES |
| SUPT5H | ENSG00000196235 | 36 | 264 | 4.29E−32 | 1.08E−30 | 0.12 | YES |
| SUPT6H | ENSG00000109111 | 10 | 171 | 9.18E−29 | 2.07E−27 | 0.05524862 | YES |
| SUPV3L1 | ENSG00000156502 | 20 | 195 | 5.00E−28 | 1.10E−26 | 0.09302326 | YES |
| SURF6 | ENSG00000148296 | 12 | 245 | 1.30E−39 | 4.34E−38 | 0.04669261 | YES |
| SUZ12 | ENSG00000178691 | 33 | 259 | 1.62E−32 | 4.17E−31 | 0.1130137 | YES |
| SYF2 | ENSG00000117614 | 4 | 138 | 2.74E−26 | 5.66E−25 | 0.02816901 | YES |
| SYMPK | ENSG00000125755 | 18 | 201 | 7.14E−30 | 1.66E−28 | 0.08219178 | YES |
| SYS1 | ENSG00000204070 | 13 | 40 | 0.00015813 | 0.00142797 | 0.24528302 | YES |
| TACC3 | ENSG00000013810 | 252 | 875 | 1.72E−45 | 7.21E−44 | 0.22360248 | YES |
| TADA1 | ENSG00000152382 | 14 | 76 | 2.03E−10 | 2.39E−09 | 0.15555556 | YES |
| TADA2A | ENSG00000108264 | 457 | 759 | 3.95E−11 | 4.81E−10 | 0.37582237 | YES |
| TAF1 | ENSG00000147133 | 192 | 771 | 3.74E−48 | 1.70E−46 | 0.19937695 | YES |
| TAF12 | ENSG00000120656 | 72 | 316 | 1.98E−27 | 4.25E−26 | 0.18556701 | YES |
| TAF13 | ENSG00000197780 | 20 | 134 | 4.94E−18 | 7.84E−17 | 0.12987013 | YES |
| TAF1B | ENSG00000115750 | 15 | 326 | 3.75E−50 | 1.81E−48 | 0.04398827 | YES |
| TAF1C | ENSG00000103168 | 6 | 47 | 2.26E−08 | 2.49E−07 | 0.11320755 | YES |
| TAF1D | ENSG00000166012 | 96 | 731 | 1.64E−69 | 1.62E−67 | 0.11608222 | YES |
| TAF5 | ENSG00000148835 | 11 | 72 | 6.68E−11 | 8.09E−10 | 0.13253012 | YES |
| TAF6 | ENSG00000106290 | 40 | 151 | 6.71E−14 | 9.16E−13 | 0.20942408 | YES |
| TAF6L | ENSG00000162227 | 109 | 200 | 2.08E−06 | 2.10E−05 | 0.35275081 | YES |
| TAF8 | ENSG00000137413 | 14 | 96 | 5.88E−14 | 8.05E−13 | 0.12727273 | YES |
| TAF9 | ENSG00000085231 | 264 | 614 | 5.47E−21 | 9.61E−20 | 0.30068337 | YES |
| TAMM41 | ENSG00000144559 | 23 | 203 | 5.17E−28 | 1.14E−26 | 0.10176991 | YES |
| TANGO6 | ENSG00000103047 | 48 | 596 | 5.34E−71 | 5.65E−69 | 0.07453416 | YES |
| TARDBP | ENSG00000120948 | 7 | 35 | 1.26E−05 | 0.00012148 | 0.16666667 | YES |
| TARS | ENSG00000113407 | 19 | 715 | 2.45E−96 | 5.09E−94 | 0.02588556 | YES |
| TARS2 | ENSG00000143374 | 56 | 312 | 8.05E−32 | 2.01E−30 | 0.15217391 | YES |
| TBCA | ENSG00000171530 | 47 | 375 | 1.49E−43 | 5.94E−42 | 0.11137441 | YES |
| TBCB | ENSG00000105254 | 3 | 27 | 8.91E−06 | 8.70E−05 | 0.1 | YES |
| TBCD | ENSG00000141556 | 190 | 2653 | 1.86E−190 | 2.03E−187 | 0.06683081 | YES |
| TBCE | ENSG00000116957 | 52 | 197 | 4.65E−17 | 7.13E−16 | 0.20883534 | YES |
| TCEB2 | ENSG00000103363 | 4 | 59 | 1.03E−11 | 1.28E−10 | 0.06349206 | YES |
| TCERG1 | ENSG00000113649 | 194 | 542 | 1.54E−25 | 3.11E−24 | 0.26358696 | YES |
| TCOF1 | ENSG00000070814 | 102 | 184 | 7.15E−06 | 7.00E−05 | 0.35664336 | YES |
| TCP1 | ENSG00000120438 | 5 | 106 | 4.78E−20 | 8.10E−19 | 0.04504505 | YES |
| TECR | ENSG00000099797 | 230 | 369 | 1.19E−06 | 1.22E−05 | 0.38397329 | YES |
| TEFM | ENSG00000172171 | 174 | 338 | 4.36E−10 | 5.08E−09 | 0.33984375 | YES |
| TEX10 | ENSG00000136891 | 45 | 410 | 5.61E−49 | 2.62E−47 | 0.0989011 | YES |
| TFAM | ENSG00000108064 | 62 | 118 | 5.65E−05 | 0.00052673 | 0.34444444 | YES |
| TFB1M | ENSG00000029639 | 84 | 196 | 9.85E−10 | 1.14E−08 | 0.3 | YES |
| TFB2M | ENSG00000162851 | 72 | 201 | 1.16E−12 | 1.51E−11 | 0.26373626 | YES |
| TFIP11 | ENSG00000100109 | 17 | 179 | 9.07E−27 | 1.91E−25 | 0.08673469 | YES |
| TFRC | ENSG00000072274 | 22 | 327 | 4.59E−47 | 2.01E−45 | 0.06303725 | YES |
| THOC1 | ENSG00000079134 | 11 | 325 | 6.04E−52 | 3.07E−50 | 0.0327381 | YES |
| THOC5 | ENSG00000100296 | 22 | 130 | 1.38E−16 | 2.09E−15 | 0.14473684 | YES |
| THOC7 | ENSG00000163634 | 16 | 380 | 3.32E−57 | 2.08E−55 | 0.04040404 | YES |
| THRAP3 | ENSG00000054118 | 304 | 438 | 3.50E−05 | 0.00033126 | 0.4097035 | YES |
| TICRR | ENSG00000140534 | 12 | 161 | 4.14E−26 | 8.50E−25 | 0.06936416 | YES |
| TIMELESS | ENSG00000111602 | 27 | 524 | 7.03E−71 | 7.30E−69 | 0.04900181 | YES |
| TIMM10 | ENSG00000134809 | 79 | 179 | 9.70E−09 | 1.08E−07 | 0.30620155 | YES |
| TIMM22 | ENSG00000177370 | 27 | 93 | 6.15E−09 | 6.90E−08 | 0.225 | YES |
| TIMM23 | ENSG00000138297 | 17 | 48 | 0.00010127 | 0.00092702 | 0.26153846 | YES |
| TIMM44 | ENSG00000104980 | 19 | 390 | 3.58E−57 | 2.23E−55 | 0.04645477 | YES |
| TIMM50 | ENSG00000105197 | 50 | 163 | 6.99E−13 | 9.15E−12 | 0.23474178 | YES |
| TIMM9 | ENSG00000100575 | 280 | 593 | 2.85E−17 | 4.40E−16 | 0.3207331 | YES |
| TINF2 | ENSG00000092330 | 8 | 34 | 4.56E−05 | 0.00042826 | 0.19047619 | YES |
| TIPRL | ENSG00000143155 | 125 | 234 | 2.25E−07 | 2.35E−06 | 0.34818942 | YES |
| TKT | ENSG00000163931 | 61 | 345 | 1.22E−34 | 3.43E−33 | 0.15024631 | YES |
| TMA16 | ENSG00000198498 | 39 | 496 | 2.08E−62 | 1.60E−60 | 0.0728972 | YES |
| TMED10 | ENSG00000170348 | 228 | 369 | 8.13E−07 | 8.33E−06 | 0.38190955 | YES |
| TMED2 | ENSG00000086598 | 119 | 194 | 8.32E−05 | 0.00076645 | 0.38019169 | YES |
| TMEM167A | ENSG00000174695 | 155 | 257 | 6.80E−06 | 6.68E−05 | 0.37621359 | YES |
| TMEM199 | ENSG00000244045 | 2 | 110 | 1.89E−22 | 3.50E−21 | 0.01785714 | YES |
| TMEM238 | ENSG00000233493 | 74 | 145 | 5.90E−06 | 5.82E−05 | 0.33789954 | YES |
| TMEM258 | ENSG00000134825 | 19 | 248 | 9.15E−37 | 2.81E−35 | 0.07116105 | YES |
| TMEM41B | ENSG00000166471 | 71 | 240 | 8.26E−18 | 1.30E−16 | 0.22829582 | YES |
| TMEM48 | ENSG00000058804 | 313 | 474 | 2.09E−06 | 2.11E−05 | 0.39771283 | YES |
| TMX2 | ENSG00000213593 | 14 | 418 | 3.04E−63 | 2.42E−61 | 0.03240741 | YES |
| TNPO3 | ENSG00000064419 | 32 | 422 | 9.19E−56 | 5.44E−54 | 0.07048458 | YES |
| TOMM20 | ENSG00000173726 | 35 | 329 | 7.68E−42 | 2.82E−40 | 0.09615385 | YES |
| TOMM22 | ENSG00000100216 | 6 | 103 | 5.71E−19 | 9.30E−18 | 0.05504587 | YES |
| TOMM40 | ENSG00000130204 | 29 | 274 | 2.39E−36 | 7.23E−35 | 0.09570957 | YES |
| TOMM70A | ENSG00000154174 | 47 | 133 | 1.52E−09 | 1.74E−08 | 0.26111111 | YES |
| TOP1 | ENSG00000198900 | 27 | 383 | 1.16E−52 | 6.06E−51 | 0.06585366 | YES |
| TOP2A | ENSG00000131747 | 17 | 94 | 2.06E−12 | 2.65E−11 | 0.15315315 | YES |
| TOP3A | ENSG00000177302 | 51 | 290 | 1.90E−30 | 4.54E−29 | 0.14956012 | YES |
| TOPBP1 | ENSG00000163781 | 187 | 319 | 3.34E−07 | 3.47E−06 | 0.36956522 | YES |
| TP53RK | ENSG00000172315 | 22 | 176 | 4.04E−24 | 7.80E−23 | 0.11111111 | YES |
| TPI1 | ENSG00000111669 | 47 | 115 | 3.30E−07 | 3.43E−06 | 0.29012346 | YES |
| TPR | ENSG00000047410 | 36 | 211 | 3.38E−24 | 6.56E−23 | 0.14574899 | YES |
| TPT1 | ENSG00000133112 | 42 | 169 | 5.99E−16 | 8.84E−15 | 0.19905213 | YES |
| TPX2 | ENSG00000088325 | 20 | 304 | 1.17E−44 | 4.79E−43 | 0.0617284 | YES |
| TRA2B | ENSG00000136527 | 48 | 424 | 1.11E−49 | 5.29E−48 | 0.10169492 | YES |
| TRAIP | ENSG00000183763 | 7 | 71 | 1.63E−12 | 2.11E−11 | 0.08974359 | YES |
| TRAPPC1 | ENSG00000170043 | 6 | 27 | 0.00017418 | 0.0015681 | 0.18181818 | YES |
| TRAPPC11 | ENSG00000168538 | 98 | 292 | 6.14E−18 | 9.71E−17 | 0.25128205 | YES |
| TRAPPC3 | ENSG00000054116 | 2 | 93 | 2.49E−19 | 4.12E−18 | 0.02105263 | YES |
| TRAPPC4 | ENSG00000196655 | 2 | 67 | 2.18E−14 | 3.05E−13 | 0.02898551 | YES |
| TRAPPC8 | ENSG00000153339 | 58 | 517 | 9.21E−58 | 5.98E−56 | 0.10086957 | YES |
| TRIAP1 | ENSG00000170855 | 11 | 242 | 1.21E−39 | 4.04E−38 | 0.04347826 | YES |
| TRIT1 | ENSG00000043514 | 275 | 485 | 4.20E−10 | 4.90E−09 | 0.36184211 | YES |
| TRMT10C | ENSG00000174173 | 39 | 201 | 1.19E−21 | 2.15E−20 | 0.1625 | YES |
| TRMT5 | ENSG00000126814 | 44 | 285 | 3.56E−32 | 9.05E−31 | 0.1337386 | YES |
| TRMT6 | ENSG00000089195 | 30 | 264 | 1.86E−34 | 5.18E−33 | 0.10204082 | YES |
| TRMT61A | ENSG00000166166 | 45 | 139 | 7.70E−11 | 9.26E−10 | 0.24456522 | YES |
| TRNAU1AP | ENSG00000180098 | 90 | 224 | 9.44E−12 | 1.18E−10 | 0.2866242 | YES |
| TRNT1 | ENSG00000072756 | 65 | 328 | 4.22E−31 | 1.02E−29 | 0.1653944 | YES |
| TRPM7 | ENSG00000092439 | 128 | 508 | 9.71E−36 | 2.88E−34 | 0.20125786 | YES |
| TRRAP | ENSG00000196367 | 23 | 103 | 8.97E−12 | 1.12E−10 | 0.18253968 | YES |
| TRUB2 | ENSG00000167112 | 111 | 238 | 1.12E−09 | 1.29E−08 | 0.31805158 | YES |
| TSC22D2 | ENSG00000196428 | 1271 | 1669 | 1.75E−06 | 1.77E−05 | 0.43231293 | YES |
| TSEN15 | ENSG00000198860 | 16 | 102 | 3.45E−14 | 4.78E−13 | 0.13559322 | YES |
| TSEN2 | ENSG00000154743 | 97 | 425 | 4.24E−34 | 1.16E−32 | 0.18582375 | YES |
| TSEN54 | ENSG00000182173 | 7 | 43 | 3.71E−07 | 3.85E−06 | 0.14 | YES |
| TSG101 | ENSG00000074319 | 56 | 329 | 3.88E−34 | 1.07E−32 | 0.14545455 | YES |
| TSR1 | ENSG00000167721 | 457 | 721 | 3.21E−09 | 3.63E−08 | 0.38794567 | YES |
| TSR2 | ENSG00000158526 | 0 | 75 | 3.88E−17 | 5.97E−16 | 0 | YES |
| TTC1 | ENSG00000113312 | 13 | 80 | 1.59E−11 | 1.97E−10 | 0.13978495 | YES |
| TTC27 | ENSG00000018699 | 40 | 244 | 1.12E−27 | 2.43E−26 | 0.14084507 | YES |
| TTC4 | ENSG00000243725 | 18 | 106 | 4.36E−14 | 6.00E−13 | 0.14516129 | YES |
| TTF1 | ENSG00000125482 | 143 | 1040 | 1.96E−86 | 3.11E−84 | 0.12087912 | YES |
| TTF2 | ENSG00000116830 | 64 | 209 | 1.60E−15 | 2.32E−14 | 0.23443223 | YES |
| TTI1 | ENSG00000101407 | 106 | 1096 | 1.18E−102 | 3.17E−100 | 0.08818636 | YES |
| TTK | ENSG00000112742 | 24 | 99 | 8.55E−11 | 1.02E−09 | 0.19512195 | YES |
| TUBA1B | ENSG00000123416 | 29 | 232 | 4.81E−30 | 1.12E−28 | 0.11111111 | YES |
| TUBB | ENSG00000196230 | 44 | 196 | 2.76E−19 | 4.56E−18 | 0.18333333 | YES |
| TUBD1 | ENSG00000108423 | 186 | 306 | 2.14E−06 | 2.15E−05 | 0.37804878 | YES |
| TUBE1 | ENSG00000074935 | 3 | 63 | 4.86E−13 | 6.39E−12 | 0.04545455 | YES |
| TUBG1 | ENSG00000131462 | 0 | 31 | 2.51E−08 | 2.76E−07 | 0 | YES |
| TUBGCP3 | ENSG00000126216 | 60 | 1928 | 2.64E−182 | 2.57E−179 | 0.03018109 | YES |
| TUBGCP4 | ENSG00000137822 | 53 | 312 | 8.01E−33 | 2.08E−31 | 0.14520548 | YES |
| TUBGCP5 | ENSG00000153575 | 6 | 132 | 3.72E−24 | 7.20E−23 | 0.04347826 | YES |
| TUBGCP6 | ENSG00000128159 | 9 | 118 | 3.79E−20 | 6.46E−19 | 0.07086614 | YES |
| TUFM | ENSG00000178952 | 23 | 163 | 1.32E−21 | 2.37E−20 | 0.12365591 | YES |
| TUT1 | ENSG00000149016 | 67 | 338 | 8.32E−32 | 2.08E−30 | 0.1654321 | YES |
| TWISTNB | ENSG00000105849 | 2 | 126 | 2.60E−25 | 5.20E−24 | 0.015625 | YES |
| TXN | ENSG00000136810 | 15 | 112 | 2.43E−16 | 3.63E−15 | 0.11811024 | YES |
| TXNL4A | ENSG00000141759 | 10 | 292 | 1.14E−47 | 5.12E−46 | 0.03311258 | YES |
| TYMS | ENSG00000176890 | 8 | 252 | 1.08E−42 | 4.07E−41 | 0.03076923 | YES |
| U2AF1 | ENSG00000160201 | 5 | 260 | 1.79E−45 | 7.46E−44 | 0.01886792 | YES |
| U2AF2 | ENSG00000063244 | 23 | 982 | 1.64E−121 | 6.84E−119 | 0.02288557 | YES |
| U2SURP | ENSG00000163714 | 125 | 867 | 7.41E−75 | 8.62E−73 | 0.12600806 | YES |
| UBA1 | ENSG00000130985 | 6 | 161 | 3.79E−29 | 8.64E−28 | 0.03592814 | YES |
| UBA2 | ENSG00000126261 | 9 | 223 | 1.09E−37 | 3.43E−36 | 0.0387931 | YES |
| UBA3 | ENSG00000144744 | 81 | 1257 | 5.06E−125 | 2.39E−122 | 0.06053812 | YES |
| UBA52 | ENSG00000221983 | 5 | 148 | 1.81E−27 | 3.89E−26 | 0.03267974 | YES |
| UBAP1 | ENSG00000165006 | 169 | 530 | 4.82E−29 | 1.09E−27 | 0.24177396 | YES |
| UBE2H | ENSG00000186591 | 1035 | 1585 | 1.63E−12 | 2.10E−11 | 0.39503817 | YES |
| UBE2I | ENSG00000103275 | 18 | 66 | 2.56E−07 | 2.67E−06 | 0.21428571 | YES |
| UBE2L3 | ENSG00000185651 | 178 | 832 | 2.54E−57 | 1.60E−55 | 0.17623762 | YES |
| UBE2M | ENSG00000130725 | 62 | 161 | 7.41E−10 | 8.59E−09 | 0.27802691 | YES |
| UBE2S | ENSG00000108106 | 0 | 13 | 0.00020486 | 0.0018405 | 0 | YES |
| UBE2T | ENSG00000077152 | 40 | 130 | 7.41E−11 | 8.95E−10 | 0.23529412 | YES |
| UBE3D | ENSG00000118420 | 183 | 273 | 0.00015275 | 0.00138156 | 0.40131579 | YES |
| UBE4B | ENSG00000130939 | 1500 | 1825 | 0.00026776 | 0.00239207 | 0.45112782 | YES |
| UBL5 | ENSG00000198258 | 19 | 73 | 3.70E−08 | 4.03E−07 | 0.20652174 | YES |
| UBR4 | ENSG00000127481 | 769 | 1396 | 4.71E−20 | 7.99E−19 | 0.3551963 | YES |
| UBTF | ENSG00000108312 | 102 | 601 | 2.79E−53 | 1.49E−51 | 0.14509246 | YES |
| UCHL5 | ENSG00000116750 | 305 | 944 | 6.35E−42 | 2.35E−40 | 0.24419536 | YES |
| UFD1L | ENSG00000070010 | 5 | 101 | 3.91E−19 | 6.43E−18 | 0.04716981 | YES |
| UMPS | ENSG00000114491 | 4 | 156 | 2.20E−29 | 5.06E−28 | 0.025 | YES |
| UPF1 | ENSG00000005007 | 6 | 107 | 1.07E−19 | 1.79E−18 | 0.05309735 | YES |
| UPF2 | ENSG00000151461 | 437 | 2284 | 4.91E−115 | 1.75E−112 | 0.16060272 | YES |
| UQCC | ENSG00000101019 | 660 | 905 | 4.28E−06 | 4.24E−05 | 0.42172524 | YES |
| UQCRC1 | ENSG00000010256 | 46 | 221 | 2.78E−22 | 5.13E−21 | 0.17228464 | YES |
| UQCRC2 | ENSG00000140740 | 74 | 369 | 1.05E−33 | 2.82E−32 | 0.16704289 | YES |
| UQCRFS1 | ENSG00000169021 | 14 | 160 | 5.37E−25 | 1.06E−23 | 0.08045977 | YES |
| UQCRQ | ENSG00000164405 | 5 | 46 | 1.20E−08 | 1.34E−07 | 0.09803922 | YES |
| URB1 | ENSG00000142207 | 10 | 262 | 3.38E−43 | 1.32E−41 | 0.03676471 | YES |
| URB2 | ENSG00000135763 | 87 | 166 | 3.59E−06 | 3.58E−05 | 0.34387352 | YES |
| URI1 | ENSG00000105176 | 164 | 1440 | 2.66E−115 | 9.66E−113 | 0.10224439 | YES |
| URM1 | ENSG00000167118 | 55 | 359 | 1.57E−38 | 5.05E−37 | 0.13285024 | YES |
| UROD | ENSG00000126088 | 44 | 141 | 2.26E−11 | 2.78E−10 | 0.23783784 | YES |
| USE1 | ENSG00000053501 | 43 | 172 | 4.34E−16 | 6.43E−15 | 0.2 | YES |
| USP10 | ENSG00000103194 | 1768 | 2103 | 0.00071369 | 0.00617675 | 0.45672953 | YES |
| USP36 | ENSG00000055483 | 251 | 1600 | 5.48E−106 | 1.57E−103 | 0.13560238 | YES |
| USP37 | ENSG00000135913 | 44 | 326 | 5.94E−38 | 1.89E−36 | 0.11891892 | YES |
| USP39 | ENSG00000168883 | 3 | 123 | 3.29E−24 | 6.40E−23 | 0.02380952 | YES |
| USP5 | ENSG00000111667 | 1 | 150 | 4.61E−30 | 1.08E−28 | 0.00662252 | YES |
| USP9X | ENSG00000124486 | 431 | 908 | 2.99E−22 | 5.51E−21 | 0.321882 | YES |
| USPL1 | ENSG00000132952 | 78 | 600 | 1.02E−60 | 7.33E−59 | 0.11504425 | YES |
| UTP11L | ENSG00000183520 | 5 | 162 | 7.46E−30 | 1.74E−28 | 0.02994012 | YES |
| UTP14A | ENSG00000156697 | 57 | 357 | 1.41E−37 | 4.44E−36 | 0.13768116 | YES |
| UTP15 | ENSG00000164338 | 9 | 160 | 2.02E−27 | 4.33E−26 | 0.05325444 | YES |
| UTP18 | ENSG00000011260 | 9 | 179 | 1.35E−30 | 3.24E−29 | 0.04787234 | YES |
| UTP20 | ENSG00000120800 | 9 | 130 | 2.91E−22 | 5.36E−21 | 0.0647482 | YES |
| UTP6 | ENSG00000108651 | 19 | 723 | 3.36E−97 | 7.61E−95 | 0.02560647 | YES |
| UVRAG | ENSG00000198382 | 1147 | 2266 | 6.83E−32 | 1.71E−30 | 0.33606798 | YES |
| UXT | ENSG00000126756 | 23 | 202 | 7.43E−28 | 1.62E−26 | 0.10222222 | YES |
| VARS | ENSG00000204394 | 214 | 297 | 0.00094589 | 0.00811394 | 0.41878669 | YES |
| VBP1 | ENSG00000155959 | 99 | 195 | 2.88E−07 | 3.00E−06 | 0.33673469 | YES |
| VCP | ENSG00000165280 | 25 | 128 | 3.75E−15 | 5.38E−14 | 0.16339869 | YES |
| VEZT | ENSG00000028203 | 88 | 266 | 4.77E−17 | 7.31E−16 | 0.24858757 | YES |
| VMP1 | ENSG00000062716 | 797 | 13120 | 0 | 0 | 0.05726809 | YES |
| VPS13D | ENSG00000048707 | 128 | 1418 | 9.43E−124 | 4.22E−121 | 0.08279431 | YES |
| VPS25 | ENSG00000131475 | 15 | 313 | 2.75E−48 | 1.26E−46 | 0.04573171 | YES |
| VPS29 | ENSG00000111237 | 59 | 308 | 2.65E−30 | 6.30E−29 | 0.16076294 | YES |
| VPS33A | ENSG00000139719 | 296 | 427 | 3.88E−05 | 0.00036595 | 0.40940526 | YES |
| VPS35 | ENSG00000069329 | 26 | 253 | 1.83E−34 | 5.11E−33 | 0.09318996 | YES |
| VPS36 | ENSG00000136100 | 84 | 279 | 1.49E−19 | 2.47E−18 | 0.23140496 | YES |
| VPS51 | ENSG00000149823 | 43 | 90 | 6.31E−05 | 0.00058708 | 0.32330827 | YES |
| VPS52 | ENSG00000223501 | 218 | 403 | 4.15E−10 | 4.84E−09 | 0.3510467 | YES |
| VPS53 | ENSG00000141252 | 300 | 975 | 2.46E−45 | 1.02E−43 | 0.23529412 | YES |
| VPS72 | ENSG00000163159 | 35 | 392 | 1.60E−50 | 7.89E−49 | 0.08196721 | YES |
| WAC | ENSG00000095787 | 118 | 731 | 7.58E−63 | 5.86E−61 | 0.13898704 | YES |
| WAPAL | ENSG00000062650 | 60 | 279 | 4.81E−26 | 9.84E−25 | 0.17699115 | YES |
| WARS | ENSG00000140105 | 98 | 705 | 4.58E−66 | 4.00E−64 | 0.12204234 | YES |
| WARS2 | ENSG00000116874 | 101 | 200 | 1.84E−07 | 1.93E−06 | 0.33554817 | YES |
| WBP11 | ENSG00000084463 | 5 | 138 | 9.68E−26 | 1.96E−24 | 0.03496503 | YES |
| WBP4 | ENSG00000120688 | 37 | 124 | 8.60E−11 | 1.03E−09 | 0.22981366 | YES |
| WBSCR22 | ENSG00000071462 | 9 | 159 | 2.98E−27 | 6.36E−26 | 0.05357143 | YES |
| WDHD1 | ENSG00000198554 | 171 | 943 | 9.44E−70 | 9.42E−68 | 0.1535009 | YES |
| WDR12 | ENSG00000138442 | 174 | 590 | 4.30E−34 | 1.18E−32 | 0.22774869 | YES |
| WDR18 | ENSG00000065268 | 17 | 241 | 1.31E−36 | 4.00E−35 | 0.06589147 | YES |
| WDR20 | ENSG00000140153 | 226 | 361 | 1.75E−06 | 1.76E−05 | 0.38500852 | YES |
| WDR25 | ENSG00000176473 | 52 | 163 | 2.23E−12 | 2.86E−11 | 0.24186047 | YES |
| WDR3 | ENSG00000065183 | 33 | 308 | 1.16E−39 | 3.89E−38 | 0.09677419 | YES |
| WDR33 | ENSG00000136709 | 51 | 485 | 2.29E−56 | 1.39E−54 | 0.09514925 | YES |
| WDR36 | ENSG00000134987 | 12 | 332 | 1.89E−52 | 9.82E−51 | 0.03488372 | YES |
| WDR4 | ENSG00000160193 | 219 | 699 | 9.21E−36 | 2.74E−34 | 0.23856209 | YES |
| WDR43 | ENSG00000163811 | 22 | 631 | 1.04E−85 | 1.62E−83 | 0.03369066 | YES |
| WDR44 | ENSG00000131725 | 339 | 772 | 2.13E−23 | 4.05E−22 | 0.30513051 | YES |
| WDR46 | ENSG00000227057 | 26 | 58 | 0.00040708 | 0.00359259 | 0.30952381 | YES |
| WDR5 | ENSG00000196363 | 24 | 81 | 6.14E−08 | 6.60E−07 | 0.22857143 | YES |
| WDR55 | ENSG00000120314 | 8 | 131 | 6.10E−23 | 1.14E−21 | 0.05755396 | YES |
| WDR59 | ENSG00000103091 | 54 | 176 | 1.23E−13 | 1.66E−12 | 0.23478261 | YES |
| WDR70 | ENSG00000082068 | 80 | 2013 | 1.21E−181 | 1.11E−178 | 0.03822265 | YES |
| WDR74 | ENSG00000133316 | 15 | 150 | 7.40E−23 | 1.38E−21 | 0.09090909 | YES |
| WDR75 | ENSG00000115368 | 3 | 180 | 5.91E−34 | 1.61E−32 | 0.01639344 | YES |
| WDR77 | ENSG00000116455 | 7 | 88 | 1.07E−15 | 1.56E−14 | 0.07368421 | YES |
| WDR82 | ENSG00000164091 | 30 | 546 | 2.77E−72 | 2.95E−70 | 0.05208333 | YES |
| WDR92 | ENSG00000243667 | 212 | 615 | 3.22E−29 | 7.34E−28 | 0.25634825 | YES |
| WEE1 | ENSG00000166483 | 8 | 215 | 6.16E−37 | 1.90E−35 | 0.03587444 | YES |
| WIBG | ENSG00000170473 | 474 | 677 | 2.91E−06 | 2.90E−05 | 0.41181581 | YES |
| WNK1 | ENSG00000060237 | 929 | 1962 | 1.81E−34 | 5.06E−33 | 0.3213421 | YES |
| WRB | ENSG00000182093 | 32 | 705 | 9.11E−90 | 1.59E−87 | 0.04341927 | YES |
| XAB2 | ENSG00000076924 | 0 | 21 | 3.47E−06 | 3.46E−05 | 0 | YES |
| XPO1 | ENSG00000082898 | 216 | 870 | 2.59E−52 | 1.33E−50 | 0.19889503 | YES |
| XPO5 | ENSG00000124571 | 49 | 294 | 1.09E−31 | 2.72E−30 | 0.14285714 | YES |
| XRCC1 | ENSG00000073050 | 636 | 858 | 1.58E−05 | 0.00015157 | 0.42570281 | YES |
| XRCC2 | ENSG00000196584 | 56 | 337 | 3.19E−35 | 9.22E−34 | 0.14249364 | YES |
| XRCC3 | ENSG00000126215 | 27 | 123 | 1.17E−13 | 1.58E−12 | 0.18 | YES |
| XRCC6 | ENSG00000196419 | 20 | 149 | 1.57E−20 | 2.72E−19 | 0.1183432 | YES |
| XRN1 | ENSG00000114127 | 172 | 656 | 3.36E−41 | 1.20E−39 | 0.20772947 | YES |
| XRN2 | ENSG00000088930 | 106 | 480 | 3.47E−38 | 1.11E−36 | 0.18088737 | YES |
| YAE1D1 | ENSG00000241127 | 5 | 114 | 1.71E−21 | 3.05E−20 | 0.04201681 | YES |
| YARS | ENSG00000134684 | 92 | 945 | 8.17E−93 | 1.62E−90 | 0.08871745 | YES |
| YARS2 | ENSG00000139131 | 30 | 179 | 1.82E−21 | 3.26E−20 | 0.14354067 | YES |
| YBX1 | ENSG00000065978 | 30 | 188 | 7.18E−23 | 1.34E−21 | 0.13761468 | YES |
| YEATS2 | ENSG00000163872 | 377 | 560 | 1.46E−06 | 1.48E−05 | 0.40234792 | YES |
| YEATS4 | ENSG00000127337 | 125 | 271 | 9.07E−11 | 1.08E−09 | 0.31565657 | YES |
| YKT6 | ENSG00000106636 | 4 | 48 | 1.53E−09 | 1.76E−08 | 0.07692308 | YES |
| YLPM1 | ENSG00000119596 | 154 | 588 | 3.00E−38 | 9.63E−37 | 0.20754717 | YES |
| YME1L1 | ENSG00000136758 | 118 | 324 | 1.86E−17 | 2.87E−16 | 0.26696833 | YES |
| YPEL5 | ENSG00000119801 | 50 | 101 | 5.26E−05 | 0.00049103 | 0.33112583 | YES |
| YRDC | ENSG00000196449 | 0 | 72 | 1.46E−16 | 2.20E−15 | 0 | YES |
| YTHDC1 | ENSG00000083896 | 19 | 392 | 1.92E−57 | 1.22E−55 | 0.04622871 | YES |
| YY1 | ENSG00000100811 | 9 | 140 | 5.29E−24 | 1.02E−22 | 0.06040268 | YES |
| ZBTB11 | ENSG00000066422 | 131 | 239 | 4.51E−07 | 4.65E−06 | 0.35405405 | YES |
| ZBTB17 | ENSG00000116809 | 53 | 328 | 5.10E−35 | 1.46E−33 | 0.13910761 | YES |
| ZBTB2 | ENSG00000181472 | 557 | 792 | 1.18E−06 | 1.20E−05 | 0.41289844 | YES |
| ZBTB8OS | ENSG00000176261 | 35 | 569 | 5.74E−73 | 6.34E−71 | 0.05794702 | YES |
| ZC3H11A | ENSG00000058673 | 193 | 332 | 1.55E−07 | 1.63E−06 | 0.36761905 | YES |
| ZC3H13 | ENSG00000123200 | 170 | 479 | 9.76E−24 | 1.86E−22 | 0.26194145 | YES |
| ZC3H18 | ENSG00000158545 | 8 | 151 | 2.11E−26 | 4.39E−25 | 0.05031447 | YES |
| ZC3H4 | ENSG00000130749 | 3180 | 8149 | 6.27E−93 | 1.26E−90 | 0.28069556 | YES |
| ZC3H8 | ENSG00000144161 | 32 | 486 | 5.04E−64 | 4.13E−62 | 0.06177606 | YES |
| ZCCHC9 | ENSG00000131732 | 4 | 117 | 1.40E−22 | 2.60E−21 | 0.03305785 | YES |
| ZCRB1 | ENSG00000139168 | 86 | 224 | 1.62E−12 | 2.09E−11 | 0.27741935 | YES |
| ZFC3H1 | ENSG00000133858 | 100 | 210 | 1.40E−08 | 1.55E−07 | 0.32258065 | YES |
| ZFR | ENSG00000056097 | 329 | 453 | 0.0001864 | 0.00167721 | 0.42071611 | YES |
| ZMAT2 | ENSG00000146007 | 3 | 91 | 2.20E−18 | 3.52E−17 | 0.03191489 | YES |
| ZMAT5 | ENSG00000100319 | 38 | 190 | 2.44E−20 | 4.19E−19 | 0.16666667 | YES |
| ZMYND8 | ENSG00000101040 | 3526 | 6052 | 6.26E−33 | 1.63E−31 | 0.36813531 | YES |
| ZNF131 | ENSG00000172262 | 58 | 372 | 3.10E−39 | 1.02E−37 | 0.13488372 | YES |
| ZNF143 | ENSG00000166478 | 173 | 1017 | 3.43E−76 | 4.37E−74 | 0.14537815 | YES |
| ZNF148 | ENSG00000163848 | 1497 | 1830 | 0.00019557 | 0.00175885 | 0.44995491 | YES |
| ZNF207 | ENSG00000010244 | 65 | 444 | 3.43E−46 | 1.48E−44 | 0.12770138 | YES |
| ZNF236 | ENSG00000130856 | 230 | 801 | 4.51E−43 | 1.74E−41 | 0.22308438 | YES |
| ZNF259 | ENSG00000109917 | 3 | 86 | 1.90E−17 | 2.93E−16 | 0.03370787 | YES |
| ZNF335 | ENSG00000198026 | 83 | 459 | 2.85E−42 | 1.07E−40 | 0.15313653 | YES |
| ZNF407 | ENSG00000215421 | 213 | 452 | 1.49E−14 | 2.09E−13 | 0.32030075 | YES |
| ZNF574 | ENSG00000105732 | 11 | 70 | 1.55E−10 | 1.84E−09 | 0.13580247 | YES |
| ZNF622 | ENSG00000173545 | 26 | 71 | 6.55E−06 | 6.45E−05 | 0.26804124 | YES |
| ZNHIT1 | ENSG00000106400 | 69 | 184 | 3.22E−11 | 3.93E−10 | 0.27272727 | YES |
| ZNHIT3 | ENSG00000108278 | 10 | 57 | 1.48E−08 | 1.63E−07 | 0.14925373 | YES |
| ZNHIT6 | ENSG00000117174 | 34 | 171 | 8.36E−19 | 1.36E−17 | 0.16585366 | YES |
| ZRANB2 | ENSG00000132485 | 183 | 258 | 0.00106916 | 0.00911765 | 0.41496599 | YES |
| ZRSR2 | ENSG00000169249 | 170 | 370 | 2.30E−13 | 3.06E−12 | 0.31481481 | YES |
| ZW10 | ENSG00000086827 | 5 | 47 | 7.64E−09 | 8.53E−08 | 0.09615385 | YES |
| ZZZ3 | ENSG00000036549 | 371 | 1090 | 6.12E−43 | 2.34E−41 | 0.25393566 | YES |

HAP1

| GENE_SYMBOL | ENSEMBL_ID | tot. sense | tot. anti | p.val | q.val | ratio | selected |
|---|---|---|---|---|---|---|---|
| AAMP | ENSG00000127837 | 12 | 106 | 1.57E−14 | 1.97E−13 | 0.10169492 | YES |
| AARS | ENSG00000090861 | 167 | 1574 | 9.26E−94 | 1.44E−91 | 0.09592188 | YES |
| AASDHPPT | ENSG00000149313 | 179 | 545 | 6.59E−23 | 1.04E−21 | 0.24723757 | YES |
| AATF | ENSG00000108270 | 299 | 4255 | 1.49E−171 | 2.60E−168 | 0.06565657 | YES |
| ABCB7 | ENSG00000131269 | 104 | 1492 | 3.69E−103 | 7.85E−101 | 0.06516291 | YES |
| ABCE1 | ENSG00000164163 | 33 | 643 | 1.43E−65 | 8.18E−64 | 0.04881657 | YES |
| ABCF1 | ENSG00000204574 | 15 | 275 | 1.14E−35 | 2.60E−34 | 0.05172414 | YES |
| ABT1 | ENSG00000146109 | 8 | 83 | 1.23E−12 | 1.45E−11 | 0.08791209 | YES |
| ACACA | ENSG00000132142 | 8907 | 11278 | 7.04E−07 | 6.82E−06 | 0.44126827 | YES |
| ACIN1 | ENSG00000100813 | 167 | 647 | 8.59E−33 | 1.80E−31 | 0.20515971 | YES |
| ACO2 | ENSG00000100412 | 616 | 1071 | 5.85E−12 | 6.71E−11 | 0.36514523 | YES |
| ACTL6A | ENSG00000136518 | 31 | 412 | 8.85E−45 | 2.63E−43 | 0.06997743 | YES |
| ACTR10 | ENSG00000131966 | 28 | 253 | 3.42E−28 | 6.27E−27 | 0.09964413 | YES |
| ACTR2 | ENSG00000138071 | 207 | 567 | 1.69E−20 | 2.47E−19 | 0.26744186 | YES |
| ACTR3 | ENSG00000115091 | 1527 | 2991 | 1.40E−25 | 2.39E−24 | 0.33798141 | YES |
| ACTR6 | ENSG00000075089 | 88 | 416 | 6.47E−29 | 1.22E−27 | 0.17460317 | YES |
| ACTR8 | ENSG00000113812 | 69 | 367 | 1.05E−28 | 1.97E−27 | 0.15825688 | YES |
| ADAR | ENSG00000160710 | 160 | 545 | 5.66E−26 | 9.79E−25 | 0.22695035 | YES |
| ADAT2 | ENSG00000189007 | 60 | 456 | 4.53E−40 | 1.19E−38 | 0.11627907 | YES |
| ADSL | ENSG00000239900 | 110 | 1025 | 4.42E−73 | 3.28E−71 | 0.0969163 | YES |
| ADSS | ENSG00000035687 | 406 | 724 | 1.59E−10 | 1.74E−09 | 0.35929204 | YES |
| AFG3L2 | ENSG00000141385 | 74 | 831 | 2.46E−68 | 1.54E−66 | 0.08176796 | YES |
| AHCTF1 | ENSG00000153207 | 157 | 873 | 8.67E−52 | 3.30E−50 | 0.15242718 | YES |
| AHCY | ENSG00000101444 | 499 | 992 | 4.91E−16 | 6.39E−15 | 0.33467471 | YES |
| AIFM1 | ENSG00000156709 | 322 | 584 | 8.30E−10 | 8.94E−09 | 0.35540839 | YES |
| AK2 | ENSG00000004455 | 78 | 887 | 1.97E−71 | 1.38E−69 | 0.08082902 | YES |
| AKIRIN2 | ENSG00000135334 | 109 | 923 | 3.81E−66 | 2.23E−64 | 0.10562016 | YES |
| ALDOA | ENSG00000149925 | 220 | 661 | 2.33E−25 | 3.93E−24 | 0.24971623 | YES |
| ALG1 | ENSG00000033011 | 41 | 482 | 2.15E−48 | 7.27E−47 | 0.07839388 | YES |
| ALG11 | ENSG00000253710 | 13 | 199 | 4.50E−27 | 7.99E−26 | 0.06132075 | YES |
| ALG13 | ENSG00000101901 | 23 | 255 | 3.25E−30 | 6.31E−29 | 0.08273381 | YES |
| ALG14 | ENSG00000172339 | 259 | 1415 | 1.35E−67 | 8.25E−66 | 0.15471924 | YES |
| ALG2 | ENSG00000119523 | 5 | 65 | 4.80E−11 | 5.37E−10 | 0.07142857 | YES |
| ALG8 | ENSG00000159063 | 2017 | 2649 | 6.16E−06 | 5.70E−05 | 0.43227604 | YES |
| ALYREF | ENSG00000183684 | 49 | 481 | 9.03E−46 | 2.76E−44 | 0.09245283 | YES |
| AMD1 | ENSG00000123505 | 278 | 528 | 2.44E−10 | 2.68E−09 | 0.34491315 | YES |
| ANAPC1 | ENSG00000153107 | 327 | 1246 | 2.34E−47 | 7.63E−46 | 0.20788303 | YES |
| ANAPC10 | ENSG00000164162 | 45 | 590 | 4.24E−57 | 1.93E−55 | 0.07086614 | YES |
| ANAPC11 | ENSG00000141552 | 56 | 337 | 4.77E−29 | 9.04E−28 | 0.14249364 | YES |
| ANAPC13 | ENSG00000129055 | 236 | 846 | 8.67E−36 | 1.98E−34 | 0.2181146 | YES |
| ANAPC15 | ENSG00000110200 | 339 | 645 | 1.41E−11 | 1.60E−10 | 0.3445122 | YES |
| ANAPC4 | ENSG00000053900 | 46 | 539 | 3.56E−52 | 1.37E−50 | 0.07863248 | YES |
| ANAPC5 | ENSG00000089053 | 60 | 896 | 4.41E−77 | 3.87E−75 | 0.06276151 | YES |
| ANKLE2 | ENSG00000176915 | 91 | 350 | 7.27E−22 | 1.12E−20 | 0.20634921 | YES |
| ANKRD17 | ENSG00000132466 | 1572 | 3085 | 5.20E−26 | 9.02E−25 | 0.33755637 | YES |
| ANLN | ENSG00000011426 | 141 | 2502 | 1.32E−141 | 7.00E−139 | 0.05334847 | YES |
| AP2S1 | ENSG00000042753 | 157 | 369 | 1.49E−12 | 1.74E−11 | 0.29847909 | YES |
| APC | ENSG00000134982 | 337 | 816 | 4.66E−21 | 6.94E−20 | 0.29228101 | YES |
| APEX1 | ENSG00000100823 | 251 | 419 | 1.14E−06 | 1.10E−05 | 0.37462687 | YES |
| API5 | ENSG00000166181 | 100 | 236 | 1.49E−09 | 1.59E−08 | 0.29761905 | YES |
| AQR | ENSG00000021776 | 61 | 548 | 1.78E−48 | 6.09E−47 | 0.1001642 | YES |
| ARCN1 | ENSG00000095139 | 44 | 719 | 2.57E−68 | 1.60E−66 | 0.0576671 | YES |
| ARGLU1 | ENSG00000134884 | 33 | 295 | 2.05E−31 | 4.12E−30 | 0.10060976 | YES |
| ARL2 | ENSG00000213465 | 161 | 457 | 8.19E−19 | 1.14E−17 | 0.2605178 | YES |
| ARMC7 | ENSG00000125449 | 122 | 730 | 1.87E−48 | 6.36E−47 | 0.14319249 | YES |
| ARMC8 | ENSG00000114098 | 615 | 798 | 0.00098044 | 0.00792012 | 0.43524416 | YES |
| ARPC2 | ENSG00000163466 | 878 | 2395 | 8.73E−44 | 2.52E−42 | 0.26825542 | YES |
| ARPC3 | ENSG00000111229 | 186 | 430 | 2.04E−13 | 2.46E−12 | 0.30194805 | YES |
| ARPC4 | ENSG00000241553 | 152 | 488 | 1.01E−22 | 1.58E−21 | 0.2375 | YES |
| ASCC3 | ENSG00000112249 | 754 | 2143 | 3.48E−44 | 1.02E−42 | 0.26026924 | YES |
| ASUN | ENSG00000064102 | 408 | 821 | 6.47E−15 | 8.22E−14 | 0.33197722 | YES |
| ATAD5 | ENSG00000176208 | 371 | 758 | 9.32E−15 | 1.18E−13 | 0.32860939 | YES |
| ATIC | ENSG00000138363 | 379 | 529 | 0.00028452 | 0.00240402 | 0.41740088 | YES |
| ATL2 | ENSG00000119787 | 267 | 880 | 1.76E−33 | 3.74E−32 | 0.23278117 | YES |
| ATP13A1 | ENSG00000105726 | 186 | 282 | 0.00033095 | 0.00278285 | 0.3974359 | YES |
| ATP1A1 | ENSG00000163399 | 557 | 1596 | 8.88E−39 | 2.23E−37 | 0.25870878 | YES |
| ATP2A2 | ENSG00000174437 | 103 | 1217 | 2.52E−87 | 3.22E−85 | 0.0780303 | YES |
| ATP5A1 | ENSG00000152234 | 137 | 583 | 2.66E−33 | 5.63E−32 | 0.19027778 | YES |
| ATP5B | ENSG00000110955 | 115 | 506 | 3.25E−31 | 6.52E−30 | 0.18518519 | YES |
| ATP5C1 | ENSG00000165629 | 113 | 383 | 1.37E−20 | 2.01E−19 | 0.22782258 | YES |
| ATP5E | ENSG00000124172 | 158 | 412 | 1.01E−15 | 1.31E−14 | 0.27719298 | YES |
| ATP5F1 | ENSG00000116459 | 154 | 500 | 2.30E−23 | 3.67E−22 | 0.23547401 | YES |
| ATP5H | ENSG00000167863 | 92 | 335 | 3.34E−20 | 4.83E−19 | 0.21545667 | YES |
| ATP5J2 | ENSG00000241468 | 227 | 874 | 4.15E−39 | 1.05E−37 | 0.2061762 | YES |
| ATP5L | ENSG00000167283 | 189 | 393 | 1.57E−10 | 1.73E−09 | 0.32474227 | YES |
| ATP5O | ENSG00000241837 | 148 | 586 | 1.99E−31 | 4.02E−30 | 0.20163488 | YES |
| ATP6AP1 | ENSG00000071553 | 39 | 244 | 1.52E−23 | 2.44E−22 | 0.13780919 | YES |
| ATP6AP2 | ENSG00000182220 | 14 | 71 | 1.61E−08 | 1.66E−07 | 0.16470588 | YES |
| ATP6V0B | ENSG00000117410 | 49 | 184 | 9.86E−14 | 1.20E−12 | 0.21030043 | YES |
| ATP6V0C | ENSG00000185883 | 22 | 176 | 1.32E−20 | 1.93E−19 | 0.11111111 | YES |
| ATP6V0D1 | ENSG00000159720 | 42 | 434 | 2.39E−43 | 6.84E−42 | 0.08823529 | YES |
| ATP6V1A | ENSG00000114573 | 430 | 1557 | 1.17E−50 | 4.20E−49 | 0.21640664 | YES |
| ATP6V1D | ENSG00000100554 | 41 | 263 | 3.62E−25 | 6.09E−24 | 0.13486842 | YES |
| ATP6V1E1 | ENSG00000131100 | 74 | 330 | 1.13E−23 | 1.82E−22 | 0.18316832 | YES |
| ATP6V1F | ENSG00000128524 | 44 | 292 | 1.40E−27 | 2.51E−26 | 0.13095238 | YES |
| ATP6V1G1 | ENSG00000136888 | 150 | 306 | 9.29E−09 | 9.61E−08 | 0.32894737 | YES |
| ATR | ENSG00000175054 | 62 | 682 | 7.13E−60 | 3.56E−58 | 0.08333333 | YES |
| ATRIP | ENSG00000164053 | 24 | 286 | 1.60E−33 | 3.41E−32 | 0.07741935 | YES |
| ATRX | ENSG00000085224 | 2324 | 3135 | 3.67E−07 | 3.62E−06 | 0.425719 | YES |
| ATXN10 | ENSG00000130638 | 1166 | 1941 | 9.87E−14 | 1.20E−12 | 0.37528162 | YES |
| AURKA | ENSG00000087586 | 47 | 300 | 1.38E−27 | 2.48E−26 | 0.13544669 | YES |
| B4GALT5 | ENSG00000158470 | 4135 | 4976 | 0.00036315 | 0.00303905 | 0.453847 | YES |
| BANF1 | ENSG00000175334 | 45 | 212 | 4.95E−18 | 6.81E−17 | 0.17509728 | YES |
| BANP | ENSG00000172530 | 194 | 3016 | 1.50E−150 | 1.46E−147 | 0.06043614 | YES |
| BARD1 | ENSG00000138376 | 84 | 970 | 8.39E−76 | 7.04E−74 | 0.07969639 | YES |
| BCAS2 | ENSG00000116752 | 12 | 175 | 2.61E−24 | 4.29E−23 | 0.06417112 | YES |
| BCCIP | ENSG00000107949 | 23 | 381 | 2.07E−44 | 6.11E−43 | 0.05693069 | YES |
| BCL2 | ENSG00000171791 | 1084 | 1651 | 1.58E−09 | 1.68E−08 | 0.39634369 | YES |
| BCL2L1 | ENSG00000171552 | 747 | 2552 | 2.07E−60 | 1.05E−58 | 0.22643225 | YES |
| BCLAF1 | ENSG00000029363 | 38 | 258 | 1.67E−25 | 2.84E−24 | 0.12837838 | YES |
| BCS1L | ENSG00000074582 | 429 | 610 | 7.43E−05 | 0.00064929 | 0.41289702 | YES |
| BDP1 | ENSG00000145734 | 384 | 805 | 4.54E−16 | 5.91E−15 | 0.32296047 | YES |
| BECN1 | ENSG00000126581 | 121 | 279 | 3.08E−10 | 3.37E−09 | 0.3025 | YES |
| BET1 | ENSG00000105829 | 16 | 244 | 1.52E−31 | 3.08E−30 | 0.06153846 | YES |
| BIRC5 | ENSG00000089685 | 12 | 252 | 3.86E−34 | 8.35E−33 | 0.04545455 | YES |
| BLM | ENSG00000197299 | 1010 | 1369 | 1.57E−05 | 0.00014221 | 0.42454813 | YES |
| BMS1 | ENSG00000165733 | 46 | 711 | 4.77E−67 | 2.88E−65 | 0.06076618 | YES |
| BNIP1 | ENSG00000113734 | 8 | 142 | 1.86E−21 | 2.82E−20 | 0.05333333 | YES |
| BOD1L1 | ENSG00000038219 | 66 | 182 | 5.82E−10 | 6.31E−09 | 0.26612903 | YES |
| BORA | ENSG00000136122 | 80 | 329 | 3.47E−22 | 5.38E−21 | 0.19559902 | YES |
| BPTF | ENSG00000171634 | 815 | 1540 | 1.22E−17 | 1.66E−16 | 0.34607219 | YES |
| BRCA1 | ENSG00000012048 | 561 | 2430 | 1.09E−74 | 8.75E−73 | 0.18756269 | YES |
| BRCA2 | ENSG00000139618 | 68 | 791 | 6.30E−67 | 3.79E−65 | 0.07916182 | YES |
| BRD4 | ENSG00000141867 | 1055 | 2244 | 2.33E−27 | 4.18E−26 | 0.31979388 | YES |
| BRD8 | ENSG00000112983 | 30 | 204 | 1.69E−21 | 2.58E−20 | 0.12820513 | YES |
| BRF1 | ENSG00000185024 | 133 | 758 | 3.24E−48 | 1.09E−46 | 0.14927048 | YES |
| BRIP1 | ENSG00000136492 | 2625 | 3496 | 6.15E−07 | 5.98E−06 | 0.42885149 | YES |
| BRIX1 | ENSG00000113460 | 17 | 272 | 1.60E−34 | 3.53E−33 | 0.05882353 | YES |
| BTAF1 | ENSG00000095564 | 131 | 1084 | 3.75E−72 | 2.70E−70 | 0.10781893 | YES |
| BTF3 | ENSG00000145741 | 129 | 493 | 2.92E−27 | 5.20E−26 | 0.2073955 | YES |
| BUB1 | ENSG00000169679 | 132 | 274 | 1.81E−08 | 1.86E−07 | 0.32512315 | YES |
| BUB1B | ENSG00000156970 | 486 | 930 | 3.18E−14 | 3.94E−13 | 0.34322034 | YES |
| BUB3 | ENSG00000154473 | 31 | 204 | 3.56E−21 | 5.34E−20 | 0.13191489 | YES |
| BUD13 | ENSG00000137656 | 56 | 707 | 1.08E−63 | 5.84E−62 | 0.0733945 | YES |
| BUD31 | ENSG00000106245 | 148 | 374 | 3.82E−14 | 4.71E−13 | 0.2835249 | YES |
| BYSL | ENSG00000112578 | 104 | 538 | 1.83E−36 | 4.26E−35 | 0.16199377 | YES |
| C12orf45 | ENSG00000151131 | 9 | 198 | 1.24E−28 | 2.32E−27 | 0.04347826 | YES |
| C14orf166 | ENSG00000087302 | 162 | 2224 | 4.17E−126 | 1.73E−123 | 0.06789606 | YES |
| C15orf41 | ENSG00000186073 | 3152 | 8868 | 2.88E−74 | 2.30E−72 | 0.26222962 | YES |
| C16orf80 | ENSG00000070761 | 154 | 457 | 6.87E−20 | 9.85E−19 | 0.25204583 | YES |
| C18orf21 | ENSG00000141428 | 108 | 394 | 1.50E−22 | 2.34E−21 | 0.21513944 | YES |
| C19orf43 | ENSG00000123144 | 277 | 458 | 8.32E−07 | 8.04E−06 | 0.37687075 | YES |
| C1orf131 | ENSG00000143633 | 36 | 465 | 2.19E−48 | 7.39E−47 | 0.07185629 | YES |
| C21orf59 | ENSG00000159079 | 145 | 518 | 2.13E−26 | 3.72E−25 | 0.21870287 | YES |
| C22orf28 | ENSG00000100220 | 19 | 144 | 2.34E−17 | 3.16E−16 | 0.11656442 | YES |
| C3orf17 | ENSG00000163608 | 32 | 554 | 4.10E−58 | 1.92E−56 | 0.05460751 | YES |
| C6orf48 | ENSG00000204387 | 861 | 1119 | 0.00030727 | 0.00259252 | 0.43484848 | YES |
| C6orf57 | ENSG00000154079 | 337 | 485 | 0.00013864 | 0.00119688 | 0.40997567 | YES |
| C7orf73 | ENSG00000243317 | 272 | 462 | 2.41E−07 | 2.40E−06 | 0.37057221 | YES |
| C9orf114 | ENSG00000198917 | 16 | 202 | 2.95E−26 | 5.14E−25 | 0.0733945 | YES |
| C9orf78 | ENSG00000136819 | 38 | 202 | 8.67E−19 | 1.21E−17 | 0.15833333 | YES |
| CAB39 | ENSG00000135932 | 182 | 363 | 2.87E−09 | 3.03E−08 | 0.33394495 | YES |
| CAD | ENSG00000084774 | 254 | 460 | 1.37E−08 | 1.41E−07 | 0.3557423 | YES |
| CALM2 | ENSG00000143933 | 234 | 476 | 2.81E−11 | 3.17E−10 | 0.32957746 | YES |
| CAMLG | ENSG00000164615 | 89 | 638 | 8.28E−49 | 2.83E−47 | 0.12242091 | YES |
| CAPNS1 | ENSG00000126247 | 375 | 524 | 0.00028434 | 0.00240368 | 0.41713014 | YES |
| CAPZB | ENSG00000077549 | 219 | 687 | 2.45E−27 | 4.39E−26 | 0.24172185 | YES |
| CARS | ENSG00000110619 | 42 | 590 | 4.74E−58 | 2.21E−56 | 0.0664557 | YES |
| CASC3 | ENSG00000108349 | 369 | 596 | 2.81E−07 | 2.79E−06 | 0.38238342 | YES |
| CASC5 | ENSG00000137812 | 107 | 793 | 6.58E−57 | 2.98E−55 | 0.11888889 | YES |
| CCAR1 | ENSG00000060339 | 763 | 1353 | 4.31E−14 | 5.30E−13 | 0.36058601 | YES |
| CCDC115 | ENSG00000136710 | 28 | 66 | 0.00024521 | 0.00208295 | 0.29787234 | YES |
| CCDC130 | ENSG00000104957 | 114 | 431 | 9.89E−25 | 1.64E−23 | 0.20917431 | YES |
| CCDC174 | ENSG00000154781 | 24 | 423 | 2.21E−48 | 7.44E−47 | 0.05369128 | YES |
| CCDC47 | ENSG00000108588 | 908 | 1312 | 4.09E−07 | 4.02E−06 | 0.40900901 | YES |
| CCDC59 | ENSG00000133773 | 19 | 255 | 1.03E−31 | 2.09E−30 | 0.06934307 | YES |
| CCDC84 | ENSG00000186166 | 61 | 576 | 5.22E−51 | 1.93E−49 | 0.09576138 | YES |
| CCDC86 | ENSG00000110104 | 51 | 389 | 5.78E−36 | 1.33E−34 | 0.11590909 | YES |
| CCDC94 | ENSG00000105248 | 15 | 288 | 3.43E−37 | 8.13E−36 | 0.04950495 | YES |
| CCNA2 | ENSG00000145386 | 31 | 124 | 6.40E−11 | 7.12E−10 | 0.2 | YES |
| CCNF | ENSG00000162063 | 50 | 247 | 9.46E−21 | 1.39E−19 | 0.16835017 | YES |
| CCNH | ENSG00000134480 | 44 | 251 | 6.94E−23 | 1.09E−21 | 0.14915254 | YES |
| CCNK | ENSG00000090061 | 21 | 205 | 9.70E−25 | 1.61E−23 | 0.09292035 | YES |
| CCP110 | ENSG00000103540 | 448 | 1244 | 1.17E−32 | 2.43E−31 | 0.26477541 | YES |
| CCT2 | ENSG00000166226 | 3 | 122 | 6.82E−21 | 1.01E−19 | 0.024 | YES |
| CCT3 | ENSG00000163468 | 16 | 198 | 9.74E−26 | 1.67E−24 | 0.07476636 | YES |
| CCT4 | ENSG00000115484 | 74 | 790 | 3.48E−65 | 1.97E−63 | 0.08564815 | YES |
| CCT5 | ENSG00000150753 | 41 | 817 | 6.90E−77 | 5.91E−75 | 0.04778555 | YES |
| CCT6A | ENSG00000146731 | 41 | 189 | 2.31E−16 | 3.04E−15 | 0.17826087 | YES |
| CCT7 | ENSG00000135624 | 52 | 860 | 9.05E−77 | 7.71E−75 | 0.05701754 | YES |
| CCT8 | ENSG00000156261 | 43 | 694 | 1.35E−66 | 8.07E−65 | 0.05834464 | YES |
| CD3EAP | ENSG00000117877 | 109 | 335 | 5.79E−17 | 7.75E−16 | 0.2454955 | YES |
| CDC123 | ENSG00000151465 | 123 | 1646 | 4.96E−107 | 1.20E−104 | 0.06953081 | YES |
| CDC16 | ENSG00000130177 | 111 | 390 | 1.33E−21 | 2.03E−20 | 0.22155689 | YES |
| CDC20 | ENSG00000117399 | 12 | 93 | 1.42E−12 | 1.66E−11 | 0.11428571 | YES |
| CDC23 | ENSG00000094880 | 17 | 306 | 1.80E−38 | 4.44E−37 | 0.05263158 | YES |
| CDC26 | ENSG00000176386 | 22 | 238 | 1.64E−28 | 3.05E−27 | 0.08461538 | YES |
| CDC27 | ENSG00000004897 | 67 | 1871 | 5.09E−131 | 2.22E−128 | 0.03457172 | YES |
| CDC37 | ENSG00000105401 | 31 | 618 | 4.19E−64 | 2.31E−62 | 0.04776579 | YES |
| CDC40 | ENSG00000168438 | 63 | 863 | 8.33E−74 | 6.55E−72 | 0.06803456 | YES |
| CDC42 | ENSG00000070831 | 891 | 2871 | 2.09E−59 | 1.02E−57 | 0.23684211 | YES |
| CDC45 | ENSG00000093009 | 87 | 893 | 1.72E−69 | 1.14E−67 | 0.08877551 | YES |
| CDC5L | ENSG00000096401 | 45 | 516 | 2.38E−50 | 8.51E−49 | 0.0802139 | YES |
| CDC6 | ENSG00000094804 | 171 | 568 | 5.96E−26 | 1.03E−24 | 0.23139378 | YES |
| CDC7 | ENSG00000097046 | 296 | 956 | 2.63E−34 | 5.77E−33 | 0.23642173 | YES |
| CDC73 | ENSG00000134371 | 118 | 1907 | 5.09E−121 | 1.74E−118 | 0.0582716 | YES |
| CDCA5 | ENSG00000146670 | 24 | 183 | 7.98E−21 | 1.18E−19 | 0.11594203 | YES |
| CDIPT | ENSG00000103502 | 46 | 181 | 4.14E−14 | 5.10E−13 | 0.20264317 | YES |
| CDK1 | ENSG00000170312 | 33 | 1081 | 1.28E−97 | 2.29E−95 | 0.02962298 | YES |
| CDK12 | ENSG00000167258 | 89 | 1061 | 9.49E−81 | 9.42E−79 | 0.0773913 | YES |
| CDK7 | ENSG00000134058 | 74 | 1423 | 1.10E−106 | 2.63E−104 | 0.0494322 | YES |
| CDK9 | ENSG00000136807 | 7 | 115 | 5.64E−18 | 7.71E−17 | 0.05737705 | YES |
| CDT1 | ENSG00000167513 | 101 | 275 | 7.17E−13 | 8.47E−12 | 0.26861702 | YES |
| CEBPD | ENSG00000221869 | 34 | 80 | 7.58E−05 | 0.00066134 | 0.29824561 | YES |
| CEBPZ | ENSG00000115816 | 193 | 2393 | 2.83E−127 | 1.20E−124 | 0.07463264 | YES |
| CENPA | ENSG00000115163 | 20 | 162 | 1.87E−19 | 2.66E−18 | 0.10989011 | YES |
| CENPC1 | ENSG00000145241 | 57 | 594 | 8.73E−54 | 3.57E−52 | 0.0875576 | YES |
| CENPH | ENSG00000153044 | 103 | 723 | 2.36E−52 | 9.23E−51 | 0.12469734 | YES |
| CENPJ | ENSG00000151849 | 19 | 303 | 2.35E−37 | 5.60E−36 | 0.05900621 | YES |
| CENPL | ENSG00000120334 | 85 | 309 | 4.23E−19 | 5.96E−18 | 0.21573604 | YES |
| CENPM | ENSG00000100162 | 70 | 739 | 2.92E−62 | 1.52E−60 | 0.08652658 | YES |
| CENPN | ENSG00000166451 | 45 | 229 | 4.94E−20 | 7.11E−19 | 0.16423358 | YES |
| CENPW | ENSG00000203760 | 17 | 144 | 4.36E−18 | 6.01E−17 | 0.10559006 | YES |
| CEP135 | ENSG00000174799 | 451 | 4832 | 2.61E−166 | 3.51E−163 | 0.08536816 | YES |
| CEP152 | ENSG00000103995 | 131 | 1344 | 1.76E−88 | 2.31E−86 | 0.08881356 | YES |
| CEP192 | ENSG00000101639 | 245 | 2163 | 5.83E−108 | 1.50E−105 | 0.10174419 | YES |
| CEP350 | ENSG00000135837 | 551 | 1622 | 1.51E−40 | 4.04E−39 | 0.2535665 | YES |
| CEP44 | ENSG00000164118 | 73 | 260 | 1.17E−16 | 1.55E−15 | 0.21921922 | YES |
| CEP55 | ENSG00000138180 | 50 | 423 | 9.65E−40 | 2.48E−38 | 0.10570825 | YES |
| CEP57 | ENSG00000166037 | 243 | 528 | 1.36E−13 | 1.65E−12 | 0.3151751 | YES |
| CEP63 | ENSG00000182923 | 304 | 885 | 6.71E−29 | 1.27E−27 | 0.25567704 | YES |
| CEP85 | ENSG00000130695 | 215 | 2392 | 3.36E−123 | 1.20E−120 | 0.08247027 | YES |
| CEP97 | ENSG00000182504 | 233 | 688 | 2.04E−25 | 3.45E−24 | 0.25298588 | YES |
| CFDP1 | ENSG00000153774 | 367 | 2209 | 2.09E−90 | 2.83E−88 | 0.14246894 | YES |
| CHAF1A | ENSG00000167670 | 114 | 800 | 8.94E−56 | 3.88E−54 | 0.12472648 | YES |
| CHAF1B | ENSG00000159259 | 250 | 396 | 1.29E−05 | 0.00011769 | 0.3869969 | YES |
| CHCHD4 | ENSG00000163528 | 62 | 682 | 7.13E−60 | 3.56E−58 | 0.08333333 | YES |
| CHEK1 | ENSG00000149554 | 163 | 702 | 8.77E−38 | 2.12E−36 | 0.18843931 | YES |
| CHERP | ENSG00000085872 | 30 | 269 | 2.08E−29 | 3.96E−28 | 0.10033445 | YES |
| CHMP2A | ENSG00000130724 | 179 | 437 | 8.68E−15 | 1.10E−13 | 0.29058442 | YES |
| CHMP3 | ENSG00000115561 | 331 | 1021 | 1.06E−33 | 2.26E−32 | 0.24482249 | YES |
| CHMP4B | ENSG00000101421 | 235 | 3051 | 9.95E−145 | 6.43E−142 | 0.07151552 | YES |
| CHMP6 | ENSG00000176108 | 25 | 225 | 8.05E−26 | 1.39E−24 | 0.1 | YES |
| CHORDC1 | ENSG00000110172 | 152 | 556 | 2.95E−28 | 5.44E−27 | 0.21468927 | YES |
| CHTF8 | ENSG00000168802 | 350 | 1404 | 5.20E−53 | 2.06E−51 | 0.1995439 | YES |
| CHTOP | ENSG00000160679 | 506 | 1166 | 2.00E−23 | 3.20E−22 | 0.30263158 | YES |
| CIAO1 | ENSG00000144021 | 19 | 215 | 9.22E−27 | 1.63E−25 | 0.08119658 | YES |
| CIAPIN1 | ENSG00000005194 | 67 | 744 | 1.67E−63 | 9.00E−62 | 0.08261406 | YES |
| CINP | ENSG00000100865 | 21 | 268 | 1.63E−32 | 3.37E−31 | 0.07266436 | YES |
| CIRH1A | ENSG00000141076 | 138 | 798 | 3.31E−50 | 1.18E−48 | 0.1474359 | YES |
| CIT | ENSG00000122966 | 118 | 804 | 3.81E−55 | 1.62E−53 | 0.12798265 | YES |
| CKAP5 | ENSG00000175216 | 88 | 1500 | 1.97E−107 | 4.90E−105 | 0.05541562 | YES |
| CLASP2 | ENSG00000163539 | 2777 | 4045 | 5.00E−11 | 5.59E−10 | 0.40706538 | YES |
| CLASRP | ENSG00000104859 | 51 | 182 | 4.92E−13 | 5.85E−12 | 0.21888412 | YES |
| CLNS1A | ENSG00000074201 | 63 | 735 | 6.38E−64 | 3.49E−62 | 0.07894737 | YES |
| CLP1 | ENSG00000172409 | 13 | 111 | 7.00E−15 | 8.88E−14 | 0.10483871 | YES |
| CLPB | ENSG00000162129 | 232 | 458 | 2.24E−10 | 2.45E−09 | 0.33623188 | YES |
| CLSPN | ENSG00000092853 | 52 | 882 | 2.20E−78 | 2.02E−76 | 0.05567452 | YES |
| CLTC | ENSG00000141367 | 337 | 1220 | 1.71E−44 | 5.05E−43 | 0.21644188 | YES |
| CMPK1 | ENSG00000162368 | 56 | 426 | 2.93E−38 | 7.14E−37 | 0.11618257 | YES |
| CNOT1 | ENSG00000125107 | 132 | 1662 | 7.16E−106 | 1.67E−103 | 0.0735786 | YES |
| CNOT10 | ENSG00000182973 | 404 | 795 | 5.74E−14 | 7.04E−13 | 0.33694746 | YES |
| CNOT11 | ENSG00000158435 | 111 | 246 | 7.00E−09 | 7.31E−08 | 0.31092437 | YES |
| CNOT2 | ENSG00000111596 | 936 | 1371 | 1.23E−07 | 1.24E−06 | 0.40572172 | YES |
| CNOT3 | ENSG00000088038 | 138 | 398 | 1.38E−17 | 1.87E−16 | 0.25746269 | YES |
| COA5 | ENSG00000183513 | 149 | 367 | 1.86E−13 | 2.24E−12 | 0.28875969 | YES |
| COASY | ENSG00000068120 | 193 | 344 | 4.67E−07 | 4.57E−06 | 0.3594041 | YES |
| COG1 | ENSG00000166685 | 33 | 90 | 3.45E−06 | 3.24E−05 | 0.26829268 | YES |
| COG2 | ENSG00000135775 | 26 | 126 | 1.36E−12 | 1.59E−11 | 0.17105263 | YES |
| COG3 | ENSG00000136152 | 114 | 480 | 4.03E−29 | 7.65E−28 | 0.19191919 | YES |
| COG4 | ENSG00000103051 | 168 | 835 | 8.16E−47 | 2.58E−45 | 0.16749751 | YES |
| COG5 | ENSG00000164597 | 773 | 1114 | 1.38E−06 | 1.32E−05 | 0.40964494 | YES |
| COG7 | ENSG00000168434 | 259 | 506 | 9.71E−11 | 1.07E−09 | 0.33856209 | YES |
| COG8 | ENSG00000213380 | 560 | 999 | 2.16E−12 | 2.52E−11 | 0.35920462 | YES |
| COPA | ENSG00000122218 | 25 | 421 | 8.11E−48 | 2.69E−46 | 0.05605381 | YES |
| COPB1 | ENSG00000129083 | 48 | 376 | 1.64E−35 | 3.69E−34 | 0.11320755 | YES |
| COPB2 | ENSG00000184432 | 17 | 431 | 8.69E−52 | 3.30E−50 | 0.03794643 | YES |
| COPE | ENSG00000105669 | 23 | 290 | 2.36E−34 | 5.19E−33 | 0.07348243 | YES |
| COPG1 | ENSG00000181789 | 40 | 516 | 5.93E−52 | 2.27E−50 | 0.07194245 | YES |
| COPS2 | ENSG00000166200 | 27 | 557 | 4.23E−60 | 2.13E−58 | 0.04623288 | YES |
| COPS3 | ENSG00000141030 | 48 | 623 | 4.73E−59 | 2.27E−57 | 0.07153502 | YES |
| COPS4 | ENSG00000138663 | 41 | 628 | 1.10E−61 | 5.64E−60 | 0.0612855 | YES |
| COPS6 | ENSG00000168090 | 131 | 257 | 2.48E−07 | 2.46E−06 | 0.33762887 | YES |
| COPS8 | ENSG00000198612 | 9 | 197 | 1.68E−28 | 3.12E−27 | 0.04368932 | YES |
| COPZ1 | ENSG00000111481 | 120 | 926 | 9.59E−64 | 5.23E−62 | 0.11472275 | YES |
| COQ3 | ENSG00000132423 | 1018 | 1451 | 5.20E−07 | 5.07E−06 | 0.41231268 | YES |
| COQ4 | ENSG00000167113 | 376 | 521 | 0.0003916 | 0.00327078 | 0.41917503 | YES |
| COQ5 | ENSG00000110871 | 562 | 852 | 4.70E−07 | 4.60E−06 | 0.39745403 | YES |
| COX10 | ENSG00000006695 | 1738 | 2799 | 3.10E−14 | 3.85E−13 | 0.38307251 | YES |
| COX11 | ENSG00000166260 | 217 | 412 | 5.40E−09 | 5.65E−08 | 0.34499205 | YES |
| COX15 | ENSG00000014919 | 284 | 561 | 1.20E−11 | 1.36E−10 | 0.33609467 | YES |
| COX17 | ENSG00000138495 | 319 | 511 | 1.39E−06 | 1.33E−05 | 0.38433735 | YES |
| COX5B | ENSG00000135940 | 67 | 224 | 3.76E−14 | 4.64E−13 | 0.23024055 | YES |
| COX6B1 | ENSG00000126267 | 259 | 427 | 1.63E−06 | 1.56E−05 | 0.37755102 | YES |
| COX7B | ENSG00000131174 | 285 | 630 | 1.58E−15 | 2.04E−14 | 0.31147541 | YES |
| COX7C | ENSG00000127184 | 69 | 218 | 4.11E−13 | 4.91E−12 | 0.24041812 | YES |
| CPSF2 | ENSG00000165934 | 113 | 1059 | 1.13E−74 | 9.09E−73 | 0.09641638 | YES |
| CPSF3 | ENSG00000119203 | 55 | 276 | 1.12E−22 | 1.75E−21 | 0.16616314 | YES |
| CPSF6 | ENSG00000111605 | 40 | 464 | 5.72E−47 | 1.83E−45 | 0.07936508 | YES |
| CRCP | ENSG00000241258 | 203 | 969 | 1.09E−49 | 3.80E−48 | 0.17320819 | YES |
| CREBBP | ENSG00000005339 | 901 | 3728 | 1.61E−86 | 1.98E−84 | 0.19464247 | YES |
| CRLS1 | ENSG00000088766 | 26 | 581 | 1.38E−62 | 7.20E−61 | 0.04283361 | YES |
| CRNKL1 | ENSG00000101343 | 7 | 187 | 4.53E−28 | 8.29E−27 | 0.03608247 | YES |
| CSDE1 | ENSG00000009307 | 307 | 776 | 6.30E−22 | 9.72E−21 | 0.28347184 | YES |
| CSE1L | ENSG00000124207 | 70 | 1414 | 3.32E−107 | 8.16E−105 | 0.04716981 | YES |
| CSNK1A1 | ENSG00000113712 | 261 | 1507 | 2.00E−72 | 1.47E−70 | 0.14762443 | YES |
| CSNK2B | ENSG00000204435 | 142 | 233 | 0.00010391 | 0.00090062 | 0.37866667 | YES |
| CSTF1 | ENSG00000101138 | 59 | 611 | 1.09E−54 | 4.58E−53 | 0.0880597 | YES |
| CSTF2 | ENSG00000101811 | 89 | 269 | 2.15E−14 | 2.68E−13 | 0.24860335 | YES |
| CSTF3 | ENSG00000176102 | 170 | 1543 | 1.89E−91 | 2.68E−89 | 0.0992411 | YES |
| CTC1 | ENSG00000178971 | 303 | 482 | 2.88E−06 | 2.73E−05 | 0.38598726 | YES |
| CTCF | ENSG00000102974 | 353 | 6449 | 1.60E−213 | 5.60E−210 | 0.0518965 | YES |
| CTDP1 | ENSG00000060069 | 20 | 84 | 1.43E−08 | 1.48E−07 | 0.19230769 | YES |
| CTDSPL2 | ENSG00000137770 | 1728 | 2779 | 3.95E−14 | 4.87E−13 | 0.38340359 | YES |
| CTNNBL1 | ENSG00000132792 | 538 | 3631 | 3.65E−121 | 1.27E−118 | 0.12904773 | YES |
| CTPS1 | ENSG00000171793 | 368 | 579 | 1.23E−06 | 1.18E−05 | 0.38859556 | YES |
| CTR9 | ENSG00000198730 | 163 | 486 | 8.54E−21 | 1.26E−19 | 0.25115562 | YES |
| CTU1 | ENSG00000142544 | 29 | 365 | 1.50E−40 | 4.01E−39 | 0.07360406 | YES |
| CTU2 | ENSG00000174177 | 39 | 166 | 4.32E−14 | 5.32E−13 | 0.1902439 | YES |
| CUL1 | ENSG00000055130 | 683 | 1704 | 1.74E−32 | 3.59E−31 | 0.28613322 | YES |
| CUL2 | ENSG00000108094 | 132 | 872 | 4.58E−57 | 2.08E−55 | 0.1314741 | YES |
| CWC22 | ENSG00000163510 | 12 | 228 | 3.48E−31 | 6.97E−30 | 0.05 | YES |
| CWC25 | ENSG00000108296 | 60 | 202 | 2.85E−13 | 3.42E−12 | 0.22900763 | YES |
| CWF19L2 | ENSG00000152404 | 200 | 526 | 1.68E−18 | 2.33E−17 | 0.27548209 | YES |
| CXorf56 | ENSG00000018610 | 63 | 259 | 8.38E−19 | 1.17E−17 | 0.19565217 | YES |
| CYCS | ENSG00000172115 | 204 | 401 | 1.56E−09 | 1.67E−08 | 0.33719008 | YES |
| DAD1 | ENSG00000129562 | 24 | 246 | 9.23E−29 | 1.73E−27 | 0.08888889 | YES |
| DAP3 | ENSG00000132676 | 383 | 901 | 3.48E−21 | 5.22E−20 | 0.2982866 | YES |
| DARS | ENSG00000115866 | 72 | 1302 | 2.80E−100 | 5.50E−98 | 0.05240175 | YES |
| DARS2 | ENSG00000117593 | 431 | 654 | 2.86E−06 | 2.70E−05 | 0.39723502 | YES |
| DBR1 | ENSG00000138231 | 37 | 374 | 8.72E−39 | 2.19E−37 | 0.09002433 | YES |
| DCAF7 | ENSG00000136485 | 151 | 876 | 3.00E−53 | 1.20E−51 | 0.14703019 | YES |
| DCP2 | ENSG00000172795 | 163 | 642 | 4.00E−33 | 8.40E−32 | 0.20248447 | YES |
| DCPS | ENSG00000110063 | 319 | 1003 | 3.75E−34 | 8.13E−33 | 0.24130106 | YES |
| DCTN1 | ENSG00000204843 | 137 | 353 | 5.02E−14 | 6.16E−13 | 0.27959184 | YES |
| DCTN2 | ENSG00000175203 | 15 | 84 | 3.88E−10 | 4.23E−09 | 0.15151515 | YES |
| DCTN3 | ENSG00000137100 | 7 | 88 | 7.51E−14 | 9.16E−13 | 0.07368421 | YES |
| DCTN4 | ENSG00000132912 | 69 | 889 | 4.88E−74 | 3.86E−72 | 0.07202505 | YES |
| DCTN5 | ENSG00000166847 | 82 | 1070 | 3.67E−83 | 4.14E−81 | 0.07118056 | YES |
| DDB1 | ENSG00000167986 | 27 | 238 | 1.01E−26 | 1.78E−25 | 0.10188679 | YES |
| DDOST | ENSG00000244038 | 14 | 295 | 2.11E−38 | 5.21E−37 | 0.04530744 | YES |
| DDX10 | ENSG00000178105 | 1081 | 2423 | 1.88E−31 | 3.79E−30 | 0.30850457 | YES |
| DDX18 | ENSG00000088205 | 23 | 231 | 2.78E−27 | 4.96E−26 | 0.09055118 | YES |
| DDX19A | ENSG00000168872 | 68 | 605 | 1.40E−51 | 5.30E−50 | 0.10104012 | YES |
| DDX20 | ENSG00000064703 | 21 | 174 | 1.07E−20 | 1.57E−19 | 0.10769231 | YES |
| DDX21 | ENSG00000165732 | 171 | 982 | 1.14E−56 | 5.09E−55 | 0.14830876 | YES |
| DDX23 | ENSG00000174243 | 71 | 884 | 4.06E−73 | 3.05E−71 | 0.07434555 | YES |
| DDX24 | ENSG00000089737 | 40 | 334 | 1.70E−33 | 3.61E−32 | 0.10695187 | YES |
| DDX27 | ENSG00000124228 | 66 | 551 | 2.55E−47 | 8.30E−46 | 0.10696921 | YES |
| DDX39B | ENSG00000198563 | 233 | 903 | 4.14E−40 | 1.09E−38 | 0.20510563 | YES |
| DDX3X | ENSG00000215301 | 397 | 649 | 7.32E−08 | 7.42E−07 | 0.37954111 | YES |
| DDX46 | ENSG00000145833 | 148 | 2479 | 2.49E−139 | 1.24E−136 | 0.05633803 | YES |
| DDX47 | ENSG00000213782 | 20 | 329 | 6.64E−40 | 1.72E−38 | 0.05730659 | YES |
| DDX49 | ENSG00000105671 | 24 | 322 | 1.24E−37 | 3.00E−36 | 0.06936416 | YES |
| DDX51 | ENSG00000185163 | 2 | 48 | 1.34E−09 | 1.43E−08 | 0.04 | YES |
| DDX52 | ENSG00000141141 | 61 | 444 | 1.31E−38 | 3.25E−37 | 0.12079208 | YES |
| DDX54 | ENSG00000123064 | 23 | 307 | 2.63E−36 | 6.10E−35 | 0.06969697 | YES |
| DDX55 | ENSG00000111364 | 50 | 912 | 3.87E−81 | 3.96E−79 | 0.05197505 | YES |
| DDX56 | ENSG00000136271 | 26 | 401 | 2.08E−45 | 6.28E−44 | 0.06088993 | YES |
| DDX59 | ENSG00000118197 | 208 | 571 | 1.20E−20 | 1.76E−19 | 0.26700899 | YES |
| DDX6 | ENSG00000110367 | 851 | 1368 | 1.37E−10 | 1.51E−09 | 0.38350608 | YES |
| DENR | ENSG00000139726 | 335 | 517 | 6.37E−06 | 5.89E−05 | 0.39319249 | YES |
| DGCR14 | ENSG00000100056 | 16 | 216 | 4.74E−28 | 8.66E−27 | 0.06896552 | YES |
| DGCR8 | ENSG00000128191 | 237 | 1296 | 1.91E−64 | 1.06E−62 | 0.15459883 | YES |
| DHDDS | ENSG00000117682 | 242 | 447 | 7.46E−09 | 7.77E−08 | 0.35123367 | YES |
| DHFR | ENSG00000228716 | 43 | 534 | 1.16E−52 | 4.57E−51 | 0.0745234 | YES |
| DHX15 | ENSG00000109606 | 91 | 967 | 8.91E−74 | 6.98E−72 | 0.08601134 | YES |
| DHX33 | ENSG00000005100 | 74 | 753 | 2.85E−62 | 1.48E−60 | 0.08948005 | YES |
| DHX35 | ENSG00000101452 | 483 | 1237 | 7.31E−29 | 1.38E−27 | 0.28081395 | YES |
| DHX36 | ENSG00000174953 | 96 | 509 | 1.01E−35 | 2.30E−34 | 0.15867769 | YES |
| DHX38 | ENSG00000140829 | 46 | 176 | 1.61E−13 | 1.94E−12 | 0.20720721 | YES |
| DHX8 | ENSG00000067596 | 32 | 445 | 9.10E−48 | 3.01E−46 | 0.06708595 | YES |
| DHX9 | ENSG00000135829 | 34 | 517 | 4.95E−54 | 2.03E−52 | 0.06170599 | YES |
| DIDO1 | ENSG00000101191 | 186 | 1221 | 2.35E−69 | 1.54E−67 | 0.13219616 | YES |
| DIEXF | ENSG00000117597 | 16 | 222 | 8.26E−29 | 1.55E−27 | 0.06722689 | YES |
| DIMT1 | ENSG00000086189 | 71 | 428 | 2.68E−34 | 5.86E−33 | 0.14228457 | YES |
| DIS3 | ENSG00000083520 | 17 | 189 | 3.58E−24 | 5.85E−23 | 0.08252427 | YES |
| DKC1 | ENSG00000130826 | 74 | 904 | 9.30E−74 | 7.25E−72 | 0.07566462 | YES |
| DLD | ENSG00000091140 | 205 | 336 | 1.14E−05 | 0.00010414 | 0.37892791 | YES |
| DLST | ENSG00000119689 | 37 | 353 | 1.54E−36 | 3.62E−35 | 0.09487179 | YES |
| DMAP1 | ENSG00000178028 | 7 | 56 | 1.09E−08 | 1.12E−07 | 0.11111111 | YES |
| DNAJA3 | ENSG00000103423 | 177 | 1128 | 3.12E−65 | 1.77E−63 | 0.13563218 | YES |
| DNAJC11 | ENSG00000007923 | 126 | 1424 | 2.91E−94 | 4.59E−92 | 0.08129032 | YES |
| DNAJC19 | ENSG00000205981 | 3 | 193 | 1.05E−30 | 2.09E−29 | 0.01530612 | YES |
| DNAJC2 | ENSG00000105821 | 346 | 900 | 6.64E−25 | 1.11E−23 | 0.2776886 | YES |
| DNAJC8 | ENSG00000126698 | 97 | 458 | 8.24E−31 | 1.64E−29 | 0.17477477 | YES |
| DNAJC9 | ENSG00000213551 | 1317 | 1651 | 0.00051895 | 0.00429549 | 0.44373315 | YES |
| DNM1L | ENSG00000087470 | 109 | 869 | 3.45E−62 | 1.78E−60 | 0.11145194 | YES |
| DNM2 | ENSG00000079805 | 177 | 950 | 2.89E−53 | 1.16E−51 | 0.15705413 | YES |
| DNMT1 | ENSG00000130816 | 559 | 1329 | 1.79E−26 | 3.13E−25 | 0.29608051 | YES |
| DNTTIP2 | ENSG00000067334 | 19 | 200 | 7.67E−25 | 1.28E−23 | 0.08675799 | YES |
| DOHH | ENSG00000129932 | 124 | 559 | 6.36E−34 | 1.36E−32 | 0.18155198 | YES |
| DONSON | ENSG00000159147 | 38 | 505 | 1.43E−51 | 5.39E−50 | 0.06998158 | YES |
| DPH5 | ENSG00000117543 | 522 | 689 | 0.00082091 | 0.00669028 | 0.43104872 | YES |
| DPY30 | ENSG00000162961 | 96 | 187 | 5.20E−06 | 4.85E−05 | 0.33922261 | YES |
| DR1 | ENSG00000117505 | 161 | 498 | 4.63E−22 | 7.17E−21 | 0.24430956 | YES |
| DSN1 | ENSG00000149636 | 153 | 1104 | 1.24E−68 | 7.88E−67 | 0.12171838 | YES |
| DTL | ENSG00000143476 | 99 | 1025 | 8.81E−76 | 7.36E−74 | 0.08807829 | YES |
| DTYMK | ENSG00000168393 | 59 | 408 | 1.39E−35 | 3.15E−34 | 0.12633833 | YES |
| DUT | ENSG00000128951 | 65 | 620 | 9.73E−54 | 3.97E−52 | 0.09489051 | YES |
| DYNC1H1 | ENSG00000197102 | 49 | 985 | 1.56E−86 | 1.93E−84 | 0.04738878 | YES |
| DYNC1I2 | ENSG00000077380 | 177 | 967 | 1.91E−54 | 7.96E−53 | 0.15472028 | YES |
| DYNLRB1 | ENSG00000125971 | 65 | 768 | 5.63E−66 | 3.24E−64 | 0.07803121 | YES |
| DYRK1A | ENSG00000157540 | 660 | 4493 | 6.14E−133 | 2.82E−130 | 0.12808073 | YES |
| E4F1 | ENSG00000167967 | 38 | 204 | 4.94E−19 | 6.94E−18 | 0.15702479 | YES |
| EAPP | ENSG00000129518 | 280 | 542 | 6.13E−11 | 6.82E−10 | 0.3406326 | YES |
| EBNA1BP2 | ENSG00000117395 | 7 | 119 | 1.44E−18 | 1.99E−17 | 0.05555556 | YES |
| ECD | ENSG00000122882 | 97 | 502 | 7.48E−35 | 1.66E−33 | 0.16193656 | YES |
| ECT2 | ENSG00000114346 | 61 | 961 | 1.98E−81 | 2.06E−79 | 0.05968689 | YES |
| EDC4 | ENSG00000038358 | 200 | 351 | 7.01E−07 | 6.80E−06 | 0.36297641 | YES |
| EED | ENSG00000074266 | 700 | 1055 | 1.34E−07 | 1.35E−06 | 0.3988604 | YES |
| EEF1A1 | ENSG00000156508 | 70 | 549 | 5.08E−46 | 1.57E−44 | 0.11308562 | YES |
| EEF1G | ENSG00000254772 | 66 | 1338 | 6.11E−104 | 1.33E−101 | 0.04700855 | YES |
| EEF2 | ENSG00000167658 | 23 | 533 | 2.50E−59 | 1.21E−57 | 0.04136691 | YES |
| EEFSEC | ENSG00000132394 | 1230 | 1685 | 2.95E−06 | 2.79E−05 | 0.4219554 | YES |
| EFTUD2 | ENSG00000108883 | 190 | 1295 | 4.09E−73 | 3.07E−71 | 0.12794613 | YES |
| EHMT1 | ENSG00000181090 | 1670 | 2464 | 7.67E−10 | 8.27E−09 | 0.4039671 | YES |
| EIF1 | ENSG00000173812 | 22 | 323 | 1.75E−38 | 4.33E−37 | 0.06376812 | YES |
| EIF1AD | ENSG00000175376 | 142 | 339 | 3.29E−12 | 3.81E−11 | 0.2952183 | YES |
| EIF1AX | ENSG00000173674 | 44 | 374 | 1.55E−36 | 3.63E−35 | 0.10526316 | YES |
| EIF2B1 | ENSG00000111361 | 23 | 444 | 7.54E−51 | 2.75E−49 | 0.04925054 | YES |
| EIF2B2 | ENSG00000119718 | 5 | 82 | 8.34E−14 | 1.01E−12 | 0.05747126 | YES |
| EIF2B3 | ENSG00000070785 | 97 | 1496 | 4.84E−105 | 1.08E−102 | 0.0608914 | YES |
| EIF2B4 | ENSG00000115211 | 24 | 139 | 5.69E−15 | 7.24E−14 | 0.14723926 | YES |
| EIF2B5 | ENSG00000145191 | 63 | 501 | 1.55E−43 | 4.46E−42 | 0.11170213 | YES |
| EIF2S1 | ENSG00000134001 | 58 | 412 | 2.84E−36 | 6.59E−35 | 0.12340426 | YES |
| EIF2S2 | ENSG00000125977 | 60 | 833 | 2.03E−72 | 1.48E−70 | 0.06718925 | YES |
| EIF2S3 | ENSG00000130741 | 232 | 1959 | 7.89E−101 | 1.57E−98 | 0.10588772 | YES |
| EIF3A | ENSG00000107581 | 60 | 930 | 1.59E−79 | 1.51E−77 | 0.06060606 | YES |
| EIF3B | ENSG00000106263 | 35 | 491 | 3.09E−51 | 1.15E−49 | 0.06653992 | YES |
| EIF3D | ENSG00000100353 | 207 | 855 | 4.07E−41 | 1.11E−39 | 0.19491525 | YES |
| EIF3I | ENSG00000084623 | 25 | 349 | 2.99E−40 | 7.91E−39 | 0.06684492 | YES |
| EIF3J | ENSG00000104131 | 98 | 571 | 8.79E−41 | 2.37E−39 | 0.14648729 | YES |
| EIF3M | ENSG00000149100 | 40 | 535 | 1.01E−53 | 4.11E−52 | 0.06956522 | YES |
| EIF4A1 | ENSG00000161960 | 106 | 495 | 3.55E−32 | 7.25E−31 | 0.17637271 | YES |
| EIF4A2 | ENSG00000156976 | 189 | 529 | 3.15E−20 | 4.55E−19 | 0.2632312 | YES |
| EIF4A3 | ENSG00000141543 | 1 | 139 | 2.38E−24 | 3.92E−23 | 0.00714286 | YES |
| EIF4B | ENSG00000063046 | 2002 | 3281 | 5.42E−16 | 7.04E−15 | 0.37895135 | YES |
| EIF4E | ENSG00000151247 | 61 | 819 | 4.53E−71 | 3.14E−69 | 0.06931818 | YES |
| EIF4G1 | ENSG00000114867 | 69 | 209 | 3.54E−12 | 4.09E−11 | 0.24820144 | YES |
| EIF4G2 | ENSG00000110321 | 74 | 508 | 3.70E−41 | 1.01E−39 | 0.12714777 | YES |
| EIF5 | ENSG00000100664 | 49 | 233 | 2.07E−19 | 2.93E−18 | 0.17375887 | YES |
| EIF5A | ENSG00000132507 | 45 | 370 | 8.42E−36 | 1.93E−34 | 0.10843373 | YES |
| EIF5B | ENSG00000158417 | 219 | 2328 | 8.59E−120 | 2.88E−117 | 0.08598351 | YES |
| EIF6 | ENSG00000242372 | 55 | 339 | 1.53E−29 | 2.93E−28 | 0.13959391 | YES |
| ELAC2 | ENSG00000006744 | 23 | 218 | 1.17E−25 | 2.00E−24 | 0.09543568 | YES |
| ELL | ENSG00000105656 | 60 | 570 | 9.23E−51 | 3.34E−49 | 0.0952381 | YES |
| ELP2 | ENSG00000134759 | 41 | 464 | 1.21E−46 | 3.78E−45 | 0.08118812 | YES |
| ELP4 | ENSG00000109911 | 84 | 1143 | 2.08E−87 | 2.67E−85 | 0.06845966 | YES |
| ELP5 | ENSG00000170291 | 70 | 492 | 9.78E−41 | 2.63E−39 | 0.12455516 | YES |
| ELP6 | ENSG00000163832 | 133 | 1414 | 4.09E−92 | 5.85E−90 | 0.08597285 | YES |
| EMC3 | ENSG00000125037 | 146 | 259 | 5.66E−06 | 5.26E−05 | 0.36049383 | YES |
| EMC4 | ENSG00000128463 | 124 | 256 | 4.74E−08 | 4.83E−07 | 0.32631579 | YES |
| EMC7 | ENSG00000134153 | 173 | 352 | 1.82E−09 | 1.93E−08 | 0.32952381 | YES |
| ENO1 | ENSG00000074800 | 455 | 883 | 2.64E−14 | 3.28E−13 | 0.34005979 | YES |
| EPRS | ENSG00000136628 | 35 | 505 | 1.44E−52 | 5.67E−51 | 0.06481481 | YES |
| ERAL1 | ENSG00000132591 | 243 | 447 | 9.04E−09 | 9.36E−08 | 0.35217391 | YES |
| ERCC2 | ENSG00000104884 | 21 | 217 | 2.89E−26 | 5.05E−25 | 0.08823529 | YES |
| ERCC3 | ENSG00000163161 | 13 | 108 | 1.94E−14 | 2.42E−13 | 0.10743802 | YES |
| ERCC4 | ENSG00000175595 | 147 | 412 | 2.46E−17 | 3.31E−16 | 0.26296959 | YES |
| ERH | ENSG00000100632 | 148 | 743 | 6.84E−44 | 1.99E−42 | 0.1661055 | YES |
| ESF1 | ENSG00000089048 | 78 | 1117 | 2.70E−87 | 3.42E−85 | 0.06527197 | YES |
| ETF1 | ENSG00000120705 | 115 | 1354 | 9.67E−93 | 1.43E−90 | 0.07828455 | YES |
| EWSR1 | ENSG00000182944 | 339 | 989 | 7.53E−31 | 1.50E−29 | 0.25527108 | YES |
| EXOC1 | ENSG00000090989 | 330 | 966 | 1.39E−30 | 2.74E−29 | 0.25462963 | YES |
| EXOC2 | ENSG00000112685 | 3068 | 4701 | 3.51E−14 | 4.34E−13 | 0.39490282 | YES |
| EXOC4 | ENSG00000131558 | 8241 | 11582 | 2.49E−12 | 2.89E−11 | 0.4157292 | YES |
| EXOC5 | ENSG00000070367 | 130 | 735 | 4.51E−47 | 1.46E−45 | 0.15028902 | YES |
| EXOSC1 | ENSG00000171311 | 118 | 301 | 1.70E−12 | 1.99E−11 | 0.28162291 | YES |
| EXOSC10 | ENSG00000171824 | 79 | 575 | 6.30E−46 | 1.95E−44 | 0.12079511 | YES |
| EXOSC2 | ENSG00000130713 | 56 | 755 | 1.48E−67 | 9.00E−66 | 0.06905055 | YES |
| EXOSC3 | ENSG00000107371 | 7 | 172 | 4.60E−26 | 7.99E−25 | 0.03910615 | YES |
| EXOSC5 | ENSG00000077348 | 40 | 493 | 8.94E−50 | 3.13E−48 | 0.0750469 | YES |
| EXOSC7 | ENSG00000075914 | 28 | 425 | 3.75E−47 | 1.21E−45 | 0.06181015 | YES |
| EXOSC8 | ENSG00000120699 | 159 | 899 | 2.94E−53 | 1.18E−51 | 0.15028355 | YES |
| EXOSC9 | ENSG00000123737 | 49 | 334 | 9.83E−31 | 1.95E−29 | 0.12793734 | YES |
| EZH2 | ENSG00000106462 | 710 | 1003 | 7.78E−06 | 7.16E−05 | 0.41447752 | YES |
| FAF2 | ENSG00000113194 | 511 | 846 | 2.74E−09 | 2.90E−08 | 0.37656595 | YES |
| FAM178A | ENSG00000119906 | 182 | 314 | 3.21E−06 | 3.03E−05 | 0.36693548 | YES |
| FAM204A | ENSG00000165669 | 118 | 277 | 1.75E−10 | 1.93E−09 | 0.29873418 | YES |
| FAM210A | ENSG00000177150 | 93 | 488 | 1.64E−34 | 3.62E−33 | 0.16006885 | YES |
| FAM32A | ENSG00000105058 | 15 | 175 | 4.28E−23 | 6.79E−22 | 0.07894737 | YES |
| FAM50A | ENSG00000071859 | 17 | 50 | 0.00013312 | 0.00115041 | 0.25373134 | YES |
| FAM60A | ENSG00000139146 | 744 | 1487 | 8.15E−20 | 1.17E−18 | 0.33348274 | YES |
| FAM96B | ENSG00000166595 | 8 | 148 | 2.63E−22 | 4.09E−21 | 0.05128205 | YES |
| FAM98B | ENSG00000171262 | 101 | 266 | 4.67E−12 | 5.38E−11 | 0.27520436 | YES |
| FANCB | ENSG00000181544 | 402 | 633 | 6.05E−07 | 5.88E−06 | 0.3884058 | YES |
| FANCC | ENSG00000158169 | 2508 | 3190 | 2.87E−05 | 0.00025634 | 0.44015444 | YES |
| FANCD2 | ENSG00000144554 | 702 | 1041 | 3.97E−07 | 3.91E−06 | 0.40275387 | YES |
| FANCL | ENSG00000115392 | 482 | 773 | 4.37E−08 | 4.46E−07 | 0.38406375 | YES |
| FANCM | ENSG00000187790 | 442 | 2099 | 4.71E−75 | 3.82E−73 | 0.17394726 | YES |
| FARS2 | ENSG00000145982 | 1137 | 1956 | 3.23E−15 | 4.15E−14 | 0.36760427 | YES |
| FARSA | ENSG00000179115 | 34 | 480 | 1.62E−50 | 5.83E−49 | 0.06614786 | YES |
| FARSB | ENSG00000116120 | 54 | 714 | 7.35E−65 | 4.13E−63 | 0.0703125 | YES |
| FAU | ENSG00000149806 | 36 | 290 | 7.47E−30 | 1.44E−28 | 0.11042945 | YES |
| FBL | ENSG00000105202 | 31 | 490 | 1.69E−52 | 6.63E−51 | 0.05950096 | YES |
| FBXO5 | ENSG00000112029 | 101 | 1522 | 1.67E−105 | 3.78E−103 | 0.06223044 | YES |
| FCF1 | ENSG00000119616 | 31 | 264 | 1.77E−28 | 3.27E−27 | 0.10508475 | YES |
| FDPS | ENSG00000160752 | 691 | 953 | 3.45E−05 | 0.00030736 | 0.4203163 | YES |
| FDX1L | ENSG00000267673 | 40 | 220 | 2.19E−20 | 3.19E−19 | 0.15384615 | YES |
| FDXR | ENSG00000161513 | 73 | 178 | 2.27E−08 | 2.33E−07 | 0.29083665 | YES |
| FEN1 | ENSG00000168496 | 199 | 1106 | 1.58E−59 | 7.78E−58 | 0.15249042 | YES |
| FGFR1OP | ENSG00000213066 | 105 | 306 | 5.07E−15 | 6.46E−14 | 0.25547445 | YES |
| FGFR1OP2 | ENSG00000111790 | 294 | 444 | 3.76E−05 | 0.00033421 | 0.39837398 | YES |
| FH | ENSG00000091483 | 76 | 138 | 0.00019212 | 0.00164314 | 0.35514019 | YES |
| FIP1L1 | ENSG00000145216 | 40 | 498 | 2.98E−50 | 1.06E−48 | 0.07434944 | YES |
| FIS1 | ENSG00000214253 | 258 | 396 | 4.00E−05 | 0.0003549 | 0.39449541 | YES |
| FLII | ENSG00000177731 | 98 | 170 | 0.00015613 | 0.00134194 | 0.36567164 | YES |
| FNBP4 | ENSG00000109920 | 303 | 766 | 9.04E−22 | 1.38E−20 | 0.28344247 | YES |
| FNTB | ENSG00000257365 | 101 | 1291 | 2.46E−92 | 3.61E−90 | 0.07255747 | YES |
| FTSJ1 | ENSG00000068438 | 243 | 376 | 4.06E−05 | 0.00036035 | 0.39256866 | YES |
| FTSJ3 | ENSG00000108592 | 48 | 170 | 2.41E−12 | 2.80E−11 | 0.22018349 | YES |
| FUBP1 | ENSG00000162613 | 228 | 964 | 5.18E−45 | 1.55E−43 | 0.19127517 | YES |
| FUS | ENSG00000089280 | 166 | 305 | 4.55E−07 | 4.47E−06 | 0.35244161 | YES |
| FXN | ENSG00000165060 | 98 | 339 | 2.20E−19 | 3.12E−18 | 0.22425629 | YES |
| G3BP2 | ENSG00000138757 | 458 | 808 | 7.92E−11 | 8.79E−10 | 0.36176935 | YES |
| GAB2 | ENSG00000033327 | 1758 | 3285 | 1.50E−23 | 2.42E−22 | 0.34860202 | YES |
| GABPA | ENSG00000154727 | 115 | 1639 | 1.82E−108 | 4.74E−106 | 0.06556442 | YES |
| GABPB1 | ENSG00000104064 | 147 | 1297 | 4.01E−82 | 4.27E−80 | 0.10180055 | YES |
| GADD45GIP1 | ENSG00000179271 | 226 | 613 | 3.39E−21 | 5.10E−20 | 0.2693683 | YES |
| GAPDH | ENSG00000111640 | 187 | 375 | 1.51E−09 | 1.61E−08 | 0.33274021 | YES |
| GAR1 | ENSG00000109534 | 171 | 402 | 3.09E−13 | 3.71E−12 | 0.29842932 | YES |
| GARS | ENSG00000106105 | 79 | 1047 | 2.06E−82 | 2.21E−80 | 0.07015986 | YES |
| GART | ENSG00000159131 | 466 | 1481 | 1.14E−42 | 3.22E−41 | 0.23934258 | YES |
| GATC | ENSG00000257218 | 275 | 465 | 2.78E−07 | 2.76E−06 | 0.37162162 | YES |
| GBF1 | ENSG00000107862 | 192 | 2301 | 1.28E−123 | 4.67E−121 | 0.07701564 | YES |
| GEMIN2 | ENSG00000092208 | 84 | 423 | 1.55E−30 | 3.04E−29 | 0.16568047 | YES |
| GEMIN4 | ENSG00000179409 | 236 | 884 | 2.35E−38 | 5.76E−37 | 0.21071429 | YES |
| GEMIN5 | ENSG00000082516 | 56 | 416 | 2.97E−37 | 7.06E−36 | 0.11864407 | YES |
| GEMIN8 | ENSG00000046647 | 255 | 773 | 3.90E−28 | 7.15E−27 | 0.24805447 | YES |
| GET4 | ENSG00000239857 | 84 | 143 | 0.00055166 | 0.00455114 | 0.37004405 | YES |
| GFER | ENSG00000127554 | 31 | 257 | 1.20E−27 | 2.16E−26 | 0.10763889 | YES |
| GFM1 | ENSG00000168827 | 178 | 472 | 1.62E−17 | 2.20E−16 | 0.27384615 | YES |
| GGPS1 | ENSG00000152904 | 1149 | 1435 | 0.00091051 | 0.00738258 | 0.44465944 | YES |
| GINS1 | ENSG00000101003 | 91 | 1056 | 6.67E−80 | 6.40E−78 | 0.0793374 | YES |
| GINS2 | ENSG00000131153 | 33 | 402 | 4.61E−43 | 1.31E−41 | 0.07586207 | YES |
| GINS3 | ENSG00000181938 | 78 | 496 | 5.54E−39 | 1.40E−37 | 0.1358885 | YES |
| GLE1 | ENSG00000119392 | 29 | 348 | 1.03E−38 | 2.56E−37 | 0.07692308 | YES |
| GLTSCR2 | ENSG00000105373 | 39 | 509 | 1.27E−51 | 4.83E−50 | 0.07116788 | YES |
| GMNN | ENSG00000112312 | 197 | 390 | 1.62E−09 | 1.72E−08 | 0.33560477 | YES |
| GMPS | ENSG00000163655 | 111 | 467 | 1.39E−28 | 2.59E−27 | 0.19204152 | YES |
| GNB1L | ENSG00000185838 | 79 | 308 | 2.76E−20 | 4.00E−19 | 0.20413437 | YES |
| GNB2L1 | ENSG00000204628 | 191 | 810 | 1.04E−40 | 2.79E−39 | 0.19080919 | YES |
| GNL2 | ENSG00000134697 | 37 | 383 | 9.78E−40 | 2.51E−38 | 0.08809524 | YES |
| GNL3 | ENSG00000163938 | 91 | 883 | 1.02E−67 | 6.26E−66 | 0.09342916 | YES |
| GNL3L | ENSG00000130119 | 140 | 932 | 1.20E−59 | 5.95E−58 | 0.13059701 | YES |
| GOLT1B | ENSG00000111711 | 23 | 256 | 2.46E−30 | 4.79E−29 | 0.08243728 | YES |
| GON4L | ENSG00000116580 | 1862 | 5657 | 9.31E−72 | 6.66E−70 | 0.24763931 | YES |
| GOSR2 | ENSG00000108433 | 9 | 414 | 3.21E−53 | 1.28E−51 | 0.0212766 | YES |
| GPATCH1 | ENSG00000076650 | 854 | 1258 | 1.56E−07 | 1.57E−06 | 0.40435606 | YES |
| GPKOW | ENSG00000068394 | 21 | 150 | 1.79E−17 | 2.43E−16 | 0.12280702 | YES |
| GPN1 | ENSG00000198522 | 38 | 579 | 2.29E−58 | 1.08E−56 | 0.06158833 | YES |
| GPN2 | ENSG00000142751 | 37 | 177 | 5.39E−16 | 7.00E−15 | 0.1728972 | YES |
| GPN3 | ENSG00000111231 | 30 | 500 | 8.54E−54 | 3.50E−52 | 0.05660377 | YES |
| GRB2 | ENSG00000177885 | 1860 | 2552 | 2.82E−07 | 2.80E−06 | 0.42157752 | YES |
| GRPEL1 | ENSG00000109519 | 21 | 397 | 8.02E−47 | 2.54E−45 | 0.05023923 | YES |
| GRWD1 | ENSG00000105447 | 43 | 346 | 7.41E−34 | 1.59E−32 | 0.11053985 | YES |
| GSPT1 | ENSG00000103342 | 55 | 1189 | 4.81E−98 | 8.67E−96 | 0.04421222 | YES |
| GTF2A1 | ENSG00000165417 | 108 | 765 | 1.75E−54 | 7.31E−53 | 0.12371134 | YES |
| GTF2A2 | ENSG00000140307 | 95 | 1113 | 1.14E−82 | 1.25E−80 | 0.07864238 | YES |
| GTF2B | ENSG00000137947 | 38 | 428 | 4.81E−44 | 1.41E−42 | 0.08154506 | YES |
| GTF2E1 | ENSG00000153767 | 52 | 433 | 3.83E−40 | 1.01E−38 | 0.10721649 | YES |
| GTF2F1 | ENSG00000125651 | 86 | 362 | 4.00E−24 | 6.50E−23 | 0.19196429 | YES |
| GTF2F2 | ENSG00000188342 | 297 | 3301 | 2.84E−143 | 1.65E−140 | 0.08254586 | YES |
| GTF2H1 | ENSG00000110768 | 265 | 1518 | 2.36E−72 | 1.71E−70 | 0.14862591 | YES |
| GTF2H3 | ENSG00000111358 | 82 | 1161 | 4.51E−89 | 6.02E−87 | 0.06596943 | YES |
| GTF3A | ENSG00000122034 | 66 | 634 | 1.16E−54 | 4.87E−53 | 0.09428571 | YES |
| GTF3C1 | ENSG00000077235 | 23 | 265 | 2.05E−31 | 4.13E−30 | 0.07986111 | YES |
| GTF3C3 | ENSG00000119041 | 23 | 214 | 3.72E−25 | 6.24E−24 | 0.09704641 | YES |
| GTF3C4 | ENSG00000125484 | 97 | 519 | 2.16E−36 | 5.02E−35 | 0.15746753 | YES |
| GTF3C5 | ENSG00000148308 | 34 | 433 | 6.94E−46 | 2.14E−44 | 0.07280514 | YES |
| GTF3C6 | ENSG00000155115 | 139 | 965 | 3.30E−62 | 1.70E−60 | 0.1259058 | YES |
| GTPBP10 | ENSG00000105793 | 215 | 487 | 5.81E−14 | 7.12E−13 | 0.30626781 | YES |
| GTPBP4 | ENSG00000107937 | 38 | 627 | 1.45E−62 | 7.53E−61 | 0.05714286 | YES |
| GTPBP5 | ENSG00000101181 | 268 | 423 | 9.64E−06 | 8.83E−05 | 0.3878437 | YES |
| GUK1 | ENSG00000143774 | 282 | 397 | 0.00067021 | 0.00550059 | 0.41531664 | YES |
| HARS | ENSG00000170445 | 14 | 180 | 3.66E−24 | 5.96E−23 | 0.07216495 | YES |
| HARS2 | ENSG00000112855 | 107 | 335 | 2.55E−17 | 3.43E−16 | 0.24208145 | YES |
| HAUS1 | ENSG00000152240 | 62 | 798 | 3.58E−69 | 2.33E−67 | 0.07209302 | YES |
| HAUS2 | ENSG00000137814 | 69 | 843 | 1.27E−70 | 8.65E−69 | 0.07565789 | YES |
| HAUS3 | ENSG00000214367 | 163 | 455 | 2.34E−18 | 3.24E−17 | 0.26375405 | YES |
| HAUS4 | ENSG00000092036 | 242 | 872 | 1.28E−36 | 2.99E−35 | 0.21723519 | YES |
| HAUS5 | ENSG00000249115 | 26 | 63 | 0.00023382 | 0.00199104 | 0.29213483 | YES |
| HAUS6 | ENSG00000147874 | 72 | 463 | 1.96E−37 | 4.69E−36 | 0.13457944 | YES |
| HAUS7 | ENSG00000213397 | 16 | 141 | 4.82E−18 | 6.64E−17 | 0.10191083 | YES |
| HAUS8 | ENSG00000131351 | 37 | 468 | 2.40E−48 | 8.05E−47 | 0.07326733 | YES |
| HBS1L | ENSG00000112339 | 79 | 462 | 1.62E−35 | 3.67E−34 | 0.14602588 | YES |
| HCFC1 | ENSG00000172534 | 365 | 764 | 1.43E−15 | 1.84E−14 | 0.32329495 | YES |
| HEATR1 | ENSG00000119285 | 68 | 261 | 7.35E−18 | 1.00E−16 | 0.20668693 | YES |
| HEATR3 | ENSG00000155393 | 137 | 314 | 6.52E−11 | 7.25E−10 | 0.3037694 | YES |
| HELQ | ENSG00000163312 | 164 | 300 | 6.18E−07 | 6.00E−06 | 0.35344828 | YES |
| HINFP | ENSG00000172273 | 46 | 208 | 2.71E−17 | 3.64E−16 | 0.18110236 | YES |
| HJURP | ENSG00000123485 | 42 | 251 | 1.80E−23 | 2.89E−22 | 0.14334471 | YES |
| HMGCR | ENSG00000113161 | 148 | 408 | 7.34E−17 | 9.78E−16 | 0.26618705 | YES |
| HMGCS1 | ENSG00000112972 | 113 | 571 | 2.55E−37 | 6.08E−36 | 0.16520468 | YES |
| HNRNPA1 | ENSG00000135486 | 494 | 962 | 4.54E−15 | 5.79E−14 | 0.33928571 | YES |
| HNRNPA2B1 | ENSG00000122566 | 505 | 908 | 4.61E−12 | 5.31E−11 | 0.35739561 | YES |
| HNRNPC | ENSG00000092199 | 85 | 1817 | 3.10E−124 | 1.18E−121 | 0.0446898 | YES |
| HNRNPF | ENSG00000169813 | 1764 | 2637 | 1.28E−10 | 1.41E−09 | 0.400818 | YES |
| HNRNPH1 | ENSG00000169045 | 43 | 245 | 1.78E−22 | 2.78E−21 | 0.14930556 | YES |
| HNRNPK | ENSG00000165119 | 150 | 547 | 6.98E−28 | 1.27E−26 | 0.21520803 | YES |
| HNRNPL | ENSG00000104824 | 137 | 1254 | 1.07E−81 | 1.12E−79 | 0.09849029 | YES |
| HNRNPM | ENSG00000099783 | 322 | 991 | 4.71E−33 | 9.84E−32 | 0.24523991 | YES |
| HNRNPU | ENSG00000153187 | 37 | 137 | 5.04E−11 | 5.62E−10 | 0.21264368 | YES |
| HSCB | ENSG00000100209 | 23 | 156 | 1.35E−17 | 1.83E−16 | 0.12849162 | YES |
| HSD17B10 | ENSG00000072506 | 145 | 309 | 1.52E−09 | 1.62E−08 | 0.31938326 | YES |
| HSD17B12 | ENSG00000149084 | 910 | 1783 | 1.39E−20 | 2.04E−19 | 0.33791311 | YES |
| HSPA14 | ENSG00000187522 | 202 | 453 | 3.82E−13 | 4.57E−12 | 0.30839695 | YES |
| HSPA5 | ENSG00000044574 | 6 | 156 | 2.52E−24 | 4.14E−23 | 0.03703704 | YES |
| HSPA8 | ENSG00000109971 | 13 | 189 | 9.19E−26 | 1.58E−24 | 0.06435644 | YES |
| HSPA9 | ENSG00000113013 | 43 | 1157 | 1.88E−99 | 3.56E−97 | 0.03583333 | YES |
| HSPD1 | ENSG00000144381 | 44 | 395 | 9.88E−39 | 2.46E−37 | 0.10022779 | YES |
| HSPE1 | ENSG00000115541 | 10 | 385 | 7.46E−50 | 2.62E−48 | 0.02531646 | YES |
| HTATSF1 | ENSG00000102241 | 49 | 175 | 1.08E−12 | 1.28E−11 | 0.21875 | YES |
| HUS1 | ENSG00000136273 | 24 | 360 | 8.33E−42 | 2.31E−40 | 0.0625 | YES |
| IARS | ENSG00000196305 | 407 | 1227 | 3.31E−36 | 7.65E−35 | 0.24908201 | YES |
| IDI1 | ENSG00000067064 | 244 | 667 | 1.57E−22 | 2.45E−21 | 0.26783754 | YES |
| IGBP1 | ENSG00000089289 | 53 | 211 | 7.15E−16 | 9.27E−15 | 0.20075758 | YES |
| IK | ENSG00000113141 | 6 | 151 | 1.25E−23 | 2.02E−22 | 0.03821656 | YES |
| IKBKAP | ENSG00000070061 | 69 | 486 | 1.93E−40 | 5.14E−39 | 0.12432432 | YES |
| ILF2 | ENSG00000143621 | 57 | 434 | 9.11E−39 | 2.28E−37 | 0.11608961 | YES |
| ILF3 | ENSG00000129351 | 798 | 1251 | 2.25E−09 | 2.38E−08 | 0.38945827 | YES |
| IMMT | ENSG00000132305 | 230 | 1678 | 4.06E−87 | 5.10E−85 | 0.12054507 | YES |
| IMP4 | ENSG00000136718 | 91 | 370 | 8.39E−24 | 1.36E−22 | 0.19739696 | YES |
| IMPDH2 | ENSG00000178035 | 296 | 466 | 5.59E−06 | 5.20E−05 | 0.38845144 | YES |
| INCENP | ENSG00000149503 | 15 | 267 | 1.01E−34 | 2.24E−33 | 0.05319149 | YES |
| INO80 | ENSG00000128908 | 335 | 2970 | 6.53E−126 | 2.65E−123 | 0.10136157 | YES |
| INO80C | ENSG00000153391 | 3485 | 4323 | 6.50E−05 | 0.00056925 | 0.44633709 | YES |
| INSIG1 | ENSG00000186480 | 39 | 86 | 0.00011923 | 0.00103137 | 0.312 | YES |
| INTS12 | ENSG00000138785 | 40 | 295 | 3.78E−29 | 7.19E−28 | 0.11940299 | YES |
| INTS2 | ENSG00000108506 | 244 | 749 | 4.79E−28 | 8.75E−27 | 0.24572004 | YES |
| INTS3 | ENSG00000143624 | 222 | 340 | 9.86E−05 | 0.00085536 | 0.39501779 | YES |
| INTS4 | ENSG00000149262 | 49 | 505 | 4.60E−48 | 1.53E−46 | 0.08844765 | YES |
| INTS5 | ENSG00000185085 | 77 | 190 | 7.12E−09 | 7.42E−08 | 0.28838951 | YES |
| INTS7 | ENSG00000143493 | 317 | 781 | 3.91E−21 | 5.86E−20 | 0.28870674 | YES |
| IPO11 | ENSG00000086200 | 345 | 1938 | 3.60E−81 | 3.70E−79 | 0.15111695 | YES |
| IPO7 | ENSG00000205339 | 93 | 972 | 1.29E−73 | 9.89E−72 | 0.08732394 | YES |
| IPO9 | ENSG00000198700 | 358 | 654 | 1.34E−10 | 1.48E−09 | 0.35375494 | YES |
| IPPK | ENSG00000127080 | 164 | 285 | 5.27E−06 | 4.91E−05 | 0.36525612 | YES |
| ISCA1 | ENSG00000135070 | 231 | 633 | 6.48E−22 | 9.96E−21 | 0.26736111 | YES |
| ISCU | ENSG00000136003 | 10 | 164 | 1.17E−23 | 1.89E−22 | 0.05747126 | YES |
| ISG20L2 | ENSG00000143319 | 127 | 438 | 7.05E−23 | 1.11E−21 | 0.22477876 | YES |
| ISY1 | ENSG00000240682 | 102 | 762 | 1.05E−55 | 4.53E−54 | 0.11805556 | YES |
| IWS1 | ENSG00000163166 | 441 | 605 | 0.00031165 | 0.00262565 | 0.42160612 | YES |
| KANSL1 | ENSG00000120071 | 473 | 7812 | 9.36E−224 | 4.09E−220 | 0.05709113 | YES |
| KANSL2 | ENSG00000139620 | 137 | 571 | 2.64E−32 | 5.42E−31 | 0.19350282 | YES |
| KANSL3 | ENSG00000114982 | 506 | 1063 | 1.09E−18 | 1.51E−17 | 0.32249841 | YES |
| KARS | ENSG00000065427 | 49 | 674 | 4.45E−63 | 2.36E−61 | 0.06777317 | YES |
| KAT7 | ENSG00000136504 | 488 | 906 | 4.67E−13 | 5.56E−12 | 0.35007174 | YES |
| KAT8 | ENSG00000103510 | 7 | 50 | 1.08E−07 | 1.09E−06 | 0.12280702 | YES |
| KCMF1 | ENSG00000176407 | 705 | 1342 | 7.71E−17 | 1.03E−15 | 0.34440645 | YES |
| KDM8 | ENSG00000155666 | 311 | 690 | 1.85E−16 | 2.44E−15 | 0.31068931 | YES |
| KIAA0391 | ENSG00000100890 | 663 | 1327 | 7.98E−19 | 1.12E−17 | 0.33316583 | YES |
| KIAA0947 | ENSG00000164151 | 34 | 484 | 6.68E−51 | 2.45E−49 | 0.06563707 | YES |
| KIAA1731 | ENSG00000166004 | 130 | 613 | 3.27E−37 | 7.77E−36 | 0.17496635 | YES |
| KIF11 | ENSG00000138160 | 128 | 2451 | 3.31E−142 | 1.80E−139 | 0.04963164 | YES |
| KIF14 | ENSG00000118193 | 146 | 849 | 2.55E−52 | 9.90E−51 | 0.14673367 | YES |
| KIF18A | ENSG00000121621 | 384 | 2394 | 5.84E−96 | 9.80E−94 | 0.13822894 | YES |
| KIF4A | ENSG00000090889 | 2236 | 4027 | 6.15E−23 | 9.72E−22 | 0.3570174 | YES |
| KIN | ENSG00000151657 | 12 | 171 | 9.05E−24 | 1.47E−22 | 0.06557377 | YES |
| KPNA4 | ENSG00000186432 | 208 | 318 | 0.00014663 | 0.00126218 | 0.39543726 | YES |
| KPNB1 | ENSG00000108424 | 163 | 1546 | 4.63E−93 | 7.03E−91 | 0.09537741 | YES |
| KRI1 | ENSG00000129347 | 42 | 150 | 2.15E−11 | 2.44E−10 | 0.21875 | YES |
| KRR1 | ENSG00000111615 | 15 | 256 | 2.11E−33 | 4.48E−32 | 0.05535055 | YES |
| LAGE3 | ENSG00000196976 | 32 | 85 | 8.49E−06 | 7.80E−05 | 0.27350427 | YES |
| LAMTOR1 | ENSG00000149357 | 295 | 703 | 7.50E−19 | 1.05E−17 | 0.29559118 | YES |
| LAMTOR2 | ENSG00000116586 | 115 | 269 | 3.10E−10 | 3.39E−09 | 0.29947917 | YES |
| LAMTOR5 | ENSG00000134248 | 346 | 606 | 3.64E−09 | 3.83E−08 | 0.36344538 | YES |
| LARS | ENSG00000133706 | 92 | 1051 | 2.60E−79 | 2.47E−77 | 0.08048994 | YES |
| LARS2 | ENSG00000011376 | 1364 | 2307 | 1.79E−15 | 2.31E−14 | 0.37156088 | YES |
| LAS1L | ENSG00000001497 | 15 | 95 | 8.96E−12 | 1.02E−10 | 0.13636364 | YES |
| LDLR | ENSG00000130164 | 721 | 1239 | 2.14E−12 | 2.49E−11 | 0.36785714 | YES |
| LENG1 | ENSG00000105617 | 29 | 262 | 6.36E−29 | 1.20E−27 | 0.09965636 | YES |
| LENG8 | ENSG00000167615 | 149 | 257 | 1.40E−05 | 0.00012691 | 0.36699507 | YES |
| LENG9 | ENSG00000182909 | 12 | 37 | 0.00053974 | 0.004457 | 0.24489796 | YES |
| LEO1 | ENSG00000166477 | 112 | 533 | 3.11E−34 | 6.76E−33 | 0.17364341 | YES |
| LETM1 | ENSG00000168924 | 81 | 806 | 1.57E−64 | 8.79E−63 | 0.09131905 | YES |
| LIAS | ENSG00000121897 | 1127 | 1590 | 5.84E−07 | 5.69E−06 | 0.41479573 | YES |
| LIG1 | ENSG00000105486 | 93 | 294 | 4.72E−16 | 6.14E−15 | 0.24031008 | YES |
| LIN52 | ENSG00000205659 | 116 | 2201 | 1.60E−134 | 7.54E−132 | 0.05006474 | YES |
| LIN54 | ENSG00000189308 | 190 | 1653 | 2.30E−93 | 3.52E−91 | 0.10309278 | YES |
| LIN9 | ENSG00000183814 | 120 | 986 | 5.30E−68 | 3.27E−66 | 0.1084991 | YES |
| LONP1 | ENSG00000196365 | 125 | 1042 | 1.06E−70 | 7.28E−69 | 0.10711225 | YES |
| LRPPRC | ENSG00000138095 | 3511 | 7126 | 1.38E−37 | 3.32E−36 | 0.33007427 | YES |
| LRR1 | ENSG00000165501 | 111 | 735 | 2.15E−51 | 8.09E−50 | 0.13120567 | YES |
| LRRFIP1 | ENSG00000124831 | 974 | 1387 | 7.32E−07 | 7.09E−06 | 0.41253706 | YES |
| LSG1 | ENSG00000041802 | 38 | 239 | 2.95E−23 | 4.70E−22 | 0.13718412 | YES |
| LSM10 | ENSG00000181817 | 314 | 462 | 7.90E−05 | 0.00068739 | 0.40463918 | YES |
| LSM11 | ENSG00000155858 | 13 | 112 | 4.99E−15 | 6.36E−14 | 0.104 | YES |
| LSM2 | ENSG00000204392 | 55 | 184 | 2.29E−12 | 2.66E−11 | 0.23012552 | YES |
| LSM3 | ENSG00000170860 | 46 | 515 | 6.10E−50 | 2.15E−48 | 0.08199643 | YES |
| LSM4 | ENSG00000130520 | 115 | 685 | 1.98E−46 | 6.18E−45 | 0.14375 | YES |
| LSM5 | ENSG00000106355 | 19 | 288 | 1.26E−35 | 2.87E−34 | 0.06188925 | YES |
| LSM6 | ENSG00000164167 | 12 | 128 | 9.32E−18 | 1.27E−16 | 0.08571429 | YES |
| LTV1 | ENSG00000135521 | 52 | 748 | 3.42E−68 | 2.13E−66 | 0.065 | YES |
| LUC7L3 | ENSG00000108848 | 83 | 1032 | 2.40E−80 | 2.34E−78 | 0.07443946 | YES |
| LYRM4 | ENSG00000214113 | 558 | 4970 | 5.23E−157 | 6.09E−154 | 0.10094067 | YES |
| MAGOH | ENSG00000162385 | 11 | 177 | 5.36E−25 | 8.97E−24 | 0.05851064 | YES |
| MALSU1 | ENSG00000156928 | 124 | 352 | 6.22E−16 | 8.07E−15 | 0.2605042 | YES |
| MAPK14 | ENSG00000112062 | 1028 | 1317 | 0.00035307 | 0.00295889 | 0.43837953 | YES |
| MARCH5 | ENSG00000198060 | 505 | 663 | 0.00112719 | 0.00905955 | 0.43236301 | YES |
| MARS | ENSG00000166986 | 67 | 877 | 1.03E−73 | 8.00E−72 | 0.07097458 | YES |
| MASTL | ENSG00000120539 | 123 | 697 | 1.36E−45 | 4.12E−44 | 0.15 | YES |
| MAT2A | ENSG00000168906 | 22 | 450 | 8.30E−52 | 3.17E−50 | 0.04661017 | YES |
| MATR3 | ENSG00000015479 | 3384 | 5409 | 2.62E−17 | 3.52E−16 | 0.38485159 | YES |
| MAU2 | ENSG00000129933 | 131 | 294 | 4.02E−10 | 4.38E−09 | 0.30823529 | YES |
| MAX | ENSG00000125952 | 119 | 829 | 8.12E−57 | 3.64E−55 | 0.12552743 | YES |
| MBNL1 | ENSG00000152601 | 5262 | 6397 | 0.00010472 | 0.0009072 | 0.45132516 | YES |
| MCL1 | ENSG00000143384 | 6 | 80 | 4.98E−13 | 5.92E−12 | 0.06976744 | YES |
| MCM10 | ENSG00000065328 | 68 | 506 | 1.33E−42 | 3.73E−41 | 0.1184669 | YES |
| MCM2 | ENSG00000073111 | 633 | 1552 | 3.74E−30 | 7.26E−29 | 0.28970252 | YES |
| MCM3 | ENSG00000112118 | 47 | 567 | 2.05E−54 | 8.52E−53 | 0.07654723 | YES |
| MCM5 | ENSG00000100297 | 128 | 724 | 1.21E−46 | 3.80E−45 | 0.15023474 | YES |
| MCM6 | ENSG00000076003 | 42 | 481 | 5.64E−48 | 1.88E−46 | 0.08030593 | YES |
| MCM7 | ENSG00000166508 | 34 | 403 | 7.95E−43 | 2.26E−41 | 0.0778032 | YES |
| MCMBP | ENSG00000197771 | 153 | 732 | 4.92E−42 | 1.37E−40 | 0.17288136 | YES |
| MCRS1 | ENSG00000187778 | 72 | 505 | 2.05E−41 | 5.64E−40 | 0.12478336 | YES |
| MCTS1 | ENSG00000232119 | 120 | 402 | 5.35E−21 | 7.95E−20 | 0.22988506 | YES |
| MDC1 | ENSG00000137337 | 863 | 1617 | 1.16E−17 | 1.58E−16 | 0.34798387 | YES |
| MDN1 | ENSG00000112159 | 93 | 893 | 6.10E−68 | 3.75E−66 | 0.09432049 | YES |
| MED1 | ENSG00000125686 | 191 | 589 | 2.12E−24 | 3.50E−23 | 0.24487179 | YES |
| MED10 | ENSG00000133398 | 13 | 185 | 3.11E−25 | 5.22E−24 | 0.06565657 | YES |
| MED12 | ENSG00000184634 | 91 | 295 | 1.58E−16 | 2.09E−15 | 0.2357513 | YES |
| MED13 | ENSG00000108510 | 1402 | 1804 | 9.86E−05 | 0.00085536 | 0.43730505 | YES |
| MED13L | ENSG00000123066 | 8435 | 10510 | 4.05E−06 | 3.79E−05 | 0.44523621 | YES |
| MED14 | ENSG00000180182 | 62 | 988 | 5.07E−83 | 5.61E−81 | 0.05904762 | YES |
| MED15 | ENSG00000099917 | 769 | 2544 | 4.01E−58 | 1.88E−56 | 0.23211591 | YES |
| MED17 | ENSG00000042429 | 58 | 282 | 1.49E−22 | 2.33E−21 | 0.17058824 | YES |
| MED18 | ENSG00000130772 | 329 | 842 | 2.09E−23 | 3.33E−22 | 0.28095645 | YES |
| MED19 | ENSG00000156603 | 9 | 120 | 7.71E−18 | 1.05E−16 | 0.06976744 | YES |
| MED20 | ENSG00000124641 | 15 | 365 | 9.39E−46 | 2.87E−44 | 0.03947368 | YES |
| MED21 | ENSG00000152944 | 7 | 200 | 9.00E−30 | 1.73E−28 | 0.03381643 | YES |
| MED22 | ENSG00000148297 | 94 | 271 | 1.09E−13 | 1.32E−12 | 0.25753425 | YES |
| MED23 | ENSG00000112282 | 221 | 373 | 1.86E−06 | 1.78E−05 | 0.37205387 | YES |
| MED26 | ENSG00000105085 | 197 | 3036 | 1.03E−150 | 1.06E−147 | 0.06093412 | YES |
| MED27 | ENSG00000160563 | 664 | 5089 | 2.86E−148 | 2.17E−145 | 0.11541804 | YES |
| MED28 | ENSG00000118579 | 16 | 327 | 3.17E−41 | 8.69E−40 | 0.04664723 | YES |
| MED29 | ENSG00000063322 | 31 | 202 | 6.34E−21 | 9.41E−20 | 0.13304721 | YES |
| MED31 | ENSG00000108590 | 28 | 364 | 8.51E−41 | 2.30E−39 | 0.07142857 | YES |
| MED4 | ENSG00000136146 | 19 | 362 | 6.92E−44 | 2.01E−42 | 0.04986877 | YES |
| MED6 | ENSG00000133997 | 20 | 197 | 4.44E−24 | 7.20E−23 | 0.0921659 | YES |
| MED7 | ENSG00000155868 | 55 | 141 | 1.07E−07 | 1.08E−06 | 0.28061224 | YES |
| MED8 | ENSG00000159479 | 11 | 114 | 4.06E−16 | 5.30E−15 | 0.088 | YES |
| MED9 | ENSG00000141026 | 46 | 550 | 3.49E−53 | 1.39E−51 | 0.07718121 | YES |
| METAP1 | ENSG00000164024 | 190 | 418 | 4.10E−12 | 4.74E−11 | 0.3125 | YES |
| METAP2 | ENSG00000111142 | 70 | 557 | 9.55E−47 | 3.01E−45 | 0.11164274 | YES |
| METTL1 | ENSG00000037897 | 75 | 314 | 8.47E−22 | 1.30E−20 | 0.19280206 | YES |
| METTL14 | ENSG00000145388 | 19 | 128 | 3.96E−15 | 5.06E−14 | 0.1292517 | YES |
| METTL16 | ENSG00000127804 | 639 | 4100 | 5.88E−124 | 2.19E−121 | 0.13483857 | YES |
| METTL3 | ENSG00000165819 | 66 | 544 | 1.11E−46 | 3.50E−45 | 0.10819672 | YES |
| MFAP1 | ENSG00000140259 | 24 | 680 | 1.12E−71 | 7.99E−70 | 0.03409091 | YES |
| MFN2 | ENSG00000116688 | 178 | 314 | 1.49E−06 | 1.42E−05 | 0.36178862 | YES |
| MGEA5 | ENSG00000198408 | 413 | 767 | 5.41E−12 | 6.21E−11 | 0.35 | YES |
| MINOS1 | ENSG00000173436 | 414 | 688 | 1.75E−08 | 1.80E−07 | 0.37568058 | YES |
| MIPEP | ENSG00000027001 | 397 | 777 | 1.24E−13 | 1.50E−12 | 0.33816014 | YES |
| MIS12 | ENSG00000167842 | 9 | 214 | 1.08E−30 | 2.14E−29 | 0.04035874 | YES |
| MIS18A | ENSG00000159055 | 25 | 376 | 3.76E−43 | 1.07E−41 | 0.06234414 | YES |
| MIS18BP1 | ENSG00000129534 | 307 | 768 | 1.90E−21 | 2.88E−20 | 0.2855814 | YES |
| MKI67IP | ENSG00000155438 | 24 | 368 | 1.16E−42 | 3.25E−41 | 0.06122449 | YES |
| MMS19 | ENSG00000155229 | 139 | 2101 | 2.16E−125 | 8.59E−123 | 0.06205357 | YES |
| MMS22L | ENSG00000146263 | 140 | 543 | 2.14E−29 | 4.07E−28 | 0.20497804 | YES |
| MNAT1 | ENSG00000020426 | 333 | 1232 | 1.01E−45 | 3.06E−44 | 0.21277955 | YES |
| MNF1 | ENSG00000137288 | 677 | 960 | 8.10E−06 | 7.46E−05 | 0.41356139 | YES |
| MOB4 | ENSG00000115540 | 24 | 302 | 2.28E−35 | 5.13E−34 | 0.07361963 | YES |
| MPDU1 | ENSG00000129255 | 858 | 1104 | 0.00050513 | 0.00418701 | 0.43730887 | YES |
| MPHOSPH10 | ENSG00000124383 | 71 | 550 | 7.76E−46 | 2.38E−44 | 0.11433172 | YES |
| MPHOSPH6 | ENSG00000135698 | 45 | 277 | 1.39E−25 | 2.37E−24 | 0.13975155 | YES |
| MRPL10 | ENSG00000159111 | 176 | 458 | 9.67E−17 | 1.28E−15 | 0.27760252 | YES |
| MRPL11 | ENSG00000174547 | 169 | 341 | 3.84E−09 | 4.04E−08 | 0.33137255 | YES |
| MRPL12 | ENSG00000262814 | 363 | 528 | 6.17E−05 | 0.00054116 | 0.40740741 | YES |
| MRPL16 | ENSG00000166902 | 66 | 234 | 1.99E−15 | 2.56E−14 | 0.22 | YES |
| MRPL17 | ENSG00000158042 | 140 | 357 | 6.43E−14 | 7.87E−13 | 0.28169014 | YES |
| MRPL18 | ENSG00000112110 | 723 | 2116 | 9.59E−46 | 2.92E−44 | 0.25466714 | YES |
| MRPL19 | ENSG00000115364 | 181 | 535 | 7.63E−22 | 1.17E−20 | 0.2527933 | YES |
| MRPL2 | ENSG00000112651 | 146 | 497 | 1.74E−24 | 2.89E−23 | 0.22706065 | YES |
| MRPL20 | ENSG00000242485 | 263 | 401 | 4.70E−05 | 0.00041566 | 0.39608434 | YES |
| MRPL21 | ENSG00000197345 | 134 | 406 | 6.58E−19 | 9.22E−18 | 0.24814815 | YES |
| MRPL22 | ENSG00000082515 | 107 | 405 | 9.22E−24 | 1.49E−22 | 0.20898438 | YES |
| MRPL23 | ENSG00000214026 | 478 | 819 | 4.45E−10 | 4.85E−09 | 0.36854279 | YES |
| MRPL24 | ENSG00000143314 | 452 | 819 | 1.12E−11 | 1.28E−10 | 0.35562549 | YES |
| MRPL27 | ENSG00000108826 | 174 | 566 | 2.62E−25 | 4.41E−24 | 0.23513514 | YES |
| MRPL3 | ENSG00000114686 | 378 | 1054 | 4.69E−30 | 9.07E−29 | 0.26396648 | YES |
| MRPL32 | ENSG00000106591 | 55 | 272 | 3.10E−22 | 4.82E−21 | 0.16819572 | YES |
| MRPL33 | ENSG00000243147 | 106 | 302 | 1.73E−14 | 2.16E−13 | 0.25980392 | YES |
| MRPL34 | ENSG00000130312 | 56 | 129 | 2.63E−06 | 2.49E−05 | 0.3027027 | YES |
| MRPL35 | ENSG00000132313 | 369 | 1138 | 9.04E−36 | 2.07E−34 | 0.24485733 | YES |
| MRPL37 | ENSG00000116221 | 81 | 271 | 3.99E−16 | 5.21E−15 | 0.23011364 | YES |
| MRPL38 | ENSG00000204316 | 163 | 372 | 5.03E−12 | 5.79E−11 | 0.3046729 | YES |
| MRPL39 | ENSG00000154719 | 119 | 326 | 1.73E−14 | 2.16E−13 | 0.26741573 | YES |
| MRPL4 | ENSG00000105364 | 159 | 402 | 8.30E−15 | 1.05E−13 | 0.28342246 | YES |
| MRPL40 | ENSG00000185608 | 464 | 696 | 3.30E−06 | 3.10E−05 | 0.4 | YES |
| MRPL42 | ENSG00000198015 | 282 | 544 | 7.03E−11 | 7.81E−10 | 0.34140436 | YES |
| MRPL44 | ENSG00000135900 | 78 | 264 | 5.02E−16 | 6.52E−15 | 0.22807018 | YES |
| MRPL45 | ENSG00000174100 | 121 | 375 | 1.95E−18 | 2.70E−17 | 0.24395161 | YES |
| MRPL47 | ENSG00000136522 | 156 | 440 | 3.21E−18 | 4.44E−17 | 0.26174497 | YES |
| MRPL48 | ENSG00000175581 | 1289 | 2431 | 2.01E−21 | 3.04E−20 | 0.34650538 | YES |
| MRPL49 | ENSG00000149792 | 129 | 406 | 9.41E−20 | 1.34E−18 | 0.2411215 | YES |
| MRPL50 | ENSG00000136897 | 165 | 434 | 1.93E−16 | 2.55E−15 | 0.2754591 | YES |
| MRPL51 | ENSG00000111639 | 189 | 362 | 1.62E−08 | 1.67E−07 | 0.3430127 | YES |
| MRPL52 | ENSG00000172590 | 57 | 199 | 1.35E−13 | 1.63E−12 | 0.22265625 | YES |
| MRPL54 | ENSG00000183617 | 284 | 643 | 2.10E−16 | 2.77E−15 | 0.30636462 | YES |
| MRPL9 | ENSG00000143436 | 96 | 224 | 4.36E−09 | 4.58E−08 | 0.3 | YES |
| MRPS10 | ENSG00000048544 | 283 | 699 | 6.44E−20 | 9.23E−19 | 0.28818737 | YES |
| MRPS12 | ENSG00000128626 | 261 | 371 | 0.0006513 | 0.00535292 | 0.41297468 | YES |
| MRPS14 | ENSG00000120333 | 52 | 166 | 5.32E−11 | 5.93E−10 | 0.23853211 | YES |
| MRPS15 | ENSG00000116898 | 245 | 485 | 8.81E−11 | 9.75E−10 | 0.33561644 | YES |
| MRPS16 | ENSG00000182180 | 273 | 412 | 5.81E−05 | 0.00051042 | 0.39854015 | YES |
| MRPS17 | ENSG00000239789 | 68 | 187 | 4.15E−10 | 4.52E−09 | 0.26666667 | YES |
| MRPS18A | ENSG00000096080 | 97 | 320 | 8.95E−18 | 1.22E−16 | 0.23261391 | YES |
| MRPS18B | ENSG00000204568 | 691 | 1318 | 8.92E−17 | 1.19E−15 | 0.34395222 | YES |
| MRPS18C | ENSG00000163319 | 106 | 333 | 2.57E−17 | 3.45E−16 | 0.24145786 | YES |
| MRPS23 | ENSG00000181610 | 179 | 368 | 6.51E−10 | 7.02E−09 | 0.32723949 | YES |
| MRPS24 | ENSG00000062582 | 90 | 182 | 2.49E−06 | 2.36E−05 | 0.33088235 | YES |
| MRPS25 | ENSG00000131368 | 145 | 437 | 1.07E−19 | 1.53E−18 | 0.24914089 | YES |
| MRPS27 | ENSG00000113048 | 2500 | 4701 | 3.28E−27 | 5.84E−26 | 0.347174 | YES |
| MRPS30 | ENSG00000112996 | 104 | 350 | 2.93E−19 | 4.14E−18 | 0.22907489 | YES |
| MRPS31 | ENSG00000102738 | 104 | 234 | 7.99E−09 | 8.31E−08 | 0.30769231 | YES |
| MRPS33 | ENSG00000090263 | 225 | 505 | 4.87E−14 | 5.99E−13 | 0.30821918 | YES |
| MRPS35 | ENSG00000061794 | 346 | 719 | 7.61E−15 | 9.65E−14 | 0.32488263 | YES |
| MRPS36 | ENSG00000134056 | 308 | 510 | 3.21E−07 | 3.17E−06 | 0.37652812 | YES |
| MRPS5 | ENSG00000144029 | 194 | 332 | 2.77E−06 | 2.62E−05 | 0.36882129 | YES |
| MRPS6 | ENSG00000243927 | 1650 | 2503 | 5.02E−11 | 5.60E−10 | 0.39730315 | YES |
| MRPS7 | ENSG00000125445 | 165 | 353 | 2.05E−10 | 2.25E−09 | 0.31853282 | YES |
| MRPS9 | ENSG00000135972 | 1797 | 3944 | 1.19E−36 | 2.79E−35 | 0.31301167 | YES |
| MRTO4 | ENSG00000053372 | 78 | 704 | 3.20E−57 | 1.46E−55 | 0.09974425 | YES |
| MSL1 | ENSG00000188895 | 177 | 384 | 3.13E−11 | 3.52E−10 | 0.31550802 | YES |
| MSL2 | ENSG00000174579 | 295 | 615 | 1.06E−13 | 1.29E−12 | 0.32417582 | YES |
| MTERFD2 | ENSG00000122085 | 158 | 331 | 1.21E−09 | 1.29E−08 | 0.32310838 | YES |
| MTG1 | ENSG00000148824 | 385 | 646 | 1.91E−08 | 1.96E−07 | 0.37342386 | YES |
| MTHFD1 | ENSG00000100714 | 628 | 1570 | 2.53E−31 | 5.08E−30 | 0.28571429 | YES |
| MTOR | ENSG00000198793 | 38 | 377 | 8.96E−39 | 2.24E−37 | 0.09156627 | YES |
| MTPAP | ENSG00000107951 | 369 | 1265 | 7.78E−43 | 2.21E−41 | 0.22582619 | YES |
| MVD | ENSG00000167508 | 61 | 381 | 2.86E−32 | 5.87E−31 | 0.13800905 | YES |
| MYBBP1A | ENSG00000132382 | 91 | 615 | 2.48E−46 | 7.74E−45 | 0.12889518 | YES |
| MYBL2 | ENSG00000101057 | 79 | 422 | 1.16E−31 | 2.35E−30 | 0.15768463 | YES |
| MZT1 | ENSG00000204899 | 29 | 207 | 3.35E−22 | 5.19E−21 | 0.12288136 | YES |
| N6AMT1 | ENSG00000156239 | 88 | 619 | 1.95E−47 | 6.38E−46 | 0.12446959 | YES |
| NAA10 | ENSG00000102030 | 6 | 73 | 6.64E−12 | 7.61E−11 | 0.07594937 | YES |
| NAA15 | ENSG00000164134 | 178 | 1161 | 3.70E−67 | 2.25E−65 | 0.13293503 | YES |
| NAA20 | ENSG00000173418 | 50 | 347 | 7.72E−32 | 1.57E−30 | 0.12594458 | YES |
| NAA25 | ENSG00000111300 | 310 | 2214 | 2.31E−99 | 4.35E−97 | 0.12282092 | YES |
| NAA30 | ENSG00000139977 | 216 | 310 | 0.00098586 | 0.00795659 | 0.41064639 | YES |
| NAA38 | ENSG00000128534 | 7 | 185 | 8.34E−28 | 1.51E−26 | 0.03645833 | YES |
| NAA50 | ENSG00000121579 | 103 | 813 | 2.00E−59 | 9.80E−58 | 0.11244541 | YES |
| NACA | ENSG00000196531 | 126 | 1257 | 2.28E−84 | 2.67E−82 | 0.09110629 | YES |
| NAE1 | ENSG00000159593 | 92 | 722 | 5.15E−55 | 2.19E−53 | 0.11302211 | YES |
| NAF1 | ENSG00000145414 | 85 | 571 | 5.23E−44 | 1.53E−42 | 0.12957317 | YES |
| NAPA | ENSG00000105402 | 22 | 216 | 9.02E−26 | 1.55E−24 | 0.09243697 | YES |
| NAPG | ENSG00000134265 | 107 | 377 | 3.50E−21 | 5.25E−20 | 0.22107438 | YES |
| NARFL | ENSG00000103245 | 14 | 112 | 1.20E−14 | 1.51E−13 | 0.11111111 | YES |
| NARS | ENSG00000134440 | 102 | 655 | 5.15E−47 | 1.66E−45 | 0.1347424 | YES |
| NARS2 | ENSG00000137513 | 8814 | 12386 | 1.69E−12 | 1.98E−11 | 0.41575472 | YES |
| NAT10 | ENSG00000135372 | 83 | 730 | 5.32E−58 | 2.47E−56 | 0.10209102 | YES |
| NBAS | ENSG00000151779 | 218 | 2864 | 5.51E−141 | 2.83E−138 | 0.07073329 | YES |
| NCAPD2 | ENSG00000010292 | 139 | 1059 | 1.11E−68 | 7.07E−67 | 0.11602671 | YES |
| NCAPG | ENSG00000109805 | 152 | 1025 | 1.51E−63 | 8.16E−62 | 0.12914189 | YES |
| NCAPH | ENSG00000121152 | 128 | 1163 | 5.27E−78 | 4.74E−76 | 0.09914795 | YES |
| NCBP1 | ENSG00000136937 | 30 | 523 | 5.95E−56 | 2.61E−54 | 0.05424955 | YES |
| NCBP2 | ENSG00000114503 | 29 | 606 | 9.61E−64 | 5.23E−62 | 0.04566929 | YES |
| NCL | ENSG00000115053 | 19 | 443 | 3.16E−52 | 1.22E−50 | 0.04112554 | YES |
| NCOA1 | ENSG00000084676 | 939 | 1441 | 2.96E−09 | 3.12E−08 | 0.39453782 | YES |
| NCOA2 | ENSG00000140396 | 6515 | 10876 | 2.62E−24 | 4.30E−23 | 0.37461906 | YES |
| NDC80 | ENSG00000080986 | 137 | 1240 | 7.85E−81 | 7.84E−79 | 0.09949165 | YES |
| NDUFA9 | ENSG00000139180 | 406 | 641 | 4.81E−07 | 4.71E−06 | 0.38777459 | YES |
| NDUFAB1 | ENSG00000004779 | 32 | 309 | 2.40E−33 | 5.09E−32 | 0.09384164 | YES |
| NDUFB4 | ENSG00000065518 | 273 | 464 | 2.26E−07 | 2.25E−06 | 0.37042062 | YES |
| NDUFV2 | ENSG00000178127 | 1630 | 2687 | 3.82E−15 | 4.88E−14 | 0.37757702 | YES |
| NEDD1 | ENSG00000139350 | 63 | 389 | 1.54E−32 | 3.18E−31 | 0.13938053 | YES |
| NEDD8 | ENSG00000129559 | 49 | 696 | 6.71E−65 | 3.78E−63 | 0.06577181 | YES |
| NELFA | ENSG00000185049 | 301 | 960 | 6.90E−34 | 1.48E−32 | 0.23869944 | YES |
| NELFB | ENSG00000188986 | 27 | 80 | 3.05E−06 | 2.88E−05 | 0.25233645 | YES |
| NELFE | ENSG00000204356 | 63 | 158 | 4.70E−08 | 4.80E−07 | 0.28506787 | YES |
| NFAT5 | ENSG00000102908 | 5189 | 6415 | 2.90E−05 | 0.00025948 | 0.44717339 | YES |
| NFS1 | ENSG00000244005 | 59 | 743 | 1.01E−65 | 5.79E−64 | 0.07356608 | YES |
| NFYA | ENSG00000001167 | 310 | 1222 | 2.21E−48 | 7.44E−47 | 0.20234987 | YES |
| NFYB | ENSG00000120837 | 666 | 980 | 9.43E−07 | 9.10E−06 | 0.40461725 | YES |
| NFYC | ENSG00000066136 | 836 | 5904 | 1.68E−150 | 1.47E−147 | 0.12403561 | YES |
| NGDN | ENSG00000129460 | 6 | 175 | 6.38E−27 | 1.13E−25 | 0.03314917 | YES |
| NGRN | ENSG00000182768 | 192 | 532 | 5.00E−20 | 7.18E−19 | 0.26519337 | YES |
| NHLRC2 | ENSG00000196865 | 510 | 1105 | 2.76E−20 | 4.00E−19 | 0.31578947 | YES |
| NHP2 | ENSG00000145912 | 19 | 334 | 7.72E−41 | 2.09E−39 | 0.05382436 | YES |
| NHP2L1 | ENSG00000100138 | 63 | 1035 | 6.15E−86 | 7.41E−84 | 0.05737705 | YES |
| NIP7 | ENSG00000132603 | 44 | 353 | 2.67E−34 | 5.85E−33 | 0.11083123 | YES |
| NIPBL | ENSG00000164190 | 322 | 3640 | 2.09E−150 | 1.74E−147 | 0.08127208 | YES |
| NKAP | ENSG00000101882 | 93 | 361 | 1.66E−22 | 2.59E−21 | 0.20484581 | YES |
| NLE1 | ENSG00000073536 | 26 | 395 | 8.72E−45 | 2.60E−43 | 0.06175772 | YES |
| NMD3 | ENSG00000169251 | 87 | 232 | 3.27E−11 | 3.68E−10 | 0.27272727 | YES |
| NMNAT1 | ENSG00000173614 | 618 | 848 | 7.50E−05 | 0.00065485 | 0.42155525 | YES |
| NOB1 | ENSG00000141101 | 73 | 714 | 2.10E−59 | 1.02E−57 | 0.09275731 | YES |
| NOC3L | ENSG00000173145 | 43 | 516 | 5.50E−51 | 2.03E−49 | 0.07692308 | YES |
| NOC4L | ENSG00000184967 | 6 | 40 | 2.13E−06 | 2.03E−05 | 0.13043478 | YES |
| NOL10 | ENSG00000115761 | 51 | 775 | 1.29E−70 | 8.75E−69 | 0.06174334 | YES |
| NOL11 | ENSG00000130935 | 60 | 939 | 3.65E−80 | 3.54E−78 | 0.06006006 | YES |
| NOL12 | ENSG00000256872 | 57 | 745 | 1.82E−66 | 1.08E−64 | 0.07107232 | YES |
| NOL6 | ENSG00000165271 | 29 | 337 | 1.65E−37 | 3.97E−36 | 0.07923497 | YES |
| NOL7 | ENSG00000225921 | 7 | 111 | 2.23E−17 | 3.02E−16 | 0.05932203 | YES |
| NOL8 | ENSG00000198000 | 74 | 481 | 1.28E−38 | 3.16E−37 | 0.13333333 | YES |
| NOL9 | ENSG00000162408 | 25 | 287 | 2.83E−33 | 5.97E−32 | 0.08012821 | YES |
| NOLC1 | ENSG00000166197 | 204 | 1064 | 7.44E−56 | 3.25E−54 | 0.16088328 | YES |
| NOM1 | ENSG00000146909 | 8 | 93 | 3.40E−14 | 4.21E−13 | 0.07920792 | YES |
| NONO | ENSG00000147140 | 978 | 1411 | 2.79E−07 | 2.77E−06 | 0.40937631 | YES |
| NOP10 | ENSG00000182117 | 31 | 362 | 1.57E−39 | 3.99E−38 | 0.07888041 | YES |
| NOP14 | ENSG00000087269 | 66 | 805 | 1.42E−68 | 8.94E−67 | 0.07577497 | YES |
| NOP16 | ENSG00000048162 | 44 | 592 | 1.36E−57 | 6.27E−56 | 0.06918239 | YES |
| NOP2 | ENSG00000111641 | 156 | 591 | 2.34E−30 | 4.58E−29 | 0.20883534 | YES |
| NOP56 | ENSG00000101361 | 92 | 345 | 3.60E−21 | 5.39E−20 | 0.21052632 | YES |
| NOP58 | ENSG00000055044 | 102 | 1120 | 2.18E−81 | 2.26E−79 | 0.08346972 | YES |
| NOP9 | ENSG00000196943 | 10 | 64 | 7.94E−09 | 8.26E−08 | 0.13513514 | YES |
| NPAT | ENSG00000149308 | 52 | 729 | 1.13E−66 | 6.76E−65 | 0.06658131 | YES |
| NPLOC4 | ENSG00000182446 | 62 | 767 | 9.35E−67 | 5.61E−65 | 0.0747889 | YES |
| NPM1 | ENSG00000181163 | 77 | 377 | 1.03E−27 | 1.87E−26 | 0.16960352 | YES |
| NRDE2 | ENSG00000119720 | 42 | 643 | 1.19E−62 | 6.25E−61 | 0.06131387 | YES |
| NRF1 | ENSG00000106459 | 111 | 1579 | 3.14E−106 | 7.42E−104 | 0.06568047 | YES |
| NSA2 | ENSG00000164346 | 14 | 156 | 6.62E−21 | 9.81E−20 | 0.08235294 | YES |
| NSF | ENSG00000073969 | 482 | 9808 | 1.92E−251 | 1.68E−247 | 0.04684159 | YES |
| NSL1 | ENSG00000117697 | 36 | 581 | 3.35E−59 | 1.62E−57 | 0.05834684 | YES |
| NSMCE1 | ENSG00000169189 | 551 | 842 | 3.29E−07 | 3.25E−06 | 0.39554917 | YES |
| NSMCE4A | ENSG00000107672 | 49 | 237 | 7.15E−20 | 1.02E−18 | 0.17132867 | YES |
| NSRP1 | ENSG00000126653 | 408 | 3057 | 1.78E−118 | 5.64E−116 | 0.11774892 | YES |
| NSUN4 | ENSG00000117481 | 299 | 437 | 0.00013157 | 0.00113753 | 0.40625 | YES |
| NUBP1 | ENSG00000103274 | 25 | 368 | 2.67E−42 | 7.46E−41 | 0.06361323 | YES |
| NUBP2 | ENSG00000095906 | 15 | 193 | 1.76E−25 | 2.98E−24 | 0.07211538 | YES |
| NUDC | ENSG00000090273 | 54 | 779 | 4.93E−70 | 3.29E−68 | 0.06482593 | YES |
| NUDCD3 | ENSG00000015676 | 79 | 587 | 5.44E−47 | 1.75E−45 | 0.11861862 | YES |
| NUDT21 | ENSG00000167005 | 50 | 543 | 2.67E−51 | 1.00E−49 | 0.08431703 | YES |
| NUF2 | ENSG00000143228 | 13 | 316 | 3.42E−41 | 9.36E−40 | 0.03951368 | YES |
| NUFIP1 | ENSG00000083635 | 46 | 303 | 3.23E−28 | 5.94E−27 | 0.13180516 | YES |
| NUP107 | ENSG00000111581 | 36 | 431 | 5.20E−45 | 1.55E−43 | 0.07708779 | YES |
| NUP133 | ENSG00000069248 | 43 | 315 | 1.85E−30 | 3.61E−29 | 0.12011173 | YES |
| NUP153 | ENSG00000124789 | 226 | 3168 | 3.10E−150 | 2.46E−147 | 0.0665881 | YES |
| NUP155 | ENSG00000113569 | 82 | 966 | 4.77E−76 | 4.05E−74 | 0.07824427 | YES |
| NUP160 | ENSG00000030066 | 118 | 1521 | 1.69E−101 | 3.43E−99 | 0.07199512 | YES |
| NUP188 | ENSG00000095319 | 380 | 606 | 4.57E−07 | 4.48E−06 | 0.38539554 | YES |
| NUP205 | ENSG00000155561 | 49 | 653 | 2.60E−61 | 1.33E−59 | 0.06980057 | YES |
| NUP214 | ENSG00000126883 | 86 | 679 | 4.94E−53 | 1.96E−51 | 0.1124183 | YES |
| NUP43 | ENSG00000120253 | 21 | 213 | 9.28E−26 | 1.59E−24 | 0.08974359 | YES |
| NUP50 | ENSG00000093000 | 361 | 504 | 0.00034373 | 0.0028834 | 0.41734104 | YES |
| NUP54 | ENSG00000138750 | 24 | 439 | 5.46E−50 | 1.93E−48 | 0.05183585 | YES |
| NUP85 | ENSG00000125450 | 127 | 1056 | 3.45E−71 | 2.41E−69 | 0.10735418 | YES |
| NUP88 | ENSG00000108559 | 39 | 501 | 7.29E−51 | 2.66E−49 | 0.07222222 | YES |
| NUP93 | ENSG00000102900 | 187 | 2766 | 6.74E−143 | 3.80E−140 | 0.06332543 | YES |
| NUP98 | ENSG00000110713 | 68 | 851 | 1.67E−71 | 1.18E−69 | 0.07399347 | YES |
| NUPL1 | ENSG00000139496 | 46 | 405 | 3.88E−39 | 9.83E−38 | 0.10199557 | YES |
| NUPL2 | ENSG00000136243 | 351 | 704 | 1.23E−13 | 1.49E−12 | 0.33270142 | YES |
| NUS1 | ENSG00000153989 | 13 | 87 | 2.72E−11 | 3.06E−10 | 0.13 | YES |
| NUSAP1 | ENSG00000137804 | 2002 | 2731 | 3.07E−07 | 3.04E−06 | 0.42298753 | YES |
| NUTF2 | ENSG00000102898 | 19 | 616 | 4.35E−68 | 2.69E−66 | 0.02992126 | YES |
| NVL | ENSG00000143748 | 75 | 765 | 5.98E−63 | 3.15E−61 | 0.08928571 | YES |
| OGDH | ENSG00000105953 | 230 | 1865 | 1.25E−96 | 2.16E−94 | 0.1097852 | YES |
| OGT | ENSG00000147162 | 363 | 1933 | 2.91E−78 | 2.64E−76 | 0.15810105 | YES |
| OIP5 | ENSG00000104147 | 69 | 947 | 3.26E−78 | 2.95E−76 | 0.06791339 | YES |
| OPA1 | ENSG00000198836 | 270 | 822 | 2.57E−29 | 4.87E−28 | 0.24725275 | YES |
| ORAOV1 | ENSG00000149716 | 7 | 140 | 1.28E−21 | 1.96E−20 | 0.04761905 | YES |
| ORC1 | ENSG00000085840 | 109 | 595 | 2.76E−40 | 7.34E−39 | 0.15482955 | YES |
| ORC2 | ENSG00000115942 | 67 | 352 | 1.12E−27 | 2.03E−26 | 0.15990453 | YES |
| ORC3 | ENSG00000135336 | 297 | 3320 | 6.62E−144 | 4.13E−141 | 0.08211225 | YES |
| ORC4 | ENSG00000115947 | 288 | 478 | 4.98E−07 | 4.86E−06 | 0.37597911 | YES |
| ORC5 | ENSG00000164815 | 84 | 1013 | 8.67E−79 | 8.10E−77 | 0.07657247 | YES |
| ORC6 | ENSG00000091651 | 28 | 347 | 5.88E−39 | 1.48E−37 | 0.07466667 | YES |
| OSBP | ENSG00000110048 | 24 | 188 | 1.79E−21 | 2.72E−20 | 0.11320755 | YES |
| OSGEP | ENSG00000092094 | 87 | 419 | 1.95E−29 | 3.72E−28 | 0.17193676 | YES |
| OXA1L | ENSG00000155463 | 33 | 305 | 1.48E−32 | 3.07E−31 | 0.09763314 | YES |
| PA2G4 | ENSG00000170515 | 623 | 971 | 3.26E−08 | 3.33E−07 | 0.39084065 | YES |
| PABPC1 | ENSG00000070756 | 34 | 188 | 3.07E−18 | 4.26E−17 | 0.15315315 | YES |
| PABPN1 | ENSG00000100836 | 37 | 340 | 4.00E−35 | 8.95E−34 | 0.09814324 | YES |
| PAF1 | ENSG00000006712 | 60 | 265 | 3.49E−20 | 5.02E−19 | 0.18461538 | YES |
| PAFAH1B1 | ENSG00000007168 | 144 | 2118 | 4.11E−125 | 1.59E−122 | 0.06366048 | YES |
| PAICS | ENSG00000128050 | 347 | 1360 | 5.26E−51 | 1.94E−49 | 0.20328061 | YES |
| PAK1IP1 | ENSG00000111845 | 66 | 674 | 4.66E−58 | 2.18E−56 | 0.08918919 | YES |
| PALB2 | ENSG00000083093 | 161 | 1232 | 3.90E−75 | 3.19E−73 | 0.11557789 | YES |
| PAM16 | ENSG00000217930 | 60 | 478 | 3.34E−42 | 9.32E−41 | 0.11152416 | YES |
| PAPOLG | ENSG00000115421 | 72 | 388 | 4.61E−30 | 8.91E−29 | 0.15652174 | YES |
| PARN | ENSG00000140694 | 6613 | 8195 | 1.26E−05 | 0.00011479 | 0.44658293 | YES |
| PARS2 | ENSG00000162396 | 159 | 277 | 6.02E−06 | 5.58E−05 | 0.3646789 | YES |
| PAXBP1 | ENSG00000159086 | 59 | 780 | 1.21E−68 | 7.70E−67 | 0.07032181 | YES |
| PCBP2 | ENSG00000197111 | 226 | 592 | 9.38E−20 | 1.34E−18 | 0.27628362 | YES |
| PCF11 | ENSG00000165494 | 38 | 350 | 6.93E−36 | 1.59E−34 | 0.09793814 | YES |
| PCID2 | ENSG00000126226 | 20 | 522 | 2.12E−59 | 1.03E−57 | 0.03690037 | YES |
| PCNA | ENSG00000132646 | 1190 | 1701 | 1.53E−07 | 1.54E−06 | 0.41162228 | YES |
| PCNP | ENSG00000081154 | 292 | 611 | 9.49E−14 | 1.15E−12 | 0.32336656 | YES |
| PCNT | ENSG00000160299 | 1190 | 2711 | 4.83E−34 | 1.04E−32 | 0.30504999 | YES |
| PCYT1A | ENSG00000161217 | 816 | 1118 | 2.27E−05 | 0.00020394 | 0.42192347 | YES |
| PDAP1 | ENSG00000106244 | 112 | 793 | 1.04E−55 | 4.51E−54 | 0.12375691 | YES |
| PDCD11 | ENSG00000148843 | 70 | 608 | 2.77E−51 | 1.04E−49 | 0.10324484 | YES |
| PDCD2 | ENSG00000071994 | 19 | 148 | 6.59E−18 | 9.01E−17 | 0.11377246 | YES |
| PDCL | ENSG00000136940 | 205 | 343 | 4.83E−06 | 4.51E−05 | 0.37408759 | YES |
| PDPK1 | ENSG00000140992 | 26 | 183 | 3.84E−20 | 5.52E−19 | 0.12440191 | YES |
| PDRG1 | ENSG00000088356 | 12 | 214 | 2.03E−29 | 3.88E−28 | 0.05309735 | YES |
| PDS5A | ENSG00000121892 | 1648 | 2932 | 9.96E−20 | 1.42E−18 | 0.35982533 | YES |
| PDSS1 | ENSG00000148459 | 731 | 933 | 0.00117642 | 0.00944216 | 0.43930288 | YES |
| PDSS2 | ENSG00000164494 | 12182 | 14954 | 7.08E−06 | 6.53E−05 | 0.44892394 | YES |
| PES1 | ENSG00000100029 | 22 | 641 | 3.83E−69 | 2.49E−67 | 0.0331825 | YES |
| PET112 | ENSG00000059691 | 692 | 1216 | 4.44E−13 | 5.29E−12 | 0.36268344 | YES |
| PET117 | ENSG00000232838 | 312 | 569 | 8.42E−10 | 9.06E−09 | 0.35414302 | YES |
| PFAS | ENSG00000178921 | 969 | 1463 | 8.75E−09 | 9.07E−08 | 0.3984375 | YES |
| PFDN1 | ENSG00000113068 | 771 | 1164 | 5.62E−08 | 5.71E−07 | 0.39844961 | YES |
| PFDN2 | ENSG00000143256 | 36 | 475 | 2.32E−49 | 8.05E−48 | 0.0704501 | YES |
| PFDN5 | ENSG00000123349 | 191 | 362 | 2.51E−08 | 2.56E−07 | 0.34538879 | YES |
| PFDN6 | ENSG00000204220 | 33 | 114 | 3.89E−09 | 4.09E−08 | 0.2244898 | YES |
| PGAM1 | ENSG00000171314 | 242 | 897 | 2.73E−38 | 6.65E−37 | 0.21246708 | YES |
| PGD | ENSG00000142657 | 99 | 328 | 3.77E−18 | 5.20E−17 | 0.23185012 | YES |
| PGGT1B | ENSG00000164219 | 87 | 452 | 1.39E−32 | 2.88E−31 | 0.16141002 | YES |
| PGK1 | ENSG00000102144 | 109 | 647 | 1.10E−44 | 3.26E−43 | 0.14417989 | YES |
| PGM3 | ENSG00000013375 | 168 | 269 | 9.17E−05 | 0.00079614 | 0.38443936 | YES |
| PGS1 | ENSG00000087157 | 49 | 350 | 1.86E−32 | 3.84E−31 | 0.12280702 | YES |
| PHB | ENSG00000167085 | 2 | 239 | 5.80E−37 | 1.37E−35 | 0.00829876 | YES |
| PHF23 | ENSG00000040633 | 215 | 455 | 7.28E−12 | 8.33E−11 | 0.32089552 | YES |
| PHF5A | ENSG00000100410 | 13 | 157 | 1.91E−21 | 2.89E−20 | 0.07647059 | YES |
| PIK3C3 | ENSG00000078142 | 196 | 600 | 1.83E−24 | 3.04E−23 | 0.24623116 | YES |
| PIK3R4 | ENSG00000196455 | 449 | 848 | 3.75E−13 | 4.48E−12 | 0.3461835 | YES |
| PLK1 | ENSG00000166851 | 13 | 120 | 3.37E−16 | 4.40E−15 | 0.09774436 | YES |
| PLK4 | ENSG00000142731 | 27 | 217 | 3.97E−24 | 6.46E−23 | 0.11065574 | YES |
| PLRG1 | ENSG00000171566 | 3 | 92 | 2.40E−16 | 3.15E−15 | 0.03157895 | YES |
| PMF1 | ENSG00000160783 | 54 | 763 | 8.83E−69 | 5.67E−67 | 0.06609547 | YES |
| PMM2 | ENSG00000140650 | 827 | 1197 | 6.70E−07 | 6.50E−06 | 0.40859684 | YES |
| PMPCA | ENSG00000165688 | 14 | 241 | 5.62E−32 | 1.15E−30 | 0.05490196 | YES |
| PMPCB | ENSG00000105819 | 8 | 223 | 2.89E−32 | 5.92E−31 | 0.03463203 | YES |
| PMVK | ENSG00000163344 | 322 | 645 | 5.81E−13 | 6.89E−12 | 0.33298862 | YES |
| PNISR | ENSG00000132424 | 63 | 524 | 1.09E−45 | 3.31E−44 | 0.10732538 | YES |
| PNN | ENSG00000100941 | 46 | 553 | 1.86E−53 | 7.54E−52 | 0.07679466 | YES |
| PNO1 | ENSG00000115946 | 26 | 383 | 1.57E−43 | 4.51E−42 | 0.06356968 | YES |
| PNPT1 | ENSG00000138035 | 108 | 413 | 2.70E−24 | 4.43E−23 | 0.20729367 | YES |
| POLA1 | ENSG00000101868 | 436 | 8535 | 1.21E−238 | 7.03E−235 | 0.04860105 | YES |
| POLA2 | ENSG00000014138 | 281 | 801 | 1.51E−26 | 2.66E−25 | 0.25970425 | YES |
| POLD1 | ENSG00000062822 | 158 | 660 | 1.85E−35 | 4.16E−34 | 0.19315403 | YES |
| POLD2 | ENSG00000106628 | 286 | 875 | 1.41E−30 | 2.77E−29 | 0.24633936 | YES |
| POLD3 | ENSG00000077514 | 47 | 771 | 1.62E−71 | 1.15E−69 | 0.05745721 | YES |
| POLE | ENSG00000177084 | 46 | 249 | 4.41E−22 | 6.83E−21 | 0.1559322 | YES |
| POLE2 | ENSG00000100479 | 64 | 694 | 2.71E−60 | 1.37E−58 | 0.08443272 | YES |
| POLG2 | ENSG00000256525 | 415 | 616 | 1.14E−05 | 0.00010428 | 0.40252182 | YES |
| POLR1A | ENSG00000068654 | 69 | 491 | 6.48E−41 | 1.76E−39 | 0.12321429 | YES |
| POLR1B | ENSG00000125630 | 61 | 788 | 1.10E−68 | 7.02E−67 | 0.07184923 | YES |
| POLR1C | ENSG00000171453 | 23 | 579 | 1.84E−63 | 9.88E−62 | 0.03820598 | YES |
| POLR1E | ENSG00000137054 | 73 | 438 | 9.44E−35 | 2.09E−33 | 0.14285714 | YES |
| POLR2A | ENSG00000181222 | 39 | 1003 | 1.03E−90 | 1.43E−88 | 0.03742802 | YES |
| POLR2B | ENSG00000047315 | 39 | 524 | 5.00E−53 | 1.99E−51 | 0.06927176 | YES |
| POLR2C | ENSG00000102978 | 36 | 328 | 3.92E−34 | 8.47E−33 | 0.0989011 | YES |
| POLR2D | ENSG00000144231 | 28 | 355 | 7.94E−40 | 2.05E−38 | 0.07310705 | YES |
| POLR2E | ENSG00000099817 | 15 | 219 | 7.90E−29 | 1.48E−27 | 0.06410256 | YES |
| POLR2F | ENSG00000100142 | 83 | 460 | 2.55E−34 | 5.59E−33 | 0.15285451 | YES |
| POLR2I | ENSG00000105258 | 11 | 39 | 0.0001462 | 0.00125903 | 0.22 | YES |
| POLR2L | ENSG00000177700 | 16 | 150 | 2.69E−19 | 3.80E−18 | 0.09638554 | YES |
| POLR3A | ENSG00000148606 | 23 | 319 | 1.16E−37 | 2.79E−36 | 0.06725146 | YES |
| POLR3B | ENSG00000013503 | 147 | 2580 | 1.97E−143 | 1.19E−140 | 0.05390539 | YES |
| POLR3C | ENSG00000186141 | 112 | 516 | 9.74E−33 | 2.03E−31 | 0.17834395 | YES |
| POLR3E | ENSG00000058600 | 46 | 492 | 9.24E−48 | 3.04E−46 | 0.08550186 | YES |
| POLR3F | ENSG00000132664 | 57 | 269 | 2.21E−21 | 3.34E−20 | 0.17484663 | YES |
| POLR3H | ENSG00000100413 | 76 | 305 | 1.20E−20 | 1.76E−19 | 0.19947507 | YES |
| POLR3K | ENSG00000161980 | 133 | 690 | 7.49E−43 | 2.13E−41 | 0.16160389 | YES |
| POMP | ENSG00000132963 | 10 | 163 | 1.61E−23 | 2.58E−22 | 0.05780347 | YES |
| POP4 | ENSG00000105171 | 269 | 462 | 1.48E−07 | 1.48E−06 | 0.36798906 | YES |
| POP7 | ENSG00000172336 | 212 | 330 | 6.94E−05 | 0.00060686 | 0.39114391 | YES |
| PPA1 | ENSG00000180817 | 83 | 807 | 4.49E−64 | 2.47E−62 | 0.09325843 | YES |
| PPA2 | ENSG00000138777 | 279 | 509 | 3.09E−09 | 3.26E−08 | 0.35406091 | YES |
| PPAN | ENSG00000130810 | 69 | 867 | 2.04E−72 | 1.49E−70 | 0.07371795 | YES |
| PPAT | ENSG00000128059 | 501 | 1301 | 2.83E−30 | 5.51E−29 | 0.27802442 | YES |
| PPIE | ENSG00000084072 | 40 | 486 | 4.19E−49 | 1.44E−47 | 0.07604563 | YES |
| PPIL1 | ENSG00000137168 | 656 | 1261 | 9.89E−17 | 1.31E−15 | 0.34220136 | YES |
| PPIL2 | ENSG00000100023 | 88 | 517 | 2.19E−38 | 5.39E−37 | 0.14545455 | YES |
| PPIL4 | ENSG00000131013 | 52 | 346 | 3.80E−31 | 7.61E−30 | 0.13065327 | YES |
| PPME1 | ENSG00000214517 | 615 | 819 | 0.00031827 | 0.00268009 | 0.42887029 | YES |
| PPP1CB | ENSG00000213639 | 84 | 712 | 2.83E−56 | 1.25E−54 | 0.10552764 | YES |
| PPP1R10 | ENSG00000204569 | 104 | 1177 | 1.45E−84 | 1.72E−82 | 0.08118657 | YES |
| PPP1R11 | ENSG00000204619 | 12 | 150 | 7.00E−21 | 1.04E−19 | 0.07407407 | YES |
| PPP1R15B | ENSG00000158615 | 73 | 196 | 4.01E−10 | 4.37E−09 | 0.27137546 | YES |
| PPP1R2 | ENSG00000184203 | 72 | 374 | 1.19E−28 | 2.22E−27 | 0.16143498 | YES |
| PPP1R7 | ENSG00000115685 | 49 | 233 | 2.07E−19 | 2.93E−18 | 0.17375887 | YES |
| PPP1R8 | ENSG00000117751 | 86 | 910 | 5.42E−71 | 3.74E−69 | 0.08634538 | YES |
| PPP2CA | ENSG00000113575 | 59 | 711 | 3.88E−63 | 2.07E−61 | 0.07662338 | YES |
| PPP2R1A | ENSG00000105568 | 66 | 845 | 1.30E−71 | 9.20E−70 | 0.07244786 | YES |
| PPP2R2A | ENSG00000221914 | 828 | 1271 | 8.76E−09 | 9.08E−08 | 0.39447356 | YES |
| PPP2R3C | ENSG00000092020 | 251 | 1027 | 2.19E−45 | 6.61E−44 | 0.19640063 | YES |
| PPP2R4 | ENSG00000119383 | 2729 | 3312 | 0.00051636 | 0.00427607 | 0.4517464 | YES |
| PPP4C | ENSG00000149923 | 74 | 276 | 4.22E−18 | 5.82E−17 | 0.21142857 | YES |
| PPP6C | ENSG00000119414 | 231 | 469 | 3.87E−11 | 4.34E−10 | 0.33 | YES |
| PPRC1 | ENSG00000148840 | 587 | 1904 | 1.39E−49 | 4.84E−48 | 0.23564833 | YES |
| PPWD1 | ENSG00000113593 | 26 | 450 | 2.31E−50 | 8.30E−49 | 0.05462185 | YES |
| PRC1 | ENSG00000198901 | 126 | 1597 | 8.71E−104 | 1.88E−101 | 0.07312826 | YES |
| PRCC | ENSG00000143294 | 133 | 837 | 3.05E−54 | 1.26E−52 | 0.1371134 | YES |
| PRDM10 | ENSG00000170325 | 226 | 798 | 4.80E−34 | 1.03E−32 | 0.22070313 | YES |
| PREB | ENSG00000138073 | 11 | 96 | 2.03E−13 | 2.44E−12 | 0.10280374 | YES |
| PRELID1 | ENSG00000169230 | 35 | 213 | 4.94E−21 | 7.35E−20 | 0.14112903 | YES |
| PRIM1 | ENSG00000198056 | 70 | 559 | 6.29E−47 | 2.01E−45 | 0.11128776 | YES |
| PRKRIR | ENSG00000137492 | 774 | 1002 | 0.00052696 | 0.00435355 | 0.43581081 | YES |
| PRMT1 | ENSG00000126457 | 76 | 401 | 2.39E−30 | 4.66E−29 | 0.15932914 | YES |
| PRMT5 | ENSG00000100462 | 35 | 341 | 6.78E−36 | 1.56E−34 | 0.09308511 | YES |
| PRPF18 | ENSG00000165630 | 16 | 148 | 5.09E−19 | 7.15E−18 | 0.09756098 | YES |
| PRPF19 | ENSG00000110107 | 18 | 244 | 9.25E−31 | 1.83E−29 | 0.06870229 | YES |
| PRPF3 | ENSG00000117360 | 58 | 929 | 5.02E−80 | 4.85E−78 | 0.05876393 | YES |
| PRPF31 | ENSG00000105618 | 5 | 74 | 1.61E−12 | 1.89E−11 | 0.06329114 | YES |
| PRPF38A | ENSG00000134748 | 13 | 368 | 7.29E−47 | 2.32E−45 | 0.03412073 | YES |
| PRPF38B | ENSG00000134186 | 43 | 388 | 2.56E−38 | 6.24E−37 | 0.09976798 | YES |
| PRPF39 | ENSG00000185246 | 386 | 630 | 1.04E−07 | 1.05E−06 | 0.37992126 | YES |
| PRPF4 | ENSG00000136875 | 37 | 493 | 9.24E−51 | 3.34E−49 | 0.06981132 | YES |
| PRPF40A | ENSG00000196504 | 70 | 604 | 6.21E−51 | 2.28E−49 | 0.10385757 | YES |
| PRPF4B | ENSG00000112739 | 160 | 707 | 9.73E−39 | 2.43E−37 | 0.18454441 | YES |
| PRPF6 | ENSG00000101161 | 36 | 393 | 4.08E−41 | 1.11E−39 | 0.08391608 | YES |
| PRPF8 | ENSG00000174231 | 33 | 303 | 2.50E−32 | 5.14E−31 | 0.09821429 | YES |
| PSMA1 | ENSG00000129084 | 84 | 664 | 2.66E−52 | 1.03E−50 | 0.11229947 | YES |
| PSMA2 | ENSG00000106588 | 16 | 293 | 2.26E−37 | 5.38E−36 | 0.05177994 | YES |
| PSMA3 | ENSG00000100567 | 42 | 649 | 3.69E−63 | 1.97E−61 | 0.06078148 | YES |
| PSMA5 | ENSG00000143106 | 26 | 422 | 1.47E−47 | 4.83E−46 | 0.05803571 | YES |
| PSMA7 | ENSG00000101182 | 24 | 239 | 6.61E−28 | 1.20E−26 | 0.09125475 | YES |
| PSMB1 | ENSG00000008018 | 13 | 190 | 6.78E−26 | 1.17E−24 | 0.06403941 | YES |
| PSMB2 | ENSG00000126067 | 296 | 3382 | 4.21E−146 | 2.94E−143 | 0.08047852 | YES |
| PSMB3 | ENSG00000108294 | 25 | 143 | 3.44E−15 | 4.41E−14 | 0.14880952 | YES |
| PSMB4 | ENSG00000159377 | 10 | 138 | 5.07E−20 | 7.28E−19 | 0.06756757 | YES |
| PSMB5 | ENSG00000100804 | 61 | 289 | 1.51E−22 | 2.37E−21 | 0.17428571 | YES |
| PSMB6 | ENSG00000142507 | 19 | 263 | 1.12E−32 | 2.34E−31 | 0.06737589 | YES |
| PSMB7 | ENSG00000136930 | 38 | 834 | 4.30E−79 | 4.04E−77 | 0.04357798 | YES |
| PSMC1 | ENSG00000100764 | 7 | 155 | 9.88E−24 | 1.60E−22 | 0.04320988 | YES |
| PSMC3 | ENSG00000165916 | 69 | 570 | 3.40E−48 | 1.14E−46 | 0.10798122 | YES |
| PSMC4 | ENSG00000013275 | 3 | 189 | 3.52E−30 | 6.83E−29 | 0.015625 | YES |
| PSMC5 | ENSG00000087191 | 18 | 417 | 5.33E−50 | 1.89E−48 | 0.04137931 | YES |
| PSMC6 | ENSG00000100519 | 27 | 338 | 2.52E−38 | 6.15E−37 | 0.0739726 | YES |
| PSMD1 | ENSG00000173692 | 198 | 1516 | 2.24E−84 | 2.65E−82 | 0.11551925 | YES |
| PSMD11 | ENSG00000108671 | 37 | 493 | 9.24E−51 | 3.34E−49 | 0.06981132 | YES |
| PSMD12 | ENSG00000197170 | 24 | 597 | 1.10E−64 | 6.13E−63 | 0.03864734 | YES |
| PSMD13 | ENSG00000185627 | 51 | 299 | 2.53E−26 | 4.42E−25 | 0.14571429 | YES |
| PSMD14 | ENSG00000115233 | 124 | 1557 | 4.29E−102 | 9.03E−100 | 0.07376562 | YES |
| PSMD2 | ENSG00000175166 | 86 | 255 | 1.38E−13 | 1.66E−12 | 0.25219941 | YES |
| PSMD3 | ENSG00000108344 | 19 | 249 | 5.50E−31 | 1.10E−29 | 0.07089552 | YES |
| PSMD4 | ENSG00000159352 | 115 | 428 | 2.89E−24 | 4.74E−23 | 0.21178637 | YES |
| PSMD6 | ENSG00000163636 | 4 | 95 | 2.52E−16 | 3.30E−15 | 0.04040404 | YES |
| PSMD7 | ENSG00000103035 | 15 | 313 | 4.72E−40 | 1.23E−38 | 0.04573171 | YES |
| PSMD8 | ENSG00000099341 | 25 | 514 | 7.34E−57 | 3.30E−55 | 0.04638219 | YES |
| PSMF1 | ENSG00000125818 | 182 | 423 | 2.11E−13 | 2.54E−12 | 0.30082645 | YES |
| PSMG1 | ENSG00000183527 | 276 | 515 | 8.51E−10 | 9.15E−09 | 0.34892541 | YES |
| PSMG2 | ENSG00000128789 | 370 | 851 | 9.73E−20 | 1.39E−18 | 0.3030303 | YES |
| PSMG4 | ENSG00000180822 | 33 | 371 | 8.28E−40 | 2.13E−38 | 0.08168317 | YES |
| PSTK | ENSG00000179988 | 147 | 287 | 9.54E−08 | 9.66E−07 | 0.33870968 | YES |
| PTAR1 | ENSG00000188647 | 882 | 1729 | 2.54E−20 | 3.69E−19 | 0.33780161 | YES |
| PTCD3 | ENSG00000132300 | 571 | 1187 | 2.43E−19 | 3.43E−18 | 0.32480091 | YES |
| PTMA | ENSG00000187514 | 163 | 286 | 3.76E−06 | 3.53E−05 | 0.36302895 | YES |
| PTP4A1 | ENSG00000112245 | 260 | 473 | 8.13E−09 | 8.45E−08 | 0.35470668 | YES |
| PTPMT1 | ENSG00000110536 | 178 | 763 | 1.30E−39 | 3.33E−38 | 0.18916047 | YES |
| PTTG1 | ENSG00000164611 | 32 | 177 | 1.87E−17 | 2.53E−16 | 0.15311005 | YES |
| PUM1 | ENSG00000134644 | 1367 | 2913 | 1.22E−30 | 2.41E−29 | 0.31939252 | YES |
| PWP1 | ENSG00000136045 | 25 | 277 | 4.17E−32 | 8.52E−31 | 0.08278146 | YES |
| PWP2 | ENSG00000241945 | 16 | 211 | 2.05E−27 | 3.70E−26 | 0.07048458 | YES |
| PYROXD1 | ENSG00000121350 | 110 | 1034 | 1.07E−73 | 8.27E−72 | 0.09615385 | YES |
| QARS | ENSG00000172053 | 54 | 323 | 4.20E−28 | 7.69E−27 | 0.14323607 | YES |
| QRSL1 | ENSG00000130348 | 226 | 519 | 7.62E−15 | 9.65E−14 | 0.3033557 | YES |
| RABGGTB | ENSG00000137955 | 54 | 1059 | 4.65E−90 | 6.25E−88 | 0.04851752 | YES |
| RAC1 | ENSG00000136238 | 165 | 697 | 5.04E−37 | 1.19E−35 | 0.19141531 | YES |
| RACGAP1 | ENSG00000161800 | 239 | 1811 | 2.55E−92 | 3.71E−90 | 0.11658537 | YES |
| RAD1 | ENSG00000113456 | 141 | 709 | 1.11E−42 | 3.15E−41 | 0.16588235 | YES |
| RAD17 | ENSG00000152942 | 38 | 307 | 3.87E−31 | 7.75E−30 | 0.11014493 | YES |
| RAD23B | ENSG00000119318 | 387 | 543 | 0.00021084 | 0.00179885 | 0.41612903 | YES |
| RAD50 | ENSG00000113522 | 496 | 1917 | 4.59E−60 | 2.30E−58 | 0.20555325 | YES |
| RAD51 | ENSG00000051180 | 123 | 830 | 5.72E−56 | 2.52E−54 | 0.12906611 | YES |
| RAD51AP1 | ENSG00000111247 | 567 | 808 | 1.62E−05 | 0.000146 | 0.41236364 | YES |
| RAD51B | ENSG00000182185 | 3574 | 5497 | 4.39E−15 | 5.61E−14 | 0.39400287 | YES |
| RAD51C | ENSG00000108384 | 119 | 1857 | 1.96E−118 | 6.12E−116 | 0.06022267 | YES |
| RAE1 | ENSG00000101146 | 12 | 215 | 1.52E−29 | 2.91E−28 | 0.05286344 | YES |
| RAN | ENSG00000132341 | 14 | 154 | 1.25E−20 | 1.83E−19 | 0.08333333 | YES |
| RANBP2 | ENSG00000153201 | 75 | 251 | 2.60E−15 | 3.34E−14 | 0.23006135 | YES |
| RANBP3 | ENSG00000031823 | 1235 | 2875 | 2.43E−36 | 5.64E−35 | 0.30048662 | YES |
| RANGAP1 | ENSG00000100401 | 79 | 515 | 1.73E−40 | 4.62E−39 | 0.13299663 | YES |
| RAPGEF6 | ENSG00000158987 | 1048 | 1535 | 5.46E−08 | 5.55E−07 | 0.40572977 | YES |
| RARS | ENSG00000113643 | 10 | 140 | 2.63E−20 | 3.82E−19 | 0.06666667 | YES |
| RARS2 | ENSG00000146282 | 514 | 1424 | 6.46E−35 | 1.44E−33 | 0.26522188 | YES |
| RBBP4 | ENSG00000162521 | 222 | 1580 | 2.36E−83 | 2.68E−81 | 0.12319645 | YES |
| RBBP5 | ENSG00000117222 | 23 | 237 | 5.05E−28 | 9.21E−27 | 0.08846154 | YES |
| RBBP6 | ENSG00000122257 | 41 | 456 | 7.37E−46 | 2.27E−44 | 0.08249497 | YES |
| RBBP8 | ENSG00000101773 | 107 | 1313 | 3.31E−92 | 4.78E−90 | 0.07535211 | YES |
| RBM10 | ENSG00000182872 | 394 | 710 | 1.06E−10 | 1.18E−09 | 0.35688406 | YES |
| RBM12 | ENSG00000244462 | 383 | 892 | 1.05E−20 | 1.55E−19 | 0.30039216 | YES |
| RBM14 | ENSG00000239306 | 34 | 206 | 1.77E−20 | 2.58E−19 | 0.14166667 | YES |
| RBM17 | ENSG00000134453 | 68 | 494 | 1.79E−41 | 4.95E−40 | 0.12099644 | YES |
| RBM19 | ENSG00000122965 | 73 | 1003 | 5.03E−81 | 5.05E−79 | 0.06784387 | YES |
| RBM22 | ENSG00000086589 | 15 | 131 | 5.20E−17 | 6.96E−16 | 0.10273973 | YES |
| RBM25 | ENSG00000119707 | 90 | 743 | 3.23E−57 | 1.47E−55 | 0.10804322 | YES |
| RBM28 | ENSG00000106344 | 64 | 528 | 8.95E−46 | 2.74E−44 | 0.10810811 | YES |
| RBM33 | ENSG00000184863 | 210 | 577 | 8.35E−21 | 1.23E−19 | 0.26683609 | YES |
| RBM39 | ENSG00000131051 | 192 | 1574 | 9.79E−89 | 1.29E−86 | 0.10872027 | YES |
| RBM42 | ENSG00000126254 | 24 | 89 | 3.73E−08 | 3.81E−07 | 0.21238938 | YES |
| RBM48 | ENSG00000127993 | 23 | 188 | 7.97E−22 | 1.22E−20 | 0.10900474 | YES |
| RBM8A | ENSG00000131795 | 2 | 141 | 3.95E−24 | 6.43E−23 | 0.01398601 | YES |
| RBMX | ENSG00000147274 | 90 | 773 | 1.35E−59 | 6.68E−58 | 0.10428737 | YES |
| RBMX2 | ENSG00000134597 | 9 | 144 | 2.65E−21 | 3.99E−20 | 0.05882353 | YES |
| RBPJ | ENSG00000168214 | 1426 | 1793 | 0.00035202 | 0.00295149 | 0.44299472 | YES |
| RBX1 | ENSG00000100387 | 171 | 1890 | 4.10E−109 | 1.10E−106 | 0.08296943 | YES |
| RCC1 | ENSG00000180198 | 1999 | 6317 | 1.78E−78 | 1.65E−76 | 0.24037999 | YES |
| RCL1 | ENSG00000120158 | 81 | 539 | 3.64E−42 | 1.01E−40 | 0.13064516 | YES |
| REV3L | ENSG00000009413 | 609 | 1488 | 2.32E−29 | 4.41E−28 | 0.29041488 | YES |
| RFC1 | ENSG00000035928 | 59 | 1087 | 1.74E−90 | 2.38E−88 | 0.05148342 | YES |
| RFC2 | ENSG00000049541 | 99 | 1075 | 3.68E−79 | 3.48E−77 | 0.08432709 | YES |
| RFC3 | ENSG00000133119 | 46 | 563 | 2.29E−54 | 9.51E−53 | 0.07553366 | YES |
| RFC4 | ENSG00000163918 | 67 | 687 | 7.38E−59 | 3.52E−57 | 0.08885942 | YES |
| RFC5 | ENSG00000111445 | 21 | 218 | 2.16E−26 | 3.78E−25 | 0.08786611 | YES |
| RFT1 | ENSG00000163933 | 86 | 904 | 1.48E−70 | 1.00E−68 | 0.08686869 | YES |
| RFWD3 | ENSG00000168411 | 638 | 929 | 2.39E−06 | 2.27E−05 | 0.40714742 | YES |
| RHOA | ENSG00000067560 | 730 | 1655 | 4.77E−27 | 8.46E−26 | 0.30607966 | YES |
| RIF1 | ENSG00000080345 | 258 | 606 | 6.85E−17 | 9.14E−16 | 0.29861111 | YES |
| RINT1 | ENSG00000135249 | 90 | 498 | 3.75E−36 | 8.64E−35 | 0.15306122 | YES |
| RIOK1 | ENSG00000124784 | 56 | 399 | 1.58E−35 | 3.58E−34 | 0.12307692 | YES |
| RIOK2 | ENSG00000058729 | 27 | 248 | 6.14E−28 | 1.12E−26 | 0.09818182 | YES |
| RMI1 | ENSG00000178966 | 494 | 1161 | 3.30E−24 | 5.39E−23 | 0.29848943 | YES |
| RNASEH2A | ENSG00000104889 | 96 | 294 | 1.69E−15 | 2.18E−14 | 0.24615385 | YES |
| RNASEH2B | ENSG00000136104 | 214 | 653 | 1.18E−25 | 2.02E−24 | 0.24682814 | YES |
| RNF168 | ENSG00000163961 | 1564 | 2162 | 4.05E−07 | 3.99E−06 | 0.41975309 | YES |
| RNF20 | ENSG00000155827 | 48 | 157 | 7.63E−11 | 8.47E−10 | 0.23414634 | YES |
| RNF4 | ENSG00000063978 | 654 | 1347 | 3.45E−20 | 4.97E−19 | 0.32683658 | YES |
| RNF40 | ENSG00000103549 | 135 | 237 | 1.47E−05 | 0.00013291 | 0.36290323 | YES |
| RNF8 | ENSG00000112130 | 186 | 414 | 2.71E−12 | 3.14E−11 | 0.31 | YES |
| RNGTT | ENSG00000111880 | 1123 | 4584 | 9.59E−93 | 1.43E−90 | 0.19677589 | YES |
| RNMT | ENSG00000101654 | 68 | 383 | 1.36E−30 | 2.68E−29 | 0.15077605 | YES |
| RNPC3 | ENSG00000185946 | 36 | 309 | 5.18E−32 | 1.06E−30 | 0.10434783 | YES |
| RNPS1 | ENSG00000205937 | 45 | 409 | 7.35E−40 | 1.90E−38 | 0.09911894 | YES |
| RP11-93B14.6 | ENSG00000167046 | 56 | 113 | 8.90E−05 | 0.00077378 | 0.33136095 | YES |
| RPA1 | ENSG00000132383 | 63 | 889 | 1.02E−75 | 8.46E−74 | 0.06617647 | YES |
| RPA2 | ENSG00000117748 | 47 | 207 | 6.49E−17 | 8.67E−16 | 0.18503937 | YES |
| RPAIN | ENSG00000129197 | 62 | 637 | 4.55E−56 | 2.01E−54 | 0.08869814 | YES |
| RPAP1 | ENSG00000103932 | 52 | 722 | 4.15E−66 | 2.41E−64 | 0.06718346 | YES |
| RPAP3 | ENSG00000005175 | 70 | 697 | 7.66E−59 | 3.64E−57 | 0.09126467 | YES |
| RPE | ENSG00000197713 | 41 | 174 | 1.53E−14 | 1.92E−13 | 0.19069767 | YES |
| RPF1 | ENSG00000117133 | 10 | 117 | 5.66E−17 | 7.58E−16 | 0.07874016 | YES |
| RPF2 | ENSG00000197498 | 65 | 593 | 2.26E−51 | 8.50E−50 | 0.09878419 | YES |
| RPIA | ENSG00000153574 | 187 | 333 | 6.43E−07 | 6.25E−06 | 0.35961538 | YES |
| RPL10A | ENSG00000198755 | 28 | 231 | 1.61E−25 | 2.74E−24 | 0.10810811 | YES |
| RPL11 | ENSG00000142676 | 24 | 1024 | 1.06E−96 | 1.86E−94 | 0.02290076 | YES |
| RPL12 | ENSG00000197958 | 56 | 571 | 4.96E−52 | 1.91E−50 | 0.08931419 | YES |
| RPL13 | ENSG00000167526 | 48 | 390 | 5.66E−37 | 1.34E−35 | 0.10958904 | YES |
| RPL13A | ENSG00000142541 | 66 | 2243 | 3.44E−147 | 2.51E−144 | 0.0285838 | YES |
| RPL14 | ENSG00000188846 | 8 | 319 | 1.35E−43 | 3.89E−42 | 0.02446483 | YES |
| RPL15 | ENSG00000174748 | 17 | 375 | 4.90E−46 | 1.52E−44 | 0.04336735 | YES |
| RPL17 | ENSG00000265681 | 61 | 1165 | 6.60E−95 | 1.06E−92 | 0.0497553 | YES |
| RPL18 | ENSG00000063177 | 23 | 817 | 1.18E−82 | 1.28E−80 | 0.02738095 | YES |
| RPL18A | ENSG00000105640 | 45 | 1191 | 5.74E−101 | 1.15E−98 | 0.03640777 | YES |
| RPL19 | ENSG00000108298 | 57 | 1020 | 1.31E−86 | 1.63E−84 | 0.05292479 | YES |
| RPL21 | ENSG00000122026 | 51 | 1390 | 7.29E−111 | 2.09E−108 | 0.03539209 | YES |
| RPL22 | ENSG00000116251 | 658 | 1065 | 1.14E−09 | 1.22E−08 | 0.38189205 | YES |
| RPL22L1 | ENSG00000163584 | 416 | 1118 | 1.95E−29 | 3.72E−28 | 0.27118644 | YES |
| RPL23 | ENSG00000125691 | 18 | 702 | 1.59E−75 | 1.31E−73 | 0.025 | YES |
| RPL23A | ENSG00000198242 | 264 | 1510 | 4.41E−72 | 3.17E−70 | 0.14881623 | YES |
| RPL24 | ENSG00000114391 | 31 | 645 | 2.10E−66 | 1.24E−64 | 0.04585799 | YES |
| RPL26 | ENSG00000161970 | 67 | 1059 | 1.94E−86 | 2.37E−84 | 0.05950266 | YES |
| RPL27 | ENSG00000131469 | 46 | 730 | 1.40E−68 | 8.82E−67 | 0.05927835 | YES |
| RPL27A | ENSG00000166441 | 131 | 840 | 6.53E−55 | 2.77E−53 | 0.13491246 | YES |
| RPL28 | ENSG00000108107 | 18 | 135 | 1.83E−16 | 2.42E−15 | 0.11764706 | YES |
| RPL29 | ENSG00000162244 | 431 | 1071 | 1.72E−25 | 2.91E−24 | 0.28695073 | YES |
| RPL3 | ENSG00000100316 | 47 | 1103 | 6.54E−95 | 1.06E−92 | 0.04086957 | YES |
| RPL31 | ENSG00000071082 | 44 | 853 | 1.19E−78 | 1.10E−76 | 0.0490524 | YES |
| RPL32 | ENSG00000144713 | 18 | 786 | 5.05E−82 | 5.34E−80 | 0.02238806 | YES |
| RPL34 | ENSG00000109475 | 23 | 796 | 4.33E−81 | 4.40E−79 | 0.02808303 | YES |
| RPL35 | ENSG00000136942 | 35 | 485 | 1.16E−50 | 4.20E−49 | 0.06730769 | YES |
| RPL37 | ENSG00000145592 | 21 | 1054 | 1.42E−99 | 2.73E−97 | 0.01953488 | YES |
| RPL37A | ENSG00000197756 | 322 | 935 | 1.03E−29 | 1.99E−28 | 0.25616547 | YES |
| RPL38 | ENSG00000172809 | 44 | 623 | 2.66E−60 | 1.35E−58 | 0.06596702 | YES |
| RPL39 | ENSG00000198918 | 50 | 1194 | 9.67E−100 | 1.88E−97 | 0.04019293 | YES |
| RPL4 | ENSG00000174444 | 1495 | 2368 | 1.26E−12 | 1.48E−11 | 0.38700492 | YES |
| RPL41 | ENSG00000229117 | 607 | 864 | 1.19E−05 | 0.00010846 | 0.41264446 | YES |
| RPL5 | ENSG00000122406 | 16 | 667 | 2.08E−73 | 1.58E−71 | 0.02342606 | YES |
| RPL6 | ENSG00000089009 | 33 | 975 | 1.19E−90 | 1.64E−88 | 0.0327381 | YES |
| RPL7A | ENSG00000148303 | 47 | 619 | 5.14E−59 | 2.47E−57 | 0.07057057 | YES |
| RPL7L1 | ENSG00000146223 | 201 | 922 | 7.86E−47 | 2.50E−45 | 0.17898486 | YES |
| RPL9 | ENSG00000163682 | 53 | 1071 | 3.95E−91 | 5.56E−89 | 0.04715302 | YES |
| RPLP0 | ENSG00000089157 | 34 | 694 | 1.78E−69 | 1.17E−67 | 0.0467033 | YES |
| RPLP1 | ENSG00000137818 | 270 | 505 | 9.60E−10 | 1.03E−08 | 0.3483871 | YES |
| RPLP2 | ENSG00000177600 | 99 | 510 | 4.11E−35 | 9.19E−34 | 0.16256158 | YES |
| RPN1 | ENSG00000163902 | 50 | 652 | 6.39E−61 | 3.26E−59 | 0.07122507 | YES |
| RPN2 | ENSG00000118705 | 139 | 1464 | 1.24E−93 | 1.92E−91 | 0.08671241 | YES |
| RPP14 | ENSG00000163684 | 34 | 509 | 2.79E−53 | 1.12E−51 | 0.0626151 | YES |
| RPP30 | ENSG00000148688 | 46 | 214 | 5.34E−18 | 7.33E−17 | 0.17692308 | YES |
| RPP38 | ENSG00000152464 | 114 | 460 | 2.46E−27 | 4.40E−26 | 0.19860627 | YES |
| RPP40 | ENSG00000124787 | 2 | 55 | 8.25E−11 | 9.15E−10 | 0.03508772 | YES |
| RPRD1B | ENSG00000101413 | 325 | 1014 | 4.82E−34 | 1.04E−32 | 0.24271845 | YES |
| RPRD2 | ENSG00000163125 | 2359 | 2909 | 0.00027488 | 0.00232672 | 0.44779803 | YES |
| RPS10 | ENSG00000124614 | 136 | 1011 | 4.45E−66 | 2.58E−64 | 0.11857018 | YES |
| RPS11 | ENSG00000142534 | 62 | 804 | 1.23E−69 | 8.16E−68 | 0.07159353 | YES |
| RPS12 | ENSG00000112306 | 17 | 314 | 2.23E−39 | 5.66E−38 | 0.05135952 | YES |
| RPS13 | ENSG00000110700 | 17 | 591 | 1.47E−56 | 6.53E−55 | 0.07366771 | YES |
| RPS14 | ENSG00000164587 | 73 | 1384 | 9.43E−105 | 2.08E−102 | 0.05010295 | YES |
| RPS15 | ENSG00000115268 | 25 | 214 | 1.91E−24 | 3.16E−23 | 0.10460251 | YES |
| RPS15A | ENSG00000134419 | 42 | 1076 | 1.26E−94 | 1.99E−92 | 0.03756708 | YES |
| RPS16 | ENSG00000105193 | 46 | 367 | 3.56E−35 | 7.99E−34 | 0.11138015 | YES |
| RPS18 | ENSG00000231500 | 43 | 686 | 6.16E−66 | 3.54E−64 | 0.05898491 | YES |
| RPS19 | ENSG00000105372 | 71 | 490 | 2.82E−40 | 7.47E−39 | 0.12655971 | YES |
| RPS19BP1 | ENSG00000187051 | 45 | 522 | 6.52E−51 | 2.39E−49 | 0.07936508 | YES |
| RPS2 | ENSG00000140988 | 17 | 239 | 1.55E−30 | 3.04E−29 | 0.06640625 | YES |
| RPS21 | ENSG00000171858 | 29 | 121 | 4.54E−11 | 5.09E−10 | 0.19333333 | YES |
| RPS23 | ENSG00000186468 | 64 | 871 | 4.09E−74 | 3.25E−72 | 0.0684492 | YES |
| RPS24 | ENSG00000138326 | 32 | 863 | 4.13E−83 | 4.63E−81 | 0.03575419 | YES |
| RPS25 | ENSG00000118181 | 479 | 865 | 6.65E−12 | 7.62E−11 | 0.35639881 | YES |
| RPS26 | ENSG00000197728 | 52 | 656 | 1.19E−60 | 6.06E−59 | 0.07344633 | YES |
| RPS27 | ENSG00000177954 | 85 | 499 | 1.78E−37 | 4.26E−36 | 0.14554795 | YES |
| RPS27A | ENSG00000143947 | 42 | 738 | 1.84E−70 | 1.24E−68 | 0.05384615 | YES |
| RPS29 | ENSG00000213741 | 105 | 481 | 3.92E−31 | 7.83E−30 | 0.17918089 | YES |
| RPS3 | ENSG00000149273 | 38 | 1113 | 3.68E−98 | 6.70E−96 | 0.03301477 | YES |
| RPS3A | ENSG00000145425 | 30 | 753 | 1.73E−75 | 1.42E−73 | 0.03831418 | YES |
| RPS4X | ENSG00000198034 | 33 | 1046 | 2.31E−95 | 3.80E−93 | 0.03058387 | YES |
| RPS6 | ENSG00000137154 | 26 | 706 | 4.22E−73 | 3.15E−71 | 0.03551913 | YES |
| RPS6KB1 | ENSG00000108443 | 351 | 556 | 1.22E−06 | 1.17E−05 | 0.38699008 | YES |
| RPS7 | ENSG00000171863 | 22 | 384 | 4.28E−45 | 1.29E−43 | 0.05418719 | YES |
| RPS8 | ENSG00000142937 | 35 | 658 | 3.57E−66 | 2.10E−64 | 0.05050505 | YES |
| RPS9 | ENSG00000170889 | 17 | 270 | 2.76E−34 | 6.02E−33 | 0.05923345 | YES |
| RPSA | ENSG00000168028 | 51 | 843 | 8.37E−76 | 7.04E−74 | 0.05704698 | YES |
| RPTOR | ENSG00000141564 | 662 | 3929 | 2.85E−116 | 8.59E−114 | 0.14419516 | YES |
| RPUSD4 | ENSG00000165526 | 79 | 241 | 1.64E−13 | 1.98E−12 | 0.246875 | YES |
| RREB1 | ENSG00000124782 | 1680 | 3044 | 4.64E−21 | 6.91E−20 | 0.35563082 | YES |
| RRM1 | ENSG00000167325 | 152 | 1161 | 1.48E−72 | 1.09E−70 | 0.11576542 | YES |
| RRM2 | ENSG00000171848 | 14 | 189 | 2.34E−25 | 3.95E−24 | 0.06896552 | YES |
| RRN3 | ENSG00000085721 | 93 | 348 | 2.98E−21 | 4.48E−20 | 0.21088435 | YES |
| RRP1 | ENSG00000160214 | 43 | 351 | 2.14E−34 | 4.71E−33 | 0.10913706 | YES |
| RRP12 | ENSG00000052749 | 44 | 256 | 1.83E−23 | 2.92E−22 | 0.14666667 | YES |
| RRP15 | ENSG00000067533 | 91 | 406 | 2.87E−27 | 5.11E−26 | 0.18309859 | YES |
| RRP36 | ENSG00000124541 | 63 | 339 | 2.31E−27 | 4.15E−26 | 0.15671642 | YES |
| RRP7A | ENSG00000189306 | 27 | 107 | 9.17E−10 | 9.85E−09 | 0.20149254 | YES |
| RRP9 | ENSG00000114767 | 62 | 238 | 9.38E−17 | 1.25E−15 | 0.20666667 | YES |
| RSL1D1 | ENSG00000171490 | 100 | 956 | 1.01E−70 | 6.95E−69 | 0.09469697 | YES |
| RSL24D1 | ENSG00000137876 | 20 | 437 | 2.94E−51 | 1.10E−49 | 0.04376368 | YES |
| RSRC2 | ENSG00000111011 | 197 | 1785 | 8.13E−99 | 1.51E−96 | 0.09939455 | YES |
| RTEL1 | ENSG00000258366 | 50 | 223 | 5.37E−18 | 7.36E−17 | 0.18315018 | YES |
| RTF1 | ENSG00000137815 | 81 | 1058 | 1.27E−82 | 1.37E−80 | 0.07111501 | YES |
| RTFDC1 | ENSG00000022277 | 28 | 203 | 4.96E−22 | 7.67E−21 | 0.12121212 | YES |
| RTTN | ENSG00000176225 | 53 | 503 | 1.18E−46 | 3.71E−45 | 0.09532374 | YES |
| RUVBL1 | ENSG00000175792 | 85 | 1307 | 3.02E−97 | 5.32E−95 | 0.06106322 | YES |
| RUVBL2 | ENSG00000183207 | 63 | 451 | 9.89E−39 | 2.46E−37 | 0.12256809 | YES |
| SACM1L | ENSG00000211456 | 204 | 2247 | 4.57E−119 | 1.48E−116 | 0.08323133 | YES |
| SAE1 | ENSG00000142230 | 550 | 7357 | 4.32E−208 | 1.26E−204 | 0.06955862 | YES |
| SAMD4B | ENSG00000179134 | 60 | 170 | 7.91E−10 | 8.52E−09 | 0.26086957 | YES |
| SAMM50 | ENSG00000100347 | 31 | 456 | 3.36E−49 | 1.16E−47 | 0.06365503 | YES |
| SAP130 | ENSG00000136715 | 32 | 301 | 1.94E−32 | 4.00E−31 | 0.0960961 | YES |
| SAP18 | ENSG00000150459 | 12 | 198 | 2.35E−27 | 4.20E−26 | 0.05714286 | YES |
| SAP30BP | ENSG00000161526 | 26 | 509 | 4.86E−56 | 2.15E−54 | 0.04859813 | YES |
| SARNP | ENSG00000205323 | 504 | 1219 | 4.33E−26 | 7.54E−25 | 0.29251306 | YES |
| SARS | ENSG00000031698 | 161 | 2958 | 1.80E−154 | 1.97E−151 | 0.05161911 | YES |
| SARS2 | ENSG00000104835 | 213 | 574 | 3.43E−20 | 4.95E−19 | 0.27064803 | YES |
| SART1 | ENSG00000175467 | 10 | 125 | 3.80E−18 | 5.24E−17 | 0.07407407 | YES |
| SART3 | ENSG00000075856 | 41 | 516 | 1.25E−51 | 4.75E−50 | 0.07360862 | YES |
| SASS6 | ENSG00000156876 | 90 | 1019 | 1.20E−77 | 1.07E−75 | 0.08115419 | YES |
| SBDS | ENSG00000126524 | 72 | 169 | 1.13E−07 | 1.14E−06 | 0.29875519 | YES |
| SCAF11 | ENSG00000139218 | 584 | 873 | 8.57E−07 | 8.28E−06 | 0.40082361 | YES |
| SCAMP4 | ENSG00000227500 | 280 | 610 | 7.68E−15 | 9.72E−14 | 0.31460674 | YES |
| SCD | ENSG00000099194 | 401 | 808 | 7.84E−15 | 9.92E−14 | 0.33167907 | YES |
| SCFD1 | ENSG00000092108 | 114 | 1410 | 3.22E−96 | 5.47E−94 | 0.07480315 | YES |
| SCO1 | ENSG00000133028 | 458 | 613 | 0.00073267 | 0.00599907 | 0.42763772 | YES |
| SDAD1 | ENSG00000198301 | 51 | 385 | 1.50E−35 | 3.40E−34 | 0.11697248 | YES |
| SDE2 | ENSG00000143751 | 30 | 397 | 1.41E−43 | 4.05E−42 | 0.07025761 | YES |
| SDHA | ENSG00000073578 | 201 | 472 | 1.46E−14 | 1.83E−13 | 0.2986627 | YES |
| SDHB | ENSG00000117118 | 296 | 1008 | 1.63E−37 | 3.91E−36 | 0.22699387 | YES |
| SDHC | ENSG00000143252 | 712 | 1686 | 1.49E−29 | 2.87E−28 | 0.2969141 | YES |
| SDHD | ENSG00000204370 | 163 | 388 | 3.26E−13 | 3.91E−12 | 0.29582577 | YES |
| SEC13 | ENSG00000157020 | 18 | 263 | 4.62E−33 | 9.68E−32 | 0.06405694 | YES |
| SEC16A | ENSG00000148396 | 19 | 69 | 9.80E−07 | 9.44E−06 | 0.21590909 | YES |
| SEC61A1 | ENSG00000058262 | 39 | 255 | 7.65E−25 | 1.28E−23 | 0.13265306 | YES |
| SEC61B | ENSG00000106803 | 32 | 111 | 5.32E−09 | 5.57E−08 | 0.22377622 | YES |
| SEC62 | ENSG00000008952 | 266 | 585 | 9.59E−15 | 1.21E−13 | 0.31257344 | YES |
| SEC63 | ENSG00000025796 | 569 | 1135 | 2.69E−17 | 3.61E−16 | 0.33392019 | YES |
| SEH1L | ENSG00000085415 | 210 | 1403 | 1.15E−75 | 9.56E−74 | 0.13019219 | YES |
| SEL1L | ENSG00000071537 | 407 | 744 | 2.33E−11 | 2.64E−10 | 0.35360556 | YES |
| SENP6 | ENSG00000112701 | 243 | 1705 | 3.44E−86 | 4.17E−84 | 0.12474333 | YES |
| SEPSECS | ENSG00000109618 | 295 | 547 | 5.60E−10 | 6.06E−09 | 0.35035629 | YES |
| SERBP1 | ENSG00000142864 | 358 | 749 | 2.17E−15 | 2.79E−14 | 0.32339657 | YES |
| SF1 | ENSG00000168066 | 13 | 141 | 3.28E−19 | 4.62E−18 | 0.08441558 | YES |
| SF3A1 | ENSG00000099995 | 15 | 315 | 2.81E−40 | 7.44E−39 | 0.04545455 | YES |
| SF3A2 | ENSG00000104897 | 83 | 891 | 2.15E−70 | 1.45E−68 | 0.08521561 | YES |
| SF3B1 | ENSG00000115524 | 22 | 572 | 3.38E−63 | 1.81E−61 | 0.03703704 | YES |
| SF3B14 | ENSG00000115128 | 26 | 331 | 6.55E−38 | 1.59E−36 | 0.07282913 | YES |
| SF3B2 | ENSG00000087365 | 24 | 393 | 2.66E−45 | 8.00E−44 | 0.05755396 | YES |
| SF3B3 | ENSG00000189091 | 205 | 1770 | 1.59E−96 | 2.72E−94 | 0.10379747 | YES |
| SFPQ | ENSG00000116560 | 32 | 83 | 1.46E−05 | 0.00013243 | 0.27826087 | YES |
| SFSWAP | ENSG00000061936 | 465 | 1570 | 3.53E−47 | 1.14E−45 | 0.22850123 | YES |
| SGOL1 | ENSG00000129810 | 22 | 186 | 6.41E−22 | 9.87E−21 | 0.10576923 | YES |
| SHFM1 | ENSG00000127922 | 11 | 298 | 5.69E−40 | 1.49E−38 | 0.03559871 | YES |
| SHMT2 | ENSG00000182199 | 406 | 602 | 1.37E−05 | 0.00012463 | 0.40277778 | YES |
| SHOC2 | ENSG00000108061 | 724 | 1017 | 9.75E−06 | 8.93E−05 | 0.41585296 | YES |
| SHQ1 | ENSG00000144736 | 192 | 750 | 3.58E−36 | 8.26E−35 | 0.20382166 | YES |
| SKA1 | ENSG00000154839 | 145 | 369 | 3.61E−14 | 4.46E−13 | 0.28210117 | YES |
| SKA2 | ENSG00000182628 | 746 | 4804 | 2.38E−132 | 1.07E−129 | 0.13441441 | YES |
| SKA3 | ENSG00000165480 | 49 | 486 | 2.98E−46 | 9.26E−45 | 0.09158879 | YES |
| SKIV2L2 | ENSG00000039123 | 148 | 1488 | 5.06E−93 | 7.62E−91 | 0.09046455 | YES |
| SKP1 | ENSG00000113558 | 86 | 381 | 5.50E−26 | 9.53E−25 | 0.18415418 | YES |
| SLBP | ENSG00000163950 | 391 | 783 | 2.15E−14 | 2.68E−13 | 0.3330494 | YES |
| SLC25A19 | ENSG00000125454 | 608 | 816 | 0.00023279 | 0.00198328 | 0.42696629 | YES |
| SLC25A26 | ENSG00000144741 | 290 | 561 | 3.91E−11 | 4.39E−10 | 0.34077556 | YES |
| SLC25A3 | ENSG00000075415 | 128 | 316 | 2.72E−12 | 3.15E−11 | 0.28828829 | YES |
| SLC35B1 | ENSG00000121073 | 25 | 246 | 2.11E−28 | 3.91E−27 | 0.09225092 | YES |
| SLC39A10 | ENSG00000196950 | 251 | 2229 | 7.56E−110 | 2.13E−107 | 0.10120968 | YES |
| SLC7A6OS | ENSG00000103061 | 21 | 324 | 5.72E−39 | 1.44E−37 | 0.06086957 | YES |
| SLIRP | ENSG00000119705 | 231 | 477 | 1.24E−11 | 1.41E−10 | 0.32627119 | YES |
| SLMAP | ENSG00000163681 | 1562 | 2134 | 1.03E−06 | 9.90E−06 | 0.42261905 | YES |
| SLMO2 | ENSG00000101166 | 61 | 620 | 6.85E−55 | 2.90E−53 | 0.08957416 | YES |
| SLU7 | ENSG00000164609 | 73 | 163 | 5.78E−07 | 5.63E−06 | 0.30932203 | YES |
| SLX4 | ENSG00000188827 | 263 | 455 | 1.23E−07 | 1.24E−06 | 0.36629526 | YES |
| SMARCA5 | ENSG00000153147 | 124 | 302 | 1.04E−11 | 1.19E−10 | 0.29107981 | YES |
| SMARCB1 | ENSG00000099956 | 87 | 462 | 1.58E−33 | 3.36E−32 | 0.15846995 | YES |
| SMARCE1 | ENSG00000073584 | 325 | 809 | 6.47E−22 | 9.95E−21 | 0.28659612 | YES |
| SMC1A | ENSG00000072501 | 48 | 800 | 1.88E−73 | 1.43E−71 | 0.05660377 | YES |
| SMC2 | ENSG00000136824 | 107 | 480 | 1.32E−30 | 2.60E−29 | 0.18228279 | YES |
| SMC3 | ENSG00000108055 | 17 | 283 | 8.11E−36 | 1.86E−34 | 0.05666667 | YES |
| SMC4 | ENSG00000113810 | 36 | 551 | 1.68E−56 | 7.45E−55 | 0.06132879 | YES |
| SMC5 | ENSG00000198887 | 114 | 604 | 6.43E−40 | 1.67E−38 | 0.15877437 | YES |
| SMC6 | ENSG00000163029 | 422 | 2587 | 1.38E−98 | 2.54E−96 | 0.14024593 | YES |
| SMG1 | ENSG00000157106 | 204 | 1037 | 4.51E−54 | 1.86E−52 | 0.16438356 | YES |
| SMG5 | ENSG00000198952 | 195 | 658 | 6.67E−29 | 1.26E−27 | 0.22860492 | YES |
| SMG6 | ENSG00000070366 | 482 | 2714 | 1.19E−95 | 1.97E−93 | 0.15081352 | YES |
| SMG7 | ENSG00000116698 | 563 | 1055 | 2.03E−14 | 2.54E−13 | 0.34796044 | YES |
| SMNDC1 | ENSG00000119953 | 69 | 341 | 4.97E−26 | 8.64E−25 | 0.16829268 | YES |
| SMU1 | ENSG00000122692 | 9 | 278 | 1.70E−38 | 4.20E−37 | 0.03135889 | YES |
| SNAPC1 | ENSG00000023608 | 88 | 888 | 7.32E−69 | 4.74E−67 | 0.09016393 | YES |
| SNAPC3 | ENSG00000164975 | 28 | 325 | 1.57E−36 | 3.67E−35 | 0.07932011 | YES |
| SNF8 | ENSG00000159210 | 171 | 400 | 4.33E−13 | 5.16E−12 | 0.29947461 | YES |
| SNIP1 | ENSG00000163877 | 6 | 79 | 7.19E−13 | 8.49E−12 | 0.07058824 | YES |
| SNRNP200 | ENSG00000144028 | 31 | 403 | 7.47E−44 | 2.17E−42 | 0.07142857 | YES |
| SNRNP27 | ENSG00000124380 | 290 | 522 | 4.57E−09 | 4.80E−08 | 0.35714286 | YES |
| SNRNP35 | ENSG00000184209 | 648 | 1846 | 2.59E−41 | 7.13E−40 | 0.25982358 | YES |
| SNRNP40 | ENSG00000060688 | 255 | 905 | 7.59E−37 | 1.79E−35 | 0.21982759 | YES |
| SNRNP48 | ENSG00000168566 | 15 | 127 | 1.94E−16 | 2.55E−15 | 0.1056338 | YES |
| SNRNP70 | ENSG00000104852 | 160 | 475 | 2.17E−20 | 3.16E−19 | 0.2519685 | YES |
| SNRPA | ENSG00000077312 | 537 | 1113 | 1.32E−18 | 1.83E−17 | 0.32545455 | YES |
| SNRPA1 | ENSG00000131876 | 32 | 369 | 6.16E−40 | 1.60E−38 | 0.0798005 | YES |
| SNRPB | ENSG00000125835 | 70 | 1037 | 3.77E−84 | 4.39E−82 | 0.06323397 | YES |
| SNRPC | ENSG00000124562 | 32 | 452 | 1.84E−48 | 6.28E−47 | 0.0661157 | YES |
| SNRPD1 | ENSG00000167088 | 57 | 1003 | 1.85E−85 | 2.22E−83 | 0.05377358 | YES |
| SNRPD2 | ENSG00000125743 | 33 | 377 | 1.91E−40 | 5.09E−39 | 0.0804878 | YES |
| SNRPD3 | ENSG00000100028 | 153 | 650 | 1.28E−35 | 2.91E−34 | 0.19053549 | YES |
| SNRPE | ENSG00000182004 | 41 | 487 | 7.09E−49 | 2.43E−47 | 0.07765152 | YES |
| SNRPF | ENSG00000139343 | 23 | 411 | 1.59E−47 | 5.22E−46 | 0.05299539 | YES |
| SNRPG | ENSG00000143977 | 60 | 894 | 6.16E−77 | 5.30E−75 | 0.06289308 | YES |
| SNW1 | ENSG00000100603 | 20 | 225 | 1.21E−27 | 2.18E−26 | 0.08163265 | YES |
| SOD1 | ENSG00000142168 | 29 | 492 | 2.21E−53 | 8.97E−52 | 0.05566219 | YES |
| SOD2 | ENSG00000112096 | 45 | 117 | 6.10E−07 | 5.93E−06 | 0.27777778 | YES |
| SON | ENSG00000159140 | 37 | 883 | 5.47E−83 | 6.01E−81 | 0.04021739 | YES |
| SP1 | ENSG00000185591 | 1180 | 1879 | 9.81E−12 | 1.12E−10 | 0.38574698 | YES |
| SPAG5 | ENSG00000076382 | 99 | 417 | 1.52E−26 | 2.67E−25 | 0.19186047 | YES |
| SPATA5 | ENSG00000145375 | 313 | 2645 | 5.84E−117 | 1.79E−114 | 0.10581474 | YES |
| SPATA5L1 | ENSG00000171763 | 16 | 180 | 2.27E−23 | 3.63E−22 | 0.08163265 | YES |
| SPC24 | ENSG00000161888 | 30 | 258 | 4.18E−28 | 7.66E−27 | 0.10416667 | YES |
| SPC25 | ENSG00000152253 | 25 | 223 | 1.43E−25 | 2.44E−24 | 0.10080645 | YES |
| SPCS2 | ENSG00000118363 | 68 | 926 | 5.40E−77 | 4.67E−75 | 0.06841046 | YES |
| SPCS3 | ENSG00000129128 | 144 | 350 | 8.37E−13 | 9.88E−12 | 0.29149798 | YES |
| SPDL1 | ENSG00000040275 | 16 | 207 | 6.69E−27 | 1.18E−25 | 0.07174888 | YES |
| SPRTN | ENSG00000010072 | 132 | 580 | 4.72E−34 | 1.02E−32 | 0.18539326 | YES |
| SRBD1 | ENSG00000068784 | 288 | 4994 | 1.36E−191 | 2.97E−188 | 0.0545248 | YES |
| SRCAP | ENSG00000080603 | 272 | 695 | 6.33E−21 | 9.40E−20 | 0.28128232 | YES |
| SRD5A3 | ENSG00000128039 | 684 | 1475 | 3.26E−23 | 5.17E−22 | 0.31681334 | YES |
| SREK1 | ENSG00000153914 | 749 | 1190 | 1.39E−09 | 1.48E−08 | 0.38628159 | YES |
| SRF | ENSG00000112658 | 134 | 517 | 2.37E−28 | 4.38E−27 | 0.20583717 | YES |
| SRFBP1 | ENSG00000151304 | 22 | 338 | 3.79E−40 | 9.98E−39 | 0.06111111 | YES |
| SRP14 | ENSG00000140319 | 41 | 293 | 1.31E−28 | 2.44E−27 | 0.12275449 | YES |
| SRP19 | ENSG00000153037 | 16 | 202 | 2.95E−26 | 5.14E−25 | 0.0733945 | YES |
| SRP68 | ENSG00000167881 | 43 | 507 | 3.87E−50 | 1.38E−48 | 0.07818182 | YES |
| SRP72 | ENSG00000174780 | 335 | 1038 | 3.35E−34 | 7.28E−33 | 0.24399126 | YES |
| SRP9 | ENSG00000143742 | 34 | 495 | 5.91E−52 | 2.27E−50 | 0.06427221 | YES |
| SRPR | ENSG00000182934 | 13 | 256 | 3.27E−34 | 7.11E−33 | 0.04832714 | YES |
| SRPRB | ENSG00000144867 | 120 | 298 | 6.01E−12 | 6.90E−11 | 0.28708134 | YES |
| SRRD | ENSG00000100104 | 309 | 515 | 2.18E−07 | 2.18E−06 | 0.375 | YES |
| SRRM1 | ENSG00000133226 | 62 | 509 | 1.42E−44 | 4.20E−43 | 0.10858144 | YES |
| SRRM2 | ENSG00000167978 | 169 | 740 | 1.48E−39 | 3.79E−38 | 0.18591859 | YES |
| SRRT | ENSG00000087087 | 65 | 328 | 1.10E−25 | 1.88E−24 | 0.1653944 | YES |
| SRSF1 | ENSG00000136450 | 105 | 637 | 8.71E−45 | 2.60E−43 | 0.14150943 | YES |
| SRSF10 | ENSG00000188529 | 462 | 1110 | 1.45E−24 | 2.40E−23 | 0.29389313 | YES |
| SRSF11 | ENSG00000116754 | 1799 | 2296 | 6.97E−05 | 0.00060902 | 0.43931624 | YES |
| SRSF2 | ENSG00000161547 | 6 | 61 | 6.13E−10 | 6.63E−09 | 0.08955224 | YES |
| SRSF3 | ENSG00000112081 | 26 | 711 | 1.68E−73 | 1.29E−71 | 0.03527815 | YES |
| SRSF7 | ENSG00000115875 | 15 | 349 | 4.93E−44 | 1.44E−42 | 0.04120879 | YES |
| SSB | ENSG00000138385 | 33 | 381 | 7.21E−41 | 1.95E−39 | 0.07971014 | YES |
| SSBP1 | ENSG00000106028 | 125 | 464 | 1.62E−25 | 2.76E−24 | 0.21222411 | YES |
| SSRP1 | ENSG00000149136 | 269 | 531 | 2.80E−11 | 3.16E−10 | 0.33625 | YES |
| SSU72 | ENSG00000160075 | 105 | 810 | 1.05E−58 | 4.97E−57 | 0.1147541 | YES |
| STIL | ENSG00000123473 | 75 | 841 | 8.08E−69 | 5.21E−67 | 0.08187773 | YES |
| STRAP | ENSG00000023734 | 141 | 512 | 1.27E−26 | 2.23E−25 | 0.21592649 | YES |
| STRIP1 | ENSG00000143093 | 59 | 165 | 1.73E−09 | 1.83E−08 | 0.26339286 | YES |
| STX18 | ENSG00000168818 | 1816 | 23790 | 7.71E−288 | 1.35E−283 | 0.07092088 | YES |
| STX5 | ENSG00000162236 | 37 | 202 | 4.42E−19 | 6.22E−18 | 0.15481172 | YES |
| SUDS3 | ENSG00000111707 | 20 | 109 | 4.11E−12 | 4.75E−11 | 0.15503876 | YES |
| SUGP1 | ENSG00000105705 | 68 | 415 | 8.28E−34 | 1.77E−32 | 0.14078675 | YES |
| SUGT1 | ENSG00000165416 | 97 | 650 | 8.13E−48 | 2.69E−46 | 0.12985274 | YES |
| SUMO2 | ENSG00000188612 | 206 | 456 | 6.74E−13 | 7.98E−12 | 0.31117825 | YES |
| SUPT16H | ENSG00000092201 | 143 | 1207 | 1.94E−77 | 1.72E−75 | 0.10592593 | YES |
| SUPT4H1 | ENSG00000213246 | 12 | 94 | 9.99E−13 | 1.18E−11 | 0.11320755 | YES |
| SUPT5H | ENSG00000196235 | 86 | 529 | 5.59E−40 | 1.46E−38 | 0.1398374 | YES |
| SUPT6H | ENSG00000109111 | 30 | 509 | 1.21E−54 | 5.07E−53 | 0.05565863 | YES |
| SUPV3L1 | ENSG00000156502 | 79 | 335 | 5.10E−23 | 8.07E−22 | 0.19082126 | YES |
| SURF6 | ENSG00000148296 | 31 | 393 | 8.17E−43 | 2.31E−41 | 0.07311321 | YES |
| SUZ12 | ENSG00000178691 | 327 | 617 | 4.32E−11 | 4.85E−10 | 0.34639831 | YES |
| SYF2 | ENSG00000117614 | 3 | 168 | 2.21E−27 | 3.97E−26 | 0.01754386 | YES |
| SYMPK | ENSG00000125755 | 62 | 518 | 2.04E−45 | 6.17E−44 | 0.10689655 | YES |
| SYS1 | ENSG00000204070 | 42 | 265 | 4.25E−25 | 7.12E−24 | 0.13680782 | YES |
| TACC3 | ENSG00000013810 | 175 | 302 | 4.29E−06 | 4.01E−05 | 0.36687631 | YES |
| TADA1 | ENSG00000152382 | 87 | 167 | 1.76E−05 | 0.00015907 | 0.34251969 | YES |
| TADA2A | ENSG00000108264 | 903 | 1853 | 2.88E−23 | 4.60E−22 | 0.32764877 | YES |
| TAF1 | ENSG00000147133 | 173 | 940 | 2.36E−53 | 9.56E−52 | 0.15543576 | YES |
| TAF12 | ENSG00000120656 | 214 | 1038 | 2.48E−52 | 9.66E−51 | 0.17092652 | YES |
| TAF13 | ENSG00000197780 | 235 | 386 | 4.01E−06 | 3.76E−05 | 0.3784219 | YES |
| TAF1B | ENSG00000115750 | 612 | 4761 | 1.74E−145 | 1.17E−142 | 0.11390285 | YES |
| TAF1C | ENSG00000103168 | 19 | 157 | 3.87E−19 | 5.46E−18 | 0.10795455 | YES |
| TAF1D | ENSG00000166012 | 246 | 1321 | 2.31E−64 | 1.28E−62 | 0.15698787 | YES |
| TAF5 | ENSG00000148835 | 27 | 356 | 2.73E−40 | 7.26E−39 | 0.07049608 | YES |
| TAF6 | ENSG00000106290 | 227 | 582 | 6.02E−19 | 8.44E−18 | 0.28059333 | YES |
| TAF6L | ENSG00000162227 | 332 | 529 | 1.36E−06 | 1.30E−05 | 0.38559814 | YES |
| TAF8 | ENSG00000137413 | 27 | 82 | 1.70E−06 | 1.62E−05 | 0.24770642 | YES |
| TAF9 | ENSG00000085231 | 235 | 771 | 7.90E−31 | 1.57E−29 | 0.23359841 | YES |
| TAMM41 | ENSG00000144559 | 36 | 276 | 3.07E−28 | 5.66E−27 | 0.11538462 | YES |
| TANGO6 | ENSG00000103047 | 351 | 4343 | 1.32E−166 | 1.92E−163 | 0.07477631 | YES |
| TARDBP | ENSG00000120948 | 30 | 128 | 1.04E−11 | 1.18E−10 | 0.18987342 | YES |
| TARS | ENSG00000113407 | 113 | 1643 | 3.96E−109 | 1.08E−106 | 0.0643508 | YES |
| TARS2 | ENSG00000143374 | 170 | 436 | 6.68E−16 | 8.66E−15 | 0.28052805 | YES |
| TBCA | ENSG00000171530 | 371 | 863 | 2.73E−20 | 3.96E−19 | 0.3006483 | YES |
| TBCB | ENSG00000105254 | 20 | 128 | 8.76E−15 | 1.11E−13 | 0.13513514 | YES |
| TBCD | ENSG00000141556 | 845 | 3491 | 4.46E−84 | 5.16E−82 | 0.19488007 | YES |
| TBCE | ENSG00000116957 | 152 | 440 | 7.85E−19 | 1.10E−17 | 0.25675676 | YES |
| TCEB2 | ENSG00000103363 | 43 | 274 | 7.80E−26 | 1.35E−24 | 0.13564669 | YES |
| TCERG1 | ENSG00000113649 | 326 | 898 | 4.89E−27 | 8.66E−26 | 0.26633987 | YES |
| TCOF1 | ENSG00000070814 | 171 | 371 | 5.29E−11 | 5.90E−10 | 0.31549815 | YES |
| TCP1 | ENSG00000120438 | 28 | 249 | 1.04E−27 | 1.88E−26 | 0.10108303 | YES |
| TECR | ENSG00000099797 | 566 | 739 | 0.00098252 | 0.00793326 | 0.43371648 | YES |
| TEFM | ENSG00000172171 | 449 | 760 | 2.18E−09 | 2.31E−08 | 0.37138131 | YES |
| TEX10 | ENSG00000136891 | 27 | 293 | 2.97E−33 | 6.26E−32 | 0.084375 | YES |
| TFAM | ENSG00000108064 | 73 | 165 | 3.78E−07 | 3.73E−06 | 0.30672269 | YES |
| TFB1M | ENSG00000029639 | 388 | 662 | 5.95E−09 | 6.23E−08 | 0.36952381 | YES |
| TFB2M | ENSG00000162851 | 131 | 316 | 7.14E−12 | 8.17E−11 | 0.29306488 | YES |
| TFIP11 | ENSG00000100109 | 52 | 159 | 3.22E−10 | 3.52E−09 | 0.2464455 | YES |
| TFRC | ENSG00000072274 | 214 | 784 | 5.82E−35 | 1.30E−33 | 0.21442886 | YES |
| THOC1 | ENSG00000079134 | 43 | 509 | 2.51E−50 | 8.96E−49 | 0.07789855 | YES |
| THOC5 | ENSG00000100296 | 66 | 414 | 3.02E−34 | 6.58E−33 | 0.1375 | YES |
| THOC7 | ENSG00000163634 | 29 | 484 | 1.29E−52 | 5.08E−51 | 0.05653021 | YES |
| THRAP3 | ENSG00000054118 | 716 | 981 | 4.17E−05 | 0.00036962 | 0.42192104 | YES |
| TICRR | ENSG00000140534 | 81 | 976 | 5.16E−77 | 4.49E−75 | 0.07663198 | YES |
| TIMELESS | ENSG00000111602 | 91 | 1064 | 1.94E−80 | 1.90E−78 | 0.07878788 | YES |
| TIMM10 | ENSG00000134809 | 64 | 238 | 2.67E−16 | 3.50E−15 | 0.21192053 | YES |
| TIMM22 | ENSG00000177370 | 31 | 366 | 5.83E−40 | 1.52E−38 | 0.07808564 | YES |
| TIMM23 | ENSG00000138297 | 31 | 81 | 1.60E−05 | 0.00014461 | 0.27678571 | YES |
| TIMM44 | ENSG00000104980 | 73 | 1038 | 2.09E−83 | 2.39E−81 | 0.06570657 | YES |
| TIMM50 | ENSG00000105197 | 159 | 374 | 1.13E−12 | 1.33E−11 | 0.29831144 | YES |
| TIMM9 | ENSG00000100575 | 156 | 618 | 1.64E−32 | 3.38E−31 | 0.20155039 | YES |
| TINF2 | ENSG00000092330 | 30 | 87 | 1.86E−06 | 1.78E−05 | 0.25641026 | YES |
| TIPRL | ENSG00000143155 | 471 | 671 | 4.30E−05 | 0.00038011 | 0.41243433 | YES |
| TKT | ENSG00000163931 | 195 | 391 | 8.83E−10 | 9.49E−09 | 0.33276451 | YES |
| TMA16 | ENSG00000198498 | 56 | 597 | 2.39E−54 | 9.89E−53 | 0.08575804 | YES |
| TMED10 | ENSG00000170348 | 507 | 934 | 5.22E−13 | 6.20E−12 | 0.351839 | YES |
| TMED2 | ENSG00000086598 | 215 | 463 | 2.21E−12 | 2.57E−11 | 0.31710914 | YES |
| TMEM167A | ENSG00000174695 | 275 | 460 | 4.88E−07 | 4.77E−06 | 0.37414966 | YES |
| TMEM199 | ENSG00000244045 | 97 | 256 | 8.57E−12 | 9.78E−11 | 0.27478754 | YES |
| TMEM238 | ENSG00000233493 | 122 | 239 | 5.22E−07 | 5.10E−06 | 0.33795014 | YES |
| TMEM258 | ENSG00000134825 | 22 | 335 | 8.11E−40 | 2.09E−38 | 0.06162465 | YES |
| TMEM41B | ENSG00000166471 | 168 | 520 | 1.10E−22 | 1.73E−21 | 0.24418605 | YES |
| TMEM48 | ENSG00000058804 | 662 | 914 | 3.99E−05 | 0.00035421 | 0.42005076 | YES |
| TMX2 | ENSG00000213593 | 306 | 1254 | 7.80E−51 | 2.84E−49 | 0.19615385 | YES |
| TNPO3 | ENSG00000064419 | 435 | 5408 | 4.17E−182 | 8.10E−179 | 0.07444806 | YES |
| TOMM20 | ENSG00000173726 | 91 | 653 | 1.43E−49 | 4.99E−48 | 0.12231183 | YES |
| TOMM22 | ENSG00000100216 | 13 | 196 | 1.11E−26 | 1.95E−25 | 0.06220096 | YES |
| TOMM40 | ENSG00000130204 | 61 | 367 | 7.99E−31 | 1.59E−29 | 0.14252336 | YES |
| TOMM70A | ENSG00000154174 | 131 | 309 | 2.61E−11 | 2.95E−10 | 0.29772727 | YES |
| TOP1 | ENSG00000198900 | 211 | 840 | 2.17E−39 | 5.52E−38 | 0.20076118 | YES |
| TOP2A | ENSG00000131747 | 34 | 352 | 1.99E−37 | 4.75E−36 | 0.0880829 | YES |
| TOP3A | ENSG00000177302 | 123 | 726 | 6.52E−48 | 2.16E−46 | 0.14487633 | YES |
| TOPBP1 | ENSG00000163781 | 344 | 847 | 4.26E−22 | 6.61E−21 | 0.28883291 | YES |
| TP53RK | ENSG00000172315 | 35 | 418 | 4.99E−44 | 1.46E−42 | 0.07726269 | YES |
| TPI1 | ENSG00000111669 | 99 | 178 | 5.28E−05 | 0.00046541 | 0.35740072 | YES |
| TPR | ENSG00000047410 | 35 | 332 | 6.61E−35 | 1.47E−33 | 0.09536785 | YES |
| TPT1 | ENSG00000133112 | 38 | 190 | 2.56E−17 | 3.45E−16 | 0.16666667 | YES |
| TPX2 | ENSG00000088325 | 69 | 1439 | 7.59E−109 | 2.01E−106 | 0.04575597 | YES |
| TRA2B | ENSG00000136527 | 75 | 808 | 2.65E−66 | 1.56E−64 | 0.08493771 | YES |
| TRAIP | ENSG00000183763 | 24 | 152 | 1.01E−16 | 1.33E−15 | 0.13636364 | YES |
| TRAPPC1 | ENSG00000170043 | 14 | 82 | 3.51E−10 | 3.84E−09 | 0.14583333 | YES |
| TRAPPC11 | ENSG00000168538 | 159 | 493 | 5.55E−22 | 8.56E−21 | 0.24386503 | YES |
| TRAPPC3 | ENSG00000054116 | 15 | 329 | 7.67E−42 | 2.13E−40 | 0.04360465 | YES |
| TRAPPC4 | ENSG00000196655 | 14 | 180 | 3.66E−24 | 5.96E−23 | 0.07216495 | YES |
| TRAPPC8 | ENSG00000153339 | 99 | 1097 | 1.28E−80 | 1.26E−78 | 0.08277592 | YES |
| TRIAP1 | ENSG00000170855 | 35 | 473 | 1.68E−49 | 5.83E−48 | 0.06889764 | YES |
| TRIT1 | ENSG00000043514 | 740 | 1257 | 4.53E−12 | 5.22E−11 | 0.37055583 | YES |
| TRMT10C | ENSG00000174173 | 90 | 319 | 4.69E−19 | 6.59E−18 | 0.2200489 | YES |
| TRMT5 | ENSG00000126814 | 52 | 501 | 9.12E−47 | 2.88E−45 | 0.09403255 | YES |
| TRMT6 | ENSG00000089195 | 66 | 693 | 1.22E−59 | 6.07E−58 | 0.08695652 | YES |
| TRMT61A | ENSG00000166166 | 126 | 257 | 6.82E−08 | 6.92E−07 | 0.32898172 | YES |
| TRNAU1AP | ENSG00000180098 | 269 | 604 | 1.36E−15 | 1.76E−14 | 0.30813288 | YES |
| TRNT1 | ENSG00000072756 | 90 | 395 | 1.94E−26 | 3.41E−25 | 0.18556701 | YES |
| TRPM7 | ENSG00000092439 | 280 | 765 | 2.15E−24 | 3.55E−23 | 0.26794258 | YES |
| TRRAP | ENSG00000196367 | 32 | 265 | 2.91E−28 | 5.37E−27 | 0.10774411 | YES |
| TRUB2 | ENSG00000167112 | 186 | 400 | 2.50E−11 | 2.83E−10 | 0.31740614 | YES |
| TSC22D2 | ENSG00000196428 | 216 | 507 | 3.45E−15 | 4.42E−14 | 0.29875519 | YES |
| TSEN15 | ENSG00000198860 | 471 | 3327 | 1.74E−119 | 5.72E−117 | 0.12401264 | YES |
| TSEN2 | ENSG00000154743 | 108 | 820 | 9.66E−59 | 4.59E−57 | 0.11637931 | YES |
| TSEN54 | ENSG00000182173 | 3 | 81 | 1.32E−14 | 1.66E−13 | 0.03571429 | YES |
| TSG101 | ENSG00000074319 | 111 | 685 | 2.34E−47 | 7.63E−46 | 0.13944724 | YES |
| TSR1 | ENSG00000167721 | 745 | 1273 | 2.38E−12 | 2.76E−11 | 0.3691774 | YES |
| TSR2 | ENSG00000158526 | 10 | 256 | 1.84E−35 | 4.15E−34 | 0.03759398 | YES |
| TTC1 | ENSG00000113312 | 81 | 827 | 3.87E−66 | 2.26E−64 | 0.08920705 | YES |
| TTC27 | ENSG00000018699 | 103 | 1119 | 4.47E−81 | 4.51E−79 | 0.08428805 | YES |
| TTC4 | ENSG00000243725 | 93 | 242 | 3.70E−11 | 4.16E−10 | 0.27761194 | YES |
| TTF1 | ENSG00000125482 | 157 | 400 | 6.28E−15 | 7.99E−14 | 0.28186715 | YES |
| TTF2 | ENSG00000116830 | 143 | 355 | 2.46E−13 | 2.95E−12 | 0.28714859 | YES |
| TTI1 | ENSG00000101407 | 140 | 1497 | 2.93E−95 | 4.78E−93 | 0.0855223 | YES |
| TTK | ENSG00000112742 | 20 | 208 | 1.71E−25 | 2.90E−24 | 0.0877193 | YES |
| TUBA1B | ENSG00000123416 | 87 | 637 | 3.09E−49 | 1.07E−47 | 0.12016575 | YES |
| TUBB | ENSG00000196230 | 365 | 781 | 1.84E−16 | 2.43E−15 | 0.31849913 | YES |
| TUBD1 | ENSG00000108423 | 210 | 581 | 4.34E−21 | 6.49E−20 | 0.26548673 | YES |
| TUBE1 | ENSG00000074935 | 13 | 157 | 1.91E−21 | 2.89E−20 | 0.07647059 | YES |
| TUBG1 | ENSG00000131462 | 12 | 165 | 5.93E−23 | 9.37E−22 | 0.06779661 | YES |
| TUBGCP3 | ENSG00000126216 | 41 | 603 | 1.62E−59 | 7.99E−58 | 0.0636646 | YES |
| TUBGCP4 | ENSG00000137822 | 110 | 428 | 2.92E−25 | 4.92E−24 | 0.20446097 | YES |
| TUBGCP5 | ENSG00000153575 | 48 | 314 | 7.62E−29 | 1.43E−27 | 0.13259669 | YES |
| TUBGCP6 | ENSG00000128159 | 30 | 359 | 1.48E−39 | 3.79E−38 | 0.07712082 | YES |
| TUFM | ENSG00000178952 | 64 | 183 | 1.96E−10 | 2.15E−09 | 0.25910931 | YES |
| TUT1 | ENSG00000149016 | 154 | 803 | 3.11E−47 | 1.01E−45 | 0.16091954 | YES |
| TWISTNB | ENSG00000105849 | 5 | 133 | 1.55E−21 | 2.37E−20 | 0.03623188 | YES |
| TXN | ENSG00000136810 | 23 | 186 | 1.45E−21 | 2.21E−20 | 0.11004785 | YES |
| TXNL4A | ENSG00000141759 | 44 | 482 | 1.97E−47 | 6.44E−46 | 0.08365019 | YES |
| TYMS | ENSG00000176890 | 48 | 625 | 3.17E−59 | 1.54E−57 | 0.07132244 | YES |
| U2AF1 | ENSG00000160201 | 20 | 409 | 1.97E−48 | 6.70E−47 | 0.04662005 | YES |
| U2AF2 | ENSG00000063244 | 206 | 2154 | 1.31E−114 | 3.83E−112 | 0.08728814 | YES |
| U2SURP | ENSG00000163714 | 183 | 1526 | 6.99E−88 | 9.11E−86 | 0.10708016 | YES |
| UBA1 | ENSG00000130985 | 28 | 381 | 1.32E−42 | 3.72E−41 | 0.06845966 | YES |
| UBA2 | ENSG00000126261 | 50 | 605 | 7.24E−57 | 3.27E−55 | 0.07633588 | YES |
| UBA3 | ENSG00000144744 | 210 | 965 | 3.61E−48 | 1.21E−46 | 0.1787234 | YES |
| UBA52 | ENSG00000221983 | 25 | 523 | 1.07E−57 | 4.95E−56 | 0.04562044 | YES |
| UBAP1 | ENSG00000165006 | 1747 | 2950 | 6.23E−17 | 8.33E−16 | 0.37193954 | YES |
| UBE2H | ENSG00000186591 | 10665 | 16799 | 1.01E−21 | 1.54E−20 | 0.38832654 | YES |
| UBE2I | ENSG00000103275 | 80 | 221 | 2.36E−11 | 2.66E−10 | 0.26578073 | YES |
| UBE2L3 | ENSG00000185651 | 1110 | 1538 | 2.31E−06 | 2.20E−05 | 0.41918429 | YES |
| UBE2M | ENSG00000130725 | 295 | 547 | 5.60E−10 | 6.06E−09 | 0.35035629 | YES |
| UBE2S | ENSG00000108106 | 11 | 115 | 2.89E−16 | 3.79E−15 | 0.08730159 | YES |
| UBE2T | ENSG00000077152 | 387 | 805 | 8.09E−16 | 1.05E−14 | 0.32466443 | YES |
| UBE3D | ENSG00000118420 | 558 | 1240 | 1.35E−22 | 2.11E−21 | 0.31034483 | YES |
| UBE4B | ENSG00000130939 | 2349 | 3009 | 2.15E−05 | 0.00019377 | 0.43840985 | YES |
| UBL5 | ENSG00000198258 | 75 | 209 | 4.65E−11 | 5.21E−10 | 0.26408451 | YES |
| UBR4 | ENSG00000127481 | 295 | 562 | 8.98E−11 | 9.94E−10 | 0.34422404 | YES |
| UBTF | ENSG00000108312 | 255 | 1447 | 4.29E−70 | 2.87E−68 | 0.14982374 | YES |
| UCHL5 | ENSG00000116750 | 819 | 1286 | 1.54E−09 | 1.64E−08 | 0.38907363 | YES |
| UFD1L | ENSG00000070010 | 82 | 820 | 2.45E−65 | 1.39E−63 | 0.09090909 | YES |
| UMPS | ENSG00000114491 | 192 | 527 | 1.16E−19 | 1.66E−18 | 0.26703755 | YES |
| UPF1 | ENSG00000005007 | 41 | 323 | 5.72E−32 | 1.16E−30 | 0.11263736 | YES |
| UPF2 | ENSG00000151461 | 405 | 1987 | 7.15E−75 | 5.78E−73 | 0.16931438 | YES |
| UQCC | ENSG00000101019 | 927 | 1591 | 7.89E−14 | 9.61E−13 | 0.36814932 | YES |
| UQCRC1 | ENSG00000010256 | 360 | 728 | 3.95E−14 | 4.87E−13 | 0.33088235 | YES |
| UQCRC2 | ENSG00000140740 | 327 | 839 | 1.89E−23 | 3.02E−22 | 0.28044597 | YES |
| UQCRFS1 | ENSG00000169021 | 107 | 297 | 7.17E−14 | 8.75E−13 | 0.26485149 | YES |
| UQCRQ | ENSG00000164405 | 42 | 130 | 5.07E−09 | 5.32E−08 | 0.24418605 | YES |
| URB1 | ENSG00000142207 | 59 | 494 | 5.06E−44 | 1.48E−42 | 0.10669078 | YES |
| URB2 | ENSG00000135763 | 140 | 397 | 3.45E−17 | 4.63E−16 | 0.26070764 | YES |
| URI1 | ENSG00000105176 | 1147 | 5536 | 5.37E−115 | 1.59E−112 | 0.17162951 | YES |
| URM1 | ENSG00000167118 | 60 | 899 | 2.67E−77 | 2.36E−75 | 0.06256517 | YES |
| UROD | ENSG00000126088 | 98 | 348 | 3.08E−20 | 4.46E−19 | 0.21973094 | YES |
| USE1 | ENSG00000053501 | 72 | 269 | 8.22E−18 | 1.12E−16 | 0.2111437 | YES |
| USP10 | ENSG00000103194 | 1519 | 1954 | 7.58E−05 | 0.00066118 | 0.43737403 | YES |
| USP36 | ENSG00000055483 | 291 | 599 | 3.69E−13 | 4.42E−12 | 0.32696629 | YES |
| USP37 | ENSG00000135913 | 60 | 649 | 1.12E−57 | 5.17E−56 | 0.08462623 | YES |
| USP39 | ENSG00000168883 | 33 | 406 | 1.78E−43 | 5.10E−42 | 0.07517084 | YES |
| USP5 | ENSG00000111667 | 8 | 247 | 3.13E−35 | 7.03E−34 | 0.03137255 | YES |
| USP9X | ENSG00000124486 | 731 | 3135 | 4.73E−83 | 5.26E−81 | 0.18908432 | YES |
| USPL1 | ENSG00000132952 | 187 | 948 | 3.31E−51 | 1.23E−49 | 0.16475771 | YES |
| UTP11L | ENSG00000183520 | 25 | 380 | 1.42E−43 | 4.09E−42 | 0.0617284 | YES |
| UTP14A | ENSG00000156697 | 83 | 591 | 2.72E−46 | 8.48E−45 | 0.1231454 | YES |
| UTP15 | ENSG00000164338 | 27 | 320 | 2.52E−36 | 5.85E−35 | 0.0778098 | YES |
| UTP18 | ENSG00000011260 | 50 | 586 | 3.45E−55 | 1.47E−53 | 0.07861635 | YES |
| UTP20 | ENSG00000120800 | 57 | 379 | 3.50E−33 | 7.36E−32 | 0.13073394 | YES |
| UTP6 | ENSG00000108651 | 71 | 702 | 5.64E−59 | 2.70E−57 | 0.09184994 | YES |
| UVRAG | ENSG00000198382 | 5391 | 7125 | 4.84E−08 | 4.93E−07 | 0.43072867 | YES |
| UXT | ENSG00000126756 | 34 | 373 | 1.11E−39 | 2.84E−38 | 0.08353808 | YES |
| VARS | ENSG00000204394 | 383 | 608 | 5.56E−07 | 5.43E−06 | 0.3864783 | YES |
| VBP1 | ENSG00000155959 | 260 | 475 | 6.34E−09 | 6.62E−08 | 0.3537415 | YES |
| VCP | ENSG00000165280 | 83 | 349 | 1.64E−23 | 2.63E−22 | 0.19212963 | YES |
| VEZT | ENSG00000028203 | 798 | 1636 | 6.28E−22 | 9.69E−21 | 0.32785538 | YES |
| VMP1 | ENSG00000062716 | 3125 | 13385 | 7.51E−137 | 3.64E−134 | 0.18927922 | YES |
| VPS13D | ENSG00000048707 | 953 | 9063 | 1.83E−199 | 4.58E−196 | 0.09514776 | YES |
| VPS25 | ENSG00000131475 | 70 | 290 | 1.83E−20 | 2.67E−19 | 0.19444444 | YES |
| VPS29 | ENSG00000111237 | 740 | 1871 | 1.02E−34 | 2.25E−33 | 0.28341632 | YES |
| VPS33A | ENSG00000139719 | 730 | 1036 | 5.07E−06 | 4.73E−05 | 0.41336353 | YES |
| VPS35 | ENSG00000069329 | 125 | 315 | 1.22E−12 | 1.43E−11 | 0.28409091 | YES |
| VPS36 | ENSG00000136100 | 93 | 174 | 2.40E−05 | 0.00021606 | 0.34831461 | YES |
| VPS51 | ENSG00000149823 | 151 | 254 | 3.14E−05 | 0.0002805 | 0.37283951 | YES |
| VPS52 | ENSG00000223501 | 664 | 861 | 0.00079606 | 0.00649986 | 0.43540984 | YES |
| VPS53 | ENSG00000141252 | 456 | 1209 | 4.48E−30 | 8.67E−29 | 0.27387387 | YES |
| VPS72 | ENSG00000163159 | 58 | 702 | 1.08E−62 | 5.67E−61 | 0.07631579 | YES |
| WAC | ENSG00000095787 | 845 | 1649 | 1.12E−19 | 1.60E−18 | 0.33881315 | YES |
| WAPAL | ENSG00000062650 | 191 | 530 | 5.08E−20 | 7.29E−19 | 0.26490985 | YES |
| WARS | ENSG00000140105 | 125 | 605 | 1.34E−37 | 3.24E−36 | 0.17123288 | YES |
| WARS2 | ENSG00000116874 | 201 | 388 | 5.24E−09 | 5.50E−08 | 0.34125637 | YES |
| WBP11 | ENSG00000084463 | 22 | 338 | 3.79E−40 | 9.98E−39 | 0.06111111 | YES |
| WBP4 | ENSG00000120688 | 44 | 300 | 1.76E−28 | 3.26E−27 | 0.12790698 | YES |
| WBSCR22 | ENSG00000071462 | 56 | 432 | 7.37E−39 | 1.86E−37 | 0.1147541 | YES |
| WDHD1 | ENSG00000198554 | 443 | 2896 | 9.42E−108 | 2.38E−105 | 0.13267445 | YES |
| WDR12 | ENSG00000138442 | 282 | 912 | 1.90E−33 | 4.03E−32 | 0.2361809 | YES |
| WDR18 | ENSG00000065268 | 69 | 557 | 5.04E−47 | 1.62E−45 | 0.11022364 | YES |
| WDR20 | ENSG00000140153 | 7931 | 9681 | 3.03E−05 | 0.0002703 | 0.45031797 | YES |
| WDR25 | ENSG00000176473 | 119 | 507 | 1.81E−30 | 3.55E−29 | 0.19009585 | YES |
| WDR3 | ENSG00000065183 | 38 | 444 | 1.19E−45 | 3.62E−44 | 0.07883817 | YES |
| WDR33 | ENSG00000136709 | 116 | 1214 | 5.03E−84 | 5.78E−82 | 0.08721805 | YES |
| WDR36 | ENSG00000134987 | 12 | 254 | 2.21E−34 | 4.86E−33 | 0.04511278 | YES |
| WDR4 | ENSG00000160193 | 462 | 1186 | 2.45E−28 | 4.52E−27 | 0.28033981 | YES |
| WDR43 | ENSG00000163811 | 58 | 888 | 4.49E−77 | 3.92E−75 | 0.06131078 | YES |
| WDR44 | ENSG00000131725 | 2860 | 3671 | 9.12E−06 | 8.36E−05 | 0.4379115 | YES |
| WDR46 | ENSG00000227057 | 38 | 87 | 6.39E−05 | 0.00055987 | 0.304 | YES |
| WDR5 | ENSG00000196363 | 46 | 259 | 3.10E−23 | 4.93E−22 | 0.15081967 | YES |
| WDR55 | ENSG00000120314 | 22 | 182 | 2.14E−21 | 3.23E−20 | 0.10784314 | YES |
| WDR59 | ENSG00000103091 | 274 | 697 | 8.09E−21 | 1.20E−19 | 0.28218332 | YES |
| WDR70 | ENSG00000082068 | 134 | 1245 | 8.19E−82 | 8.62E−80 | 0.09717186 | YES |
| WDR74 | ENSG00000133316 | 53 | 368 | 3.47E−33 | 7.32E−32 | 0.12589074 | YES |
| WDR75 | ENSG00000115368 | 15 | 334 | 2.15E−42 | 6.01E−41 | 0.04297994 | YES |
| WDR77 | ENSG00000116455 | 10 | 113 | 2.21E−16 | 2.91E−15 | 0.08130081 | YES |
| WDR82 | ENSG00000164091 | 207 | 1073 | 6.80E−56 | 2.98E−54 | 0.16171875 | YES |
| WDR92 | ENSG00000243667 | 47 | 428 | 3.63E−41 | 9.93E−40 | 0.09894737 | YES |
| WEE1 | ENSG00000166483 | 31 | 542 | 2.32E−57 | 1.06E−55 | 0.05410122 | YES |
| WIBG | ENSG00000170473 | 1365 | 1851 | 3.57E−06 | 3.35E−05 | 0.4244403 | YES |
| WNK1 | ENSG00000060237 | 1158 | 2205 | 4.51E−21 | 6.75E−20 | 0.34433541 | YES |
| WRB | ENSG00000182093 | 503 | 1385 | 4.02E−34 | 8.69E−33 | 0.26641949 | YES |
| XAB2 | ENSG00000076924 | 5 | 68 | 1.54E−11 | 1.74E−10 | 0.06849315 | YES |
| XPO1 | ENSG00000082898 | 415 | 1829 | 5.08E−66 | 2.94E−64 | 0.18493761 | YES |
| XPO5 | ENSG00000124571 | 176 | 565 | 6.55E−25 | 1.09E−23 | 0.23751687 | YES |
| XRCC1 | ENSG00000073050 | 153 | 581 | 4.25E−30 | 8.24E−29 | 0.20844687 | YES |
| XRCC2 | ENSG00000196584 | 103 | 557 | 2.26E−38 | 5.56E−37 | 0.15606061 | YES |
| XRCC3 | ENSG00000126215 | 158 | 530 | 4.12E−25 | 6.90E−24 | 0.22965116 | YES |
| XRCC6 | ENSG00000196419 | 130 | 594 | 1.25E−35 | 2.84E−34 | 0.17955801 | YES |
| XRN1 | ENSG00000114127 | 139 | 815 | 2.81E−51 | 1.05E−49 | 0.14570231 | YES |
| XRN2 | ENSG00000088930 | 402 | 1264 | 9.23E−39 | 2.31E−37 | 0.24129652 | YES |
| YAE1D1 | ENSG00000241127 | 12 | 244 | 3.64E−33 | 7.65E−32 | 0.046875 | YES |
| YARS | ENSG00000134684 | 168 | 1470 | 8.54E−88 | 1.10E−85 | 0.1025641 | YES |
| YARS2 | ENSG00000139131 | 168 | 364 | 7.50E−11 | 8.32E−10 | 0.31578947 | YES |
| YBX1 | ENSG00000065978 | 165 | 346 | 6.40E−10 | 6.91E−09 | 0.32289628 | YES |
| YEATS2 | ENSG00000163872 | 881 | 1187 | 4.16E−05 | 0.0003687 | 0.42601547 | YES |
| YEATS4 | ENSG00000127337 | 166 | 589 | 1.96E−28 | 3.63E−27 | 0.21986755 | YES |
| YKT6 | ENSG00000106636 | 6 | 160 | 7.05E−25 | 1.18E−23 | 0.03614458 | YES |
| YLPM1 | ENSG00000119596 | 187 | 392 | 1.12E−10 | 1.24E−09 | 0.32297064 | YES |
| YME1L1 | ENSG00000136758 | 267 | 676 | 2.64E−20 | 3.83E−19 | 0.28313892 | YES |
| YPEL5 | ENSG00000119801 | 73 | 189 | 1.96E−09 | 2.08E−08 | 0.27862595 | YES |
| YRDC | ENSG00000196449 | 12 | 166 | 4.33E−23 | 6.86E−22 | 0.06741573 | YES |
| YTHDC1 | ENSG00000083896 | 49 | 803 | 2.22E−73 | 1.68E−71 | 0.05751174 | YES |
| YY1 | ENSG00000100811 | 45 | 538 | 2.13E−52 | 8.36E−51 | 0.07718696 | YES |
| ZBTB11 | ENSG00000066422 | 137 | 351 | 7.30E−14 | 8.91E−13 | 0.2807377 | YES |
| ZBTB17 | ENSG00000116809 | 55 | 386 | 1.77E−34 | 3.89E−33 | 0.12471655 | YES |
| ZBTB2 | ENSG00000181472 | 489 | 663 | 0.00034172 | 0.00286793 | 0.42447917 | YES |
| ZBTB8OS | ENSG00000176261 | 472 | 4906 | 1.38E−165 | 1.73E−162 | 0.08776497 | YES |
| ZC3H11A | ENSG00000058673 | 673 | 1182 | 6.91E−13 | 8.17E−12 | 0.36280323 | YES |
| ZC3H13 | ENSG00000123200 | 242 | 876 | 6.89E−37 | 1.62E−35 | 0.21645796 | YES |
| ZC3H18 | ENSG00000158545 | 41 | 466 | 7.69E−47 | 2.45E−45 | 0.08086785 | YES |
| ZC3H4 | ENSG00000130749 | 3147 | 3844 | 0.0002465 | 0.00209193 | 0.45015019 | YES |
| ZC3H8 | ENSG00000144161 | 58 | 644 | 7.75E−58 | 3.59E−56 | 0.08262108 | YES |
| ZCCHC9 | ENSG00000131732 | 17 | 363 | 9.24E−45 | 2.74E−43 | 0.04473684 | YES |
| ZCRB1 | ENSG00000139168 | 278 | 743 | 3.02E−23 | 4.80E−22 | 0.27228208 | YES |
| ZFC3H1 | ENSG00000133858 | 108 | 202 | 8.22E−06 | 7.56E−05 | 0.3483871 | YES |
| ZFR | ENSG00000056097 | 581 | 1086 | 1.55E−14 | 1.94E−13 | 0.34853029 | YES |
| ZMAT2 | ENSG00000146007 | 10 | 145 | 5.14E−21 | 7.65E−20 | 0.06451613 | YES |
| ZMAT5 | ENSG00000100319 | 75 | 393 | 8.36E−30 | 1.61E−28 | 0.16025641 | YES |
| ZMYND8 | ENSG00000101040 | 5658 | 8047 | 5.26E−12 | 6.05E−11 | 0.41284203 | YES |
| ZNF131 | ENSG00000172262 | 149 | 672 | 3.87E−38 | 9.41E−37 | 0.18148599 | YES |
| ZNF143 | ENSG00000166478 | 3240 | 5265 | 3.47E−18 | 4.79E−17 | 0.38095238 | YES |
| ZNF148 | ENSG00000163848 | 1650 | 2443 | 5.99E−10 | 6.49E−09 | 0.40312729 | YES |
| ZNF207 | ENSG00000010244 | 75 | 765 | 5.98E−63 | 3.15E−61 | 0.08928571 | YES |
| ZNF236 | ENSG00000130856 | 519 | 1387 | 1.10E−32 | 2.30E−31 | 0.27229801 | YES |
| ZNF259 | ENSG00000109917 | 8 | 141 | 2.58E−21 | 3.88E−20 | 0.05369128 | YES |
| ZNF335 | ENSG00000198026 | 203 | 1053 | 2.58E−55 | 1.10E−53 | 0.1616242 | YES |
| ZNF407 | ENSG00000215421 | 1001 | 2909 | 3.22E−52 | 1.24E−50 | 0.25601023 | YES |
| ZNF574 | ENSG00000105732 | 22 | 161 | 1.31E−18 | 1.82E−17 | 0.12021858 | YES |
| ZNF622 | ENSG00000173545 | 17 | 108 | 5.63E−13 | 6.68E−12 | 0.136 | YES |
| ZNHIT1 | ENSG00000106400 | 124 | 244 | 3.73E−07 | 3.68E−06 | 0.33695652 | YES |
| ZNHIT3 | ENSG00000108278 | 25 | 124 | 1.27E−12 | 1.49E−11 | 0.16778523 | YES |
| ZNHIT6 | ENSG00000117174 | 45 | 332 | 1.02E−31 | 2.07E−30 | 0.1193634 | YES |
| ZRANB2 | ENSG00000132485 | 255 | 378 | 0.00016427 | 0.00140978 | 0.4028436 | YES |
| ZRSR2 | ENSG00000169249 | 244 | 732 | 6.72E−27 | 1.19E−25 | 0.25 | YES |
| ZW10 | ENSG00000086827 | 23 | 146 | 3.01E−16 | 3.94E−15 | 0.13609467 | YES |
| ZZZ3 | ENSG00000036549 | 472 | 1391 | 1.49E−37 | 3.57E−36 | 0.2533548 | YES |

While the disclosure has been particularly shown and described with reference to specific embodiments (some of which are preferred embodiments), it should be understood by those having skill in the art that various changes in form and detail may be made therein without departing from the spirit and scope of the present disclosure as disclosed herein.

1. A method for treating cancer in an individual in need thereof, the method comprising:
i) obtaining one or more biological samples from the individual;
ii) determining different nucleotide sequences in cancer and non-cancer cells from the biological sample to identify a candidate target sequence that is present in the cancer cells and not present in the non-cancer cells, further characterizing the candidate target sequence to determine it is suitable for use as a target sequence, wherein the further characterizing the candidate target sequence comprises determining in the cancer cells but not the non-cancer cells that the candidate target sequence comprises:
a) a homozygous mutation in the cancer cells that is a segment of a chromosome that is within 3 Kb-10 kb of an exon of an essential gene, wherein optionally the exon is not an alternatively spliced exon;
b) identifying a protospacer adjacent motif (PAM) in the segment of a); and
c) identifying a sequence in the chromosome that is preferably separated from the PAM by 13 to 17 nucleotides, and more preferably separated from the PAM by 1 to 11 nucleotides, wherein the identified sequence is approximately 32 nucleotides in length, to thereby identify a suitable target sequence; and
iii) based on the different nucleotide sequences in the cancer cells relative to the non-cancer cells, introducing into cells of the individual a CRISPR Cas3 system comprising a guide RNA targeted to a segment of the chromosome that is linked to the target sequence such that the chromosome comprising the target sequence is degraded, thereby treating the cancer.

2. The method of claim 1, wherein the further characterizing the candidate target sequence also comprises determining that the different nucleotide sequences in the cancer cells comprises a mutation in the cancer cells in the candidate target sequence, said mutation comprising at least one of: a breakpoint sequence in a chromosomal translocation sequence; a mutation that comprises an insertion or a deletion; a di-nucleotide mutation; or a single nucleotide mutation to thereby determine the candidate target sequence is a suitable target sequence.

3. The method of claim 1, further comprising repeating introducing into cells of the individual the CRISPR Cas3 system comprising the guide RNA such that the cancer cells are killed.

4. The method of claim 2, further comprising repeating introducing into cells of the individual the CRISPR Cas3 system comprising the guide RNA such that the cancer cells are killed.

5. The method of claim 1, wherein identifying the candidate target sequence and/or further characterizing the candidate target sequence is performed using a computer comprising a digital processor running software that compares DNA sequences from the cancer cells to DNA sequences from the non-cancer cells.

6. The method of claim 2, wherein identifying the candidate target sequence and/or further characterizing the candidate target sequence is performed using a computer comprising a digital processor running software that compares DNA sequences from the cancer cells to DNA sequences from the non-cancer cells.

7. The method of claim 3, wherein identifying the candidate target sequence and/or further characterizing the candidate target sequence is performed using a computer comprising a digital processor running software that compares DNA sequences from the cancer cells to DNA sequences from the non-cancer cells.

8. The method of claim 4, wherein identifying the candidate target sequence and/or further characterizing the candidate target sequence is performed using a computer comprising a digital processor running software that compares DNA sequences from the cancer cells to DNA sequences from the non-cancer cells.

9. The method of claim 1, wherein the CRISPR Cas system comprises the guide RNA targeted to the target sequence, and a combination of proteins or one or more polynucleotides that express the combination of proteins after introduction into the cell, wherein the combination of proteins comprises Cas3, Cse1/CasA, Cse2/CasB, Cas7/CasC, Cas5e/CasD and Case/CasE (Cascade).

10. A method for identifying a target sequence for use with a CRISPR Cas3 system comprising determining different nucleotide sequences in cancer and non-cancer cells from a biological sample to identify a candidate target sequence that is present in the cancer cells and not present in the non-cancer cells, and further characterizing the candidate target sequence to determine it is suitable for use as a target sequence, as in claim 1.

11. A system configured to perform the method of claim 10, the system comprising a DNA sequencer and a computer comprising a digital processor running software that compares DNA sequences from cancer cells to DNA sequences from non-cancer cells to identify candidate target sequences.


SEQUENCE LISTING (19 sequences)

SEQ ID NO: 1 - RNA, 61 nt, synthetic construct
  source 1..61: mol_type = other RNA
atggaccgccagtgataagtggaatgccatgtgggctgtcgtgagccccacgcacgtgggg

SEQ ID NO: 2 - DNA, 55 bp, synthetic construct
  source 1..55: mol_type = other DNA
gcgcgcctgcttcggtcactattcaccttacggtacacccgacagcggacctgcg

SEQ ID NO: 3 - DNA, 55 bp, synthetic construct
  source 1..55: mol_type = other DNA
cgcaggtccgctgtcgggtgtaccgtaaggtgaatagtgaccgaagcaggcgcgc

SEQ ID NO: 4 - DNA, 32 bp, synthetic construct
  source 1..32: mol_type = other DNA
ccccccaaatgggtgctgagaccgagcgtggg

SEQ ID NO: 5 - DNA, 32 bp, synthetic construct
  source 1..32: mol_type = other DNA
ccccccaaatgggtgctgagaccgagcgtcgg

SEQ ID NO: 6 - DNA, 32 bp, synthetic construct
  source 1..32: mol_type = other DNA
tccagactcgtggatccgcctccgccgcgtcc

SEQ ID NO: 7 - DNA, 32 bp, synthetic construct
  source 1..32: mol_type = other DNA
gtgctacagtagagaaaagtcaaatgtttcct

SEQ ID NO: 8 - DNA, 32 bp, synthetic construct
  source 1..32: mol_type = other DNA
tttcgatgatatggtagactctaggtttaaaa

SEQ ID NO: 9 - DNA, 32 bp, synthetic construct
  source 1..32: mol_type = other DNA
aagaatctatggaggaagtgactctggaaaag

SEQ ID NO: 10 - DNA, 43 bp, synthetic construct
  source 1..43: mol_type = other DNA
cgattaaaaatttgtaaaaatatctctgccccctccgaccgaa

SEQ ID NO: 11 - DNA, 43 bp, synthetic construct
  source 1..43: mol_type = other DNA
ttctgaaatcacctgtgactgtctgttaccacacaggttttct

SEQ ID NO: 12 - DNA, 43 bp, synthetic construct
  source 1..43: mol_type = other DNA
ttcccttcctttcctgtcttactcctggtacccctgctctacc

SEQ ID NO: 13 - DNA, 43 bp, synthetic construct
  source 1..43: mol_type = other DNA
ttgttagtaatactccaaagggaggacttttaaccccggtgaa

SEQ ID NO: 14 - DNA, 43 bp, synthetic construct
  source 1..43: mol_type = other DNA
catggggacaaaatgtttcatctcgtgactctaagcaactgaa

SEQ ID NO: 15 - DNA, 32 bp, synthetic construct
  source 1..32: mol_type = other DNA
aatttgtaaaaatatctctgccccctccgacc

SEQ ID NO: 16 - DNA, 32 bp, synthetic construct
  source 1..32: mol_type = other DNA
tgtgtggtaacagacagtcacaggtgatttca

SEQ ID NO: 17 - DNA, 32 bp, synthetic construct
  source 1..32: mol_type = other DNA
aggggtaccaggagtaagacaggaaaggaagg

SEQ ID NO: 18 - DNA, 32 bp, synthetic construct
  source 1..32: mol_type = other DNA
aatactccaaagggaggacttttaaccccggt

SEQ ID NO: 19 - DNA, 32 bp, synthetic construct
  source 1..32: mol_type = other DNA
caaaatgtttcatctcgtgactctaagcaact